(19) United States
(12) Patent Application Publication
(10) Pub. No.: US 2010/0015126 A1
(43) Pub. Date: Jan. 21, 2010

(54) Methods of Binding of Cross-Beta Structures By Chaperones

(76) Inventors: Martijn Frans Ben Gerard Gebbink, Eemnes (NL); Barend Bouma, Houten (NL)

Correspondence Address: TRASKBRITT, P.C., P.O. BOX 2550, SALT LAKE CITY, UT, 84110, US

(21) Appl. No.: 12/225,291

(22) PCT Filed: Mar. 16, 2007
(86) PCT No.: PCT/IN2007/000078

§ 371 Date: Jan. 7, 2009

(30) Foreign Application Priority Data
Mar. 17, 2006 (EP) 06075630.1

(51) Int. Cl. A61K 39/395; C07K 1/10; A61K 38/16; A01N 1/02; C07K 1/14; G01N 33/00; G01N 33/53; A61K 39/00; A61P 19/02; A61P 31/00

(52) U.S. Cl. 424/130.1; 530/402; 514/12; 435/2; 530/415; 436/86; 435/7.1; 424/184.1

(57) ABSTRACT

The invention relates to the field of biochemistry, biophysical chemistry, molecular biology, structural biology, immunology, cellular biology and medicine. More in particular, the invention relates to the capability (or property) of chaperones to bind a crossbeta structure. Even more in particular, the invention relates to extracellular chaperones such as BiP, haptoglobin, hsp72 or clusterin. The present invention provides the insight that a chaperone molecule and more in specific an extra-cellular chaperone molecule (such as for example BiP, clusterin, hsp72 or haptoglobin) is capable of interacting with a crossbeta structure and/or a molecule comprising a crossbeta structure and/or a molecule comprising a crossbeta structure precursor. Based on this insight, the present inventors have developed multiple methods and means.

The invention relates to the field of biochemistry, biophysical chemistry, molecular biology, structural biology, immunology, cellular biology and medicine. More in particular, the invention relates to the capability (or property) of chaperones to bind a crossbeta structure. Even more in particular, the invention relates to extra-cellular chaperones such as BiP, haptoglobin, hsp72 or clusterin.

Cells have post-translational quality control systems meant to repair or remove obsolete and/or damaged proteins. Therefore, as a consequence, misfolded and obsolete intracellular proteins can go three ways, i.e. proteins with a non-native fold can be assisted by chaperones to fold back to a native conformation, or chaperones can direct the misfolded proteins to proteolytic breakdown pathways, or misfolded proteins can aggregate.

Molecular chaperones are a diverse class of proteins comprising heat shock proteins, chaperoning, chaperokines and stress proteins, that are contributing to one of the most important cell defence mechanisms that facilitates protein folding, refolding of partially denatured proteins, protein transport across membranes, cytoskeletal organization, degradation of disabled proteins, and apoptosis, but also act as cytoprotective factors against deleterious environmental stresses. Individual members of the family of these specialized proteins bind non-native states of one or several or whole series or classes of proteins and assist them in reaching a correctly folded and functional conformation. Alternatively, when the native fold cannot be achieved, molecular chaperones contribute to the effective removal of misfolded proteins by directing them to the suitable proteolytic degradation pathways. Chaperones selectively bind to non-natively folded proteins in a stable non-covalent manner. To direct correct folding of a protein from a misfolded form to the required native conformation, mostly several chaperones work together in consecutive steps.

Chaperonins are molecular machines that facilitate protein folding by undergoing energy (ATP)-dependent movements that are coordinated in time and space by complex allosteric regulation. Examples of chaperones that facilitate refolding of proteins from a misfolded conformation to a native form are heat shock protein (hsp) 90, hsp60 and hsp70. Chaperones also participate in the stabilization of unstable protein conformers and in the recovery of proteins from aggregates. Molecular chaperones are mostly heat- or stress-induced proteins (hsp's), that perform critical functions in maintaining cell homeostasis, or are transiently present and active in regular protein synthesis. Hsp's are among the most abundant intracellular proteins. Chaperones that act in an ATP-independent manner are for example the intracellular small hsp's, calreticulin, calnexin and extracellular clusterin. Under stress conditions such as elevated temperature, glucose deprivation and oxidation, small hsp's and clusterin efficiently prevent the aggregation of target proteins. Interestingly, both types of hsp's can hardly chaperone a misfolded protein to refold back to its native state. In patients with Creutzfeldt-Jakob, Alzheimer's disease and other diseases related to protein misfolding and accumulation of amyloid, increased expression of clusterin and small hsp's has been seen. Molecular chaperones are essential components of the quality control machineries present in cells. Due to the fact that they aid in the folding and maintenance of newly translated proteins, as well as in facilitating the degradation of misfolded and destabilized proteins, chaperones are essentially the cellular sensors of protein misfolding and function. Chaperones are therefore the gatekeepers in a first line of defence against deleterious effects of misfolded proteins, by assisting a protein in obtaining its native fold or by directing incorrectly folded proteins to a proteolytic breakdown pathway. Notably, hsp's are over-expressed in many human cancers. It has been established that hsp's play a role in tumor cell metastasis, proliferation, differentiation, invasion, death, and in triggering the immune system during cancer.

One of the key members of the quality control machinery of the cell is the ubiquitous molecular chaperone hsp90. Hsp90 typically functions as part of large complexes, which include other chaperones and essential cofactors that regulate its function. Different cofactors seem to target hsp90 to different sets of substrates. However, the mechanism of hsp90 function in protein misfolding biology remains poorly understood.

Intracellular pathways that are involved in sensing protein misfolding comprise the unfolded protein response machinery (UPR) in the endoplasmic reticulum (ER). Accumulation of unfolded and/or misfolded proteins in the ER induces ER stress resulting in triggering of the UPR. Environmental factors can transduce the stress response, like for example changes in pH, starvation, reactive oxygen species. During these episodes of cellular stress, intracellular heat shock proteins levels increase to provide cellular protection. Activation of the UPR includes the attenuation of general protein synthesis and the transcriptional activation of the genes encoding ER-resident chaperones and molecules involved in the ER-associated degradation (ERAD) pathway. The UPR reduces ER stress by restoration of the protein-folding capacity of the ER. A key protein acting as a sensor of protein misfolding is the chaperone BiP (also referred to as grp78; Immunoglobulin heavy chain-binding protein/Endoplasmic reticulum luminal Ca²⁺-binding protein).

In addition to functioning as intracellular molecular chaperones, heat shock proteins also function as initiators of the host's immune response. Mechanisms by which intracellular heat shock proteins leave cells are still incompletely understood, but may involve the shedding of vesicles containing cytoplasmic constituents (exosomes). Apparently, heat shock proteins are released by both passive (necrotic) and active (physiological) mechanisms. It is clear that binding of hsp to specific surface receptors is a prerequisite for the initiation of an immune response. In one postulated model, extra-cellular chaperones like for example clusterin and haptoglobin bind to exposed hydrophobic regions on non-native extra-cellular proteins to target them for receptor-mediated endocytosis and intracellular, lysosomal degradation. The intracellular quality control systems have been characterized quite extensively and in detail. In contrast, little is known about how the folding of extra-cellular proteins is monitored. We now propose that at least in the extra-cellular space it is not the exposure of hydrophobic patches at the surface of incorrectly folded proteins that triggers chaperone binding, though that it is the crossbeta structure, that is attracting crossbeta structure binding molecules to activate our postulated “Crossbeta Pathway” for removal of unwanted and obsolete proteins and cells, which do not have a correctly folded native state. One class of crossbeta structure binding molecules that are most likely involved in the Pathway, are the chaperones, and even more precisely, the heat shock proteins or stress proteins. At forehand it is more likely that the extra-cellularly more abundant chaperones play a more prominent role in monitoring protein misfolding as part of the Pathway than those chaperones that are known for their intracellular activities and that are only present in the extra-cellular milieu at relatively low concentration, like for example hsp70. Abundant extra-cellular chaperones are haptoglobin and clusterin. The fact that these two chaperones assist in prevention of formation of aggregates of misfolded proteins in an ATP independent manner make them even further likely candidates to play an important role in the Pathway. Extra-cellular ATP concentrations are low, which is not favourable for the ATP-dependent extra-cellular chaperones, which are in addition only present at relatively low concentrations. It is clear that amyloid-like crossbeta structure has a cytotoxic nature and can induce the pathology seen with deleterious protein misfolding diseases such as Alzheimer's disease, diabetes type II, sepsis, chronic inflammation, autoimmune diseases, encephalopaties, Huntington's disease and tauopathies. Therefore, a precisely fine-tuned mechanism is proposed to exist to monitor extra-cellular protein stability. At the very first moment of protein unfolding/misfolding, the members of the Pathway sense the occurrence of the crossbeta structure protein conformation which induces targeting of crossbeta structure comprising molecules for receptor-mediated endocytosis and intracellular, lysosomal degradation. For this purpose a series of proteins that sample protein conformation are proposed to act in concert. Amongst these proteins that act in the Crossbeta Pathway are chaperones, like for example clusterin, haptoglobin, gp96, BiP, other extra-cellularly located heat-shock proteins, proteases, like for example hepatocyte growth factor activator, plasminogen, tissue-type plasminogen activator, factor XII, antibodies like for example of the immunoglobulin G type and immunoglobulin M type, and cell surface receptors like for example low density lipoprotein receptor related protein/CD91, CD36, scavenger receptor A, scavenger receptor B-I, receptor for advanced glycation end-products. An important quality of the proteins that serve in the Pathway is their binding to a broad range of misfolded protein ligands. Currently, little to no evidence is available which shows that the extra-cellular Crossbeta Pathway can actively assist in refolding of misfolded proteins to a native conformation. It seems that in contrast to the intra-cellular Crossbeta Pathway, the extra-cellular Crossbeta Pathway is only capable of preventing misfolded proteins to form toxic structures like for example amyloid crossbeta structure oligomers and fibrils, and is capable of directing misfolded proteins to multiligand crossbeta structure receptors, facilitating endocytosis and subsequent proteolytic breakdown.

For hsp70 evidence is accumulating that the hsp not only enters extra-cellular space by passive release from necrotic cells but also by a process involving its active release in response to stresses including altered levels of cytokine concentrations, acute psychological stress and exercise. A clear extra-cellular function for hsp70 has become evident from the fact that cell surface receptors that have been identified as the receptor for hsp70 include scavenger receptor A (SR-A), Toll-like receptors 2 and 4 (TLR2, TLR4, receptor for Gram-positive bacteria and receptor for Gram-negative bacteria, respectively) with their cofactor CD14, the scavenger receptor CD36, the low-density lipoprotein receptor-related protein CD91 or LRP, the C-type lectin receptor LOX-1, and the co-stimulatory molecule, CD40. Binding of Hsp70 to these surface receptors specifically activates intracellular signalling cascades, which in turn exert immune-regulatory effector functions; a process known as the chaperokine activity of Hsp70. Cellular receptors that interact with extracellular hsp's have also been identified for hsp60, hsp90, gp95 and calreticulin. Typically, interaction of the hsp's with the receptors expressed on antigen presenting cells (APC) initiates the observed immune-modulatory activity of hsp's. Free hsp's are involved in cellular receptor transduced innate immune effects, whereas hsp's with bound misfolded protein antigen are involved in triggering an adaptive immune response, corresponding to a humoral response. Interestingly, CD36, SR-A, LRP, CD40 and LOX-I have been identified as multiligand receptors with affinity for amyloid-like crossbeta structure comprising proteins. Based on our recent finding that lipopolysaccharide (LPS) induces crossbeta structure in exposed proteins, we hypothesize that also LPS-binding TLR4 interacts with crossbeta structure comprising proteins.

One of the molecules with a dual function in the ER and outside the cells is BiP. This ER-located BiP functions as a intracellular sensor for cell stress accompanied by protein misfolding, and BiP also exhibits anti-inflammatory and immune-modulatory properties when present in the extracellular environment by the stimulation of an anti-inflammatory gene program from human monocytes and by the development of T-cells that secrete regulatory cytokines such as interleukin-10 and interleukin-4. Also endoplasmic reticulum chaperone heat-shock protein gp96 has been identified as a molecule with dual activity inside and outside the cell. Gp96 not only functions as a regulatory hsp in intracellular protein homeostasis, but gp96 is also instrumental in the initiation of both the innate and adaptive immunity. Yet another example of an hsp which serves key regulatory functions in limiting protein aggregation, facilitating protein refolding, and chaperoning proteins, is hsp72. Apart from its role inside the cell, it has been recognized that, in addition to other hsp's, particularly hsp72 is found extra-cellularly. Extra-cellular hsp72 exhibits potent immune-modulatory effects on innate and acquired immunity. When a host experiences an episode of stress, like for example during infection or during another pathological event, extra-cellular circulating hsp72 levels also greatly increase. These observations point to an important protective role for hsp72 during pathological conditions like for example tissue damage, pathogen invasion, inflammation, by facilitating immunological responses.

Interestingly, BiP has also been identified as an important auto-antigen in rheumatoid arthritis (RA) patients. Titers of anti-BiP auto-antibodies are commonly seen in sera of RA patients. More in general, in almost all inflammatory diseases (auto-)immune responses to certain hsp's occur. The hsp's are involved in presenting antigen to antigen presenting cells (APC) like macrophages and dendritic cells (DC's) to induce a cellular immune response and/or a humoral immune response. In addition, hsp's are apparently also capable of inducing anti-inflammatory pathways by promoting the production of anti-inflammatory cytokines.

We propose that the antigens that bind to extra-cellular hsp's have to fulfil the same structural requirements before an hsp can bind, as what occurs in the cell. Therefore, in this respect also extra-cellular ligands of hsp's are so-called misfolded proteins. Due to the active role of hsp's in modulating the host's response towards pathological conditions, by binding of hsp's to misfolded antigens and presenting them to endocytic receptors like CD36, CD91 or SR-A, we will not exclude the possibility that the host induces an auto-immune response against self-hsp's due to the intimate complex formation between the hsp's and the bound misfolded non-self pathogenic antigens. Perhaps, pathogen antigens and bound hsp chaperones are endocytosed together, and subsequently degraded and processed for antigen presentation purposes. In this view, an auto-immune response against hsp's is perhaps nothing more than the unwanted outcome of a so-called ‘bystander’ role for the hsp's. An important role for extra-cellular heat shock proteins and their cell-surface multiligand receptor counterparts in host protection against pathogenic infection has become more and more evident.

The present invention provides the insight that a chaperone molecule and more in specific an extra-cellular chaperone molecule (such as for example BiP, HSP70, clusterin, hsp72, hsp60, hsp90, gp95, calreticulin, gp96 or haptoglobin) is capable of interacting with a crossbeta structure and/or a molecule comprising a crossbeta structure and/or a molecule comprising a crossbeta structure precursor. Based on this insight, the present inventors have developed methods and means which will be discussed in this description.

A crossbeta structure is a secondary/tertiary/quarternary structural element in peptides and proteins. A crossbeta structure (also referred to as a “cross beta” or a “crossbeta” structure or a “cbs”) is defined as a protein or peptide or a part of a protein or peptide, or a part of an assembly of peptides and/or proteins, which comprises single β-strands (stage 1) and/or a(n ordered) group of β-strands (stage 2), typically a group of β-strands arranged in a β-sheet (stage 3), in particular a group of stacked β-sheets (stage 4), also referred to as “amyloid”. A crossbeta structure precursor is defined as a protein conformation that precedes the formation of any of the aforementioned structural stages of a crossbeta structure. Examples of peptides with crossbeta structure precursor conformation are human fibrin α-chain fragments, yeast prion protein Sup32 fragment and human amyloid-β peptides (Dr. Loes Kroon-Batenburg and Prof. Piet Gros, Utrecht University; manuscript in preparation in collaboration with the present inventors). A typical form of stacked β-sheets is in a fibril-like structure in which the β-sheets are stacked in either the direction of the axis of the fibril or perpendicular to the direction of the axis of the fibril. The direction of the stacking of the β-sheets in crossbeta structures is perpendicular to the long fiber axis. A typical form of a crossbeta structure precursor is a partially or completely misfolded protein, a partially or completely unfolded protein, a partially or completely refolded protein, a partially or completely aggregated protein, an oligomerized or multimerized protein, a partially or completely denatured protein. A crossbeta structure or a crossbeta structure precursor can appear as monomeric molecules, dimeric, trimeric, up till oligomeric assemblies of molecules, and can appear as multimeric structures and/or assemblies of molecules. Crossbeta structure (precursor) in any state from monomeric molecule up till multimeric assembly of molecules can appear in soluble form in aqueous solutions and/or organic solvents and/or any other solutions, like for example as soluble oligomers, and/or crossbeta structure in any state from monomeric molecule up till multimeric assembly of molecules can be present as solid state material in solutions, like for example as insoluble aggregates, fibrils, particles, like for example as a suspension or separated in a solid crossbeta structure phase and a solvent phase. Soluble crossbeta structure or crossbeta structure precursor is defined as the fraction of molecules that are present in a solution after applying 100,000*g to the solution for 1 hour. A crossbeta structure conformation is a signal that triggers a cascade of events that induces clearance and breakdown of the obsolete protein or peptide. When clearance is inadequate, unwanted proteins and/or peptides aggregate and form toxic structures ranging from soluble oligomers up to precipitating fibrils and amorphous plaques. Such crossbeta structure conformation comprising aggregates underlie various diseases, such as for instance Huntington's disease, amyloidosis type disease, atherosclerosis, diabetes, bleeding, thrombosis, cancer, sepsis, inflammatory diseases, rheumatoid arthritis, transmissible spongiform encephalopathies such as Creutzfeldt-Jakob disease, Multiple Sclerosis, auto-immune diseases, diseases associated with loss of memory such as Alzheimer's disease, Parkinson's disease and other neuronal diseases (epilepsy), encephalopathy, encephalitis, cataract and systemic amyloidoses.

A crossbeta structure is for instance formed during unfolding and refolding of proteins and peptides. Unfolding of peptides and proteins occur regularly within an organism. For instance, peptides and proteins often unfold and refold spontaneously during intracellular protein synthesis and/or during and/or at the end of their life cycle. Moreover, unfolding and/or refolding is induced by environmental factors such as for instance pH, glycation, oxidative stress, heat, irradiation, mechanical stress, proteolysis and so on. The terms unfolding, refolding and misfolding relate to the three-dimensional structure of a protein or peptide. Unfolding means that a protein or peptide loses at least part of its three-dimensional structure. The term refolding relates to the coiling back into some kind of three-dimensional structure. By refolding, a protein or peptide can regain its native configuration, or an incorrect refolding can occur. The term “incorrect refolding” refers to a situation when a three-dimensional structure other than a native configuration is formed. Incorrect refolding is also called misfolding. Unfolding and refolding of proteins and peptides involve the risk of crossbeta structure formation. Formation of crossbeta structures sometimes also occurs directly after protein synthesis, without a correctly folded protein intermediate.

A misfolded protein is defined herein as a protein with a structure other than a native, non-amyloid, non-crossbeta structure. Hence, a misfolded protein is a protein having a non-native three dimensional structure, and/or a crossbeta structure, and/or an amyloid structure. Protein misfolding is of etiological importance to a large number of diseases, often related to aging (such as amyloid diseases). Misfolding diseases are also referred to as conformational diseases. At present over 30 misfolding diseases, including but not limited to localized and systemic amyloidoses, like Alzheimer's disease and dialysis related amyloidosis, Parkinson's disease, and Huntington's diseases, have been described as such. We previously disclosed that, in addition to these known misfolding diseases, many other diseases, of which a number with still partly or largely unknown etiology, including (auto-)immune diseases and atherosclerosis, are associated with protein misfolding (patent WO 2004 004698 (EP1536778) and related patents). For many of these diseases no adequate treatment or cure is available. We also disclosed that other processes, of which several can be disease related, such as clearance from the body of obsolete proteins at the end of their life-time, blood coagulation, platelet aggregation and fibrinolysis, are associated with protein misfolding.

Besides the role of misfolded proteins in disease initiation and/or disease progression, protein misfolding also underlies complications, such as adverse generation of auto-antibodies, anaphylactic responses and other inflammatory or allergic reactions, associated with the use of protein pharmaceuticals. For this reason protein misfolding is of major concern during production, storage and use of protein-based drugs.

Finally, misfolded proteins contribute to induction of immunity, and misfolded proteins can be used to trigger and/or potentiate an immune response, for example for the use in vaccines.

Misfolded proteins tend to multimerize and can initiate fibrillization. This can result in the formation of amorphous aggregates that can vary greatly in size. In certain cases misfolded proteins are more regular and fibrillar in nature. The term amyloid has initially been introduced to define the fibrils, which are formed from misfolded proteins, and which are found in organs and tissues of patients with the various known misfolding diseases, collectively termed amyloidoses. Commonly, amyloid appears as fibrils with indefinite length and with a mean diameter of 10 nm, is deposited extracellularly, stains with the dyes Congo red and Thioflavin T (ThT), shows characteristic green birefringence under polarized light when Congo red is bound, comprises B-sheet secondary structure, and contains the characteristic crossbeta conformation (see below) as determined by X-ray fibre diffraction analysis. However, since it has been determined that protein misfolding is a more general phenomenon and since many characteristics of misfolded proteins are shared with amyloid, the term amyloid has been used in a broader scope. Now, the term amyloid is also used to define intracellular fibrils and fibrils formed in vitro. Also the terms amyloid-like and amylog are used to indicate misfolded proteins with properties shared with amyloids, but that do not fulfill all criteria for amyloid, as listed above.

In conclusion, misfolded proteins are highly heterogeneous in nature, ranging from monomeric misfolded proteins, to small oligomeric species, sometimes referred to as protofibrils, larger aggregates with amorphous appearance, up to large highly ordered fibrils, all of which appearances can share structural features reminiscent to amyloid. As used herein, the term “misfoldome” encompasses any collection of misfolded proteins.

Amyloid and misfolded proteins that do not fulfill all criteria for being identified as amyloid can share structural and functional features with amyloid and/or with other misfolded proteins. These common features are shared among various misfolded proteins, independent of their varying amino acid sequences. Shared structural features include for example the binding to certain dyes, such as Congo red, ThT, Thioflavin S, either accompanied by enhanced fluorescence of the dyes, or not, multimerization, and the binding to certain proteins, such as tissue-type plasminogen activator (tPA), the receptor for advanced glycation end-products (RAGE) and chaperones, such as heat shock proteins, like BiP (grp78 or immunoglobulin heavy chain binding protein), HSP60, HSP90. Shared functional activities include the activation of tPA and the induction of cellular responses, such as inflammatory responses and/or immune responses, and induction of cell toxicity.

A unique hallmark of a subset of misfolded proteins such as for instance amyloid is the presence of the crossbeta conformation or a precursor form of the crossbeta conformation.

In addition to what has been described above for the term “crossbeta structure” and/or “crossbeta conformation”, the following part is provided to further clarify this term. A crossbeta structure is a secondary/tertiary/quarternary structural element in peptides and proteins. A crossbeta structure (also referred to as a “crossbeta”, a “cross beta” or a “crossbeta” structure”) is defined as a part of a protein or peptide, or a part of an assembly of peptides and/or proteins, which comprises single β-strands (stage 1) and/or a(n ordered) group of β-strands (stage 2), and/or typically a group of β-strands arranged in a-sheet (stage 3), and/or in particular a group of stacked β-sheets (stage 4), also referred to as “amyloid”. A crossbeta structure is formed following formation of a crossbeta structure precursor form upon protein misfolding like for example denaturation, proteolysis or unfolding of proteins. A crossbeta structure precursor is defined as any protein conformation that precedes the formation of any of the aforementioned structural stages of a crossbeta structure. These structural elements present in crossbeta structure (precursor) are typically absent in globular regions of (native parts of) proteins. The presence of crossbeta structure is for example demonstrated with X-ray fibre diffraction or binding of Thioflavin T or binding of Congo red, accompanied by enhanced fluorescence of the dyes.

A typical form of a crossbeta structure precursor is a partially or completely misfolded protein. A typical form of a misfolded protein is a partially or completely unfolded protein, a partially refolded protein, a partially or completely aggregated protein, an oligomerized or multimerized protein, or a partially or completely denatured protein. A crossbeta structure or a crossbeta structure precursor can appear as monomeric molecules, dimeric, trimeric, up till oligomeric assemblies of molecules, and can appear as multimeric structures and/or assemblies of molecules.

Crossbeta structure (precursor) in any of the aforementioned states can appear in soluble form in aqueous solutions and/or organic solvents and/or any other solutions. Crossbeta structure (precursor) can also be present as solid state material in solutions, like for example as insoluble aggregates, fibrils, particles, like for example as a suspension or separated in a solid crossbeta structure phase and a solvent phase.

Protein misfolding, formation of crossbeta structure precursor, formation of aggregates or multimers and/or crossbeta structure can occur in any composition comprising peptides, of at least 2 amino acids, and/or protein(s). The term “peptide” is intended to include oligopeptides as well as polypeptides, and the term “protein” includes proteinaceous molecules including peptides, with and without post-translational modifications such as glycosylation, citrullination, oxidation, acetylation and glycation. It also includes lipoproteins and complexes comprising a proteinaceous part, such as protein-nucleic acid complexes (RNA and/or DNA), membrane-protein complexes, etc. As used herein, the term “protein” also encompasses proteinaceous molecules, peptides, oligopeptides and polypeptides. Hence, the use of “protein” or “protein and/or peptide” in this application have the same meaning.

A typical form of stacked β-sheets is in a fibril-like structure in which the β-sheets are stacked in either the direction of the axis of the fibril or perpendicular to the direction of the axis of the fibril. The direction of the stacking of the β-sheets in crossbeta structures is perpendicular to the long fiber axis. A crossbeta structure conformation is a signal that triggers a cascade of events that induces clearance and breakdown of the obsolete protein or peptide. When clearance is inadequate, unwanted proteins and/or peptides aggregate and form toxic structures ranging from soluble oligomers up to precipitating fibrils and amorphous plaques. Such crossbeta structure conformation comprising aggregates underlie various diseases, such as for instance, Huntington's disease, amyloidosis type disease, atherosclerosis, diabetes, bleeding, thrombosis, cancer, sepsis and other inflammatory diseases, rheumatoid arthritis, transmissible spongiform encephalopathies such as Creutzfeldt-Jakob disease, Multiple Sclerosis, auto-immune diseases, diseases associated with loss of memory such as Alzheimer's disease, Parkinson's disease and other neuronal diseases (epilepsy), encephalopathy and systemic amyloidoses.

A crossbeta structure is for instance formed during unfolding and refolding of proteins and peptides. Unfolding of peptides and proteins occur regularly within an organism. For instance, peptides and proteins often unfold and refold spontaneously at the end of their life cycle. Moreover, unfolding and/or refolding is induced by environmental factors such as for instance pH, glycation, oxidative stress, heat, irradiation, mechanical stress, proteolysis and so on. As used herein, the term “crossbeta structure” also encompasses any crossbeta structure precursor and any misfolded protein, even though a misfolded protein does not necessarily comprise a crossbeta structure. The term “crossbeta binding molecule” or “molecule capable of specifically binding a crossbeta structure” also encompasses a molecule capable of specifically binding any misfolded protein.

The terms unfolding, refolding and misfolding relate to the three-dimensional structure of a protein or peptide. Unfolding means that a protein or peptide loses at least part of its three-dimensional structure. The term refolding relates to the coiling back into some kind of three-dimensional structure. By refolding, a protein or peptide can regain its native configuration, or an incorrect refolding can occur. The term “incorrect refolding” refers to a situation when a three-dimensional structure other than a native configuration is formed. Incorrect refolding is also called misfolding. Unfolding and refolding of proteins and peptides involves the risk of crossbeta structure formation. Formation of crossbeta structures sometimes also occurs directly after protein synthesis, without a correctly folded protein intermediate.

### Crossbeta Pathway: Response to Misfolded Proteins

We previously disclosed a biological mechanism that senses occurrence of misfolded proteins, resulting in breakdown and clearance of the misfolded proteins, termed the Crossbeta Pathway (patent WO 2004 004698). We experimentally identified a number of proteins, including tPA and the closely related proteins factor XII, hepatocyte growth factor activator (HGFA) and fibronectin, that recognize misfolded proteins, with structural features common to proteins comprising crossbeta structure or a crossbeta structure precursor form. We also disclosed that, based on analysis of the literature, a number of additional proteins, including cell surface receptors, are implicated in the response of the body to misfolded proteins, including clearance of misfolded proteins, and thus are part of the Crossbeta Pathway. We disclosed that a number of these proteins, like tPA and its relatives, are able to recognize misfolded proteins directly. Said proteins were known to bind a large number of ligands that seemed unrelated with respect to 3D structure and/or amino-acid sequence. These protein ligands are often implicated in diseases, but the presence of a common structural or sequential mode of recognition was not identified earlier. Collectively, tPA, its relatives and other proteins that recognize misfolded proteins are thus part of mechanisms that facilitate the clearance of misfolded proteins, i.e. the Crossbeta Pathway. Examples of physiological processes in which the Crossbeta Pathway is involved are long term potentiation, innate immunity, adaptive immunity, angiogenesis, blood coagulation, thrombus formation and fibrinolysis. Malfunctioning of the Crossbeta Pathway will result in proteins that form dangerous misfolded proteins, either or not accompanied by structural features commonly seen in amyloid, like for example aggregates or fibrils with crossbeta conformation. As stated above and before in patent application WO 2004 004698, misfolded proteins underlie various health problems and diseases, some of which are previously associated with protein misfolding and others that have not yet been associated as such. These health problems and diseases include Huntington's disease, localized amyloidoses, atherosclerosis, diabetes, bleeding, thrombosis, cancer, sepsis, inflammatory diseases, rheumatoid arthritis (RA), multiple sclerosis (MS), other auto-immune diseases, diseases associated with loss of memory such as Alzheimer's disease (AD), Parkinson's disease and other neuronal diseases like for example epilepsy, encephalopathy, encephalitis, cataract, systemic amyloidoses, transmissible spongiform encephalopathies, such as Creutzfeldt-Jakob disease, and amyloidosis related to dialysis with patients suffering from renal insufficiency.

In conclusion, the Crossbeta Pathway comprises molecules, some of which directly bind misfolded proteins, termed crossbeta structure binding compounds or crossbeta binding compounds or misfolded protein binding compounds, which contribute to the sensing, the breakdown and/or the clearance of misfolded proteins. The Crossbeta Pathway senses any non-native 3D fold of a protein and responds by means of various modes.

The Crossbeta Pathway also comprises molecules, such as chaperones, that are able to interact with misfolded proteins in order to assist in folding and/or refolding, in order to prevent accumulation of aggregates, fibrils, and/or precipitates of misfolded proteins.

For example, tPA is a serine protease that is activated in response to direct binding to misfolded proteins. One such misfolded protein is fibrin, present in a blood clot. Upon activation, tPA generates plasmin from the zymogen plasminogen. The serine protease plasmin in turn cleaves many substrates, such as proenzymes, like procollagenases, as well as extracellular matrix proteins, like fibrin. As such tPA initiates a cascade of events to degrade aggregates of misfolded proteins, such as blood clots.

Another example is RAGE. This receptor is involved in binding glycated proteins, amyloid and other ligands, that comprise amyloid properties, and is implicated in the pathology of many diseases, such as amyloidosis, diabetes and auto-immune diseases. Administration of a soluble form of this receptor has beneficial effects in animal models of several of the aforementioned protein misfolding diseases.

Yet another example of misfolded protein binding molecules that are involved in the Crossbeta Pathway are the chaperones, or heat shock proteins (HSPs), or stress proteins. The fact that chaperones like for example haptoglobin and clusterin, assist in prevention of formation of aggregates of misfolded proteins in an ATP independent manner make them candidates to play an important role in the Crossbeta Pathway. It is likely that a series of proteins that sample protein conformation act in concert. Amongst these proteins that act in concert in the Crossbeta Pathway are chaperones, like for example HSP60, HSP90, DNAK, clusterin, haptoglobin, gp96, BiP, other (extracellularly located) HSPs, proteases, like for example HGFA, tPA, plasminogen, factor XII, IVIg, and cell surface receptors. Cell surface receptors implicated in the Crossbeta Pathway include low density lipoprotein receptor related protein (LRP, CD91) and relatives, CD36, scavenger receptor A, scavenger receptor B-I, RAGE, collectively also referred to in literature as multiligand receptors.

In summary, the Crossbeta Pathway is capable of preventing misfolded proteins to form toxic structures like for example amyloid crossbeta structure oligomers and fibrils, and is capable of degrading and clearance of (aggregates of) misfolded proteins. As part of the Crossbeta Pathway misfolded proteins bind to multiligand misfolded protein binding receptors, resulting in endocytosis and subsequent proteolytic breakdown.

Hence, modulation of the Crossbeta Pathway provides treatment opportunities for protein misfolding diseases.

### Misfolding Diseases

As mentioned above, diseases associated with protein misfolding, termed protein misfolding diseases, misfolded protein diseases, protein misfolding disorder, conformational diseases, misfolded protein related and/or associated diseases, or protein folding disorders, include amyloidoses, and protein misfolding is also associated with many other diseases and health problems and physiological processes, not necessarily defined by the term amyloidosis or protein misfolding disorder, of which several are mentioned above.

In a first embodiment, the invention provides a method for binding a crossbeta structure comprising protein, comprising contacting said protein with chaperone or a functional equivalent and/or a functional fragment thereof. This method can be performed in vitro as well as in vivo.

The invention further discloses that a chaperone is also able to bind to a crossbeta structure precursor comprising protein and hence the invention further provides a method for binding a crossbeta structure precursor comprising protein, comprising contacting said protein with chaperone or a functional equivalent and/or a functional fragment thereof.

Preferably, the invention provides a method for binding a crossbeta structure (precursor) comprising protein, comprising contacting said protein with a chaperone or a functional equivalent and/or a functional fragment thereof, which method further comprises allowing said crossbeta structure (precursor) comprising protein and said chaperone or a functional equivalent and/or a functional fragment thereof to interact with each other. The interacting step is preferably performed under proper conditions (for example temperature, pH, etc.) that allow said protein and said chaperone to bind to each other.

As used herein the term “binding” comprises short term as well as long term binding between a crossbeta structure (precursor) comprising protein and a chaperone or a functional equivalent and/or a functional fragment thereof (i.e. the binding might be short or might continue for extended periods of time) and comprises strong, as well as intermediate and weak binding interactions. In general, non-covalent binding occurs. Hydrophobic interactions and/or electrostatic interactions and/or hydrogen bonds and/or salt bridges and/or van der Waals bonds and/or alternative non-covalent protein-protein interactions can all contribute to the binding. An effector molecule can modulate the strength of binding. For example, ATP induces decreased binding capacity of BiP, HSP60 or HSP90 (examples of a chaperone) towards a misfolded protein ligand.

Preferably the contacting phase is performed in a liquid medium (for example a buffered liquid medium) in which a crossbeta structure (precursor) comprising protein and a chaperone or a functional equivalent and/or a functional fragment thereof are present. As will be discussed in more detail later, either or both of the compounds may be immobilised on a carrier.

It is clear to the skilled person that the effectivity of the binding or the binding strength between a crossbeta structure (precursor) comprising protein and a chaperone (or a functional equivalent and/or a functional fragment thereof) for example depends on the particular (external) circumstances, for example the concentration of said compounds, the temperature, presence of excipients, the pH, the presence or absence of an effector molecule etc.

The term “chaperone” is herein used to refer to a so-called classical chaperone and includes a (molecular) chaperone and/or a co-chaperone and/or a chaperonin and/or a chaperokine and/or a heat shock protein and/or a stress protein and or a small heat shock protein and/or a protein disaggregase and/or a pharmacoperone. Detailed information in respect of these different chaperones is now provided.

### (Molecular) Chaperone

The function of chaperones is to assist other proteins to achieve proper folding. Chaperones recognize and bind to nascent polypeptide chains, partially folded intermediates, or denatured proteins to prevent their misfolding and/or aggregation and are thus involved in protein folding, assembly, disassembly and transport accros membranes. Chaperones are also essential in ‘rescuing’ proteins which have become misfolded or aggregated. This misfolding or aggregation can arise spontaneously or as a consequence of a cell being subject to environmental stresses such as heat shock. Specific chaperones unfold the misfolded or aggregated protein and, in conjunction with other chaperones, rescue the protein by sequential unfolding and refolding the protein back to its native and biologically active form. Chaperones also participate in the elimination of abnormal polypeptides, due to mutations or damage by stress, if they are beyond repair. So chaperones also are involved in protection of cells from the effects of heat or other stresses. Chaperones comprise several highly conserved families of unrelated proteins; many chaperones are also heat shock/stress proteins, but not all. For example; some peptidyl prolyl isomerases (PPIs) are not stress/heat induced. SecB is another good example of a chaperone which is not inducible by stress. Although many chaperones also are stress/heat shock proteins, most are expressed during physiological functioning of an organism or cell (non stress) because they have an essential role in protein maintenance.

Some chaperones can act solely intracellular, other chaperones can act solely in extra-cellular space, whereas again other chaperones can be found both in cells and outside cells.

Thus chaperones are defined as proteins that assist other macromolecules in folding/unfolding and in assembly/disassembly of higher order structures without being components of these final structures.

### Co-Chaperone

Co-chaperones are proteins which can interact with and/or bind to chaperones and regulate and/or assist in their activity.

### Chaperonin

Chaperonins is the name given to chaperone protein complexes of a specific structure. The 3D structure of these chaperonins resemble two donuts stacked on top of one another to create a barrel. Each ring is composed of either 7, 8 or 9 subunits depending on the organism in which the chaperonin is found. All chaperoning belong to the group of HSP60 proteins and are divided (reminiscent to the HSP60 proteins) in two different groups. Group I chaperoning are found in prokaryotes as well as organelles of endosymbiotic origin: chloroplasts and mitochondria. The GroEL/GroES complex in E. coli is a good example of a Group I chaperonin and is the best characterized large (˜1 MDa) chaperonin complex. Group II chaperoning, found in the eukaryotic cytosol and in archaebacteria, are more poorly characterized. Group II chaperonins are thought not to utilize a GroES-type cofactor to fold their substrates. They instead contain a “built-in” lid that closes in an ATP-dependent manner to encapsulate their substrates, a process that is required for the protein ligands to fold.

### Chaperokine

Alexzander Asea and Stuart Calderwood proposed the term chaperokine for heat shock protein 70 (HSP70). It seems the well known molecular chaperone acts as a cytokine, signalling a potent inflammatory response in monocytes. Chaperokine is a description given to chaperones in general which function as a cytokine, like for example HSP60, HSP10, HSP27, BiP and HSP70. They can be divided in two functional groups: pro-inflammatory chaperokines like for example HSP60 and HSP70, and anti-inflammatory chaperokines like for example BiP, HSP10, HSP27.

### Heat Shock Proteins (HSPs)/Stress Proteins

The term heat shock protein (HSP) is a misnomer but remains as a legacy of Ritossa's discovery that heat shock produced chromosomal puffs of salivary gland cells in Drosophila. Diverse stresses, including heavy metals, amino acid analogues, inflammation and oxidative/ischemic stress, induce the expression of HSP genes. A good example is the HSP70 protein ‘binding protein’ (BiP), which is also named glucose related protein 78 (GRP78), due to its elevated expression under starving conditions. Consequently, the terms stress proteins or heat shock family of stress proteins are preferred, although many of these proteins have essential functions during unstressed conditions. All living organisms from archaebacteria to eubacteria, yeast, plants, invertebrates and vertebrates respond at the cellular level to unfavourable conditions, such as the stress conditions mentioned, by the rapid, vigorous, and transient acceleration in the rate of expression of a small number of specific genes. Consequently, the amount of products of these genes (stress proteins/heat shock proteins) increase and accumulate in cells to reach, in some instances, fairly high concentrations. Most stress proteins/heat shock proteins are constitutively expressed, but have higher expression under the influence of stress factors. Some stress proteins function as molecular chaperones, but not all stress proteins/heat shock proteins are chaperones. HSP32 is a stress induced protein for which there is no evidence that it's regulated by a stress response. The regulators of the heat shock genes, like the heat shock factors (HSFs), also belong to the group of stress proteins, though which have no chaperone function.

### Small Heat Shock Protein

One of the stress protein/heat shock protein families is the small HSP family. Proteins in this family range in monomer size from 12-48 kDa (smaller then 34 kDa according to an alternative definition), are characterized by a conserved—crystallin domain in their C-terminal part, and form oligomeric stuctures ranging from 9-50 subunits. One of the main functions of most small HSPs is their ability to behave as molecular chaperones. This property does not depend on ATP supply and is associated with the formation of large oligomers, at least in mammal cells. Thus, small HSPs bind several non-native proteins per oligomeric complex, therefore representing the most efficient chaperone family in terms of the quantity of substrate binding. They also interact with intermediate filaments and the actin cytoskeleton, modulate intracellular oxidative stress and prevent apoptotic cell death. Analysis of the developmental expression pattern of small HSPs in various tissues demonstrates a complex and coordinated regulation in both non-mammals and mammals. This developmental regulation occurs primarily at the level of transcription, but seems to be independent of heat shock transcription factors (HSFs) and their heat shock element (HSE) target-sequence; for example the developmental expression of Drosophila small HSPs is regulated mainly by steroid hormones, whereas hormone-regulated mammalian HSP27 expression is limited to specific tissues in mammals. Examples of small HSPs are HSP10, PPIase, PDIase, prefoldin subunits and the a-crystallin family.

### Protein Disaggregase

In 1990 Lindquist and co-workers discovered a new heat shock protein of the yeast Saccharomyces cerevisiae, termed HSP104. HSP104 was shown to be essential for development of thermo-tolerance, a physiological status that allows cells to survive severe stress after mild heat treatment. It was further shown that the substrates of HSp104 are large electron-dense aggregates generated during severe heat stress. S. cerevisiae cells lacking Hsp104 function were no longer able to solubilise and reactivate proteins from an aggregated state. The function of Hsp104 is conserved in eubacteria, plants and mitochondria, as its homologues ClpB, Hsp101 and Hsp78 are essential for both thermo-tolerance and protein dysaggregation. Unlike Hsp70 and most other chaperones such as the small heat shock proteins, the role of HsP104 is not to prevent the aggregation of denatured or partially unfolded proteins, but rather, in conjunction with both Hsp70 and the co-chaperone Hsp40, to act as a ‘protein disaggregase’ leading to the resolubilisation of protein aggregates. Hsp104 acts as a molecular ‘crowbar’ to shear high molecular weight aggregates into smaller aggregates that can then be more effectively dealt with by the Hsp70/Hsp40 chaperone system. Therefore the term protein disaggregase is used for proteins which solubilise/break down protein aggregates.

### Pharmacoperone

A pharmacoperone (from pharmacological chaperone) is a small molecule that enters cells and serves as a molecular scaffolding in order to induce folding and correct routing within the cell of otherwise-misfolded mutant proteins.

Pharmacoperones correct the folding of misfolded proteins, allowing them to pass through the cell's quality-control system and helping them to become correctly routed. Since mutations often cause disease by causing misfolding and misrouting, pharmacoperones are drug candidates, since they are able to correct this defect.

Alternatively, the term “chaperone” refers to any of proteins listed in Table 4. Table 4 provides an overview of a literature survey of chaperones/(small) heat shock proteins/(co-)chaperonins/co-chaperones/chaperokines/stress proteins/protein disaggregases, identified in various species. Table 4 is subdivided into the mentioned chaperones and hence in yet another preferred embodiment, the term chaperone as used herein refers to any of these subparts of Table 4.

Based on the herein provided description it is clear that the so-called classical chaperones can at least perform two different functions. The first one being their role in protein folding (or the removal of misfolded proteins). The second function is the binding to misfolded proteins and delivering the misfolded protein to an antigen presenting cell. In the first function, the protein is in principle provided with its normal/native/wild type conformation such that the protein can perform it's normal/native/wild type function. In the second function the protein is misfolded and is provided with a function different from it's normal/native/wild type function. In a yet another preferred embodiment, the herein used chaperone comprises at least two functions, i.e. a chaperone as used by the present invention is capable to assist other proteins to achieve proper folding (or to route improper folded proteins to clearance and degradation pathways), and is capable of interacting with a crossbeta structure. Alternatively, a chaperone as used by the present invention is capable of interacting with an antigen with crossbeta structure and/or a crossbeta structure induced conformation, and is capable of aiding in eliciting an immune response against the antigen.

As disclosed herein within the experimental part, the binding of a chaperone to a crossbeta structure can be at least partly inhibited by providing a crossbeta binding compound (for example any of the compounds as described in Table 1-3) and hence in yet another preferred embodiment the term “chaperone” refers to a (molecular) chaperone and/or a co-chaperone and/or a chaperonin and/or a chaperokine and/or a heat shock protein and/or a stress protein and or a small heat shock protein and/or a protein disaggregase and/or a pharmacoperone which binding to a crossbeta structure is at least partly inhibited by a(nother) crossbeta binding compound (for example any of the compounds as described in Table 1-3).

It is clear from Tables 1-3 that a large amount of crossbeta binding compounds exist. However, not all crossbeta binding compounds are a chaperone. For example tissue-type plasminogen activator (tPA) is a crossbeta binding compound but not a chaperone. Another example is Congo red, a fluorescent dye with the ability to bind to crossbeta structure.

A series of chaperones was used for the herein described examples showing that the invention is not limited to a certain member of the class of HSPs, exemplified by currently presenting data for an HSP60, two HSP70s and an HSP90, or restricted to a certain species (exemplified by currently presenting data for human BiP, HSP60, HSP90 versus E. coli DnaK). I.e., examples of a suitable chaperone are BiP, HSP60, HSP90 and DnaK, however it is clear to skilled persons that the invention is not limited to these chaperones. A brief non-limiting description about HSPs selected for the examples is provided below.

### HSP70

HSP70 homologues form a large family of highly related proteins with chaperone activity and are present in all groups of organisms. Bacterial HSP70 (also known as DnaK) is generally present in one form. Eukaryotes have several homologues, and these are organelle (nucleus, cytosol, endoplasmic reticulum, mitochondrion or chloroplast) specific. HSP70 homologues bind transiently to unfolded polypeptide structures, and this can be reversed by ATP. HSP70 is one of the most conserved HSPs. A study comparing the sequences of two members of the HSP70 family of Saccharomyces cerevisiae with a set of 29 HSP70 molecules from 24 other eukaryotic and prokaryotic species clearly shows the high level of conservation of HSP70 molecules: proteins from the most distantly related species have at least 45% sequence identity. HSP70 family members contain an N-terminal ATPase domain and a C-terminal substrate binding domain, and act in a monomeric form. The peptide-binding cleft consists of a β-sandwich motif. During the chaperone's ATP-hydrolysis cycle, the peptide is locked into place by an α-helical lid structure. In the ATP-bound state, (part of) a misfolded protein has access to the open binding pocket. ATP hydrolysis closes the lid, clamping down and trapping the polypeptide backbone. The structure of full-length bovine Hsc70 has been solved, revealing how the ATPase and substrate-binding domains connect and may communicate with one another allosterically. Hsp70 forms clamp-like enclosures as monomers rather than as oligomers.

HSP70 molecular chaperones are major primary association partners, which in turn, are able to bind numerous unrelated protein structures, thereby forming ternary complexes. Hsp70s are thus engaged in a plethora of folding processes including the folding of newly synthesized proteins, the transport of proteins across membranes, the refolding of misfolded and aggregated proteins, and the control of activity of regulatory proteins. This versatility is achieved through the evolutionary amplification and diversification of hsp70 genes, which has generated both specialized Hsp70 chaperones and more diverged Hsp110 and Hsp170 proteins. Versatility is also achieved through extensive employment of co-chaperones, J proteins, and nucleotide exchange factors (NEFs), which regulate Hsp70 activity.

Human BiP and Escherichia coli DnaK

Human BiP (Ig heavy chain binding protein), also referred to as GRP78, is a HSP70 family member. BiP was discovered as a glucose regulated protein (GRP78) because low glucose leads to ER stress and thus to the up-regulation of BiP expression. BiP is localized in the ER were its function is to facilitate protein folding and translocation. It is an ATP-ase containing an N-terminal nucleotide-binding domain (ATP) and a C-terminal substrate binding domain. It uses its ATP-ase activity to assist (re)folding of unfolded proteins. The nature of the interaction of BiP with its many known substrates is unclear.

Like other HSP70 chaperones, BiP assists folding of the substrate by repeated cycles of binding and release, driven by ATP-dependent conformational changes of the chaperone. The regulation of this ATPase cycle is best understood for the E. coli HSP70 homologue DnaK (see below). ATP binding leads to a conformational change in the substrate binding domain which modulates substrate affinity. The rate limiting step of the ATPase cycle of DnaK has been shown to be the hydrolysis step. Binding of short peptides to BiP stimulates the hydrolysis of ATP. Consistent with findings for DnaK, it has been shown for different peptides, that ATP hydrolysis is not necessary for dissociation of BiP-peptide complexes, but that the release is achieved by a conformational change upon ATP binding.

### Human HSP60

HSP60 was identified in Escherichia coli to be the molecule known as GroEL (also known as Cpn60), and at the same time, GroES (now known as HSP10 or Cpn10) was also identified. HSP60 forms multimeric structures of two stacked heptameric rings to create a central cage in which polypeptide folding is assisted. This inner cavity can hold proteins with a molecular weight up to 50 kDa. The heptameric co-chaperonin HSP10 complexes function as ‘lids’ for this cavity. The opening and closing of the cage and the folding of polypeptides depends on the ATPase activity of HSP60. Folding intermediates are the preferential substrate for HSP60 and HSP60 is essential for acquiring the native conformation of many mitochondrial proteins. Bacteria have one or two variants of HSP60, whereas eukaryotes have one, which is located in the mitochondrial or chloroplast matrix. All HSP60 molecules have a high degree of sequence similarity and especially have conserved sequence positions essential to protein function and/or structure. Human HSP60 has about 50% sequence identity with GroEL. In contrast to GroEL, human HSP60 also can exert its function as a monomeric ring. Eukaryotes also have another very similar protein, namely TCP-1, which is found in the cytosol. TCP-1 also forms multimeric rings structures that resembles the GroEL ring, but can function independently of co-chaperonins like HSP10. HSP60 and its chaperonin HSP10 are the most important components of the protein folding system in the mitochondrial matrix.

### Human HSP90

Hsp90 consists of three distinct regions: an N-terminal ATP-binding domain, a middle domain and a C-terminal dimerization domain. The crystal structures of the N-terminal and middle domains, and of the C-terminal domain of the E. coli Hsp90 homolog HtpG were solved separately. More recently, the structure of full-length yeast Hsp90 was solved. ADP-bound Hsp90, the ‘open’ state, dimerizes only through its C-terminus and is receptive to binding substrate (often referred to as “client” or “cargo”) proteins. Exchange of ADP for ATP induces a conformational change leading to a homotypic interaction between the N-terminal domains and conversion to the ‘closed’ state, thus clamping down on client proteins. The closed state represents the substrate-bound state. The structure showed that client proteins could not be physically enclosed within the closed Hsp90 dimer. Numerous substrates (ligands or clients), including but not limited to kinases, transcription factors and extra-cellualr proteins, for HSPs are known and several substrate-binding sites on Hsp90 have been proposed, but it is unclear how Hsp90 precisely facilitate client proteins to mature. Recent data suggest that substrates contact multiple sites on Hsp90 such that the conformational changes in Hsp90 during closure would directly affect the conformation, and ultimately the activity, of the client protein. Up till now, the details of the Hsp90 functional cycle remain elusive even with the considerable structural information now available. Hsp90 has been implicated as a possible drug target. Hsp90 is involved in cell cycle signaling, of which some proteins are known to be oncogenic. It was shown in different human and mammalian models that Hsp90 inhibition acts additively or synergistically with other cancer therapies. Hsp90 inhibition had clinical efficacy in melanoma, breast cancer, prostate cancer and different leukemias. Hsp90 inhibition is also implicated as a possible anti-viral therapy. Inhibition of Hsp90 would result in less efficient folding of viral proteins. This was shown for viral infection of certain cell cultures.

As outlined above, chaperones are a diverse class of proteins and reference to a chaperone herein include (but is not limited to) (small) heat shock proteins (hsp's), chaperokines, chaperonins and stress proteins and they may be located intra-cellular as well as extra-cellular. Preferred hsp's are hsp60, hsp70, hsp90 or gp96. Even more preferred are extracellular chaperones, such as BiP, haptoglobin, hsp72 or clusterin and even more preferred are extra-cellular chaperones that are ATP-independent, such as haptoglobin or clusterin. Included are also the chaperones that are derived from another species and have a different name/nomenclature but which are comparable to any of the mentioned chaperones. In yet another preferred embodiment, the preferred chaperone is selected from BiP, HSP60, HSP90 and DnaK.

A functional equivalent is for example a chaperone which is derived from another species. For example, the eukaryotic cell hsp90 or hsp83 homologue in higher eukaryotes is grp94 or endoplasmin, and in prokaryotic cells htpG. The eukaryotic hsp70 counterpart in prokaryotes is DnaK (E. coli) and Kar2p in yeast. The bacterium DnaJ homologue in yeast is Sec63p. The term functional refers to the fact that said equivalent must at least be capable of binding a crossbeta structure (precursor), preferably a crossbeta structure (precursor) comprising protein. Hence, a functional part of any of the mentioned chaperones is any part derived from a chaperone that is still capable of binding to a crossbeta structure (precursor), preferably a crossbeta structure (precursor) comprising protein, although the strength of the binding may be different (either decreased or increased). As already mentioned, some chaperones have a dual function, i.e. they are able to bind a crossbeta structure (precursor) and also have immune modulating capacities. An example of such a chaperone is BiP. Depending on its specific application, BiP or a functional equivalent and/or a functional fragment thereof may or may not include the immune modulating domain. If one for example uses BiP or a functional equivalent and/or a functional fragment thereof in a vaccine composition, said immune modulating domain has certain advantages and is preferably included. However, in for example neutralizing applications, said immune modulating part is preferably impaired (for example by deleting the complete domain or by deleting and/or substituting key amino-acid residues that is/are responsible for the immune modulating function). Hence, a functional part and/or a functional equivalent of a dual chaperone may or may not include an immune modulating domain depending on the specific application.

Besides a method for binding a crossbeta structure comprising protein, the invention also provides a method for detecting a crossbeta structure comprising protein (i.e. binding to form a complex followed by visualising said complex) as well as a method for removing a cross-beta structure comprising protein (i.e. binding to form a complex followed by removing said complex). These embodiments are now discussed in more detail.

Hence, in one of the embodiments, the invention provides a method for detecting a crossbeta structure in a sample, comprising contacting said sample with a chaperone or a functional equivalent and/or a functional fragment thereof, allowing for binding of a crossbeta structure to said chaperone and detecting the complex formed through binding. Preferably, said crossbeta structure is part of a protein, i.e. a crossbeta structure comprising protein. As outlined above, this embodiment is also applicable for a crossbeta structure precursor (comprising protein).

Examples of a sample are a body fluid or tissue, food, fluid, a vaccine, or a pharmaceutical composition.

As described above, crossbeta structure in proteins are often related to, and/or associated with, a risk and/or presence of disease, such as for instance Huntington's disease, amyloidosis type disease, atherosclerosis, diabetes, bleeding, thrombosis, cancer, sepsis, inflammatory diseases, rheumatoid arthritis, transmissible spongiform encephalopathies such as Creutzfeldt-Jakob disease or BSE, Multiple Sclerosis, auto-immune diseases, diseases associated with loss of memory such as Alzheimer's disease, Parkinson's disease and other neuronal diseases (epilepsy), encephalopathy, encephalitis, cataract and systemic amyloidoses. Hence, a method for detecting a crossbeta structure in a sample as described herein is for example very useful in the diagnosis of the mentioned diseases. In such a case said sample is preferably a body fluid, like for example blood, serum, plasma, lymph fluid, cerebrospinal fluid, synovial fluid, sputum or urine. In another preferred embodiment, said sample is derived from a tissue (for example a brain tissue). Optionally said tissue is homogenised by any known method in the art to obtain a (partly) fluid sample.

A method for detecting a crossbeta structure in a sample may be performed in vivo or in vitro. Hence, the invention also provides use of a chaperone (or a functional equivalent and/or a functional fragment thereof) in the preparation of a diagnostic for the diagnosis of any of the above-mentioned diseases.

Optionally, a sample (suspected of) comprising a crossbeta structure comprising protein is contacted with a combination of a chaperone (or a functional equivalent and/or a functional fragment thereof) and at least one other crossbeta structure binding compound. Non-limiting examples of other crossbeta structure binding compounds are an antibody (or a fragment and/or a derivative thereof) directed against a crossbeta structure, a finger domain (also referred to as fibronectin type I domain) of tissue-type plasminogen activator (tPA), hepatocyte growth factor activator (HGFA), factor XII, or fibronectin, or members of the multiligand receptor family such as receptor for advanced glycation end-products (RAGE), or low density lipoprotein receptor related protein (LRP) or CD36. Such a crossbeta structure binding compound may even be a non-proteinaceous molecule, for example a dye (Congo red or Thioflavin). Other non-limiting examples of crossbeta structure binding compounds are provided in Table 1 or 2 or 3.

In yet another preferred embodiment, a chaperone (or a functional equivalent and/or a functional fragment thereof) is attached to a (solid) support or phase (i.e. is immobilised) such as for example a sphere or a particle or a bead or a sheet or a strand of latex or agarose or glass or plastic or metal or any other suitable substance for immobilisation of molecules. Such immobilisation is especially useful when bound and unbound proteins must be separated. Depletion of a fluid from crossbeta structure and/or a protein comprising a crossbeta structure can be assessed, and/or enrichment of a solid support with bound chaperone or a functional equivalent and/or a functional fragment thereof with crossbeta structure and/or a protein comprising a crossbeta structure can be assessed, after contacting a fluid with the chaperone or a functional equivalent and/or a functional fragment thereof that is immobilized on a solid support. For example, a spike of a reference crossbeta structure can be applied to a tester sample and a control or reference sample. When contacting the samples with a chaperone the amount of crossbeta structure originally present in the sample will determine the amount of reference crossbeta structure that will bind to the chaperone. The differences in amount of reference crossbeta structure in a control sample and in a tester sample after contacting both samples to the chaperone can be assessed, for example with a(n) (sandwich) ELISA specific for the reference crossbeta structure, or for example by fluorescence measurement when a fluorescent label is coupled to the reference crossbeta structure. Alternatively, the amount of reference crossbeta structure bound to the chaperone can be assessed similarly. In yet an alternative approach, all proteins in a tester sample and in a reference or control sample can be labelled for example with biotin or a fluorescent label, prior to exposure to a chaperone. The amount of labelled protein comprising crossbeta structure bound to the chaperone can subsequently be quantified and compared. In yet another approach, misfolded protein comprising crossbeta structure that is bound to chaperone after contacting a reference sample and a tester sample with the chaperone, can be quantified after elution from the chaperone immobilized on a solid support, using a chromogenic assay. In the chromogenic assay, for example dilution series of eluates of chaperone ligands are mixed with tissue-type plasminogen activator, plasminogen, a chromogenic substrate for plasmin and a suitable reaction buffer, and conversion of the substrate is followed in time upon 37° C. incubation.

In yet another embodiment, a chaperone is used to prepare a diagnostic kit. Said diagnostic kit is particularly suitable for diagnosis of a disease that is related to, and/or associated with, the presence of misfolded proteins and/or crossbeta structures. Said kit preferably comprises at least one chaperone, capable of interacting with a misfolded protein and/or a crossbeta structure and/or with a protein comprising a crossbeta structure, and a way of visualization of an interaction of said misfolded protein and/or crossbeta structure and/or said protein with said chaperone.

With such diagnostic kit, not only diseases that are generally related to and/or associated with the presence of misfolded proteins and/or crossbeta structures are diagnosed, but also a more defined diagnosis is possible, dependent of the specificity of the chaperone in the kit. A diagnostic kit capable of specifically diagnosing one kind of disorder is for instance generated by providing said kit with a chaperone that is capable of specifically binding a given misfolded protein and/or crossbeta structure and/or a given protein comprising a crossbeta structure that is specific for said one kind of disorder, such as for example proteins related to rheumatoid arthritis, SLE or other autoimmune diseases, or inflammatory reactions. Therefore, in one embodiment, the invention provides a diagnostic kit as described above, wherein said misfolded protein and/or crossbeta structure is a disease-related misfolded protein and/or crossbeta structure.

Since misfolded proteins and/or crossbeta structures and proteins comprising a crossbeta structure are effectively bound to a chaperone, they are effectively separated and/or isolated from a sample and/or an animal's or human's body and subsequently identified. In yet another embodiment therefore, a chaperone is used to isolate misfolded proteins and/or crossbeta structures and/or proteins comprising a crossbeta structure. Preferably, misfolded proteins and/or crossbeta structures and/or proteins comprising a crossbeta structure present in a body fluid, like for example blood, serum, plasma, cerebrospinal fluid, synovial fluid, sputum and/or urine, is identified. For instance, the presence and/or identity of a misfolded protein and/or a crossbeta structure, and/or protein comprising a crossbeta structure, of healthy individuals is compared with the presence and/or identity of a misfolded protein and/or a crossbeta structure, and/or protein comprising a crossbeta structure, from individuals with a disease related to and/or associated with a misfolded protein and/or a crossbeta structure and/or a protein comprising a crossbeta structure. The identity and the relative concentration of a misfolded protein and/or a crossbeta structure and/or protein comprising a crossbeta structure is determined using any method known to a person skilled in the art, like for example, but not limited to, 2D gel electrophoresis and/or mass-spectrometric analyses. The results of a sample originating from a healthy individual and a sample originating from a patient are preferably compared. In this way, information is obtained, for instance about the identity and/or susceptibility of proteins prone to misfold and/or adopt crossbeta structure conformation during defined disease states. This obtained information subsequently serves as a diagnostic tool, for instance to monitor disease state, to monitor effectiveness of therapy, to monitor occurrence of disease, and provides valuable leads for development of therapeutics targeted at misfolded proteins and/or crossbeta structures and/or protein(s) comprising a crossbeta structure which are preferably specific for a defined disease.

The invention therefore provides a method for determination of the identity of a misfolded protein and/or a crossbeta structure or a protein comprising a crossbeta structure in a sample comprising a protein, said method comprising:
contacting said sample with a chaperone, resulting in bound misfolded proteins and/or crossbeta structures and/or bound protein(s) comprising a crossbeta structure, and
identifying a bound misfolded protein and/or crossbeta structure and/or a bound protein comprising a crossbeta structure. Said bound misfolded protein and/or crossbeta structure and/or bound protein comprising a crossbeta structure is preferably identified by analyzing at least part of the amino acid sequence of said misfolded protein and/or crossbeta structure and/or protein using any method known in the art. Said sample preferably comprises an aqueous solution, more preferably a body fluid. In one preferred embodiment body fluids originating from healthy individuals (preferably humans) and body fluids originating from individuals suffering from, or suspected to suffer from, a disease related to and/or associated with the presence of a misfolded protein and/or a crossbeta structure are used in order to compare a healthy state with a diseased state (or a state wherein the risk of disease is enhanced).

As described, the invention also provides a method for removing crossbeta structure comprising proteins, i.e. binding of a crossbeta structure comprising protein followed by removal of the formed complex. As described before, crossbeta structures and/or (misfolded) proteins comprising a crossbeta structure are an underlying cause of disease symptoms of many diseases. Again, this part of the invention may also be used for removal of a cross-structure precursor (comprising protein).

Said disease symptoms, related to the presence of crossbeta structures, are at least partly diminished by the administration of a chaperone (or a functional equivalent and/or a functional fragment thereof). Because crossbeta structure precursor can also be removed said method also has prophylactic application, because the formation of the toxic crossbeta structure is at least in part prevented.

A chaperone (or a functional equivalent and/or a functional fragment thereof is particularly suitable for removing proteins and/or peptides comprising a crossbeta structure (precursor), preferably related to and/or associated with a disease, from a sample such as for instance a body fluid or tissue sample, thereby decreasing the amount of (circulating) proteins and/or peptides comprising a crossbeta structure. As used herein, the term “removing a protein and/or peptide comprising a crossbeta structure” comprises separating said protein and/or peptide from a sample, as well as binding, covering, shielding and/or neutralizing a crossbeta structure and/or any other part of a protein or peptide comprising a crossbeta structure, thereby at least in part preventing interaction of said crossbeta structure and/or protein or peptide comprising a crossbeta structure with other binding molecules. This way, adverse effects related to the presence of a crossbeta structure and/or to the presence of a protein or peptide comprising a crossbeta structure, such as for instance infections and/or inflammation in AIDS, and/or disease symptoms of such painful and devastating diseases like for example rheumatoid arthritis and multiple sclerosis, are at least in part decreased. The same principle is also applicable to inflammatory conditions in which proteins are altered by the presence of a crossbeta structure (be it a crossbeta structure generated by the body or generated and/or induced by a pathogen). Thus, the invention further provides a method for removing a crossbeta structure comprising protein from a sample, said method comprising contacting said sample with a chaperone or a functional equivalent and/or a functional fragment thereof, allowing for binding of a crossbeta structure to said chaperone and removing the complex formed through binding. As described before, crossbeta structures in proteins are often related to, and/or associated with, a risk and/or presence of disease, such as for instance Huntington's disease, amyloidosis type disease, atherosclerosis, diabetes, bleeding, thrombosis, cancer, sepsis, inflammatory diseases, rheumatoid arthritis, transmissible spongiform encephalopathies such as Creutzfeldt-Jakob disease or BSE, Multiple Sclerosis, auto-immune diseases, diseases associated with loss of memory such as Alzheimer's disease, Parkinson's disease and other neuronal diseases (epilepsy), encephalopathy and systemic amyloidoses. Hence, a chaperone (or a functional equivalent and/or a functional fragment thereof) is particularly suitable for at least in part preventing and/or treating such crossbeta structure related and/or associated diseases. One embodiment therefore provides a chaperone (or a functional equivalent and/or a functional fragment thereof) for use as a medicament and/or prophylactic agent.

The invention furthermore provides use of a chaperone (or a functional equivalent and/or a functional fragment thereof) for the preparation of a medicament and/or prophylactic agent for the treatment of any of the mentioned diseases. Said medicament and/or prophylactic agent is particularly suitable for at least in part preventing, treating and/or stabilizing diseases that are related to and/or associated with occurrence of crossbeta structures, blood coagulation disorders, inflammation, and/or an infection by a microbe, pathogen, bacterium, parasite and/or virus. Further provided is therefore use of a chaperone (or a functional equivalent and/or a functional fragment thereof) for the manufacture of a medicament for at least partial prevention and/or treatment of a crossbeta structure related and/or associated disease, a blood coagulation disorder, immunological disorder, inflammation and/or a microbial/pathogen/parasite/bacterial/viral infection. A method for at least partial prevention and/or treatment of a crossbeta structure related and/or associated disease, a blood coagulation disorder and/or a microbial/pathogen/parasite/bacterial/viral infection in an individual, comprising administering an chaperone (or a functional equivalent and/or a functional fragment thereof) to said individual, is also herewith provided. In one preferred embodiment said microbial/pathogen/parasite/bacterial/viral infection comprises an opportunistic infection. This is an infection by an organism such as for instance a pathogen and/or virus that does not ordinarily cause disease but that, under certain circumstances (such as an impaired immune system), becomes pathogenic. An impaired immune system is for instance caused by medication such as chemotherapy. In a particularly preferred embodiment said microbial/pathogen/parasite/bacterial/viral infection comprises an HIV-related opportunistic infection. Since opportunistic infections are the major cause of death in HIV patients, it is highly desired to provide medicaments and/or prophylactic agents against such infections. Many opportunistic infections involve the presence of a crossbeta structure. For instance, amyloid structures occur on the surface of microbial organisms like fungi, yeast and bacteria. Said amyloid-like structures are generally called hydrophobins on fungi, chaplins on gram-positive bacteria, and curli or tafi or aggregative fimbriae on gram-negative bacteria. Since chaperone (or a functional equivalent and/or a functional fragment thereof) is particularly suitable for binding such crossbeta structures and/or proteins comprising a crossbeta structure, said chaperone (or a functional equivalent and/or a functional fragment thereof) is particularly suitable for counteracting and/or at least in part preventing HIV-related opportunistic infections. The invention therefore provides a method for at least partial prevention and or treatment of an HIV-related opportunistic infection in an individual, comprising administering a chaperone (or a functional equivalent and/or a functional fragment thereof) to said individual.

In a preferred embodiment, said chaperone (or a functional equivalent and/or a functional fragment thereof) is combined with at least one of any of the other known crossbeta structure binding compounds. Non-limiting examples of such compounds are listed in Table 1 or 2 or 3. The invention therefore also provides a (pharmaceutical) composition comprising a chaperone (or a functional equivalent and/or a functional fragment thereof) with at least one of the crossbeta structure binding compounds as mentioned in Table 1 or 2 or 3. Such a (pharmaceutical) composition is optionally further provided with a (pharmaceutical acceptable) suitable carrier, diluent and/or excipient. In order to be able to administer a medicament according to the present invention to a patient in need of treatment, said medicament must fulfil the needs for a pharmaceutically acceptable formulation. This means that a medicament according to the invention comprises a chaperone (or a functional equivalent and/or a functional fragment thereof) and at least one of any of the other known crossbeta structure binding compounds are of pharmaceutical grade, physiologically acceptable and tested for extraneous agents.

In another embodiment, the invention provides use of a chaperone (or a functional equivalent and/or a functional fragment thereof) as a crossbeta structure (precursor) binding compound and in yet another preferred embodiment the invention provides a chaperone (or a functional equivalent and/or a functional fragment thereof) with at least one improved crossbeta structure (precursor) binding domain or with multiple (improved) crossbeta structure (precursor) binding domains. For example, the C-terminal peptide-binding domain of BiP that is involved in binding to misfolded proteins serves as a starting point for development of improved crossbeta structure (precursor) binding compound.

The present invention furthermore provides means and methods for in general modulating and in specific increasing (preferably extra-cellular) protein degradation and/or protein clearance and/or protein neutralization in an individual. In a natural situation, the formation of crossbeta structure initiates and/or participates in a physiological cascade of events, dealing with removal of unwanted molecules and/or cells, such as for instance misfolded proteins, apoptotic cells, necrotic cells, died cells, cell debris or even pathogens. This pathway regulates the removal of unwanted biomolecules during several processes, including protein misfolding during synthesis in the endoplasmic reticulum, fibrinolysis, formation of neuronal synaptic networks, clearance of used, unwanted and/or destroyed (denatured) proteins, induction of apoptosis and clearance of apoptotic cells, necrotic cells, aged cells and/or pathogens. Since a chaperone (or a functional equivalent and/or functional fragment thereof is particularly suitable for binding crossbeta structure (or a precursor thereof) and proteins comprising crossbeta structure, (extra-cellular) protein degradation and/or protein clearance and/or protein neutralization is increased. Further provided is therefore a method for increasing (extra-cellular) protein degradation and/or protein clearance and/or protein neutralization in an individual, comprising administering a chaperone or a functional equivalent and/or a functional fragment thereof to said individual.

In yet another embodiment, the invention provides use of a chaperone or a functional equivalent and/or a functional fragment thereof for diminishing accumulation of misfolded protein comprising a crossbeta structure (precursor). In one embodiment, said misfolded protein comprising a crossbeta structure (precursor) is involved in a conformational disease. Diminishing accumulation of such proteins results in alleviation of symptoms of said conformational disease and/or at least partial treatment and/or prevention of the course of disease. The accumulation may be at least partly, but is preferably completely diminished. Said conformational disease preferably comprises an amyloidosis type disease, atherosclerosis, diabetes, bleeding, thrombosis, cancer, sepsis, inflammatory diseases, rheumatoid arthritis, transmissible spongiform encephalopathies, Multiple Sclerosis, auto-immune diseases, disease associated with loss of memory or Parkinson's disease and other neuronal diseases (epilepsy), encephalopathy, encephalitis, and/or rheuma.

In a preferred embodiment said chaperone or a functional equivalent and/or a functional fragment thereof is an hsp's such as hsp70, hsp90 or gp96. Even more preferred are extra-cellular chaperones, such as BiP, haptoglobin, hsp72 or clusterin and even more preferred are extra-cellular chaperones that are ATP-independent, such as haptoglobin or clusterin. In yet another preferred embodiment, an improved chaperone is used (i.e. a chaperone molecule with improved crossbeta structure (precursor) binding affinity).

The invention also provides use of a chaperone or a functional equivalent and/or a functional fragment thereof for determining the presence of a plaque and/or a deposition and/or accumulated misfolded protein involved in a conformational disease. Preferably, said disease is an amyloidosis-type of disease, for example Alzheimer's Disease or diabetes or a systemic amyloidosis.

In yet another preferred embodiment, the invention provides use of a chaperone (or a functional equivalent and/or a functional fragment thereof) for the removal of crossbeta structures from a sample and even more preferably from an individual. In one preferred embodiment said sample comprises a body fluid. This embodiment is particularly suitable for at least in part preventing and/or treating a crossbeta structure related and/or associated disorder of an animal, preferably of a human individual. In one preferred embodiment extracorporeal dialysis is applied. For example, a patient suffering from a crossbeta structure related and/or associated disorder is subjected to dialysis of his/her blood. A chaperone (or a functional equivalent and/or functional fragment thereof) is for instance coupled to a carrier or support and/or to the inside of a tube used for dialysis. This way, crossbeta structure and proteins comprising a crossbeta structure are removed from the blood stream of said patient, thereby at least in part relieving said patient of negative effects related to, and/or associated with, said crossbeta structure and/or proteins comprising a crossbeta structure. As another example, such use is applied in haemodialysis of renal disease patients. A separation device for carrying out a method according to the invention is also provided. One embodiment thus provides a separation device for carrying out a method according to the invention, said device comprising a system for transporting (circulating) fluids (preferably ex vivo), said system being provided with means for connecting to a flowing fluid, preferably to an individual's circulation, means for entry of fluid into said system and return of fluid from said system, preferably to an individual's circulation, said system further comprising a solid phase, said solid phase comprising a chaperone or a functional equivalent and/or functional fragment thereof. Optionally said chaperone is combined with any one or more of the crossbeta structure binding compounds as listed in Table 1 or 2 or 3. Said separation device preferably comprises a dialysis apparatus. Besides ex vivo uses, said separation device is also very suitable for in vitro application, for example for treating pharmaceutical compositions.

Coagulation of blood and blood platelet clot formation also involves the presence of crossbeta structure. Examples of the role of (misfolded) protein comprising crossbeta structure are activation of platelets and induction of platelet aggregation and agglutination, activation of endothelium resulting in tissue factor expression and exposure to blood, resulting in blood coagulation, and activation of the contact system of blood coagulation via activation of factor XII. In addition, during blood coagulation fibrin polymers with crossbeta structure conformation are formed. The crossbeta structure building block of a fibrin network subsequently serves as the binding site for tPA to localize tPA at the site where fibrinolytic activity is required. Since a chaperone (or a functional equivalent and/or a functional fragment thereof) is capable of specifically binding and/or removing crossbeta structure(s) (precursors) and proteins comprising crossbeta structure or precursors thereof, said chaperone is particularly suitable for interfering in coagulation of blood and/or clot formation and/or activation of tissue factor. Further provided is therefore a method for interfering in coagulation of blood and/or in aggregation of platelets and/or in fibrinolysis and/or clot formation comprising providing to blood a chaperone (or a functional equivalent and/or a functional fragment thereof). Interfering in blood clot formation is for example established by inhibiting fibrin polymer formation upon binding of chaperone to an early-stage fibrin polymer. In this way, thrombus formation is prevented during a hyper-coagulable state, which reduced the risk for thrombosis. In yet another approach, during circumstances accompanied by a bleeding tendency, providing a chaperone prevents interaction of tPA with a preformed blood clot, thereby reducing (hyper)fibrinolytic activity. Tissue factor expression on cells upon exposure of cells to crossbeta structure may result in a hyper-coagulable state or even a thrombogenic state. Therefore, inhibiting the interaction between cellular receptors involved in activation of the tissue factor pathway and crossbeta structure by administering a chaperone will reduce the extent of blood coagulation. Blood platelet aggregation that is induced by crossbeta structure may result in thrombotic events. Therefore, inhibiting the contact between cell surface receptors for crossbeta structure on the platelets and crossbeta structure by chaperone reduces the risk for thrombosis, as a result of reduced crossbeta structure mediated platelet activation.

Such a method may be performed in vitro as well as in vivo. Thus, the invention also provides use of a chaperone or a functional equivalent and/or a functional fragment thereof in the preparation of a medicament for the treatment of thrombosis, bleeding disorders, hyper-fibrinolysis, crossbeta structure induced platelet aggregation or a hyper-coagulable state.

In a preferred embodiment, a chaperone (or a functional equivalent and/or a functional fragment thereof) is combined with any one or more of the other known crossbeta structure binding compounds that are non-limiting listed in Table 1 or 2 or 3, such as for example combinations of chaperone with CD36, LRP, apoER2′, scavenger receptor A, scavenger receptor B-I, RAGE, FEEL-1, FEEL-2, SREC-1, LOX-1, stabilin-1 or stabilin-2.

In yet another preferred embodiment a chaperone (or a functional equivalent and/or a functional fragment thereof) is used in the field of (bio)pharmaceutical preparations. Important categories of nowadays pharmaceutical compositions comprising a protein or a proteinaceous compound as an active substance include, but are not limited to hormones, enzymes, vaccines and antigens, cytokines and antibodies. In addition to the above-mentioned proteinaceous pharmaceutical compositions, a large number of pharmaceutical compositions are manufactured with the help of a production and/or purification step comprising proteins. For example, many pharmaceutical compositions comprise one or more proteins as a stabilizing agent. Health problems related to the use of pharmaceutical compositions are for example related to the fields of hematology, fibrinolysis and immunology. An incomplete list of observed side-effects after administration of pharmaceutical compositions comprises for example fever, anaphylactic responses, (auto)immune responses, disturbance of haemostasis, inflammation, fibrinolytic problems, including sepsis and disseminated intravascular coagulation (DIC), which can be fatal. Said side effects are for instance caused by either an alteration of a protein or a proteinaceous compound present in said pharmaceutical composition, or by added diluents, excipients or carrier substances of said pharmaceutical composition. Alteration of a proteinaceous compound of a pharmaceutical composition comprises for example denaturation, multimerization, proteolysis, acetylation, glycation, oxidation, unfolding or misfolding of proteins. Unfolding or misfolding of initially properly folded native proteins leads to the formation of toxic structures in said proteins. Toxic structures of pharmaceutical compositions often comprise crossbeta structures. Now that the present inventors have shown that a chaperone (or a functional equivalent and/or a functional fragment thereof) is capable of binding crossbeta structure (or precursors thereof), this feature of a chaperone (or a functional equivalent and/or a functional fragment thereof) is for example used to for example (i) detect a protein and/or a peptide comprising a crossbeta structure in a pharmaceutical composition or to (ii) control a manufacturing process, and/or storage process of a pharmaceutical composition or to (iii) remove or shield a protein and/or peptide comprising a crossbeta structure from a (bio)pharmaceutical composition or to (iv) decrease and/or prevent an undesired side effect of a (bio)pharmaceutical composition and or to increase the specific activity per gram protein.

Thus, in a preferred embodiment the invention provides a method for removing a crossbeta structure and/or protein comprising a crossbeta structure from a pharmaceutical composition or any of its constituents comprising a protein, said method comprising:
contacting said pharmaceutical composition or any of its constituents comprising a protein with a chaperone (or a functional equivalent and/or a functional fragment thereof);
allowing binding of said crossbeta structure and/or protein comprising a crossbeta structure to said chaperone (or a functional equivalent and/or a functional fragment thereof); and
separating bound crossbeta structure and/or bound protein comprising a crossbeta structure from said pharmaceutical composition or any of its constituents comprising a protein. Such a method is also applied for removing a crossbeta structure precursor and/or a protein comprising a crossbeta structure precursor.

By removing or shielding a crossbeta structure (precursor) and/or a protein comprising a crossbeta structure (precursor) from a pharmaceutical composition, undesired side effects are at least in part decreased and/or prevented. Also provided is therefore a method for decreasing and/or preventing undesired side effects of a pharmaceutical composition and/or increasing the specific activity per gram protein, said method comprising removing an unfolded protein, an unfolded peptide, a misfolded protein, a denatured protein, an aggregated protein, an aggregated peptide, a multimerized protein and/or a multimerized peptide, comprising a crossbeta structure (precursor), from said pharmaceutical composition or any of its constituents, by using the steps as listed above.

In yet another embodiment, the invention provides a method for controlling a manufacturing process, and/or storage process of a pharmaceutical composition or any of its constituents comprising a protein, said method comprising:
contacting said pharmaceutical composition or any of its constituents comprising a protein with at least one chaperone or a functional equivalent and/or a functional fragment thereof resulting in a bound protein and/or peptide comprising a crossbeta structure,
detecting whether bound protein and/or peptide comprising a crossbeta structure is present in said pharmaceutical composition or any of its constituents comprising a protein at various stages of said manufacturing and/or storage process

Optionally, the conditions are adjusted such as to minimize the amount of formed crossbeta structure during the manufacturing process and/or storage process. Again, such a method is also useful for a precursor of a crossbeta structure.

A pharmaceutical composition or any of its constituents comprising a protein, obtainable by a method according to the invention is also herewith provided. Said pharmaceutical composition involves a reduced risk of undesired side effects as compared to untreated pharmaceutical compositions.

To for example determine whether a crossbeta structure is present in a pharmaceutical or to determine the amount of present crossbeta structure, the invention further provides a method for detecting a protein and/or peptide comprising a crossbeta structure in a pharmaceutical composition or any of its constituents comprising a protein, said method comprising:
contacting said pharmaceutical composition or any of its constituents comprising a protein with at least one chaperone or a functional equivalent and/or a functional fragment thereof resulting in a bound protein comprising a crossbeta structure,
detecting whether bound protein comprising a crossbeta structure is present in said pharmaceutical composition or any of its constituents comprising a protein.

This method is also suitable for determining the amount of present crossbeta structure precursors.

In a preferred embodiment said chaperone or a functional equivalent and/or a functional fragment thereof is an hsp such as hsp70, hsp90 or gp96. Even more preferred are extra-cellular chaperones, such as BiP, haptoglobin, hsp72 or clusterin and even more preferred are extra-cellular chaperones that are ATP-independent, such as haptoglobin or clusterin. In yet another preferred embodiment, an improved chaperone is used (i.e. a chaperone molecule with improved crossbeta structure (precursor) binding affinity) and in yet another preferred embodiment, said chaperone is combined with a known crossbeta structure binding compound, for example one or more of the compounds that are listed in Table 1 or 2 or 3.

In yet another preferred embodiment, a chaperone (or a functional equivalent and/or functional fragment thereof) is attached to a solid phase after or before binding a protein comprising a crossbeta structure. As a solid phase, many materials are suitable for binding a crossbeta structure-binding compound, such as for example, glass, silica, polystyrene, polyethylene, nylon, vinyl, agarose beads, Sepharose matrix, beads containing iron or other metals and so on. In one embodiment of the invention, said solid phase has the physical form of beads. In another embodiment said solid phase has the shape of a tube or a plate or a well in, for instance an ELISA plate, or a dipstick. Numerous binding techniques are available for coupling the crossbeta structure-binding compounds to said solid phase, like for example, CyanogenBromide (CNBr), NHS, Aldehyde, epoxy, Azlactone, biotin/streptavidin, Universal Linkage System, and many others.

In yet another embodiment, the invention provides applications in the field of vaccines. The present invention provides methods and means which improve the immunogenicity of compositions intended to elicit an immune response. In order to determine whether a vaccine composition can be improved in immunogenicity by providing said composition with (further) crossbeta structure, one determines the amount of crossbeta structure already present therein by means as disclosed herein, particularly by binding with a crossbeta structure binding compound, such as BiP or clusterin or haptoglobin. In a preferred manner said amount of crossbeta structure is determined by binding of a crossbeta structure binding compound (BiP, hsp72, clusterin, haptoglobin), and detecting the amount of bound crossbeta structure in a manner known per se and determining whether adding further crossbeta structure improves the immune response. It is clear to a skilled person, that also an improved chaperone or a combination of a chaperone (or a functional equivalent and/or a functional fragment thereof) with any one or more of the crossbeta structure binding compounds as listed in Table 1 or 2 or 3 can be used in a method according to the invention. Thus the invention further provides a method for determining the amount of crossbeta structure in a vaccine composition, comprising contacting said vaccine composition with at least one chaperone or a functional equivalent and/or a functional fragment thereof and relating the amount of bound crossbeta structure to the amount of crossbeta structure present in the vaccine composition. The invention further provides a method for enhancing immunogenicity of a vaccine composition comprising at least one peptide, polypeptide, protein, glycoprotein and/or lipoprotein, comprising contacting at least one of said peptide, polypeptide, protein, glycoprotein and/or lipoprotein with a crossbeta structure inducing agent, thereby providing said vaccine composition with additional crossbeta structure. The amount of induced crossbeta structure is then for example established by a method for determining the amount of crossbeta structure in a vaccine composition as described herein.

Because the present invention provides the insight that a chaperone (or a functional equivalent and/or a functional fragment thereof) is capable of binding to a crossbeta structure (precursor) a skilled person is now also capable of using said chaperone, in order to determine whether a protein or peptide comprising a crossbeta structure (precursor) is present in a sample. Provided is therefore a method for determining whether a protein and/or peptide comprising a crossbeta structure and/or a molecule comprising a crossbeta structure and/or a molecule comprising a crossbeta structure precursor is present in an aqueous solution comprising a protein, said method comprising:
contacting said aqueous solution comprising a protein with at least one chaperone or a functional equivalent and/or a functional fragment thereof
detecting whether bound protein and/or peptide comprising a crossbeta structure is present.

Said protein and/or peptide is preferably detected in an aqueous solution by contacting said aqueous solution with a chaperone or a functional equivalent and/or a functional fragment thereof and detecting bound peptides and/or proteins. Binding of said chaperone or a functional equivalent and/or a functional fragment thereof to a crossbeta structure is preferably detected by means of a visualization reaction as for example by fluorescent staining or an enzymatic or calorimetric detection, or by any other visualization system available to a skilled person.

Said aqueous solution preferably comprises a detergent, a food product, a food supplement, a cell culture medium, a commercially available protein solution used for research purposes, blood, a blood product, a body fluid like for example urine, cerebrospinal fluid, synovial fluid, lymph fluid and/or sputum, a cosmetic product, a cell, a pharmaceutical composition or any of its constituents comprising a protein, or a combination of any of these.

The invention also relates to the field of microbiology, more specifically to antimicrobial medicines and antimicrobial vaccines.

Amyloid structures occur on the surface of microbial organisms like fungi and bacteria. Although the proteins in amyloid differ, the resulting fibrils of amyloid-like structures contain crossbeta structure. Said amyloid-like structures are generally called hydrophobins on fungi, chaplins on Gram-positive bacteria, and curli or tafi or aggregative fimbriae on Gram-negative bacteria. Since resistance of micro-organisms to antibiotic and bacteriostatic compounds is an ever-increasing problem, researchers are always looking for new methods for combating micro-organisms. The presence of said amyloid proteins comprising a crossbeta structure renders a bacterium or fungus more virulent for a host. The identification of crossbeta structure in said surface proteins of micro-organisms opens new methods for decreasing the virulence of a micro-organism and offers new methods for inhibiting infection of a host by said micro-organism. Now that the inventors have disclosed that a chaperone (or a functional equivalent and/or a functional fragment thereof) is capable of binding to crossbeta structure, a chaperone (or a functional equivalent and/or a functional fragment thereof) also decreases the pathogenicity and is therefore a potent inhibitor of a pathogen infection. Therefore, the present invention also provides the use of a chaperone (or a functional equivalent and/or a functional fragment thereof) in the preparation of a medicament for the treatment of a microbial infection. In a preferred embodiment, said microbial infection is caused by a micro-organism that is a pathogenic micro-organism, such as a fungus or a Gram-positive bacterium (for example an actinomycete or a streptomycete). In a more preferred embodiment, said crossbeta structure comprises a hydrophobin or a chaplin. In yet another embodiment, said micro-organism is a Gram-negative bacterium, such as is an E. coli bacterium or a Salmonella bacterium. In a preferred embodiment, the invention also provides a composition comprising a chaperone (or a functional equivalent and/or a functional fragment thereof) and at least one of the crossbeta structure binding compounds as listed in Table 1 or 2 or 3. The invention further provides a kit for detecting microbial contamination of a solution and/or a substance, said kit comprising chaperone (or a functional equivalent and/or a functional fragment thereof) and a means for detecting binding of said crossbeta structure to said binding compound.

Nowadays, the list of proteins and peptides that are known to be able to adopt the amyloid-like crossbeta structure conformation is tremendous. This has led to the idea that refolding of polypeptides from a native fold to an amyloid-like structure is an inherent property, independent of the amino-acid sequence of the polypeptides. To be able to further study the role of the crossbeta structure in (patho)physiology it is necessary that (more) compounds capable of binding to a protein comprising a crossbeta structure, amongst others crossbeta structure binding compounds, are identified. Such compounds are not only useful to be able to better understand crossbeta structure, but are also very useful in respect of understanding the refolding from a native state, assembly and toxicity, and are also useful for the development of diagnostic and therapeutic agents or useful as component of a diagnostic or therapeutic agent.

Now that the inventors have disclosed that a chaperone or a functional equivalent and/or a functional fragment thereof is capable of binding to a crossbeta structure (precursor), this feature of a chaperone is also used to identify new crossbeta structure binding compounds.

Therefore, the invention provides in yet another embodiment a method for selecting a compound capable of binding to a crossbeta structure in a protein, comprising
contacting said compound with a first protein comprising a crossbeta structure and allowing said compound and said protein to interact
determining with a chaperone or a functional equivalent and/or a functional fragment thereof whether said compound at least in part binds to said crossbeta structure
selecting the compound that at least in part binds to said crossbeta structure.

Such a method is optionally combined with already known methods to determine whether a protein comprises a crossbeta structure conformation. Examples of such methods include, but are not limited to staining with Congo red, Thioflavin S (ThS) or Thioflavin T (ThT) (or by using any of the compounds listed in Table 1 or 2 or 3), an ELISA binding assay using tPA or a functional fragment thereof, or an enzymatic assay such as a tPA activation assay, a factor XII activation assay or a X-ray fiber diffraction analysis.

In a preferred embodiment, said determining step is a competition assay between said compound, a first protein comprising a crossbeta structure and chaperone or a functional equivalent and/or a functional fragment thereof. In a preferred embodiment an enzymatic competition assay is performed. An example of an enzymatic assay is the measurement of ATPase activity in a solution. ATPase activity of a chaperone bound to crossbeta structure will be reduced when a competitor molecule releases a chaperone from the crossbeta structure.

Besides a compound that is capable of binding to a crossbeta structure in a protein or at least to a part thereof, the above outlined method is also used to select a binding compound that does not bind to the crossbeta structure itself but to another structure in a protein which other structure is only present in a protein that comprises a crossbeta structure and which other structure is absent if said protein does not comprise a crossbeta structure. Such other structure is further referred to as a crossbeta structure induced conformation. Hence, the invention also provides a method for selecting a compound capable of binding to a crossbeta structure induced conformation in a protein comprising a crossbeta structure, comprising
contacting said compound with a first protein comprising a crossbeta structure and allowing said compound and said protein to interact,
determining with a chaperone or a functional equivalent and/or a functional fragment thereof whether said compound binds to a crossbeta structure induced conformation
selecting a compound that binds to a crossbeta structure induced conformation.

In another embodiment said first protein comprising a crossbeta structure is provided with a label to, for example, facilitate identification. Examples of suitable labels are Universal Linkage System (ULS™), maltose binding protein, glutathione S-transferase (GST), secreted human placental alkaline phosphatase (SEAP), His-tag, biotin, green fluorescent protein, (horse raddish) peroxidase, FLAG, myc, VSV. Immobilization and labelling of a chaperone or a functional equivalent and/or a functional fragment thereof is also possible.

In a preferred embodiment, a method according to the invention further comprises performing a subtraction or inhibition assay with a second protein comprising a crossbeta structure and selecting the compound that specifically binds to said first protein. In addition, a method according to the invention further comprises selecting the compound that at least in part binds to said compound with crossbeta structure, further comprising performing binding assays with a series of different compounds comprising a crossbeta structure and selecting the compound that specifically binds to said first protein.

With this method a crossbeta structure comprising protein binding compound specific for said first compound is selected. Preferably multiple second proteins are tested to improve/establish the selectivity of said crossbeta structure binding compound for said first protein. Such a specific compound is extremely useful for diagnostic and therapeutic application and will be discussed in more detail below.

A crossbeta structure binding compound or a crossbeta structure induced conformation binding compound obtainable according to a method of the invention provides means and methods for the detection or treatment of diseases associated with the formation of cross-β structure, such as, but not limited to, amyloidosis, and include Alzheimer's disease (AD), light-chain amyloidosis, type II diabetes and spongiform encephalopathies.

A crossbeta structure binding compound or a crossbeta structure induced conformation binding compound obtainable according to a method of the invention is useful in methods to detect a compound with cross-β structure. In one embodiment such a binding compound is bound or affixed to a solid surface, preferably a microtiter plate or preferably a chip of a surface plasmon resonance apparatus or preferably a microarray chip. The solid surfaces useful in this embodiment would be known to one of skill in the art. For example, one embodiment of a solid surface is a bead, a column, a plastic or polymer dish, a plastic or polymer plate, a microscope slide, a nylon membrane, a chip, etc. (After blocking) the surface is incubated with a sample. (After removal of unbound sample) bound molecules comprising the cross-β structure are subsequently detected using a second cross-β structure binding compound, preferably an anti-cross-β structure antibody or a molecule containing a finger module or a chaperone. The second cross-β structure binding compound is bound to a label, preferably an enzyme, such as peroxidase. The detectable label may also be a fluorescent label, biotin, digoxigenin, a His-tag, a SEAP tag, a Myc tag, a VSV tag, a FLAG tag, an MPB tag, a GST tag, a radioactive atom, a paramagnetic ion, or a chemiluminescent label. It may also be labelled by covalent means such as chemical, enzymatic or other appropriate means with a moiety such as an enzyme or radioisotope. Portions of the above mentioned compounds of the invention may be labelled by association with a detectable marker substance, preferably radiolabeled with ¹²⁵I or biotin to provide reagents useful in detection and quantification of compound or its receptor bearing cells or its derivatives in solid tissue and fluid samples such as blood, cerebrospinal spinal fluid, urine or other. Such samples may also include serum used for tissue culture or medium used for tissue culture.

In another embodiment the solid surface can be microspheres for, for example, agglutination tests.

Since misfolded proteins comprising a crossbeta structure are effectively bound to chaperones, they are effectively separated and isolated from a sample and/or an animal's or human's body. In yet another embodiment therefore, a selected chaperone according to the invention is used to isolate misfolded proteins comprising a crossbeta structure from body fluids, like for example blood, serum, plasma, cerebrospinal fluid, synovial fluid, sputum, urine, of healthy individuals and from individuals with any disease accompanied by crossbeta structure and/or protein comprising crossbeta structure. The identity and the relative concentration of each of the misfolded proteins comprising crossbeta structure are subsequently determined using methods known to a person skilled in the art of Proteomics, like for example 2D gel electrophoresis and/or mass-spectrometric analyses after (partial) proteolytic digestion, and results with samples originating from healthy individuals and from patients are compared. In this way, information is obtained about the identity and susceptibility of proteins prone to misfold and adopt cross-β structure conformation during defined disease states. This information may serve as a diagnostic tool to monitor disease state, to monitor effectiveness of therapy, to monitor occurrence of diseases, and provides valuable leads for development of therapeutics targeted at misfolded protein(s), perhaps specific for a defined disease.

In one embodiment a crossbeta structure binding compound or a cross-structure induced conformation binding compound is used to stain tissue samples.

In one of the embodiments the above sample is obtained from tissue from patients with or expected to suffer from a conformational disease, e.g. rheumatoid arthritis or systemic AL amyloidosis.

A crossbeta structure binding compound or a crossbeta structure induced conformation binding compound obtainable according to a method of the invention is also useful as part of a new diagnostic tool. Such use is particular useful for diagnostic identification of conformational diseases or diseases associated with amyloid formation, like AD or diabetes. It is clear that this diagnostic use is also useful for other diseases and processes which involve cross-β structure formation, like all amyloidosis type diseases, atherosclerosis, diabetes, bleeding, thrombosis, renal failure with kidney dialysis regime, cataract, multiple myeloma, lymphoma or sepsis and complications thereof such as disseminiated intravascular coagulation (DIC).

Based on the finding that a chaperone or a functional equivalent and/or a functional fragment thereof binds to a crossbeta structure, the invention furthermore provides a method for determining the effect of a certain condition on the crossbeta structure content of a protein. Such a method is for example extremely useful in determining the biocompatibility of materials.

In a first embodiment, the invention provides a method for determining a difference in the crossbeta structure content of a protein in a reference sample compared to said protein in a test sample wherein the test sample has been subjected to a treatment that is expected to have an effect on the crossbeta structure content of said protein, the method comprising
determining in a reference sample the crossbeta structure content of a protein
subjecting said protein to a treatment that is expected to have an effect on the crossbeta structure content of said protein, thus obtaining a test sample
determining in the obtained test sample the crossbeta structure content of said protein
determining whether the crossbeta structure content of the reference sample is different from the crossbeta structure content in the test sample, characterised in that at least one of the determination steps is performed by using a chaperone or a functional equivalent and/or fragment thereof.

Preferably the determined difference is considered to be significantly as judged by standard statistical techniques.

The method according to the invention can be performed qualitatively as well as quantitatively and hence reference to crossbeta structure content of a protein or reference/test value or point is herein defined as to cover both a quantitative assay as well as a qualitative assay.

As described above, a particular useful embodiment is a method for selecting a circumstance and/or a treatment and/or a condition that does not induce crossbeta structure conformation in a protein or that does not change the crossbeta structure content of a protein. Hence, in a preferred embodiment the invention provides a method for selecting a treatment that essentially preserves the structure of a protein comprising
determining in a reference sample the crossbeta structure content of said protein
subjecting said protein to a treatment that is expected to have an effect on the crossbeta structure content to obtain a test sample
determining in said test sample the crossbeta structure content of said protein
selecting the treatment that essentially preserves the structure of said protein, characterised in that at least one of the determination steps is performed by using a chaperone or a functional equivalent and/or fragment thereof.

In yet another preferred embodiment, the invention provides a method for selecting a treatment that essentially preserves the crossbeta structure content of a protein comprising
determining in a reference sample the crossbeta structure content of said protein
subjecting said protein to a treatment that is expected to have an effect on the crossbeta structure content to obtain a test sample
determining in said test sample the crossbeta structure content of said protein
selecting the treatment that essentially preserves the crossbeta structure content of said protein, characterised in that at least one of the determination steps is performed by using a chaperone or a functional equivalent and/or fragment thereof.

In yet another preferred embodiment, the invention provides a method for selecting a treatment that essentially increases the crossbeta structure content of a protein comprising
determining in a reference sample the crossbeta structure content of said protein
subjecting said protein to a treatment that is expected to have an effect on the crossbeta structure content to obtain a test sample
determining in said test sample the crossbeta structure content of said protein
selecting the treatment that essentially increases the crossbeta structure content of said protein, characterised in that at least one of the determination steps is performed by using a chaperone or a functional equivalent and/or fragment thereof.

This selection method is for example useful for the selection of an antigen composition, for example for the use as a vaccine, in which at least one of the proteinaceous components of the composition comprises crossbeta structure conformation.

The sample (or the to be tested) protein can take different forms. For example, said protein may be in a dried, solid form and the to be tested treatment comprises different reconstitution buffers or different storage conditions (for example different humidity conditions and the effect of said humidity on for example the activity of said protein). In a preferred embodiment, said protein is a protein in a solution. In an even more preferred embodiment said solution is a body fluid, such as blood or lymph fluid, or cerebrospinal fluid or synovial fluid or a part derived thereof (for example plasma). In yet another preferred embodiment the protein is part of a cell (for example a surface protein) or a constituent of tissue or an extra-cellular matrix protein. In case the protein is part of a more solid sample, said sample may further be subjected to a homogenization step.

Said protein (in solution or as part of a cell, either or not in tissue or in matrix) may be a single type of protein or a mixture of proteins (possibly in solution). Detection of single types of proteins and mixtures of protein is described in more detail later on.

As outlined above, at least one of the determination steps is performed by using a chaperone or a functional equivalent and/or fragment thereof. It is clear that also both determination steps may be performed by using a chaperone or a functional equivalent and/or fragment thereof If only one determination steps is performed by using chaperone or a functional equivalent and/or fragment thereof, the other step is preferably performed by using any one or more of the crossbeta structure binding compounds as listed in Table 1 or 2 or 3.

The step of determining the crossbeta structure content generally comprises the immobilisation of a crossbeta structure binding compound (for example a chaperone or a functional equivalent and/or fragment thereof) on a solid surface followed by contacting a sample (either or not exposed to a treatment that is expected to have an effect on the crossbeta structure content) with said immobilised crossbeta structure binding compound and detection of the bound crossbeta structure comprising protein with (another) crossbeta structure binding compound, or via specific detection of the crossbeta structure comprising protein, for example by applying a specific antibody, or via a-specific protein detection of the protein comprising crossbeta structure, for example by using protein quantification methods available to the skilled person.

As an example, in complex mixtures, the crossbeta structure content of each individual protein can be assessed by contacting the mixture to, for example, a solid surface with an immobilized crossbeta structure binding compound (for example a chaperone or a functional equivalent and/or fragment thereof), followed by an isolation step and a washing step, finalized by contacting the solid surface with an immobilized crossbeta structure binding compound and putatively various bound proteins, individually with antibodies specific for the putatively various bound proteins, that comprise crossbeta structure conformation.

The successful (medical) application of solid surfaces like for example the application of solid surfaces in heart valves, heart aid devices (pacemaker), heart pumps, haemodialysis membranes, (closed loop) insulin delivery system, artificial implant applications, medical devices, equipment used during heart operations, extracorporeal device, cardiopulmonary bypass devices, prosthetic devices, bone implants, artificial organs, vascular grafts, vascular prostheses, stents, storage vials/containers, syringes, tubings, bags, depend largely on their biocompatibility. Such devices are for example prepared from carbons, glass, ceramics polymers, hydrogels, collagen, polyurethanes, negatively charged polyamide, polystyrene, stainless steel, (carbon-coated) polytetrafluoroethylene, titanium, aluminium, iridium, indium, nickel, tantalum, tin, zirconium, Dacron, and presently, heparin or albumin-heparin conjugate is widely used as a clinical anticoagulant on such devices. The invention now provides a method to test (known and established or newly designed/produced) solid surfaces for their biocompatibility.

In one preferred embodiment, the invention provides a method for selecting a biocompatible material that essentially preserves the crossbeta structure content of a protein comprising
determining in a reference sample the crossbeta structure content of said protein
contacting said protein with a biocompatible surface that is expected to have an effect on the crossbeta structure content to obtain a test sample
determining in said test sample the crossbeta structure content of said protein
selecting the biocompatible material that essentially preserves the crossbeta structure content of said protein, i.e. selecting the material that does not increase the crossbeta structure content of a protein, preferably a protein solution (for example blood). Such a method is characterised by that at least one determining step is performed by using a chaperone or a functional equivalent and/or fragment thereof.

A suitable/selected (coated) biocompatible material obtainable by a method according to the invention or a biocompatible material designed on the above described findings is preferably used for preparing a biocompatible part/device/material/product. Non-limiting examples of a biocompatible part/device/material/product is a stent, heart valves, heart aid devices (pacemaker), heart pumps, haemodialysis membranes, (closed loop) insulin delivery system, vascular grafts, artificial implant applications, medical devices, equipment during heart devices, extracorporeal (circulation) device, cardiopulmonary bypass devices, prosthetic devices, bone implants, artificial organs, organ/body fluid storage devices or vascular prostheses

In yet another embodiment, the invention provides a method for selecting a material suitable for the interior of a storage device that essentially preserves the crossbeta structure content of a protein comprising
determining in a reference sample the crossbeta structure content of said protein
subjecting said protein to a material suitable for the interior of a storage device that is expected to have an effect on the crossbeta structure content to obtain a test sample
determining in said test sample the crossbeta structure content of said protein
selecting the material suitable for the interior of a storage device that essentially preserves the crossbeta structure content of said protein, characterised in that at least one determining step is performed by using a chaperone or a functional equivalent and/or fragment thereof.

In a preferred embodiment, the subjecting step comprises contacting said protein with a material suitable for the interior of a storage device.

Examples of useful applications of a method according to the invention are provided above and even more examples are provided below. In general it can be said that if one wants to study or obtain a protein with a particular property, it is important to check (if possible) each and every treatment on their crossbeta structure inducing capabilities on said protein. If for example a protein is used in the food industry it is important to check the production, purification and storage conditions. If one wants to study the activity of a protein (for example an enzyme) it is important to study all the conditions to which such a protein is subjected.

Other, non-limiting, applications of a method according to the invention are
testing of conditions for growing crystals for protein crystallography purposes; some of the presently used conditions result in the formation of crossbeta structure conformation in a protein and hence hamper the growth of high-quality crystals of said protein; conditions (to be) used in crystallography are now tested for their crossbeta structure inducing capability and a selection is made for conditions that do not or hardly not induce the formation of crossbeta structure conformation in a protein;
testing of materials used in protein purifications; independent of the source of protein (naturally expressed or recombinantly expressed) proteins are typically subjected to one or multiple purification steps to obtain high grade (pharmaceutical) preparations. All material used in such purifications, such as column material, tubings, vials, buffers, dialysis membranes, membranes used for concentration, is checked with a method according to the invention and materials and conditions are selected that do not or hardly not induce crossbeta structure conformation formation in the to be purified protein;
testing of conditions for protein refolding from an aggregated state to a native fold; independent of the source of the protein with non-native fold (naturally expressed or recombinantly expressed; for example Escherichia coli inclusion bodies), proteins are typically subjected to exposure to one or more solutions that putatively aid the folding from a non-native fold to a native fold. The solutions are now checked with a method according to the invention for their propensity to induce the crossbeta structure conformation in proteins by testing the content of crossbeta structure conformation in the proteins after the exposure to the solutions. Solutions can now be selected that do not result in crossbeta structure conformation and thus may aid the adoption of a native fold.
selection and development of cell culture disposables or laboratory equipment in general.

It is clear that if a certain crossbeta structure inducing treatment cannot be avoided it is possible to remove induced crossbeta structures in a protein. This is explained in more detail in this application and in our co-pending applications.

Examples of a treatment are a physical or mechanical treatment or a biochemical or chemical treatment. Examples of a physical or mechanical treatment comprises freezing or thawing or lyophilization of said protein or subjecting said protein to cold or heat or radiation such as X-rays, UV, IR, or subjecting said protein to pressure or air or vortexing or sonication or stirring or swirling or shaking or any combination thereof. Examples of a biochemical or chemical treatment comprises subjecting said protein to water or high pH or low pH or to a buffer solution or to a liquid comprising a protein or to a liquid medium or to ion strength or to osmosis or to an organic or inorganic detergent or to a radical or contacting said protein with a solid surface (such as metal or plastic or wooden or glass or cotton or silk surface or any combination thereof), or a (coated) biocompatible material or any combination thereof.

As mentioned earlier, HSPs are also found extra-cellular, and HSPs have immunological properties. This property was first described for the endoplasmic-reticulum resident—glucose-regulated HSP gp96 and subsequently also observed for hsp70, hsp90, calreticulin, hsp170 and hsp110. Importantly, the antigenic specificity of the HSPs was shown to derive not from the HSP molecules per se, but from the peptides chaperoned by them. First suggested in 1989-1991, this idea has been validated through a large number of structural and immunological studies. The mechanism, through which HSPs, or more accurately HSP-peptide complexes, elicit immunological responses, has been delineated in some detail. The interaction of HSPs with HSP receptors on antigen-presenting cells (APCs) lies at the centre of this mechanistic understanding. The following receptors have been identified on APCs: CD36, LOX-1, CD91 (LRP), scavenger receptor A (SR-A), CD14, CD40, Toll-like repceptor 2 and 4 (TLR-2, TLR-4). Intriguingly, almost all of these receptors are multiligand receptors which are implicated in the binding and/or uptake of misfolded amyloidogenic proteins, and belong to the Crossbeta Pathway as disclosed previously by the current inventors in 2002 in patent application EP1536778.The exact nature of the interaction of HSPs as well as other receptors with its ligands was unknown and is part of the invention(s) disclosed.

It is known that certain compositions of a molecular chaperone and an antigen can lead to delivery of said antigen to an antigen presenting cell. However, many antigens do not bind sufficiently well to a chaperone for them to be efficiently delivered to an antigen-presenting cell. The present invention provides a method for providing an immune response in a subject against a certain antigen. This is achieved by the step of inducing a crossbeta structure (precursor) in the used antigen and hence by promoting non-covalent binding between said antigen and said chaperone. Said chaperone is preferably a mammalian chaperone and even more preferably originate from the same species as that of the subject to be treated.

In yet another embodiment, the invention provides a method for producing an immunogenic composition, wherein said composition comprises at least one chaperone or a functional equivalent and/or a functional fragment thereof and at least one protein, said method comprising the step of providing said protein with at least one crossbeta structure and in yet another embodiment, the invention provides a method for producing an immunogenic composition, wherein said composition comprises at least one chaperone or a functional equivalent and/or a functional fragment thereof and at least one protein and at least one linker molecule, said method comprising the step of providing said linker molecule with at least one crossbeta structure. Examples of said linker molecule with at least one crossbeta structure are (synthetic) BiP binding peptides, glycated albumin, glycated hemoglobin, amyloid-β (fragments), fibrin peptides, oxidized LDL, misfolded ovalbumin, misfolded IgG.

In a preferred embodiment said chaperone or a functional equivalent and/or a functional fragment thereof is an hsp such as hsp70, hsp90 or gp96. Even more preferred are extra-cellular chaperones, such as BiP, haptoglobin, hsp72 or clusterin and even more preferred are extra-cellular chaperones that are ATP-independent, such as haptoglobin or clusterin. In yet another preferred embodiment, an improved chaperone is used (i.e. a chaperone molecule with improved crossbeta structure (precursor) binding affinity).

In yet another preferred embodiment, said protein is an antigen. Examples of suitable antigen are a melanoma antigen, (avian) influenza antigen, classical swine fever antigen, Neisseria meningitides antigen, or mastitis antigen.

The induction of a crossbeta structure (precursor) (i.e. providing a molecule with at least one crossbeta structure) may be accomplished in a variety of ways, for example by a heat treatment, glycation, oxidation or alkylation.

Preferably, the amount of crossbeta structure (precursor) is determined before and after the induction of a crossbeta structure. In this way the efficiency of the crossbeta structure (precursor) induction can be determined.

In a preferred embodiment said chaperone and said protein or said chaperone, said linker and said protein are non-covalently or covalently bound to each other.

A treatment meant to induce crossbeta structure in an antigen of interest, in order to introduce (more) crossbeta structure that binds efficiently to a chaperone selected for its immunomodulatory capacities, is also disclosed herein. The treatment is selected that introduces the crossbeta structure in the antigen that most efficiently binds to the chaperone. In immunization trials it is determined which fraction of the antigen in the crossbeta structure conformation has to be supplied to a subject, and which ratio between total antigen, antigen with crossbeta structure conformation and chaperone is most efficient in inducing protective immunity. For example, Neisseria meningitides antigen PorA is subjected to various conditions that introduce crossbeta structure in order to be able to select the crossbeta structure that binds most efficiently to an immunomodulatory chaperone, like for example BiP. This will provide a lead antigen preparation for induction of a protective immune response.

In yet another embodiment, the invention provides a composition obtainable by any of the above mentioned methods.

In a preferred embodiment, the invention provides a vaccine, comprising a composition according to the invention and a pharmaceutical acceptable carrier, diluent or excipient.

Now that the present invention discloses that a chaperone is capable of binding a crossbeta structure (precursor) and moreover that this feature may be used to produce immunogenic compositions, the application also provides the means to vary the amount of crossbeta structure (precursor) in an antigen and to determine, in combination with a particular chaperone, which amount of crossbeta structure is optimal for said particular chaperone.

The antigen used in the above described vaccine is different from its natural or normal equivalent, because said antigen comprises (additional) crossbeta structures and is thus not in its original or normal or wildtype conformation.

An antigen as used in a vaccine as described herein preferably comprises, besides (additional) crossbeta structures, the next features:
it provides an optimal binding with a chaperone of interest;
it is capable of binding to the receptor of an antigen presenting cell (APC), either via direct interaction with the receptor, or via indirect interaction upon binding of the chaperone with the bound antigen to the receptor;
the (additional) crossbeta structures are present in a part of said antigen that will not be presented by an APC;
a part of said antigen that will be presented by an APC is preferably accompanied by at least one anchor residue and cleavage sites around said to be presented part.

Whether an optimal binding between an antigen and a chaperone involves increased or decreased binding depends on the circumstances.

It is well known that peptides which are to be presented to T cell receptors need to fulfil a number of requirements. For different haplotypes different anchor residues are required, only peptides of a certain length can be presented, specific cleavage sites must be present around the peptide, signal to transport the peptide to the surface in the right context must be present, the stability of the bond between peptide and presenting molecule is relevant etc. Suitable methods for designing peptide epitopes are for example outlined in WO 97/41440 and WO 01/52614.

In a preferred embodiment, the antigen to be used in a vaccine composition interacts with one specific chaperone. And in yet another preferred embodiment, the vaccine composition comprises an antigen in which (additional) crossbeta structures have been introduced and which composition is devoid of a chaperone. In this latter vaccine composition, use is made of the already available chaperones in the to be vaccinated subject.

By in vitro testing the interaction of an antigen with (additional) crossbeta structures and a chaperone and the processing by an APC, the most suitable antigen can be selected without using a laboratory animal. I.e. the testing in animals can be postponed. Such a vaccine can further be adapted to a certain group of patients. It is for example known that a large part of RA patients develop anti-BiP antibodies. For such patients, preferably a vaccine is developed which antigen does not or hardly not to BiP.

Concluding, for vaccine development based on crossbeta-adjuvation, monitoring the interaction of the crossbeta-adjuvated vaccine with chaperones is a valuable tool for optimization of efficacy and specificity of the vaccine with respect to dosing and directing the desired immune response. The best crossbeta antigen ligand can be designed for optimal interaction with a target HSP, or the opposite, i.e. binding of an HSP with a crossbeta-adjuvated antigen can be avoided by adjusting the crossbeta adjuvant in a way that it is no longer a binding partner for the HSP. Subsequently, it can be analysed whether desired interaction with a cell surface receptor like for example CD36, CD91, SRA, is optimized.

In yet another embodiment, the invention provides a method for selecting an antigen suitable for vaccination comprising:
providing an antigen with crossbeta structure and/or crossbeta induced conformation or with additional crossbeta structure
determining the interaction of the antigen with a chaperone
determing the interaction of the antigen or an antigen-chaperone complex with a receptor from an antigen presenting cell
select the antigen that has optimal interaction with a chaperone as well as with a receptor of an antigen presenting cell.

The invention further provides an antigen obtainable via the above described method. Such an antigen is different from prior art antigens in which crossbeta structures have been induced, because the antigen of the present invention is designed to be optimal for a chaperone and/or designed to be optimal for a receptor of an antigen presenting cell. The invention also provides an immunogenic composition comprising an antigen optimised for interaction with a chaperone and/or a receptor from an antigen presenting cell. Optionally said immunogenic composition is further provided with a chaperone of interest.

The invention is further explained in the following examples, without being restricted to them.

### EXPERIMENTAL PART

### 1. Determination of the Misfolded Protein Binding Characteristics of Chaperones

To analyze the capacity of chaperones to bind to crossbeta structure and/or (misfolded) proteins comprising a crossbeta structure (precursor), several binding studies are conducted. At first binding of chaperones to crossbeta structure (precursor) is assessed in ELISA set-ups. For this purpose crossbeta structure comprising proteins are immobilized onto the wells of 96-wells plates. Examples of crossbeta structure comprising proteins or crossbeta structure precursor comprising proteins that are used are glycated proteins, heat-denatured proteins or alkylated-proteins like for example haemoglobin, albumin, lysozyme, ovalbumin, γ-globulins, Endostatin, amyloid-β, fibrin fragments, like for example peptides FP6, FP10, FP12, FP13, β2-microglobulin. After blocking, the immobilized crossbeta structures are overlayed with concentrations series of (recombinant) chaperone proteins and binding is analyzed using specific antibodies or antibodies directed to a tag that is incorporated in the chaperone. Tags that can be used are Universal Linkage System coupled to a fluorescent probe or biotin, a FLAG tag, a His tag, a glutathione S transferase tag, a maltose binding protein tag, a Myc tag, a VSV tag, a growth hormone tag, or other tags known to a person skilled in the art of protein chemistry. In a next series of experiments competition binding experiments are performed with chaperones that bind to crossbeta structure (precursor) and known crossbeta structure binding compounds like for example tissue-type plasminogen activator (tPA), factor XII, fibronectin, finger domains derived from tPA, factor XII, fibronectin or hepatocyte growth factor activator (HGFA), soluble fragment of receptor for advanced glycation endproducts (sRAGE), soluble extracellular fragments of low density lipoprotein receptor related protein (sLRP, LRP cluster 2, LRP cluster 4), (hybridoma) antibodies, intravenous immunoglobulins (IgIV or IvIg, either or not a fraction that is enriched by applying a crossbeta structure affinity column), Congo red, Thioflavin T or Thioflavin S. The ELISA set-up with immobilized misfolded protein can also be substituted with a set-up in which the chaperone protein is immobilized and overlayed with misfolded protein. Subsequently, binding of crossbeta structure to immobilized chaperone is assessed by analysis of binding of another known crossbeta structure binding compound. Alternatively, also the crossbeta structure binding compound can first be immobilized and overlayed with crossbeta structure. In this approach, binding of chaperone to the captured crossbeta structure is monitored.

Alternative to the direct binding ELISA's, binding of chaperones to misfolded proteins comprising crossbeta structure (precursor) is assessed using chromogenic tPA and factor XII activation assays. Concentration series of crossbeta structure are mixed with 100-1000 pM tPA, 5-200 μg/ml plasminogen and 0.1-1 mM chromogenic plasmin substrate S2251 (Chromogenix), and conversion of plasminogen to plasmin upon tPA activation by crossbeta structure is followed in time during 37° C.-incubation. For factor XII activity measurement, concentration series of crossbeta structure are mixed with 0.1-50 pg/ml factor XII, 0-5 μg/ml prekallikrein, 0-5 μg/ml high molecular weight kininogen and either chromogenic factor. XII substrate S2222 (Chromogenix) for direct measurement of factor XII activity, or chromogenic kallikrein substrate Chromozym PK (Boehringer-Mannheim) for indirect factor XII activity, and substrate conversion is followed in time spectrophotometrically during 37° C. incubation. To study binding of chaperones to misfolded proteins, concentration series of chaperones are included in the chromogenic assays.

All of the above listed analyses are performed with crossbeta structure (precursor) solutions before and after centrifugation for 1 h at 100,000*g, or before and after filtration using a 0.2 μm filter. This is performed to obtain insight into the capacity of chaperones to bind to soluble misfolded protein oligomers vs. to bind to insoluble (fibrilar) crossbeta structure aggregates.

Chaperones that are tested in these set-ups are for example (recombinant) BiP, CHAPERONE70, CHAPERONE90, gp96, grp170, chaperone72 (hsp72), macrophage migration inhibiting factor, chaperone27, chaperone60, GroEL, GroES, hsc70, grp94, chaperone90, chaperone16, chaperone40, α-crystallin, clusterin, haptoglobin or hsc73. Preferably, chaperones are selected for analysis, for which the extracellular localization and/or activity has been established, like for example gp96, BiP, grp170, calreticulin, chaperone72, macrophage migration inhibiting factor, chaperone70, α-crystallin, chaperone90, dnaK, chaperone70L1, haptoglobin or clusterin.

For a next series of experiments it is determined whether chaperones originating from various species all interact with misfolding protein in a similar way. To analyse whether chaperones originating from various species can interact with crossbeta structure and/or misfolded proteins comprising crossbeta structure and/or proteins comprising crossbeta structure precursor, we select chaperones from the following series for comparison of binding characteristics: chaperonin 10 from E. coli, and/or Chaperonin 60 from E. coli, and/or DnaJ Heat Shock Protein from Escherichia coli, and/or Heat Shock Cognate 70-interacting Protein from rat, and/or Heat Shock Cognate Protein 70 from bovine, and/or Biotinylated Heat Shock Cognate Protein 70 from bovine, and/or Heat Shock Protein 25 from mouse, and/or Heat Shock Protein 27 from human, and/or Heat Shock Protein 32 from rat, and/or Heat Shock Protein 32 from human, -and/or Heat Shock Protein 40 from human, and/or Heat Shock Protein 47 from rat, and/or Heat Shock Protein 60 from human, and/or Heat Shock Protein 65 from bovine, and/or biotin-labeled Heat Shock Protein 70 from bovine, and/or Heat Shock Protein 70 from bovine brain, and/or Heat Shock Protein 70 from human, and/or Heat Shock Protein 70B′ from human, and/or Heat Shock Protein 90 from bovine brain.

### 2. Detection of Misfolded Protein Using Chaperone

When binding characteristics of chaperones to crossbeta structure (precursor) is established with the crossbeta structure standard compounds listed above, presence of crossbeta structure in tester solutions or homogenates or cell suspensions can be assessed using the ELISA set-ups. Tester solutions may be biopharmaceuticals, blood, plasma, serum, cerebrospinal fluid, lymph fluid, synovial fluid, any protein solution. Binding of chaperones in an ELISA serves as a measure for the presence of misfolded protein in a sample. In one set-up proteins in a tester compound are immobilized onto the wells of an ELISA plate, and subsequently overlayed with a chaperone. In another approach, a crossbeta structure binding compound, for example tPA, a finger domain of tPA, factor XII, fibronectin or HGFA, sRAGE, LRP cluster 2, an antibody or a chaperone, like for example (recombinant) BiP, haptoglobin, CHAPERONE70, CHAPERONE90, gp96, grp170, chaperone72, macrophage migration inhibiting factor, chaperone27, chaperone60, GroEL, GroES, hsc70, grp94, chaperone90, chaperone16, chaperone40, α-crystallin, clusterin or hsc73 is first immobilized onto wells, overlayed with tester sample, and binding of misfolded protein is subsequently determined by assessing binding of another crossbeta structure binding compound or an alternatively labelled crossbeta structure binding compound, like for example tPA, a finger domain of tPA, factor XII, fibronectin or HGFA, sRAGE, LRP cluster 2, an antibody or a chaperone, like for example (recombinant) ((biotin) labelled) BiP, CHAPERONE70, CHAPERONE90, gp96, grp170, chaperone72, macrophage migration inhibiting factor, chaperone27, chaperone60, GroEL, GroES, hsc70, grp94, chaperone90, chaperone16, chaperone40, α-crystallin, clusterin, haptoglobin or hsc73. Other chaperones that may be included in the aforementioned detection assays for misfolded protein are (recombinant) chaperonin 10, Chaperonin 60, DnaJ Heat Shock Protein, Heat Shock Cognate 70-interacting Protein, Heat Shock Cognate Protein 70, Biotinylated Heat Shock Cognate Protein 70, Heat Shock Protein 25, Heat Shock Protein 27, Heat Shock Protein 32, Heat Shock Protein 40, Heat Shock Protein 47, Heat Shock Protein 65, Heat Shock Protein 70B′ (Sigma).

3. Clearance of Misfolded Proteins Comprising Crossbeta Structure from Solution: Identification of Misfolded Proteins Bound to Chaperones

With the selection of chaperones that show best binding characteristics towards soluble and insoluble misfolded proteins comprising crossbeta structure (precursor), affinity matrices are prepared. Such affinity matrices with (recombinant) (labelled/tagged) chaperone immobilized onto a solid support, like for example (magnetic) beads, agarose, Sepharose, the wells of an ELISA plate, are used to clear protein solutions or homogenates or cell suspensions from misfolded proteins comprising crossbeta structure (precursor). Tester solutions can be biopharmaceuticals, blood, plasma, serum, cerebrospinal fluid, lymph fluid, synovial fluid, any protein solution. For this purpose, chaperones are for example tagged with Universal Linkage System-biotin for coupling to Streptavidin-agarose, for example Streptavidin-Sepharose, or chaperones will be directly coupled to for example NHS-agarose or -Sepharose, CNBr-Sepharose, or Carboxylink matrix. The chaperone matrix with affinity for crossbeta structure are used in a batch mode and are incorporated in columns to allow for a continuous flow system. The efficiency of the separation technology is assessed by comparing the crossbeta structure load of a tester solution before and after contacting said solution with the chaperone matrix. Comparison of the crossbeta structure load is for example assessed by testing for tPA activation in a chromogenic assay, or for example by analysis of binding of a crossbeta structure binding compound in an ELISA set-up as described above. Alternatively, the efficiency of a chaperone to clear a solution from misfolded protein is assessed in bioassays (see below).

Clearance of a solution from misfolded proteins comprising crossbeta structure has another important application. Since crossbeta structures and proteins comprising a crossbeta structure are effectively bound to chaperones according to the invention, they are effectively separated and/or isolated from a sample and/or an animal's or human's body and subsequently identified. Therefore, a selected chaperone is used to isolate crossbeta structures and/or proteins comprising a crossbeta structure. Preferably, crossbeta structures and/or proteins comprising a crossbeta structure present in a body fluid, like for example blood, serum, plasma, cerebrospinal fluid, lymph fluid, synovial fluid, sputum and/or urine, are identified. For instance, the presence and/or identity of a crossbeta structure, and/or protein comprising a crossbeta structure, of healthy individuals is compared with the presence and/or identity of a crossbeta structure, and/or protein comprising a crossbeta structure, from individuals with a disease related to and/or associated with a crossbeta structure and/or a protein comprising a crossbeta structure. The identity and the relative concentration of a crossbeta structure and/or protein comprising a crossbeta structure is determined using any method known to a person skilled in the art, like for example, but not limited to, 2D gel electrophoresis and/or mass-spectrometric analyses. The results of a sample originating from a healthy individual and a sample originating from a patient is preferably compared. In this way, information is obtained, for instance about the identity and/or susceptibility of proteins prone to misfold and adopt crossbeta structure conformation during defined disease states. This obtained information subsequently serves as a diagnostic tool, for instance to monitor disease state, to monitor effectiveness of therapy, to monitor occurrence of disease, and provides valuable leads for development of therapeutics targeted at crossbeta structures and/or protein(s) comprising a crossbeta structure which are preferably specific for a defined disease.

Therefore we determine the identity of a crossbeta structure or a protein comprising a crossbeta structure in a sample comprising a protein, by performing:
contacting said sample with a crossbeta structure binding chaperone, resulting in bound crossbeta structures and/or bound protein(s) comprising a crossbeta structure, and
identifying a bound crossbeta structure and/or a bound protein comprising a crossbeta structure. Said bound crossbeta structure and/or bound protein comprising a crossbeta structure are preferably identified by analyzing at least part of the amino acid sequence of said crossbeta structure and/or protein using any method known in the art. Said sample preferably comprises an aqueous solution, more preferably a body fluid. In one preferred embodiment body fluids originating from healthy individuals (preferably humans) and body fluids originating from individuals suffering from, or suspected to suffer from, a disease related to and/or associated with the presence of a crossbeta structure are used in order to compare a healthy state with a diseased state (or a state wherein the risk of disease is enhanced).

### 4. Cell-Based Bioassays for Determination of Misfolded Protein Clearance/Neutralization Efficiency of Chaperones

Once the crossbeta structure (precursor) binding capacities of chaperones have been established, the putative use of the crossbeta structure (precursor) binding chaperones in medicine are tested in cell-based bioassays and in coagulation tests. The series of standard crossbeta structures as listed above are used as reference compounds that induce cellular toxicity, inflammatory responses, immune responses, or trigger the haemostatic system, for example by inducing tissue factor expression or influencing blood coagulation, in the bioassays mentioned below. In addition, pathogens with crossbeta structure comprising core proteins are included in the assays. The selected crossbeta structure (precursor) binding chaperones are tested for their neutralizing capacities with respect to crossbeta structure pathogenicity. The chaperones are co-administered in the bioassays, or crossbeta structure solutions are applied on chaperone-based affinity matrices for binding of crossbeta structure in order to deplete solutions from crossbeta structure, before these solutions are used in the bioassays. Beneficial effects of these approaches are determined by comparing the effects on cells and coagulation with respect to the effects of crossbeta structures alone when chaperones are not co-administered, or the effects of solutions that are not pre-treated on an affinity matrix. These approaches reveal whether crossbeta structure (precursor) binding compounds have the capacity to reverse adverse effects of crossbeta structure, either in a direct way by neutralizing crossbeta structures in vivo, or in an indirect way by extracting crossbeta structure from solutions that are subsequently applied (back) to a subject (biopharmaceuticals, extracorporal circulations, kidney dialysis apparatuses).

In vitro Murine Dendritic Cell Assay

Immunity against crossbeta structure and pathogens with exposed crossbeta structure is dependent on the presentation of antigens by antigen presenting cells (APC), such as dendritic cells. Cultured murine dendritic cells (DC's) are applied as a model for immunogenicity of crossbeta structure and crossbeta structure bearing pathogens. For this purpose, DC's are isolated from the hind legs of for example 8-12 weeks old Black-6 mice. Bones are isolated and rinsed in 70% ethanol, rinsed in RPMI-1640 medium with 25 mM HEPES, with 10% fetal calf serum, penicillin and Streptomycin. Then the bone is flushed with this buffer, in both directions. Eluates are cleared from erythrocytes by adding erythrocyte specific lysis buffer (to be obtained from the local UMC Utrecht Pharmacy Dept., catalogue number 97932329). Eluates are analyzed for viable cells by culturing them in cell culture plates. At this stage, the medium is enriched with 10 ng/ml GM-CSF. DC's grow in suspension or on a layer of macrophage cells. Using a FACS and specific antibodies, it is determined whether DC's are present and activated. Preferably the levels of so-called co-stimulatory molecules, such as B7.1, B7.2, MHC class II, CD40, CD80, CD86 will be determined on preferably CD11c positive cells. Alternatively, activation of NF-κB and/or expression of cytokines is used as indicators of activation of cells involved in immunogenicity, such as APC and DC. Preferably, the following cytokines are quantified: TNFα, IL-1, IL-2, IL-6, and/or IFNγ. Preferably, the cytokine levels are quantified by ELISA.

Alternatively, the mRNA levels are quantified. For a person skilled in the art it is evident that function of APC and DC are tested as well.

Alternatively, a stable DC line or other antigen presenting cells are used to test beneficial effects of depletion or neutralisation of misfolded proteins with crossbeta structure (on pathogens) (Citterio et al., 1999).

In vitro Human Blood Derived Dendritic Cell Assay

Human DC's are generated from non-proliferating precursors in peripheral blood mononuclear cells (PBMCs), essentially by the method described before (Sallusto and Lanzavecchia, 1994). To obtain purified PBMCs, the cells are first depleted from erythrocytes and T-cells. In brief, the haematocryte fraction of 50 ml freshly drawn citrated human blood or of buffy coat is used. Using the Ficol-based separation-centrifugation method, PBMCs are separated. Isolated cells are resuspended at a concentration of 1*10⁶ cells/ml in medium+1% FCS and are incubated for 45 min. at 37° C. The CD14+ cells adhere to the bottom of the flask in this time. Supernatant is discarded and cells are cultured in medium+10% FCS+800 U/ml GM-CSF+500 U/ml IL-4 (37° C., 5% CO₂), or the cells are cultured without FCS. After maturation time with GM-CSF and IL-4, cells are for example incubated with a concentration series of standard crossbeta structure comprising compounds like for example glycated haemoglobin, heat-denatured ovalbumin, or like for example pathogens with a amyloid core protein like for example cultured Staphylococcus aureus Newman and Escherichia coli TOP10 (Invitrogen, 44-0301). Crossbeta structures and pathogens comprising crossbeta structures are applied to the DC's in PBS or in buffer comprising crossbeta structure binding compounds like for example Thioflavin T, tPA and IgIV. To determine the influence of the crossbeta structures and of bacterial cells comprising core proteins with crossbeta structure on the DC's, (amongst others) surface density of (a subset of) surface molecules CD86, CD36, CD40, HLA-DR, CD1a, CD80, CD14, LRP, LOX-1, Scavenger receptor A, CD83 or mannose receptor is measured using FACS.

In vitro Human Umbilical Vein Endothelial Cell Bioassay and Murine Microvascular bEnd.3 Endothelial Cell Bioassay

Glycated proteins comprising crossbeta structure and amyloid-β induce inflammatory response, are believed to contribute to pathogenesis of certain protein misfolding diseases. In general, misfolded proteins induce cellular dysfunction with enhanced expression or activation of inflammatory signals. The effect of misfolded proteins on endothelial cell (dys)function is for example measured by determining the levels of reactive oxygen species or nitric oxide or tissue factor in response to misfolded proteins. Human umbilical vein endothelial cells that are isolated and cultured, according to standard protocols, are used, or other endothelial cells such as the murine microvascular bEnd.3 endothelial cell line. The levels of reactive oxygen species (ROS) levels are monitored using fluorescent probes, such as CM-H2DCF-DA. Alternatively, cell viability is monitored by standard MTT-assay. The levels of tissue factor expression is determined using a chromogenic assay with chromogenic substrate S-2765 (Chromogenix). The cultured primary cells and the cell line provide the opportunity to perform in vitro cell assays that are accepted in research community as model systems for certain disease states.

### Phagocytosis of Crossbeta Structure and Crossbeta Structure Comprising Pathogens

The uptake of crossbeta structure and crossbeta structure comprising pathogens, and the effect of crossbeta structure binding chaperones are studied in vitro using cultured cells, preferably monocytes, dendritic cells, or macrophages or similar cells, for example U937 or THP-1 cells. Preferably, crossbeta structure and crossbeta structure comprising pathogens are labelled, preferably with 125I or a fluorescent label, preferably FITC, covalently attached to the molecule by a linker molecule, preferably ULS (universal Linkage system) or by applying an alternative coupling method. Cells are preferably labelled with mepacrin or other fluorescent labels, such as rhodamine. Phagocytic cells are incubated in the presence of labelled crossbeta structure or crossbeta structure comprising cells in the presence or absence of a crossbeta structure binding chaperone. After incubation, preferably during several hours, the uptake of labelled molecules or cells is measured preferably using a scintillation counter (for 125I) or by FACS-analysis (with fluorescent probes) or immunofluorescent microscopy. The uptake of pathogen cells is also counted under a light microscope with visual staining of these cells.

Alternatively, the response of cells that are involved in phagocytosis to crossbeta structure or crossbeta structure comprising pathogens are also assessed by measuring expression levels of several markers for an inflammatory/activation/thrombogenic response. Using commercially available ELISA's, expression levels of tissue necrosis factor-α and interleukin-8 are determined upon exposure of for example macrophages to crossbeta structure or crossbeta structure comprising pathogens. Expression levels of tissue factor are determined using a chromogenic assay with chromogenic substrate S2765 (Chromogenix).

Ex vivo Human Blood Platelet Aggregation Assay

The influence of crossbeta structure binding chaperones on blood platelet aggregation induced by crossbeta structure or crossbeta structure comprising pathogens is tested with washed platelets or with platelet rich plasma in an aggregometric assay. Freshly drawn human aspirin free blood is mixed gently with citrate buffer to avoid coagulation. Blood is spinned for 15′ at 150*g at 20° C. and supernatant is collected; platelet rich plasma (PRP). Buffer with 2.5% trisodium citrate, 1.5% citric acid and 2% glucose, pH 6.5 is added to a final volume ratio of 1:10 (buffer-PRP). After spinning down the platelets upon centrifugation for 15′ at 330*g at 20° C., the pellet is resuspended in HEPES-Tyrode buffer pH 6.5. Prostacyclin is added to a final concentration of 10 ng/ml, and the solution is centrifuged for 15′ at 330*g at 20° C., with a soft brake. The pellet is resuspended in HEPES-Tyrode buffer pH 7.2 in a way that the final platelet number is adjusted to 200,000-250,000 platelets/μl. Platelets are kept at 37° C. for at least 30′, before use in the assays, to ensure that they are in the resting state. Platelets of approximately five donors are isolated separately.

For the aggregometric assays, platelet solution is added to a glass tube and prewarmed to 37° C. A stirring magnet is added and rotation is set to 900 rpm, and the apparatus (Whole-blood aggregometer, Chrono-log, Havertown, Pa., USA) is blanked. A final volume of 1/10 of the volume of the platelet suspension is added, containing the agonist of interest and/or the premixed antagonist of interest, prediluted in HEPES-Tyrode buffer pH 7.2. Aggregation is followed in time by measuring the absorbance of the solution, that is decrease in time upon platelet aggregation. As a positive control, either 10 μg/ml collagen (Kollagenreagens Horm, NYCOMED Pharma GmbH, Linz, Austria; lot 502940), or 5 μM of synthetic thrombin receptor activating compound TRAP. In tests to analyse the effect of chaperones, 10-100 μg/ml glycated haemoglobin or 10-100 μg/ml amyloid-β is used to induce platelet aggregation. Aggregation is recorded for at least 15′.

Ex vivo Human Plasma Coagulation Assays

For analysis of the influence of crossbeta structure or crossbeta structure comprising pathogens on the characteristics of blood coagulation, and for analysis of the effects of crossbeta structure binding chaperones on the influence of crossbeta structure and pathogens with crossbeta structure on coagulation, two standard coagulation tests are performed on for example a KC10 Coagulometer. Pooled human plasma of approximately 40 apparently healthy donors is clotted by adding either negatively charged phospholipids, CaCl₂ and kaolin when an activated partial thromboplastin time (aPTT) is considered, or tissue factor rich thromboplastin and CaCl₂ when prothrombin time (PT) determinations are considered. APTT's and PTs are performed as follows. Plasma is incubated with concentration series of crossbeta structure comprising pathogen for, for example, 15′ to 120′ at room temperature or at 37° C. Pathogen cells are pelleted by centrifugation and plasma supernatant is subsequently applied in either an aPTT or a PT. At conditions that influence the coagulation tests, preincubations of pathogens with concentration series of crossbeta structure binding chaperones are performed, before applying the pathogens to plasma, or in an alternative way, pathogens and crossbeta structure binding chaperones are applied to plasma together. For an aPTT analysis, 50 μl of plasma is mixed with 50 μl of a physiological buffer. Next 25 μl of 900 μg/ml Kaolinum Ponderosum (Genfarma) and 120 μM lipid vesicles (phosphatidyl serine/phosphatidyl choline/phosphatidyl ethanolamine) in a 20/40/40% (v/v) ratio is added, and the mixture is prewarmed to 37° C. To start the assay, 25 μl of a 50 mM CaCl₂ solution is added. For a PT analysis, 50 μl of (pretreated) plasma is combined with 50 μl H₂O and is incubated for 5′ at 37° C. The PT analysis is started by adding 50 μl of a Thromborel S stock, which is prepared at twice the concentration as recommended by the manufacturer (DADE Behring).

The influence of crossbeta structure binding chaperones on coagulation is further assessed in a more direct way. PT and aPTT analyses are performed with untreated plasma, in the presence of concentration series of crossbeta structure binding chaperones. Typically, chaperone concentrations are in the range 0-10-100-1000 μg/ml.

5. In vivo Models for Determination of the Beneficial Effects of Chaperones during Pathogenicity
In vivo Mouse Sepsis Model

Sepsis is mediated by crossbeta structure. One of the in vivo mouse sepsis models that is applied to test effects of chaperones, is the ‘cecal ligation and puncture’ model. For this model, female Balb/c mice are anesthetized before an abdominal incision is made to bring the cecum outside the abdomen. After puncturing the cecum an amount of luminal contents is transferred outside through the punctures, before the cecum is returned in the adomen and the mouse is closed. Infection progression is monitored by measuring the body temperature and by scoring the mobility of mice. One considers mice lethally infected when they are hypothermic (T<33° C.) and when mice are unable to right themselves. Effects of administering chaperones with affinity for misfolded proteins with crossbeta structure conformation after the puncture of the cecum are assessed by monitoring a group of untreated mice and a group of mice that received chaperones.

In vivo Mouse/Rat Experimental Autoimmune Encephalomyelitis Model

To test whether chaperones that bind crossbeta structure (precursor) provide a beneficial effect during a multiple sclerosis (MS) relapse, an in vivo mouse model for MS, the experimental autoimmune (or allergic) encephalomyelitis (EAE) model is used. For this purpose, myelin basic protein (MBP) or myelin oligodendrocyte glycoprotein peptide 35-55 (MOG35-55) is emulsified in incomplete Freund's adjuvant (IFA) with mycobacterium. The presence of misfolded proteins with crossbeta structure is determined using Thioflavin T and Congo red fluorescence assays, as well as tPA binding and activation assays. Binding of chaperones to the emulsified MBP or MOG35-55 is assessed. To induce EAE in mice or rats, the emulsified MBP or MOG35-55 is injected in for example the hind footpad. In mice a subcutaneously injected amount of MOG35-55 is preferably accompanied with an intraperitoneal injection of Bordetella pertussis toxin, which is repeated after 48 h. For example, Lewis female rats are used, or female Balb/c mice. Measures for clinical disease are example scored as follows: 0, normal; 1, limp tail; 2, impaired righting reflex; 3, paresis of hind limbs; 4, complete paralysis of hind limbs; 5, death. The effect of any chaperone preparation is analyzed by administering the drug at one or more time points after inducing EAE. Several chaperones that are tested are for example clusterin, haptoglobin and BiP. Secondly, antigen preparations are applied to an affinity matrix comprising immobilized chaperone in order to deplete the antigen solution from misfolded protein comprising crossbeta structure. The remaining solution is used for induction of EAE and the efficacy is compared with EAE induced in mice upon administering antigen that is not purified on a chaperone affinity matrix.

### 6. Adjuvation by Chaperones

The crossbeta structure is involved in triggering of the immune system and initiating a humoral response. Chaperones like for example gp98, chaperone70 and chaperone90 have immunomodulating activity, mediated at least in part by their interaction with multiligand (crossbeta structure binding) cellular receptors CD36, scavenger receptor A (SRA), low density lipoprotein receptor related protein (LRP or CD91), LOX-1, Toll-like receptor 2 (TLR2), Toll-like receptor-4 (TLR4) and CD40 on antigen presenting cells (APC) like dendritic cells or macrophages. Some of these receptors are involved in endocytosis of chaperones and their bound cargo misfolded protein, a process that facilitates processed misfolded protein comprising crossbeta structure antigen presentation by the APC, resulting in the adaptive immune effects of chaperones. Other receptors facilitate activation of the innate immune system upon binding to holo-chaperones. Some chaperones are known for their pro-inflammatory activity, whereas other chaperones exhibit anti-inflammatory activities. For strategies aiming at developing vaccines, triggering of the adaptive immune system by chaperone—multiligand receptor interactions, with a misfolded protein antigen bound to the chaperone, facilitates an efficient and potent way of obtaining protection against infections. Suggested variations of efficient vaccine compositions comprise, amongst others, a selected chaperone, for example a member of the gp96 family, chaperone70 family, chaperone90 family of chaperones or BiP, (non)covalently bound to an antigen of interest. The antigen is supplied solely in a crossbeta structure comprising form, or may be supplied with a part in a native conformation and a part in crossbeta structure conformation. Alternatively, another crossbeta structure selected for its potent immuno-stimulating activity, like for example glycated protein, alkylated protein, human β2-glycoprotein I exposed to cardiolipin or oxidized human interferon-α or heat-denatured ovalbumin or amyloid-β, is combined with a chaperone and antigen, with or without crossbeta structure, or a combination thereof. Vaccination trials are for example conducted in mice with antigens, with 0/50/100% crossbeta structure comprising molecules, for example with classical swine fever E2 antigen, influenza antigen H3, H5 or H7, Neisseria meningitidis antigen PorA, Fasciola hepatica antigen L3, HIV related antigen gp120. In a subset of immunisations, the antigens are mixed with another protein which comprises crossbeta structure conformation, like for example glycated protein, alkylated protein, human or mouse β2-glycoprotein I exposed to cardiolipin or oxidized human interferon-α or heat-denatured ovalbumin or amyloid-β. Immunisations are conducted with and without the addition of a chaperone protein, like for example BiP, chaperone70, chaperone90, gp96, hsp72, calreticulin. Titers against the antigens are determined after one, two and three weeks post-injection, and based on observed titers it is decided whether a second immunisation is required.

In summary, we will analyze the influence of a crossbeta structure binding chaperone and/or a crossbeta structure precursors binding chaperone and/or a combination of chaperones and/or functional equivalents thereof and/or fragments thereof on induction of a humoral immune response by an antigen preparation comprising at least one protein component with crossbeta structure conformation.

### Materials & Methods

### Cloning and Expression of Recombinant Human BiP

The human BiP gene except the signal peptide encoding region was obtained from Geneart (Germany). The gene was extended in a way that the transcribed protein will have a C-terminal extension with amino-acid sequence KSKSKSMMAA, for purposes related to couplings to matrices. A BamHI restriction site was added to the 5′ region, a NotI restriction site to the 3′ region. The gene was supplied in a vector and digested with BamHI and NotI for ligation in the PABC674 expression vector of the local Expression Facility Utrecht (the Netherlands). Expression of BiP in this vector will result in addition of a C-terminal His-tag and a C-terminal FLAG-tag. For expression, 2 μg of vector with BiP was transiently transfected in HEK 293E cells. Cells were allowed to grow for 4 days. Cell culture supernatant was analyzed for the presence of BiP using SDS-PAGE and Western blotting. The blot was incubated with mouse monoclonal anti-His-tag antibody (Novagen, 70796) and RAMPO (DAKOCytomation). Presence of BiP-FLAG-His was determined upon incubation of the blot with ECL reagent (Perkin Elmer, NEL104) and subsequent photographic imaging.

Misfolded Proteins with Crossbeta Structure Conformation

### Glycation of Proteins

Glycation of bovine serum albumin and human haemoglobin (Hb) for preparation of advanced glycation endproducts (BSA-AGE, HB-AGE) was performed as follows. For preparation of BSA-AGE, 100 mg ml⁻¹ of albumin was incubated with phosphate-buffered saline (PBS, 140 mM sodium chloride, 2.7 mM potassium chloride, 10 mM disodium hydrogen phosphate, 1.8 mM potassium di-hydrogen phosphate, pH 7.3) containing 1 M of D-glucose-6-phosphate disodium salt hydrate (anhydrous) (g6p, ICN, Aurora, Ohio, USA) and 0.05% m/v NaN₃, at 37° C. in the dark. The solution was glycated for 70 weeks. Human Hb at 10 mg/ml was incubated for 75 weeks at 37° C. with PBS containing 1 M of g6p and 0.05% m/v of NaN₃. After incubations, albumin and Hb solutions were extensively dialysed against distilled water and, subsequently, aliquoted and stored at −20° C. Protein concentrations were determined with Advanced protein-assay reagent ADV01 (Cytoskeleton, Denver, Colo., USA).

### Control Proteins

Non-modified protein control solutions were prepared from lyophilized protein stocks stored at 4° C. Bovine serum albumin (Sigma, A7906), human γ-globulins (G4386, Sigma, Zwijndrecht, The Netherlands) and Hb (Sigma-Aldrich, H7379) were all dissolved at 1 mg/ml in HBS (10 mM HEPES, 4 mM KCl, 137 mM NaCl, pH 7.3). Solutions were kept at a roller device for 15-45 minutes at room temperature before use.

### Heat-Denaturation

Hen egg-white lysozyme (ICN, Irvine, Calif., USA; lyophilized, catalogue number 100831) and purified chicken ovalbumin (OVA, Sigma; catalogue number A5503, lot 071k7094) in 67 mM NaPᵢ buffer pH 7.0, 100 mM NaCl, was heated for five cycles in PCR cups in a PTC-200 thermal cycler (MJ Research, Inc., Waltham, Mass., USA). In each cycle, proteins were heated from 30 to 85° C. at a rate of 5° C./min. Solutions were stored at −20° C. Lyophilized human amyloid-β(1-40) with E22Q mutation ‘Dutch type’ (Peptide facility, Dutch Cancer Institute, Amsterdam, the Netherlands) and lyophilized human y-globulins were first dissolved in 1,1,1,6,6,6-hexafluoro-2-propanol and trifluoroacetic acid in a 1:1 volume ratio. Solvent was evaporated under an air stream and Aβ or γ-globulins were dissolved in H₂O to a final concentration of 1 mg/ml, and incubated for 72 h at 37° C. After the incubation the Aβ solution was stored at room temperature and the amyloid γ-globulins solutions was stored at −20° C.

### Protein Alkylation

Modified bovine serum albumin (BSA) was obtained by reducing and alkylation. BSA (Sigma, A7906) was dissolved in 8 M urea, 100 mM Tris-HCl pH 8.2, at 10 mg ml⁻¹ final concentration. Dithiothreitol (DTT) was added to a final concentration of 10 mM. Air was replaced by N₂ and the solution was incubated for 2 h at room temperature. Then, the solution was transferred to ice and iodoacetamide was added from a 1 M stock to a final concentration of 20 mM. After a 15 min. incubation on ice, reduced-alkylated BSA (alkyl-BSA) was diluted to 1 mg ml⁻¹ by adding H₂O. Alkyl-BSA was dialyzed against H2O before use.

### Determination of Crossbeta Structure in Denatured Proteins

To establish that crossbeta structure was induced during the above mentioned protein treatments, enhancement of Thioflavin T fluorescence or Congo red fluorescence was assessed as well as tPA binding in an ELISA and tPA activation in a chromogenic tPA activation assay. Presence of large protein assemblies in amyloid γ-globulins and presence of fibrilar peptide multimers in Aβ was visualized using transmission electron microscopy imaging.

### ELISA

Binding of BiP to Misfolded Proteins with Crossbeta Structure

Proteins at 5 μg/ml were coated for 1 h at room temperature with agitation, in Microlon high-binding ELISA plates (Greiner) in 50 mM NaHCO₃ pH 9.6, except for Aβ that was coated at 20 μg/ml. Buffer was coated as negative control. A twofold dilutions series of cell culture supernatant of HEK 293E cells overexpressing BiP was also coated to the plate for anti-FLAG-tag antibody control purposes. Plates were washed and blocked with ½*Blocking reagent (Roche). Undiluted cell culture supernatant enriched with 0.1% Tween20 was added to the wells with immobilized protein ligands and incubated for 1 h at room temperature with agitation. Medium was discarded and the plate was washed with PBS with 0.1% v/v Tween20. Mouse monoclonal anti-FLAG-tag antibody (Sigma, A8592, anti-FLAG M2PO conjugate) was diluted 1000× in PBS/0.1% Tween20 and added to all wells, including those that are coated with cell culture supernatant. After a 1 h incubation at room temperature with agitation and after washing, wells were overlayed with 3000× diluted RAMPO (DAKOCytomation) in PBS/0.1% Tween20. After 30 minutes the plate was washed and bound peroxidase was visualised with tetramethylbenzidine (TMB, #45.103.20/#45.014.01, Biosource, Nivelles, Belgium). The reaction was stopped after 5 minutes with 10% H₂SO₄ in H₂O. Plates were read at 450 nm.

Inhibition of BiP Binding to Crossbeta Structure with tPA

In a similar set-up as described above, the influence of 1 μM tPA (Actilyse, Boehringer-Ingelheim) or 1 μM K2P-tPA, a tPA deletion mutant lacking the N-terminal crossbeta structure binding finger domain, EGF-like domain and first kringle domain (Reteplase, Boehringer-Ingelheim), on binding of BiP to misfolded proteins with crossbeta structure was determined in an ELISA set-up. For this purpose, cell culture supernatant with expressed and secreted BiP was diluted threefold in PBS/0.1% Tween20/10 mM ε-amino caproic acid, and either tPA or K2P-tPA was added whereas PBS was added to a control sample. Hb-AGE, Hb, BSA-AGE, BSA and buffer were coated. The threefold diluted cell supernatants were applied to the ELISA plate in duplicates and BiP binding was subsequently measured as described above. Coat efficiency was checked with specific anti-AGE antibody, anti-albumin antibody and anti-Hb antibody.

### RESULTS

### Cloning and Expression of Recombinant Human BiP, and Analysis of BiP Binding to Crossbeta Structure

### Cloning and Expression

The human BiP gene was enlarged with several tags at the C-terminus. The synthetic gene was designed in a way that at the C-terminus sequences were incorporated that may aid in efficient and oriented coupling of the BiP protein molecule to (chromatography) matrices, like for example CNBr-Sepharose, NHS-Sepharose, Carboxy-link, any Ni²⁺-based affinity matrix. In addition, the linker sequence may be used to couple labels to the protein molecule, like for example NHS-fluorescent probe, or Universal Linkage System-biotin, which can be used for detection purposes and/or for coupling purposes using for example Streptavidin-Sepharose. By using the PABC674 vector a FLAG-tag and a His-tag will be added to this C-terminus. In total, the original 71 kDa BiP was extended with a linker meant for matrix coupling purposes, KSKSKSMMAA, a peptide with sequence DYKDDDDK (FLAG-tag) and HHHHHH (His-tag), with a total molecular mass of 2.9 kDa. Indeed, recombinant BiP has an apparent molecular mass of approximately 75 kDa as seen on a Western blot (FIG. 1). Based on the Western blot analysis we conclude that BiP is secreted from the HEK 293E cells. BiP is purified using for example Ni²⁺-based affinity chromatography, anion exchange chromatography and/or gel filtration chromatography.

### Crossbeta Structure Binding ELISA

To assess the binding capacity of BiP towards misfolded proteins comprising a crossbeta structure, misfolded proteins and native controls were immobilized on ELISA plates and overlayed with cell culture supernatant of 293E cells overexpressing recombinant human BiP. BiP binds to glycated haemoglobin (Hb-AGE) and to a lesser extent to coated native haemoglobin, but not to amyloid-β(1-40) aggregates (Aβ) (FIG. 2A). BiP binds to glycated albumin (BSA-AGE) and to a lesser extent to reduced and alkylated albumin (alkyl-BSA), and not to native BSA (FIG. 2B). BiP binding is more pronounced with organic solvent/heat denatured human γ-globulins (amyloid Ig) and heat-denatured lysozyme (d-lysozyme) than with native γ-globulins (native Ig) (FIG. 2C). From these observations we conclude that the overexpressed BiP has the ability to bind to misfolded proteins comprising crossbeta structure.

In a second experiment the influence of multiligand crossbeta structure binding tPA and of K2P-tPA, which lacks the N-terminal crossbeta structure binding finger domain, on binding of recombinant BiP to immobilized Hb-AGE, Hb, BSA-AGE and BSA was assessed in an ELISA set-up with coated ligands for BiP. Binding of BiP occurred in the presence of 10 mM ε-amino caproic acid, that prevents the interaction of the kringle2 domain (K2) of tPA and K2P-tPA with free amino groups at the exterior of the ligands. With Hb-AGE, tPA at 1 μM reduces BiP binding from 100% to 69%, whereas K2P-tPA seems to promote BiP binding to some extent. Also some binding of BiP is seen with freshly dissolved Hb, which may comprise a fraction misfolded protein due to for example lyophilization. Similar to Hb, BiP binds to BSA-AGE and hardly to BSA. When tPA is introduced in the BiP solution, BiP binding is inhibited for 57%, whereas again K2P-tPA seems to facilitate to some extent BiP binding.

From our experiments we conclude that recombinant human BiP with a C-terminal extension is expressed and secreted by HEK 293E cells. The BiP is biologically active, based on the observation that BiP binds to a series of misfolded proteins comprising a crossbeta structure. That tPA inhibits binding of-BiP to proteins comprising crossbeta structure shows the role of crossbeta structure in the interaction of BiP with its misfolded protein ligands.

### EXAMPLES 7-16

### General Materials and Methods for Examples 7-16

Preparation of Misfolded Proteins with Crossbeta Structure
Heat Denaturation of IgIV Gammagard (dIgIV-86)

Human immunoglobulin intravenous (IgIV) Gammagard (Baxter) was dissolved under sterile conditions to 5 mg/ml in 20 mM sodium phosphate pH 5.0, and heat denatured from 25° C. to 86° C. at 5° C./minute. After heat denaturing, dIgIV-86 was immediately stored at −80° C. and its structure was analyzed using various assays as described below. As native control, freshly dissolved IgIV Gammagard at a concentration of 5 mg/ml in 20 mM sodium phosphate pH 5.0 was kept at room temperature for 10 minutes, and stored at −80° C. In the text and figures dIgIV-86 is also referred to as IgIV-86.

### Misfolding of Octagam IgIV

Octagam IgIV (Octapharma, Brussel, Belgium) was used for preparation of misfolded human IgG with crossbeta structure. The endotoxin concentration in IgIV was low, i.e. 0.13 E.U./ml in the 50 mg/ml Octagam stock, as determined using a standardized Limulus Amebocyte Lysate (LAL) assay (Cambrex). IgIV was diluted in 10 mM NaPi buffer (pH 8.1) to 20 mg/ml and stepwise heated (0.5° C./minute) from 25° C. to 65° C., kept at room temperature for 1 hour and 40 minutes and subsequently stored at −80° C. Referred to as dIgIV-65.

### Misfolding of a Composition of Human IgGs

Human IgGs (γ-globulins, Sigma, G4386) were dissolved to 5 mg/ml in HEPES buffer (20 mM HEPES, 137 mM NaCl, 4 mM KCl, 3 mM CaCl₂, pH 7.2). Then the pH was increased by adding a volume from a 5 M NaOH stock and kept for 40 minutes at 37° C. Then, an equal amount from a 5 M HCl stock was added to adjust pH to its initial value, and stored at −80° C. Large aggregates were observed by eye. Referred to as (h)IgG-Base.

Heat Denaturation of Ovalbumin (dOVA Standard)

Ovalbumin (OVA, from chicken egg white, Sigma, A5503 grade V, lot 07147094) was dissolved in PBS at a concentration of 1 mg/ml, and heated from 30° C. to 85° C. for 5 cycles in a PCR machine with temperature steps of 5° C. per minute. This misfolded OVA is referred to as dOVA or dOVA standard (std).

Preparation of Crossbeta Amyloid-β 1-42 (Aβ42t=0)

Lyophilized synthetic human amyloid-β(1-42) peptide (DAEFRHDSGYEVHHQKLVFFAEDVGSNKGAIIGLMVGGVVIA; NKI Amsterdam, The Netherlands; 4514 Da, SEQ-ID 1) (Aβ1-42) was first monomerized by dissolving at 1 mM in 1,1,1,6,6,6-hexafluoro-isopropanol (HFIP) and aliquoted in sterile micro-centrifuge tubes. HFIP was removed with nitrogen gas, and the peptide film was resuspended in dry dimethyl sulfoxide (DMSO, Pierce, 20684) to a concentration of 5 mM, snap-frozen in liquid nitrogen and stored at −80° C. (monomerized Aβ1-42 stock). Thawed monomerized Aβ1-42 stock in DMSO was dissolved in PBS at a final concentration of 400 μg/ml (89 μM), and stored directly at −80° C. Referred to as Aβ1-42 or Aβ42 or Aβ42t=0.

### Transmission Electron Microscopy (TEM)

TEM images were collected using a Jeol 1200 EX transmission electron microscope (Jeol Ltd., Tokyo, Japan) at an excitation voltage of 80 kV. For each sample, the formvar and carbon-coated side of a 100-mesh copper or nickel grid was positioned on a 5 μl drop of protein solution for 5 minutes. Afterwards, it was positioned on a 100 μl drop of PBS for 2 minutes, followed by three 2-minute incubations with a 100 μl drop of distilled water. The grids were then stained for 2 minutes with a 100 μl drop of 2% (m/v) methylcellulose with 0.4% uranyl acetate pH 4. Excess fluid was removed by streaking the side of the grids over filter paper, and the grids were subsequently dried under a lamp. Samples were analysed at a magnification of 10K.

### Congo Red (CR) Fluorescence Assay

Enhancement of Congo red fluorescence is a characteristic of misfolded proteins that comprise structural features common to proteins with crossbeta conformation. Fluorescence of Congo red (CR) (Aldrich Chemical Company, Inc., Milwaukee, Wis., USA, 86,095-6) was measured in duplo on a Thermo Fluoroskan Ascent 2.5 microplate fluorometer (Vantaa, Finland) in black 96-wells plates at an emission wavelength of 590 nm and an excitation wavelength of 544 nm. Protein and peptide stocks were diluted to 100 μg/ml for dOVA and IgIV samples and 40 μg/ml for Aβ samples in 25 μM CR in PBS, and incubated for 5 minutes at room temperature. Background fluorescence from buffer and protein solution without CR and from CR in buffer were subtracted form corresponding measurements of protein solution incubated with CR. Positive control for the measurements was 100 μg/ml dOVA (dOVA std).

### Thioflavin T (ThT) Fluorescence Enhancement Assay

Enhancement of ThT fluorescence is a characteristic of misfolded proteins that comprise structural features common to misfolded proteins with crossbeta conformation. Fluorescence of Thioflavin T (ThT) (Sigma, St. Louis, Mo., USA, T-3516) was measured similarly to the procedure described for CR. The emission wavelength was now 485 nm and the excitation wavelength was 435 nm. Protein and peptide stocks were diluted in 25 μM ThT in 50 mM Glycine buffer pH 9.0.

8-Anilino-1-naphthalenesulfonic acid (ANS) Fluorescence Assay

ANS fluorescence is enhanced when bound to clusters of hydrophobic amino-acyl residues. Upon binding to solvent-exposed hydrophobic regions of proteins, the emission wavelength (λ_{EM}) shifts from 514 nm to 460 nm when excited at a wavelength of 380 nm (λ_{EX}), accompanied by a dramatic enhancement in fluorescence intensity. Fluorescence of ANS (Sigma, A1028) was measured at an emission wavelength of 460 nm and an excitation wavelength of 380 nm. The various tester protein and peptide stock solutions were dissolved in 40 μM ANS in PBS and incubated for 5 minutes at room temperature. Background fluorescence from buffer and protein solution without ANS and of ANS in buffer were subtracted form corresponding measurements of protein solution incubated with ANS. Positive control for the measurements was 100 μg/ml dOVA (dOVA std).

4,4′ dianilio-1,1′ binaphthyl-5,5′ disulfonic acid di-potassium salt (Bis-ANS) Fluorescence Enhancement Assay

Similar to CR, ThT and ANS, the enhancement of Bis-ANS (Sigma) fluorescence was measured. The emission wavelength was 485 nm and the excitation wavelength was 435 nm. Protein and peptide stocks were diluted in 25 pM Bis-ANS in PBS.

### Thioflavin S (ThS) Fluorescence Enhancement Assay

Enhancement of ThS fluorescence is a characteristic of misfolded proteins that comprise structural features common to proteins with crossbeta conformation. Fluorescence of ThS (Sigma, 033k1076) was measured according to the procedure described for CR and ThT. The emission wavelength was 542 nm and the excitation wavelength was 435 nm. Protein and peptide stocks were diluted in 25 μM ThS in PBS.

### Intrinsic Tryptophan Fluorescence Assay

Intrinsic tryptophan (Trp) fluorescence measurements were performed on a Gemini Spectramax XPS, (Molecular Devices) using Softmax pro v5.01 software, with 100 μl samples, in black 96-wells plates, at an excitation wavelength of 283 nm. Emission spectra were collected at room temperature in the 360-850 nm range. A natively folded protein either displays increased or decreased fluorescence compared to its misfolded counterpart. The absolute values of the Trp fluorescence intensity is not very informative. However, changes in the magnitude serve as a probing parameter for monitoring perturbations of the protein fold. A shift in the fluorescence emission wavelength is a better indication for local changes in the environment of the Trp fluorophore. Solvent exposed Trp residues display maximal fluorescence at 340-350 nm, whereas totally buried residues fluoresce at about 330 nm.

tPA/Plasminogen Activation Assay

Enhancement of tPA/plasminogen activity upon exposure of the two serine proteases to misfolded proteins was determined using a standardized chromogenic assay (see for example patent application WO2006101387, paragraph [0195], and (Kranenburg et al., 2002)). Both tPA and plasminogen act in the Crossbeta Pathway (See Table 2, 3). Enhancement of the activity of the crossbeta binding proteases is a measure for the presence of misfolded proteins comprising crossbeta structure.

### Results

TEM Analysis of dOVA Standard

TEM analysis of heat-denatured ovalbumin, used as a standard misfolded protein in indicated assays (dOVA std.), shows that the misfolded protein aggregates into non-fibrillar multimers (not shown). For all fluorescence enhancement assays described above, as well as for the tPA/plasminogen activation assay, the dOVA std. concentration has been identified that results in maximum fluorescence enhancement, or maximum tPA/plasminogen activation, respectively. For the fluorescence enhancement assays, this concentration has been set to 100 μg/ml. For the tPA/plasminogen activation assay, 40 μg/ml dOVA std. is used as a reference. When appropriate, fluorescence enhancement and tPA/plasminogen activation induced by dOVA std. has been arbitrarily set to 100% for comparison purposes.

### TEM Analysis of Glycated BSA and Hb

FIG. 4 illustrates that misfolding of BSA and haemoglobin by glycation induces non-fibrillar amorphous aggregates.

dIgIV-65 Octagram

FIG. 5 shows that denaturation of Octagam IgIV (dIgIV-65) induces crossbeta structure. It is seen that the misfolding condition results in misfolded Ig with appearance as aggregates on TEM images and enhanced Thioflavin T fluorescence. Fibrils are not observed.

dIgIV-86 Gammagard and Base-Denatured γ-Globulins

Enhanced fluorescence of Thioflavin T, Congo red, ANS, Bis-ANS and Thioflavin S was observed with the misfolded IgIV Gammagard sample dIgIV-86 in comparison with native IgIV (FIG. 6A-E). Similar characteristics were observed when Trp fluorescence is measured (FIG. 6F). TEM images at a magnification of 10K show that native IgIV Gammagard barely harbors any aggregates, and the aggregates present are amorphous and small in size (FIG. 7A). When denaturing temperature is set to 86° C. the aggregation size and abundance of the aggregates dramatically increase (FIG. 7B). The potency of the misfolded preparation dIgIV-86 Gammagard to activate tPA/plasminogen in a tPA mediated plasmin generation assay was examined (FIG. 7C). No tPA/plasminogen activation was observed with native IgIV Gammagard. Based on the tPA/plasminogen activation potency of the denatured IgIV-86 Gammagard preparation, dIgIV-86 is a potent activator.

The misfolded base-denatured IgG-base enhances Congo red fluorescence, ThT fluorescence and shows increased Trp fluorescence (FIG. 6A, B, F). Misfolded base-denatured human y-globulins IgG-base appear as aggregates on a TEM image (FIG. 7D). The number of aggregates is relatively high and the average size of the multimeric assemblies is relatively large. These markers altogether show the amyloid-like misfolded protein character of IgG-base, comprising crossbeta structure.

Aβ42t=0 Preparation

The. Aβ42t=0 preparation shows enhanced ThT and CR fluorescence levels (FIG. 8A-B). Aβ42t=0 appears as amorphous aggregates on a TEM image (FIG. 8C).

### Synthetic Fibrin Fragments FP13 and FP10

Preparation of synthetic fibrin fragment FP13 sample (KRLEVDIDIKIRS, SEQ ID 12), comprising crossbeta structure, and synthetic fibrin peptide FP10 (KRLEVDIDIK, SEQ ID 13) and subsequent structure analysis revealed the presence of crossbeta structure in FP13, whereas FP10 solutions do not display any sign for the presence of crossbeta structure (negative control), and has been described elsewhere (Kranenburg et al., 2002).

### HSP Binding Peptides: Structural Analysis

A series of peptides was selected based on literature and patent data showing that the peptides bind to HSPs HSP70 or BiP (Table 5). 6BB7 (referred to as np53) is known for being not a ligand for BiP. In addition, two Aβ peptides were selected for crossbeta binding studies, synthetic human Aβ16-22 (code 6BB12) and Aβ25-35 (code 6BB6), based on available information showing readily formation of crossbeta structure. The peptide sequences, sequence identity numbers and codes are given in Table 5. Peptides were purchased from NKI-Amsterdam (The Netherlands) and had unmodified N- and C-termini. The theoretical iso-electric points (pIs) of the peptides were calculated. Peptides were dissolved at 2.5 mg/ml in HCl solution pH 2, NaOH solution pH 12, or in buffer with pH=pI of the peptide. Freshly prepared peptide stocks were incubated for 30 minutes at 37° C. and subsequently aliquots were stored at −80° C. With the series of peptides, fluorescence of ThT and Congo red was assessed (FIG. 9A, B). In addition, the potency of the peptides to activate tPA/plasminogen was analysed (FIG. 9C). Finally, with a subset of the peptides, a TEM analysis was performed (FIG. 9).

### Results: Structural Analysis of Aβ16-22, A25-35 and HSP Binding Peptides

Results of the above described structural analyses of peptides described in literature as ligands for BiP or HSP70, and of Aβ16-22 and Aβ25-35 are summarized in Table 5 and depicted in FIG. 9. Non-BiP binding peptide 6BB7 does not display characteristics of crossbeta structure. All other peptides tested display one or more characteristics showing the presence of peptide conformation comprising crossbeta structure, i.e. enhanced ThT fluorescence, enhanced Congo red fluorescence, appearance on TEM images (formation of aggregates/fibers), activation of tPA/plasminogen.

### Endotoxin Levels in Samples Used for Example

Endotoxin levels in various solutions used for the experiments described in Examples 7 to 16 were determined with the Limulus Amebocyte Lysate (LAL) kit (Cambrex, QCL-1000). The kit was used according to the manufacturer's protocol, except that now measurements were performed using half of the described assay volume. As a reference lipopolysaccharide (LPS, Sigma, 2.5 mg/ml L-2630 clone 011:B4) was incorporated in several measurements. With the signals obtained with an LPS standard curve, an estimate of the endotoxin content in mass/volume was calculated with signals in endotoxin units (EU) obtained with unknown samples. In Table 6, endotoxin levels in EU are presented for the stock solutions. When required, cell based assays were performed with protein tester compound solutions comprising indicated final Polymixin B (PMXB, Sigma, P1004, 8070 units/mg) concentrations (stock of 20 mg/ml in PBS).

### Oxidation of Low Density Lipoprotein Particles Contributes to the Pathogenesis of Artherogenesis

Misfolding and aggregation of the apolipoprotein B100 (apoB) protein fraction of low density lipoprotein (LDL) upon oxidation, its resistance to proteolysis and its cytotoxicity are motifs commonly seen for amyloid-like protein aggregates. Moreover, multiligand receptors with affinity for amyloid-like structures, i.e. CD36, RAGE, scavenger receptor A and scavenger receptor B-I, are also receptors for oxidized adducts of lipoproteins. Therefore, it has been suggested that the role of amyloid-like apoB in oxidized LDL (oxLDL) particles during atherosclerosis is similar to the pathogenic role of amyloid-like aggregates in protein misfolding diseases. We now determined the structural characterstics of oxidized human plasma LDL with a focus on the presence of misfolded crossbeta protein markers.

Preparation of Oxidized Human Plasma Low Density Lipoprotein, oxLDL

Oxidized LDL was kindly provided by Dr Suzanne Korporaal and prepared as follows. Low density lipoproteins (LDL) were isolated from fresh (<24 h) human plasma that was kept at 10° C., obtained from the Netherlands bloodbank. Plasma was centrifuged in an ultracentrifuge for three subsequent cycles. The LDL fraction was isolated and stored under N₂, at 4° C. Before experiments, native LDL (nLDL) was dialyzed overnight at 4° C. against 0.9% w/v NaCl. To obtain oxLDL, native LDL was first dialyzed against 0.15 M NaCl solution containing 1 mM NaNO₃, overnight at 4° C. Then, nLDL was diluted to 3-5 mg/ml, and CuSO₄ was added to a final concentration of 25 μM and incubated at 37° C. In a similar way LDL was oxidized using FeSO₄ instead of CuSO₄. Oxidation with FeSO₄ was also preceded by the dialysis step. Next, LDL was dialyzed against 5 μM FeSO4 in PBS with additional 150 mM NaCl and 1 mM NaN₃, pH 7.2. The degree of oxidation is controlled by choosing a certain number of oxidation buffer refresh cycles. The more often FeSO₄ in buffer is refreshed each 10-12 h, the higher the degree of oxidation will be. To stop oxidation, the oxLDL sample is dialyzed against a buffer of 150 mM NaCl, 1 mM NaN₃, 1 mM EDTA for 4 h at 4° C. The degree of oxidation was determined by measurement of diene-formation at λ=234 nm (Ultrospec 3000 Spectrophotometer (Pharmacia Biotech)). oxLDL solution was stored at 4° C. under N₂. Presence of crossbeta structure conformation in the ApoB100 protein portion of LDL was analyzed using a Thioflavin T fluorescence assay, a Congo red fluorescence assay and a tPA/plasminogen activation assay (see above), as well as two variants of a factor XII activation assay (see below).

### Factor XII Activation Assay

Conversion of the zymogen factor XII (#233490, Calbiochem) to proteolytic active factor XII (factor XIIa) was assayed indirectly by measurement of the conversion of chromogenic substrate Chromozym-PK (Roche Diagnostics, Almere, The Netherlands) by kallikrein formed by factor XIIa cleavage of prekallikrein. Chromozym-PK was used at a concentration of 0.3 mM. Factor XII and human plasma-derived prekallikrein (#529583, Calbiochem) were used at concentrations of 1 and 7.7 nM, respectively. The assay buffer was HBS (10 mM HEPES, 4 mM KCl, 137 mM NaCl, 5.8 μM ZnCl₂, pH 7.2). Assays were performed using microtiter plates (#2595, Costar, Cambridge, Mass., USA). oxLDL was tested for its ability to activate factor XII. Between 3.13 and 25 μg/ml oxLDL was tested in duplicate, and 100 μg/ml glycated albumin as positive control and buffer only as negative control. The conversion of Chromozym-PK was recorded kinetically at 37° C., using a Spectramax 340 Microplate Reader. In control wells factor XII was omitted from the assay solutions.

Alternatively, activation of factor XII was assessed directly by omitting prekallikrein and using a factor XII chromogenic substrate, S-2222 (Chromogenix, Italy).

Results: Structural Analysis of oxLDL

### Oxidized LDL Displays Amyloid-Like Features Showing to the Presence of Crossbeta Structure Conformation

LDL oxidized using FeSO₄ was analyzed for its potency to activate crossbeta binding serine protease factor XII. When oxLDL that is oxidized for 59%, and which displays Thioflavin T fluorescence (not shown), is used, a dose dependent activation of factor XII and prekallikrein is determined (FIG. 10A), indicative for the presence of misfolded ApoB100 comprising crossbeta. Alternatively, freshly isolated LDL was oxidized upon incubation with 25 μM CuSO₄, for various incubation times. In time, the degree of oxidation was determined by reading the absorbance of diene structures at 234 nm, as well as the fluorescence upon incubation of oxLDL with Congo red or Thioflavin T (FIG. 10B, C). With a 24% oxidized oxLDL preparation, the ability to activate tPA in the chromogenic plasmin activation assay was determined and compared to native LDL. It was clearly seen that upon oxidation LDL gains tPA activating properties (FIG. 10D). In an additional experiment, the ability of oxidized LDL to activate factor XII in plasma was determined in a chromogenic assay using substrate S-2222. Like crossbeta comprising fibrin-derived peptide FP13, oxLDL stimulates the conversion of S-2222 by activated fibrin, indicative for the ability of oxLDL to induce factor XII activation and thus the presence of crossbeta structure (FIG. 10E).

The ability of oxLDL to both activate tPA and factor XII, is similar to what is observed with amyloid-like peptides and proteins with crossbeta structure. Therefore, together with the binding of Congo red and ThT, the ability of oxLDL to activate tPA and factor XII shows the presence of crossbeta structure in the apoB protein part of the oxidized LDL particles.

### Chaperone Stocks

### Chaperones Used for the Examples

Recombinant Escherichia coli (E. coli) DnaK was purchased from Stressgen (Ann Arbor, Mich., USA; SPP-630). For the binding studies first lot B502468 (100 μg, lot A) and then lot 05240609 (200 μg, lot B) was used.

Recombinant human HSP60 with N-terminal His-tag was a kind gift of Dr. R. van der Zee and Prof. Dr. W. van Eden (Institute of Infectious Diseases and Immunology, Faculty of Veterinary Medicine, Utrecht University, Utrecht, The Netherlands), and was supplied in lyophilized form. The HSP60 was produced in E. coli and purified using Nickel-Sepharose. The HSP60 was lyophilized from 20-40 mM ammonium-bicarbonate solution, after purification, and stored at −20° C. The construct lacks the mitochondrial signal sequence. The endotoxin content is 50 I.U./mg protein (Cambrex LAL assay) and HSP60 has been tested at 10 μg/ml on cultured intestine epithelial cells, which are TLR4 positive; no activation of cells by endotoxin. Before use, the lyophilized protein is dissolved at room temperature at 1 mg/ml in 20-40 mM NH₄HCO₃, resulting in a pH of 7.0. Aliquots are stored at −20° C. or −80° C. The mitochondrial HSP60 is a functional dimer of heptamers (MW(HSP60 monomer)=˜58 kDa), and is involved in multimer protein assembling and aids in transport of unfolded proteins across membranes. For the binding studies, at two occasions separate HSP60 solutions were prepared originating from the same batch of purified protein, lyophilized in separate Eppendorf cups.

Recombinant human HSP90beta was bought from Dr. S. Rüdiger (Cellular Protein Chemistry, Department of Chemistry, Faculty of Science, Utrecht University). HSP90beta eluted from an anion exchange column was supplied in 35 mM Bis-Tris/25mM Tris, 5 mM DTT, approximately 385 mM NaCl, approximately pH 9.0. The HSP90beta concentration was 5.0 μM (450 μg/ml with a MW of 90.000 Da; extinction coefficient of 53740 l/(mol*cm)). The supplied frozen stock is kept at −80° C. Before use, the stock is quickly thawed almost completely in a 37° C. water bath, and kept at wet ice. Before use, the HSP90beta solution is centrifuged for 5 minutes at 16,000*g at 4° C. During dilution of HSP90beta solution, 5 mM DTT should be incorporated in the dilution buffer, to keep free Cys residues. Thawed HSP90beta was aliquoted and snap frozen in liquid nitrogen before re-storage at −80° C.

### Cloning, Expression and Purification of the Soluble Extracellular Domains of Receptor for Advanced Glycation Endproducts

The soluble extracellular part of the receptor for AGE (sRAGE) was cloned, expressed and purified as follows (Q.-H. Zeng, Prof. P. Gros, Dept. of Crystal- & Structural Chemistry, Bijvoet Center for Biomolecular Research, Utrecht University, Utrecht, the Netherlands). Human cDNA of RAGE was purchased from RZPD (clone IRALp962E1737Q2, RZPD, Berlin, Germany). For PCRs, the gagatctGCTCAAAACATCACAGCCCGG forward primer was used comprising a BglII site, and the gcggccgcCTCGCCTGGTTCGATGATGC reverse primer with a NotI site. The soluble extracellular part of RAGE comprises three domains spanning amino-acid residues 23-325. The PCR product was cloned into a pTT3 vector, containing an amino-terminal His-tag and a thrombin cleavage site. The sRAGE was expressed in 293E hamster embryonic kidney cells at the ABC-protein expression facility (Utrecht University, Utrecht, the Netherlands). Concentrated cell culture medium was applied to a Hi-trap Chelating HP Ni²⁺-NTA column (Amersham Biosciences Europe, Roosendaal, The Netherlands). The running buffer was 25 mM Tris-HCl, 500 mM NaCl, pH 8.0. The protein was eluted by using a step gradient of 0 to 500 mM imidazole. Purity of the His-sRAGE was depicted from Coomassie stained SDS-PAGE gels. Purified sRAGE-His stock was 284 μg/ml (7 μM with MW(sRAGE)=40 kDa) in PBS, stored at −80 ° C.

Cloning and Expression of Recombinant Fibronectin Finger Domains that Bind Misfolded Proteins

For a description of cloning, expression and purification of recombinant human fibronectin finger domains 4-5 (Fn F4-5), now with a C-terminal FLAG-tag and His-tag, see patent application WO2006101387 (paragraph [0137]-[0165] and [0192-0194]). Protein expression in human embryonic kidney cells and purification was performed with the aid of the ABC-Expression Facility (University of Utrecht, The Netherlands; see below).

Purification of BiP-FLAG-His, sRAGE-FLAG-His and Fn F4-5-FLAG-His

The 293 cells were pelleted after 5 days culturing by centrifugation and the supernatant was concentrated on a Quixstand concentrator, using a 30 kDa cut-off filter (BiP) [GE Healthcare], or a 5 kDa cut-off filter (FN4,5) (GE Healthcare). A dialysis step was performed on the same concentrator, and the proteins were dialysed either against PBS+0.85 M NaCl pH 7.4 (BiP) or against 25 mM Tris pH 8.2+0.5 M NaCl (Fn F4-5). The concentrated and dialysed medium was filtered (0.45 μm, Millipore) and incubated with Ni-Sepharose beads (GE-Healthcare, 17-5318-02) in the presence of 10-20 mM imidazole, for either 3 h at room temperature or overnight at 4° C. under constant motion. A column was made of the beads and the proteins were extracted by increasing amounts of imidazole. The proteins purified in this way had a purity of 80-90%, as established by SDS-PAGE electrophoresis (Invitrogen, NuPage 4-12% BisTris, NP0323), using MOPS buffer (Invitrogen, NP0001) for BiP or MES buffer (Invitrogen, NP0002) for Fn F4-5, and Coomassie staining (Fermentas PageBlue, R0571). The purest fractions were pooled and dialysed in a 3.5 kDa cut-off membrane (Spectra/Por, 132720) against the indicated buffers without imidazole and with 5-% glycerol. Protein concentrations were determined using a BCA kit (Pierce). Purified Fn F4-5-Flag-His, at 288 μg/ml in PBS containing 5% glycerol, is stored at −80 ° C. Purified BiP-FLAG-His, at 1.16 mg/ml in PBS/5% glycerol was also stored at −80° C. The sRAGE was purified similarly and also aliquots were stored at −80° C. The sRAGE-FLAG-His concentration was 790 μg/ml, 20 μM.

### Enzyme Linked Immunosorbent Assay for Testing of Chaperone Binding to Misfolded Proteins

Binding of chaperones to immobilized (misfolded) protein ligands was determined using an enzyme linked immuno sorbent assay (ELISA) approach. For this purpose 50 μl/well of potential ligands at indicated concentrations or coat buffer only for control and background measurement purposes, were coated overnight at 4° C. or coated for 1 h at room temperature, with motion, in 50 mM NaHCO₃ pH 9.6 (coat buffer). In general, ligands were coated at 5 μg/ml, unless stated otherwise. For example, BiP binding peptides 6BB# and oxLDL were coated at 50 μg/ml. Native haemoglobin (Sigma-Aldrich, H7379), albumin, IgIV Gammagard and ovalbumin controls were prepared from lyophilized stocks. The controls were prepared by dissolving lyophilized proteins at 1 mg/ml in PBS upon resuspending by pipetting, followed by a 30 minutes period at the roller device, at room temperature. The protein solutions were centrifuged for 10 minutes at 16,000*g and diluted in coat buffer. After coating the plates were washed at least three times with 50 mM Tris-HCl pH 7.3, 150 mM NaCl, 0.1% v/v Tween20, and blocked with Blocking reagent (Roche Diagnostics, Almere, The Netherlands; 11112589001), for 1 h at room temperature, with motion. Plates were washed three times and incubated in triplicate with indicated chaperone dilution series, at 50 μl/well, for 1 h at room temperature, with constant motion. For HSP90, 5 mM DTT is incorporated in the binding buffer (PBS/0.1% Tween20) to avoid unwanted disulfide bond formation of the HSP90. Plates were washed three times and incubated with anti-FLAG antibody (mouse antibody, M2, peroxidase conjugate; Sigma, A-8592) for BiP-FLAG-His, anti-His-tag antibody (Mouse Monoclonal Antibody, Cat. No. 70796, Novagen [0.2 mg/ml]) for HSP60, a 1:1 mix of 500-fold diluted anti-HSP90beta antibodies (Stressgen, SPA-643 [mouse monoclonal IgM], SPA-646 [rabbit polyclonal]) for HSP90 and an anti-DnaK antibody (Stressgen, 023SPA-880D [mouse monoclonal antibody]) for DnaK. After four wash cycles, secondary antibodies were added to the wells when required, 50 μl/well, for 45′ at room temperature, with motion. RAMPO and SWARPO were used at 3000 times dilution. For detection of HSP90beta binding, in a first experiment with coated HbAGE and control Hb, the rabbit polyclonal anti-HSP90 antibody is used, followed by 1:3000 SWARPO. In a second experiment with more immobilized misfolded proteins and controls, the detection of bound HSP90 with the mix of two anti-HSP90 antibodies was achieved by mixing 1:1500 SWARPO with 1:3000 RAMPO. After 5 washes with wash buffer followed by two washes with PBS, binding of antibodies was assessed. Bound anti-FLAG-PO, peroxidase-conjugated rabbit anti-mouse immunoglobulins (RAMPO, P0260, DAKOCytomation, Glostrup, Denmark) or peroxidase-coupled swine anti-rabbit immunoglobulins (SWARPO, P0217, DAKOCytomation) was visualized with tetramethylbenzidine (TMB, #45.01.20,/#45.014.01, Biosource, Nivelles, Belgium). The reaction was stopped after 5 minutes with 1% H₂SO₄ in H₂O. Plates were read at 450 nm. Data reduction was performed as follows. Triplicates were averaged and standard deviations calculated. Background signals obtained with buffer-coated wells were subtracted (binding of primary antibody to empty wells), as well as background signals obtained with ligand-coated wells in which the chaperones were omitted (binding of primary and secondary antibody to coated ligands). Subsequently, sub-optimal concentrations of chaperones for binding to misfolded protein ligands are determined, and these concentrations are used for inhibition studies. For this purpose, the sub-optimal chaperone concentration is applied to coated misfolded protein ligands in the presence of indicated concentration series of tPA, K2P tPA, Congo red, ThT, ThS, sRAGE, Fn F4-5, BiP, IgIV Octagam, HSA, or geldanamycin (HSP90 only, 10 μM final concentration from a 10 mM 1000× stock in 100% DMSO; Biomol International, supplied by TebuBio, The Netherlands, 034EI-280-1000). The influence of tPA and K2P tPA was tested in the presence of 10 mM ε-amino caproic acid, to avoid binding of the kringle2 domain of tPA and K2P tPA to lysine- and arginine residues (tPA binding to amyloid-like structures is mediated by its finger domain, that is lacking in truncated K2P tPA; the kringle2 domain binds to exposed side chains of lysines and arginines). We have previously identified that a sub-fraction of IgIV molecules has affinity for misfolded proteins comprising crossbeta structure (pending patent application). Moreover, we identified that the sub-fraction of IgIV molecules directly binds to crossbeta structure and/or crossbeta structure induced conformation (not shown; pending patent application). Binding buffer, HSA and K2P tPA serve as negative controls in these inhibition studies.

### ELISA: Binding of HSPs to Fibrin

We previously demonstrated that the well-known ligand for crossbeta-binding protein tPA, localized in a blood clot, i.e. fibrin, comprises crossbeta structure (patent application US2007003552). For testing the binding of chaperones to immobilized fibrin with crossbeta conformation, the following protocol was applied to obtain wells of 96-wells ELISA plates with immobilized fibrin:
1. Prepare a 2 U/ml factor IIa stock in H₂O from a standard factor IIa/thrombin stock (human plasma, High Activity, Calbiochem, Germany, prod. nr 605195)
2. Prepare a 50 μg/ml fibrinogen solution (Fib3L 2170L in 20 mM sodium citrate-HCl pH 7.0, Kordia, The Netherlands) in PBS (3 g di-sodium hydrogen phosphate, 0.33 g sodium dihydrogen phosphate and 8.2 g sodium chloride per liter) from a stock solution that is centrifuged for 10 minutes at 16.000*g before use.
3. Pipet 5 μl of factor IIa solution into the wells, add 100 μl of fibrinogen solution, or add 100 μl PBS to control wells. Final concentrations: [factor IIa]≈0.1 U/ml, [fibrinogen]≈47.5 μg/ml.
4. Incubate for 2 hours at room temperature with gentle agitation. Coat controls are performed using anti-human fibrinogen antibody (DAKO-Cytomation, A0080).
5. Emptied wells are washed twice with TBS/0.1% Tween20. TBS: Tris-buffered saline with 150 mM NaCl, 50 mM Tris-HCl, pH 7.3 [10× TBS buffer stock containing for 10 liter, 1211 g Tris, 1752 g NaCl, 750 ml HCl (˜37%), pH 7.0-7.4].

Then, a regular ELISA binding experiment is performed, as outlined above.

### EXAMPLE 7

Interaction of Human BiP with Misfolded Proteins Comprising Crossbeta Structure

To test whether human ER- and extra-cellularly localized HSP70 family member BiP, which acts in the UPR, and which extra-cellular concentration is increased in the synovium in 75% of RA patients, and which has been identified as a target auto-antigen in RA patients, can interact specifically with misfolded proteins comprising crossbeta structure, a series of direct ELISAs has been performed using coated misfolded ligands and native controls. FIG. 11 depicts the results of these binding studies. Binding of concentration series of BiP to misfolded proteins with crossbeta structure has been established for proteins HbAGE, BSA-AGE, oxLDL, dOVA standard, dIgIV, fibrin and peptides Aβ1-40, Aβ1-42, Aβ25-35, fibrin fragment FP13, synthetic peptides comprising crossbeta structure and that has been described as BiP ligands, 6BB9, 6BB10, 6BB11 (See Table 5). BiP did not bind to control proteins and peptides ovalbumin, fibrin fragment FP10, haemoglobin, thrombin, 6BB7, a synthetic peptide that has been documented in literature as a non-BiP binding peptide, and albumin. With the applied experimental conditions, no BiP binding was observed with synthetic peptides 6BB8, 6BB13 (Table 5 for sequences) and Aβ16-22. These latter three peptides comprise characteristics of crossbeta structure (Table 5).

From these extensive binding studies we conclude that BiP specifically interacts with a series of misfolded proteins and peptides that comprise crossbeta structure. Aβ16-22, 6BB8 and 6BB13 either comprise types of crossbeta structure and/or comprise crossbeta structure induced conformation that are no binding partner for BiP, or the experimental conditions did not allow interaction of BiP with the peptides comprising crossbeta structure and/or crossbeta induced conformation.

In a subsequent study we analysed whether BiP interacts directly with the crossbeta structure, by performing ELISA binding studies with a fixed sub-optimal concentration of BiP and concentration series of crossbeta binding compounds. The results are depicted in FIGS. 12, 13 and Table 7. Binding of BiP without added crossbeta binding compounds was set to 100%. We conclude that tPA and Congo red are general inhibitors of the interaction of BiP with misfolded proteins comprising crossbeta structure. K2P tPA was implied in the study as a negative control for crossbeta binding tPA, because K2P tPA lacks the finger domain that interacts with misfolded proteins. Indeed, K2P tPA did not modulate binding of BiP to the crossbeta ligands. Both ThT and ThS can either potentiate BiP binding, or inhibit the interaction of BiP with the crossbeta ligands. Interestingly, ThT and ThS show the same ability for a certain crossbeta ligand: either both crossbeta binding dyes potentiate binding, or both crossbeta binders inhibit binding. Notably, all three tested BiP-binding crossbeta peptides 6BB9-11 (Table 5 for sequences) show decreased BiP binding under influence of ThT and ThS. Regularly, we observe stimulated binding of a crossbeta binding protein to crossbeta ligands, e.g. for tPA-BSA-AGE, tPA-Aβ, IgIV-HbAGE (not shown).

From the results of the competition binding studies using crossbeta binding compounds ThT, Congo red, ThS, IgIV and tPA (see Table 1, 2) we conclude that BiP interacts at least in part directly with crossbeta structure and/or crossbeta structure induced conformation in misfolded ligands comprising crossbeta structure, e.g. Aβ25-35, HbAGE, 6BB9, 6BB10, 6BB11, oxLDL and dOVA standard. Therefore, we designate BiP as a newly identified crossbeta binding protein, from now on listed in our table of crossbeta binding compounds (Table 2).

### EXAMPLE 8

Interaction of Human HSP60 with Misfolded Proteins Comprising Crossbeta Structure

To test whether human mitochondrial HSP60 can interact specifically with misfolded proteins comprising crossbeta structure, a series of direct ELISAs has been performed using coated misfolded ligands and native controls. FIG. 14 depicts the results of these binding studies. Binding of concentration series of HSP60 to misfolded proteins with crossbeta structure has been established for HbAGE, BSA-AGE, dOVA standard, dIgIV, Aβ1-42, Aβ25-35 and fibrin. HSP60 did not bind to native controls ovalbumin, haemoglobin, IgIV and albumin.

Similar to BiP, HSP60 did not bind to Aβ16-22, and in addition HSP60 did also not bind to oxLDL.

The role of crossbeta structure in the interaction between HSP60 and the misfolded protein ligands was analysed by determining the binding properties of HSP60 to BSA-AGE, Aβ42t=0 and dIgIV-86 under influence of concentration series of ThT or Congo red (FIG. 15). Binding of HSP60 to BSA-AGE is inhibited by Congo red. Both ThT and Congo red inhibit binding of HSP60 to Aβ42. ThT stimulates binding of HSP60 to the misfolded IgIV. These results disclose HSP60 as a protein with affinity for crossbeta structure and/or crossbeta structure induced conformation(s) in misfolded protein ligands (Table 2).

### EXAMPLE 9

Interaction of Human HSP90 with Misfolded Proteins Comprising Crossbeta Structure

To test whether human intra-cellularly localized HSP90 can interact specifically with misfolded proteins comprising crossbeta structure, a series of direct ELISAs has been performed using coated misfolded ligands and native controls. FIG. 16 depicts the results of these binding studies. Binding of concentration series of HSP90 to misfolded proteins with crossbeta structure has been established for HbAGE and Aβ25-35, whereas little binding was observed with oxLDL, Aβ42t=0 and non-BiP binding peptide 6BB7. HSP90 did not bind to native controls haemoglobin and IgIV. Similar to BiP, HSP90 did not bind to Aβ16-22 and the BiP-binding peptides 6BB8-11, 13, and in addition HSP90 did also not bind to dIgIV-86 and dOVA standard. From these results we conclude that HSP90 interacts with several misfolded proteins comprising crossbeta structure, but that the crossbeta structures in Aβ16-22, misfolded IgIV, BiP-binding peptides and dOVA do not expose a HSP90 binding site with the current experimental parameters.

To determine whether the crossbeta structure is part of the binding site on misfolded proteins for interaction with HSP90, we performed binding studies with 20 μg/ml HSP90 and coated HbAGE or Aβ25-35, under influence of Congo red and ThT. Geldanamycin, a known inhibitor of the interaction between HSP90 and its ligands, was also incorporated in the competition ELISAs. We observed that for HbAGE, ThT had no influence on HSP90 binding, whereas fibronectin F4-5 and Congo red inhibited HSP90 binding (FIG. 17). For Aβ25-35, ThT and Congo red, stimulatory activity was observed with respect to HSP90 binding. In contrast, sRAGE and fibronectin finger 4-5 inhibited binding of HSP90 to Aβ25-35, whereas control albumin did not influence binding (FIG. 17). Geldanamycin did not influence binding of HSP90 to HbAGE or Aβ25-35. That is to say, the consistent inhibition of HSP90 binding to HbAGE and the consistent potentiation of HSP90 binding to Aβ25-35 could be attributed to geldanamycin and 0.1% DMSO (FIG. 17).

These data altogether show that HSP90 interacts with proteins comprising crossbeta structure and that the crossbeta structure and/or crossbeta structure induced conformation contributes to the ability of HSP90 to bind to a misfolded protein comprising crossbeta structure. Based on the observed modulating activity of geldanamycin, crossbeta structure induced conformation in a misfolded protein contributes to the ability of HSP90 to bind. Alternatively, it can be concluded that geldanamycin is a crossbeta binding compound, thereby modulating the binding of HSP90 to misfolded proteins comprising crossbeta structure. In conclusion, HSP90 is a crossbeta binding protein, and as such is part of the Crossbeta Pathway (Table 1-4). In addition, Alzheimer peptide and glycated protein are identified as misfolded ligands comprising crossbeta structure for HSP90.

### EXAMPLE 10

Interaction of Escherichia coli HSP70 Family Member DnaK with Misfolded Proteins Comprising Crossbeta Structure

To test whether Escherichia coli HSP70 family member DnaK can interact specifically with misfolded proteins comprising crossbeta structure, a series of direct ELISAs has been performed using coated misfolded ligands and native controls. FIG. 18 depicts the results of these binding studies. Binding of concentration series of DnaK to misfolded proteins with crossbeta structure has been established for fibrin, HbAGE, BSA-AGE, oxLDL, dIgIV-86, Aβ1-42 and Aβ25-35. Low binding is observed for the highest concentration DnaK tested with native Hb and native IgIV. With the current experimental parameters, DnaK did not bind to Aβ16-22 and BiP-binding peptides 6BB8, 10, 11, 13, that comprise crossbeta structure. No binding is observed with non-crossbeta protein albumin and peptide 6BB7. From these results we conclude that similar to BiP, also this HSP70 can interact with misfolded proteins comprising crossbeta structure. Apparently, at least not all crossbeta-comprising molecules are ligands for DnaK. In the datasheet accompanying IgIV, it is stated that at most 3% of the molecules is aggregated, providing an explanation for DnaK binding. When dissolving lyophilized Hb we regularly observe Hb particles that hardly dissolve, which is also indicative for the presence of a fraction of incorrectly folded molecules, explaining the medium binding of DnaK to ‘native’ Hb.

In a subsequent experiment we determined the influence of crossbeta binding molecules ThT and Congo red on the binding of the sub-optimal concentration of 20 μg/ml DnaK to immobilized HbAGE and dIgIV-86 (FIG. 19). Both crossbeta binding dyes ThT and Congo red inhibit the interaction of DnaK with both tested misfolded proteins comprising crossbeta structure. These data show that crossbeta structure contributes to the binding site on the misfolded proteins for DnaK. Therefore, in conclusion, the analyses revealed that DnaK is a member of the family of crossbeta binding proteins and proteins binding to crossbeta induced protein conformations, at least with the experimental conditions, that altogether build up the Crossbeta Pathway (Table 1-4).

### Summary of Examples 7-10

The above described binding analyses with a series of misfolded proteins comprising crossbeta structure and human HSP70 family member BiP, that is localized intra-cellularly in the ER and is localized extra-cellularly, the E. coli HSP70 family member DnaK, human mitochondrial HSP60 and intra-cellular human HSP90, revealed that chaperones as diverse as the tested series of chaperones incorporated in our studies, interact with crossbeta structure and crossbeta structure induced protein conformation. The analysed chaperones differ in their localisation in the body (in cells, extracellular, sub-cellular localisation). The structure and proposed mode of action of the HSP60, HSP70 and HSP90 differ to a great extent as well, as do the identified number and identity of binding partners for each of the four HSPs. Moreover, comparative binding characteristics were observed with HSP70 family members from rather diverse species, i.e. mammalian BiP and bacterial DnaK. From our studies it is revealed that for various misfolded proteins comprising crossbeta structure, the analysed HSPs share overlapping binding sites for crossbeta structure and/or crossbeta structure induced conformation with crossbeta binding molecules Congo red, ThT, ThS, IgIV, tPA, sRAGE and fibronectin F4-5, and the HSPs have unique binding properties for other crossbeta structure comprising proteins, when binding patterns of the HSPs are compared with crossbeta binding molecules like ThT and Congo red. From our observations altogether, therefore, we conclude that the family of chaperones are crossbeta binding proteins and/or proteins binding to crossbeta structure induced conformations in proteins.

Misfolded glycated proteins, oxLDL and misfolded IgG comprising crossbeta structure are auto-antigens in rheumatoid arthritis (RA). We now demonstrate that these auto-antigens are ligands for chaperones, at least in part through the presence of crossbeta binding sites.

### EXAMPLE 11

Analysis of the Binding of HSPs with Crossbeta Adjuvated Vaccines

Previously, we demonstrated that the presence of crossbeta structure in a vaccine is sufficient and effective in inducing a protective immune response, circumventing the need of a vaccine-potentiating adjuvant (patent application WO2007008070). Moreover, we demonstrated that a series of proteins was only then immunogenic in mice, when administered in a crossbeta structure conformation, whereas the native counterparts of the antigens were not immunogenic (Maas et al., 2006). Notably, we were able to break tolerance for a self-protein by administering the protein with crossbeta conformation, without the use of any additive, whereas the self-protein with a native conformation did not break tolerance. These data demonstrate that the presence of crossbeta structure in an antigen is a requirement for activation of the immune system. In several of our vaccination and immunization trials, highest antibody titers against a natively folded antigen were reached when the crossbeta antigen was administered when mixed with a fraction of the antigen with its native conformation. The crossbeta structure is required to activate the immune system, whereas presence of native antigen allows also for mounting a refined immune response against the desired native antigen, as it will appear at the surface of a pathogen during an infection.

In literature it has been stated that several chaperones possess immuno-modulating activity. For example, it has been demonstrated that binding of gp96 to tumor antigens aids in eliciting an anti-tumor immune response. Additionally, it has been reported that several HSPs only then interact with cell surface receptors when the HSPs are loaded with misfolded protein/peptide cargo. Interestingly, the indicated receptors are amongst others multiligand receptors TLR4, LOX-1, CD36, SR-A, CD91 and CD40, that all also have been identified as receptors for misfolded proteins directly (Table 2). Other investigators reported about the so-called ‘chaperokine’ activity of several HSPs. Some HSPs, e.g. HSP60 and HSP70, display pro-inflammatory activity during auto-immune disease RA, whereas other chaperones like BiP possess anti-inflammatory activities. Combined with our observations that auto-antigens in RA, e.g. glycated proteins and misfolded IgG, a role in the pathology of the disease for the interaction between the extra-cellular localized auto-antigens and extra-cellular localized chaperones is evident. Notably, in 75% of RA patients, BiP levels are increased in the synovium during inflammatory activity.

In this Example we now demonstrate that BiP interacts with crossbeta-adjuvated vaccines prepared for immunization of mice and subsequent titer determinations against native antigen lacking crossbeta. For this purpose, we used the vaccine solutions used for immunization for coating on Greiner Microlon high binding plates. Following the standard protocol as depicted above, binding of concentration series of BiP, tPA, HSP60 and K2P tPA to the three vaccines was assessed. The three vaccines comprise 0/50/100% crossbeta antigen and accordingly 100/50/0% native antigen, respectively. Antigens under consideration during the immunization trial were sub-unit vaccines H5 and E2. See for details about the structural analysis of the preparations patent application WO2007008070, Example 11 (paragraph [0234]). In WO2007008070 also the immunization trial is depicted. At indicated time intervals, blood was drawn and putative anti-E2 antibody titers and anti-H5 antibody titers were assessed using native antigens as ligands (FIG. 20). Now, in addition, binding of tPA, K2P tPA negative control, BiP and HSP60 to immobilized E2/H5 vaccine is assayed. Results are summarized in FIG. 20J.-L.

From the ELISAs we learn that none of the three vaccines comprising 0/50/100% crossbeta antigens exposes binding sites for K2P tPA, a truncated tPA lacking the crossbeta binding finger domain (FIG. 20L.), and HSP60 (not shown). For tPA, binding is seen for 50% crossbeta antigens and even more pronounced when 100% crossbeta vaccine is used as a coated ligand for tPA. Not only the maximum signal that can be obtained by using 100% as a ligand is higher with respect to the 50(:)50 solution, but in addition the affinity of tPA for 100% crossbeta antigen mixture is also approximately two-fold lower. In addition, BiP binding was established for the 100% crossbeta vaccine and not for vaccines comprising 0% or 50% crossbeta-adjuvated antigens. These data together demonstrate that the used protein antigens expose BiP binding sites and tPA binding sites, when crossbeta structure is present. Apparently, with the current experimental parameters, the immobilized antigens do not expose binding sites for pro-inflammatory HSP60.

In conclusion, our studies demonstrate that chaperones interact with known RA auto-antigens like for example glycated protein and aggregated immunoglobulins, and that BiP binds to crossbeta-adjuvated vaccine antigens. Therefore, for vaccine development based on crossbeta-adjuvation, monitoring the interaction of the crossbeta-adjuvated vaccine with chaperones is a valuable tool for optimization of efficacy and specificity of the vaccine with respect to dosing and directing the desired immune response. The best crossbeta antigen ligand can be designed for optimal interaction with a target HSP, or the opposite, i.e. binding of an HSP with a crossbeta-adjuvated antigen can be avoided by adjusting the crossbeta adjuvant in a way that it is no longer a binding partner for the HSP. Subsequently, it can be analysed whether desired interaction with a cell surface receptor like for example CD36, CD91, SRA, is optimized.

For drug development strategies for discovery and refinement of for example RA drugs, also the disclosed interaction between misfolded RA auto-antigens comprising crossbeta structure and chaperones is a new parameter of interest useful for pre-clinical adjustment of the target drugs towards a desired activity; being it interfering activity of the drug candidate with respect to the interaction of pro-inflammatory/anti-inflammatory HSPs with misfolded auto-antigens, or the opposite, potentiation of the interaction of pro-inflammatory/anti-inflammatory HSPs with misfolded auto-antigens by a drug candidate.

### EXAMPLE 12

Identification of Misfolded Proteins from Plasma of AL Amyloidosis Patients and Serum and Synovial Fluid of RA Patients, Using BiP-Sepharose with Affinity for Misfolded Proteins Comprising Crossbeta for Extraction of Misfolded Proteins from Protein Solutions like for Example Complex Patient Fluids

Since crossbeta structure containing molecules are effectively bound to a chaperone, they can now be effectively separated and/or isolated from a sample and/or an animal's or human's body and subsequently identified. BiP, a protein that we now identified as a crossbeta binding protein that can interact with misfolded proteins comprising crossbeta (see Example 7 and 11), was used to isolate proteins binding to crossbeta structure or crossbeta structure induced conformations in proteins and/or crossbeta structures and/or proteins comprising a crossbeta structure. Proteins binding to a crossbeta structure and/or a crossbeta induced conformation in proteins are identified by the fact that when bound to proteins containing crossbeta structure and/or crossbeta induced conformation in an unsaturated manner, BiP matrices can bind to the free binding sites on the protein containing crossbeta structure and/or crossbeta induced conformation, thereby indirectly binding to the proteins binding crossbeta structure and/or crossbeta structure induced conformation bound to the proteins containing crossbeta structure or crossbeta structure induced conformation. The presence and/or identity of a crossbeta structure, and/or protein comprising a crossbeta structure and/or proteins binding to crossbeta structure or crossbeta structure induced conformations in proteins, of healthy individuals was compared with the presence and/or identity of a crossbeta structure, and/or protein comprising a crossbeta structure and/or proteins binding to crossbeta structure or crossbeta structure induced conformations in proteins, from individuals with a disease or health problem related to and/or associated with a crossbeta structure and/or a protein comprising a crossbeta structure and/or proteins binding to crossbeta structure or crossbeta structure induced conformations in proteins, like for example from individuals with primary AL amyloidosis or rheumatoid arthritis (RA). The identity of proteins isolated with the use of BiP coupled to a solid support, i.e. Sepharose, was identified by mass-spectrometric analyses. The results of a sample originating from a healthy individual and a sample originating from a patient were compared. Furthermore, results obtained with a sample from a patient or a healthy individual contacted to BiP-matrix was compared to results obtained after contacting the same samples to control matrix without immobilised BiP. The results of these experiments are depicted in Table 8-13. In this way, information was obtained about the identity of proteins susceptible for misfolding and/or of protein(s) that preferentially bind(s) to those misfolded protein(s) and protein(s) prone to misfold and adopt crossbeta structure conformation during defined disease states. This provides key information for development of diagnostic tools that are disease specific, for instance to monitor disease state, to monitor effectiveness of therapy, to monitor occurrence of disease, and provides valuable leads for development of therapeutics targeted at crossbeta structures and/or protein(s) comprising a crossbeta structure and/or proteins binding to crossbeta structure and/or crossbeta structure induced conformations in proteins, which are preferably specific for the exemplary disorders. The therapeutics can be aimed at neutralising in the sense of covering/shielding, and/or clearing the misfolded proteins in situ, or at clearing the misfolded proteins extra-corporally, using for example affinity matrix during dialysis regimes.

### Material and Methods

BiP was coated on CNBr-Sepharose (GE-Healthcare, Amersham Biosciences). Immobilization of BiP was performed essentially as described elsewhere in this application for NHS-Sepharose. CNBr-matrix was dissolved at 200 mg/ml in 1 mM HCl and treated the same as the NHS-matrix, except for an additional 5 minutes activation step in 1 mM HCl on a roller device before washing in this buffer. BiP was diluted in immobilization buffer (50 mM NaCl and 40 mM NaHCO₃) to a concentration of 820 μg/ml. A total volume of 2 ml BiP solution (1640 μg BiP) was incubated with 360 mg beads. Negative control matrix was exposed to immobilization buffer, only. After overnight immobilization matrix was blocked with Tris and washed. The coat efficiency with BiP was approximately 75%.

For control purposes, the same series of six samples was also contacted with an affinity matrix composed of IgG-Sepharose, and eluates were obtained and analyzed by mass spectrometry after tryptic digestion for the identity of bound proteins, similarly as described above and below. The IgG was obtained by contacting intravenous immunoglobulins (IgIV, Octagam, Octapharma) with crossbeta HbAGE-Sepharose, eluting bound IgIV molecules with buffer comprising 1 M NaCl, dialysing the enriched IgIV fraction, and subsequently coupling of the enriched IgIV fraction with affinity for misfolded proteins to Sepharose, similarly as described for BiP.

Six samples were incubated with the BiP-Sepharose, IgIV-Sepharose and the control-Sepharose: Normal pooled plasma, plasma of a patient I or of a patient II, with AL amyloidosis, serum of a patient III with RA (Rheumatoid Factor (RF) titer 682), control serum and synovial fluid of a patient IV with RA (RF titer 23). All samples were diluted 20× in HBS and applied to 200 μl beads in two volumes of 500 μl. One volume was incubated for 4 h at RT and supernatant was discarded after centrifugation (2 minutes at 1400 rpm). Subsequently, the second volume was applied to the same matrix and incubated overnight on a roller device at 4° C. The affinity matrix or control matrix were washed 12 times with HBS and bound proteins were eluted with 2×50 μl of 8 M Urea in PBS, in two subsequent incubation steps of 1 h each. To collect the eluates, the matrices were centrifuged and the two eluates were pooled for each sample.

Sample codes:
A1, B1, C1, normal pooled plasma
A2, B2, C2 AL amyloidosis plasma of patient I
A3, B3, C3 AL amyloidosis plasma of patient II
A4, B4, C4 serum of patient III with RA (RF titer 682)
A5, B5, C5 control serum
A6, B6, C6 synovial fluid of patient IV with RA (RF titer 23)
A-series: affinity matrix of IgIV-Sepharose
B-series: affinity matrix of BiP-Sepharose
C-series: negative control matrix (activated/de-activated Sepharose)
   Eluted proteins were reduced with dithiothreitol (DTT) (60 minutes, final concentration 6.5 mM) and then alkylated with iodoacetamide (30 minutes, final concentration 54 mM), followed by overnight tryptic digestion (10 ng/μl). Protein digests were desalted as described (Rappsilber et al., 2003), vacuum dried and dissolved in 2.5% formic acid.

For analysis of peptide mixtures, an Agilent 1100 HPLC system (Agilent Technologies) connected to a Thermo Finnigan LTQ-MS (Thermo Electron, Bremen, Germany) was used. Protein digests were injected on a trap column (Reprosil C18 RP (Dr Maisch, Germany), 20 mm×100 μm I.D.) at 5 μl/minute. Subsequently, the peptides were transferred with a split-reduced flow rate of 100 nL/minute solvent A (0.1 M acetic acid) on the analytical column (Reprosil C18 RP, 20 cm×50 μm I.D.). Elution of the peptides was achieved with a linear gradient from 0 to 40% B (0.1 M acetic acid in 80% (v/v) acetonitrile) in 40 minutes. The column effluent was directly introduced into the ESI source of the mass spectrometer via a butt-connected nano-ESI emitter (New Objectives, Woburn, Mass.). The mass spectrometer was operated in the positive ion mode and parent ions were selected for fragmentation in data-dependent mode.

After mass spectrometric measurements, peak lists were generated using BioWorks software (Thermo Electron, Bremen, Germany). Protein identification was performed using Mascot software (www.matrixscience.com) by searching the IPIhuman database (version 3.24, downloaded from ftp://ftp.ebi.ac.uk/pub/databases/IPI/current) using the following settings: fully tryptic peptides, peptide tolerance 0.8 Da, MS/MS tolerance 0.9 Da, 1 missed cleavage allowed, carbamidomethyl (Cys) and oxidation (Met) as fixed and variable modification, respectively. The Scaffold software package (www.proteomesoftware.com) was used to parse the data and to filter peptides at a confidence level of 95%, allowing only protein identification with at least 2 peptides identified.

### Results

In Table 8-13 the results are displayed for the different samples, obtained after contacting fluids with BiP-Sepharose or enriched IgIV-Sepharose. The data obtained with enriched IgIV-Sepharose is discussed more thoroughly in a separate patent application (priority date Feb. 16, 2006). For the amyloidosis patients human pooled plasma was used as a control. For the RA patient, serum from a healthy subject was used as a control. The results for control serum and normal pooled plasma are used for identification of peptides that are uniquely present in peptide compositions obtained with patient samples. The proteins displayed are the proteins or protein fragments which bound specifically from patient serum or plasma, compared to the control serum or plasma. Since there was no synovial fluid from a healthy subject available, only the control-matrix was used as a negative control for the synovial fluid from a RA patient. As mentioned, protein identification was performed by searching the IPIhuman database. IPI stands for ‘International Protein Index’, and is used to identify proteins, protein precursors and protein fragments in different databases, such as Swiss-Prot, TrEMBL, and PIR (these databases are all coupled in UniProt). IPI protein sets are made for a limited number of higher eukaryotic species whose genomic sequence has been completely determined but for which there are a large number of predicted protein sequences that are (not yet) listed in UniProt. IPI takes data from UniProt and also from sources comprising predictions, and combines them non-redundantly into a comprehensive proteome set for each species. This information was all accessed through the website of the European Bioinformatics Institute (EBI) which is accessible via: www.ebi.ac.uk. In general, only those protein fragments that appear uniquely in samples obtained with IgIV-matrix or BiP-matrix are further investigated. Three proteins (IPI00748158, IPI00449920 and IPI00430820) for which one or two peptide(s) were identified in the eluate of control matrix that was contacted with synovial fluid is listed because multiple peptides (≧6) of this protein were identified in the eluate of the BiP matrix. As seen, there are several ‘hypothetical’ proteins and proteins indicated by the molecular weight of the detected proteins. Because relatively short amino-acid sequences cannot always be attributed uniquely to a specific protein, which is especially seen among immunoglobulins (Igs), multiple results are possible for some of the protein fragments identified. In some other cases the IPI number of the hypothetical protein refers to an already identified protein. The same sera were also screened for binding of potential misfolded proteins to an IgIV affinity matrix. In this experiment IgIV was enriched using a HbAGE crossbeta-affinity matrix. The resulting proteins identified are displayed in Table 12 and 13. These data were used to compare the results revealed upon the use of the BiP affinity matrix.

### Plasma of AL Amyloidosis Patient I (Sample B2)

In sample B2, the plasma of AL amyloidosis patient I, three hypothetical proteins were identified, one of which (IPI00760678) had a gene reference to the immunoglobulin lambda locus and a protein reference to the immunoglobulin lambda constant regions (Table 8). This protein was also identified in the serum and synovial fluid of the two RA patients, binding the IgIV affinity matrix.

The second protein (IPI00382938) had a gene and a protein reference to Ig lambda variable 4-3, this protein also bound to the IgIV affinity matrix, in the plasma of the amyloidosis patients I and II.

The third protein (IPI00807428) only had references as a hypothetical protein, but it contained structural characteristics of an Ig. This protein also was identified in the synovial fluid of RA patient IV, binding to the IgIV affinity matrix. It was also found binding the BiP affinity matrix in AL amyloidosis patient II.

There were two proteins named by there molecular weight, one a 25 kDa protein (IPI00154742) and one a 26 kDa protein (IPI00738024). The 25 kDa protein had a specific protein reference to the Rheumatoid Factor G9 light chain, a lambda variable 3 region apparently specific for Rheumatoid Factor. This protein also bound to the IgIV affinity matrix from the amyloidosis patients as well as from the serum and synovial fluid of the two RA patients. Rheumatoid factor, as is known, is present in many misfolding diseases.

The 26 kDa protein had gene references to immunoglobulin kappa variable 1-5. This protein bound to the IgIV affinity matrix from the synovial fluid of RA patient IV.

There were three other proteins or peptides identified as immunoglobulin. One (IPI00550162) was identified as Ig lambda variable 3-25. This protein also was identified in the serum of RA patient III, binding the BiP affinity matrix.

The second was Ig lambda constant 2 (IPI00555945), with gene and protein references to immunoglobulin lambda. Both Ig lambda variable 3-25 and immunoglobulin lambda constant 2 were identified binding the IgIV affinity matrix from the serum from RA patient III.

The third (IPI00472345) was an Ig heavy chain, Ig heavy constant gamma 3. Igs consist of two heavy chains, each with a constant region and an antigen binding variable region, and of two light chains also each with a constant region and an antigen binding variable region. Because the patient suffers from primary AL amyloidosis, the identified light chains are most likely the misfolded immunoglobulin light chains related to the pathology of the disease. Some amyloidogenic light chains like SMA, have known binding sites for BiP. It is interesting to see that some of the same Igs are prone to misfolding or binding to misfolding proteins in RA patients as well as in the amyloidosis patients.

There were three non immunoglobulin proteins identified in the plasma sample of this AL amyloidosis patient. The first (IPI00021842) was identified as apolipoprotein E(3). Apolipoprotein E co-localizes with many amyloid deposits, as is for example shown in AL amyloidosis, but also in AD and prion disease. It induces fibril formation of amyloid-P peptides.

The second protein (IPI00242956) was identified as the Fc fragment of IgG binding protein (Fcγ binding protein). Plasma levels of Fcγ binding protein are increased in certain auto-immune disorders, like Crohn's disease, systemic lupus erythematosus and RA.

The final non-Ig protein identified (IPI00004233) was the antigen to the monoclonal antibody Ki-67. This antigen is used as a proliferation marker. In some cases it is used as a marker for tumor growth. It is also a proliferation marker in RA, used for assessing the proliferation of inflammatory cell types in the synovium. This protein was also identified in the eluate of the IgIV affinity matrix after contacting with serum of the RA patients. This implies proliferating cells in this AL amyloidosis patient and in the RA patients. These cells are for example lymphoid cells, or cells from a tumor.

### Plasma of AL Amyloidosis Patient II (Sample B3)

In sample B3, the plasma of AL amyloidosis patient II, six hypothetical proteins were identified (Table 9). Two (IPI00807428 and IPI00784983) had no other gene or protein references but contained structural characteristics of an Ig. One of the two was also identified in the eluate after contacting the serum of RA patient III with IgIV affinity matrix. The other, found in the synovial fluid of RA patient IV, also bound to the IgIV affinity matrix. Hypothetical protein IPI00784983 was also identified in the eluate after contacting synovial fluid of RA patient IV with the BiP affinity matrix.

The third protein (IPI00550731) had a protein reference to the Ig kappa chain V-II region RPMI 6410 precursor.

The fourth (IPI00440577) was identified as hypothetical protein LOC651928 and had gene references to Ig κ variable 1-5. This protein was also identified in the eluate after contacting serum of RA patient III with affinity matrix.

The fifth (IPI00382938) also had gene and protein references to an Ig light chain region, but now to the lambda variable 4-3 region.

The final identified protein (IPI00384938) was hypothetical protein DKFZp686N02209 and had gene references to Ig heavy constant gamma 1 (G1m marker).

There were three protein identified by their molecular weight. Two proteins identified as 25 kDa proteins (IPI00154742 and IPI00747752), both had a gene reference to the Ig λ locus.

One had a specific protein reference to the Rheumatoid Factor (RF) G9 light chain, a lambda variable 3 region apparently specific for RF.

The third was identified as a 26 kDa protein (IPI00738024) and had gene references to Ig κ variable 1-5.

Another protein was identified as CDNA FLJ90170 fis, clone MAMMA1000370, highly similar to Ig alpha-1 chain C region, which had gene references to Ig heavy constant alpha 1. This protein was also identified in the eluate after contacting serum of RA patient III with the affinity matrix.

There were five proteins directly identified as Igs or fragments thereof. There were two different heavy chains identified, one (IPI00748158) as Ig heavy constant mu, with according gene references, but also a protein reference to Full-length cDNA clone CS0DD006YL02 of Neuroblastoma of Homo sapiens (human).

The second (IPI00472345) was identified as Ig heavy constant gamma 3 (G3m marker), with according gene and protein references.

One protein (IPI00719373) was identified as Ig lambda constant 1. This protein was also identified in the eluate of the serum and synovial fluid of the two RA patients that was contacted with the affinity matrix.

The other two proteins were identified as Ig fragments, one (IPI00470652) as a single chain Fv fragment (a small protein consisting of the fused variable regions of the light and heavy chains, with the same specificity as the parent immunoglobulin). The other protein (IPI00552874) was identified as a fragment of the Ig variable 1-3 region.

In the eluate of the plasma of this patient that was contacted with the affinity matrix, four other proteins were identified. One of these proteins (IPI00783464) was dynein heavy chain domain 3. This protein also bound to the IgIV affinity matrix in the plasma sample of this patient. Dynein is a ‘motor protein’, which moves intracellular cargo's from the cell membrane into the cell. This is for instance occurring during autophagy and axonal transport. Dynein is involved in transport of protein aggregates. So if it was bound to a protein aggregate in the plasma, it could eventually end up binding the BiP matrix.

The second (IPI00021842) was identified as apolipoprotein E(3). As described above, apolipoprotein E co-localizes with many amyloid deposits, i.e. in AL amyloidosis, AD, prion diseases. Apolipoprotein E induces fibril formation of amyloid-β peptides.

The third protein was identified as complement factor H related protein 3 (CFHR3/FHRP3/FHR3). FHRs are abundant in plasma and are highly similar to complement factor H. FHR3 binds C3b and C3d, which in turn bind amyloid. No exact function has been described for FHR3 thus far.

The last protein identified (IPI00014898) in the plasma of this patient was isoform 1 of plectin 1, also known as intermediate filament binding protein 500 kDa or hemidesmosomal protein 1. Plectin 1 binds to intermediate filaments and is involved in the organisation of microtubules, actin and intermediate filaments by cross-linking and regulation of their dynamics. Plectin 1 associates with vimentin, which in turn is an auto-antigen upon citrullination in rheumatoid arthritis. Since citrullination results in misfolding of the protein, vimentin could well be bound to BiP in its citrullinated form, in complex with plectin 1. Vimentin itself was not identified in these sera after contacting with the affinity matrices. To our knowledge, vimentin biology has not yet been associated with amyloidosis pathology. Plectin 1 was also identified in the eluate of serum of RA patient III upon contacting with the affinity matrix.

Five proteins are identified in both the plasma samples AL amyloidosis patients I and II. Hypothetical protein IPI00807428 already was mentioned. Furthermore, Ig heavy constant gamma 3 (IPI00472345), the 26 kDa protein (IPI00738024), hypothetical protein IPI00382938 (identified as Ig lambda variable 4-3 region) and the 25 kDa protein (IPI00154742, identified as the RF G9 light chain), were identified in both samples. These proteins are of extra interest, because they could be proteins typical for AL amyloidosis patients, which could be good markers to monitor the disease and/or could be used as possible drug targets.

### Serum of RA Patient III, (Sample B4)

In sample B4, the serum of RA patient III, two hypothetical proteins were identified. One was identified as hypothetical protein LOC651928 (IPI00440577), and had gene references to Ig κ variable 1-5 (Table 10). This protein also was identified in the eluate after contacting plasma of AL amyloidosis patient II with BiP-Sepharose.

The other protein, with identifier IPI00399007, is identified as hypothetical protein DKFZp686I04196 and had gene references to Ig heavy constant gamma 2.

Two proteins were identified as cell line clones. The first (IPI00328493) as a cDNA clone of a lymphoma line, clone CS0DL004YM19 of lymphoma B cells (Ramos cell line). Most likely this is an Ig heavy chain, which is very similar in amino acid sequence, compared to this cDNA clone.

The other protein was identified as CDNA FLJ90170 fis, clone MAMMA1000370 which is highly similar to Ig alpha-1 chain C region. It also had gene references to Ig heavy constant alpha 1. As mentioned, this protein was also identified in the eluate of the serum of AL amyloidosis patient II that was contacted with BiP-Sepharose.

There were nine different proteins identified as Igs or fragments thereof. The first (IPI00550162) was identified as Ig λ variable 3-25, with according gene and protein references. This protein also was identified in the eluate after contacting plasma of AL amyloidosis patient II to the IgIV affinity matrix and it was identified in the eluate after contacting plasma of AL amyloidosis patient I to the BiP matrix.

The second protein (IPI00382478) was identified as Ig heavy chain V-III region TIL, with according protein references.

The third protein (IPI00382436) was also identified as an Ig chain V-III, but this one as light chain V-III region SH.

Myosin reactive Ig light chain was the fourth Ig identified (IPI00024138), with protein references to Ig kappa chain V-III region VH precursor. Myosin is a known auto-antigen in RA.

The fifth protein was Ig variable 2-11 (V1-3 protein, IPI00552874).

The sixth protein (IPI00748158) was identified as Ig heavy constant mu.

The seventh protein (IPI00178926) was identified as the Ig J polypeptide. This is the linker Ig region for the Ig α and μ polypeptides.

The eighth protein (IPI00719373) was identified as Ig λ constant 1. This protein was also identified in samples B3 and B6, the plasma of AL amyloidosis patient I and the synovial fluid serum of RA patient IV. Furthermore, it bound to the IgIV affinity matrix, also in the serum of this latter patient IV.

The ninth protein that was identified as an Ig or fragment thereof, was identified as Ig κ chain V-I region WEA (IPI00003111). This protein had a gene reference to ‘similar to Ig kappa chain V-I region HK102 precursor’ and protein references to Ig kappa chain V-I region AU. The lambda regions identified are a known part of RF. These regions can also be misfolded Ig molecules, e.g. the RF auto-antigen, which comprises the Fc region of Igs, that can display characteristics of a misfolded protein comprising crossbeta structure. Ig light chains are over-expressed in certain autoimmune disorders and amyloidoses, including RA and AL amyloidosis, in which their expression correlates with disease activity.

There were several non-Ig proteins identified in the eluate obtained after contacting BiP-Sepharose with the serum sample of RA patient III. One was identified by its molecular weight, a 187 kDa protein (IPI00164623). This protein had gene references to complement component 3 (C3). C3 is over-expressed in the amyloid plaques of patients with AD. In RA, C3 expression is increased and found bound to microparticles and in deposits on the synovial tissue in the synovium. Our data points to the presence of misfolded C3 or C3 bound to misfolded protein ligands in serum of the RA patient. Plasma levels of the active complex C3d-C reactive protein (resulting from C3 activation) are increased in various patients studies, further substantiating our observation. Moreover, in collagen induced arthritis in mice, the lack of C3 ameliorates the disease. All the data together reflect an important role for the complement system in the aetiology of the disease.

In the current experiment, C3 was not the only complement factor identified in the serum of RA patient III. In addition, also complement component 4b binding protein alpha chain (IPI00021727) was identified. This protein had according gene and protein references. C4b binding protein acts as a cofactor for factor I in the degradation of C4b. C4b binding protein has no known association with RA. C4b binding protein binds to crossbeta binding protein serum amyloid P (SAP), when the SAP is presented in an aggregated form (aggregated by immobilized antibodies in vitro). Furthermore, C4b binding protein is localized in the amyloid deposits in patients with AD.

The next non-Ig protein identified was the CD5 antigen like precursor (IPI00025204), which had according gene and protein references. This protein is also referred to as Spa (Sp alpha). Sp alpha is expressed by macrophages in lymphoid tissues and binds to lymphoid cell types. Recombinant Sp alpha binds to Gram-positive as well as Gram-negative bacteria and induces aggregation of these bacteria. A citrullinated form of Sp alpha is present in the synovial exosomes of RA patients together with various other citrullinated proteins, most of which are identified as auto-antigens. Because citrullination induces misfolding in proteins, the Sp alpha that bound to BiP-Sepharose is likely misfolded.

The next protein identified was the homolog of dapper 1 (IPI00171594) with according gene and protein references. This protein is an antagonist of beta-catenin (Wnt-signaling pathway). It is downregulated in various types of cancer. To our knowledge, dapper 1 is not associated with RA or any other protein misfolding disease.

Little is known about the next protein identified, general control of amino-acid synthesis 1 like 1 protein (GCN1-like 1, IPI00001159). In yeast it is involved in transcriptional activation under starving conditions, i.e. stress conditions. Little is known about its role in humans.

The same is true for the next protein identified (IPI00217851), isoform 1 of the phosphatase actin regulator 4 (PHACTR4). It had according gene and protein references, and a protein reference to ‘Hypothetical protein DKFZp686L07205’. Other PHACTR family members are inhibitors of protein phosphatase 1 (PP1) activity, and the homolog of this gene in mice interacts with actin and PP1.

Isoform 1 of plectin 1 was the next protein identified. This protein was also identified in the eluate obtained upon contacting plasma of AL amyloidosis patient II with the affinity matrix. As mentioned before, Isoform 1 of plectin 1 associates with vimentin, an auto-antigen when citrullinated, in RA. Since citrullination results in misfolding of the protein, citrullinated vimentin, and perhaps (either or not citrullinated and misfolded) plectin I in complex with vimentin, can be bound to BiP. Vimentin itself was not identified in the eluates from the BiP-Sepharose or IgIV-Sepharose.

The next protein identified is well known for its higher serum levels in RA patients, namely alpha-1-antitrypsin (IPI00553177), also known as alpha 1 antiprotease, alpha 1 protease inhibitor and serpin peptidase inhibitor clade A. As mentioned, RA patients have elevated serum levels of this protein, were it mostly is in complex with IgA (IgA). High serum levels are associated with a more erosive form of the disease. Alpha-1-antitrypsin is an inhibitor of elastase, which is released by neutrophils on sites of inflammation. Neutrophil elastase can degrade a broad range of substrates, especially connective tissue components such as elastin, proteoglycans and collagens. Oxidization of alpha-1-antitrypsin promotes complex formation with IgA, and these complexes have no inhibitory activity against elastase. Since oxidation of proteins in inflammatory conditions happens more readily, the complex formation of alpha-1-antitrypsin probably considerably contributes to the aetiology of the disease.

The final protein identified was vitronectin (IPI00298971). Vitronectin interacts with plasminogen activator inhibitor-1, plasminogen activators, the urokinase plasminogen activator receptor, and plasminogen, resulting in the inhibition of plasmin generation. Increased levels of antibodies to vitronectin and increased plasmin generating activity is shown in the synovium of RA patients. Plasmin is well known for its activation of matrix metalloproteases, which in turn contribute to the destruction of connective tissue in the joints.

### Synovial Fluid of RA Patient IV (Sample B6)

In the synovial fluid of RA patient IV, six different proteins specifically binding to the BiP affinity matrix were identified in the eluate (Table 11). One was identified as BiP itself (IPI00003362). When not stripped from the column material, the BiP most likely originates from complexes of the HSP70 with misfolded proteins present in the synovial fluid, which are then the true ligands for the BiP-Sepharose. In approximately 75% of RA patients increased levels of BiP are determined in their synovial fluid.

The next protein identified was a hypothetical protein (IPI00784983). This protein was also identified in the eluate after contacting the BiP affinity matrix with the plasma of AL amyloidosis patient II and in the eluate after contacting the IgIV affinity matrix with the serum of RA patient III. As mentioned, this protein has no other gene or protein references, but contained structural characteristics of an Ig.

Furthermore, Ig heavy chain V-III region CAM was identified (IPI00382482), with according protein references.

There was one other Ig heavy region identified, now a constant region, Ig heavy constant mu (IPI00479708).

The last two proteins were both light chains. One was identified by its molecular weight, a 26 kDa protein (IPI00430820). This had gene and protein references to Ig κ variable 1-5.

The last protein was identified as Ig λ constant 1 (IPI00719373), which was also found extracted from the plasma of AL amyloidosis patient II and the serum of RA patient III after contacting the BiP affinity matrix. The Ig λ constant 1 was also found binding the IgIV affinity matrix from the serum of RA patient III.

Proteins Specifically Binding from Control Samples

Besides the proteins from the AL amyloidosis patient plasma and RA patient serum samples that bound specifically to the BiP matrix compared to the controls, there were also proteins from the control plasma or control serum that specifically bound to the BiP matrix compared to the patient samples. This provides interesting information on proteins which are down-regulated or have increased clearance in the two diseases.

Alpha-2-macroglobulin (IPI00478003), haptoglobin (IPI00431645) and (sero)transferrin (IPI00022463) specifically bound to BiP-Sepharose from normal pooled plasma compared to AL amyloidosis patient I. Alpha-2-macroglobulin (IPI00478003), complement C4a (IPI00032258), complement C4b binding protein (IPI00021727), complement factor H isoform I (IPI00029739), clusterin isoform 1 (IPI00291262) and haptoglobin (IPI00431645), specifically bound to BiP-Sepharose from normal pooled plasma compared to AL amyloidosis patient II. Haptoglobin (IPI00431645), (sero)transferrin (IPI00022463) and clusterin isoform 1 (IPI00291262), specifically bound from control serum compared to RA patient III.

Alpha-2-macroglobulin was identified in the eluate obtained after contacting BiP-Sepharose with pooled plasma control sample, however it is not detected in either of the samples of the AL amyloidosis patients. It is well known that this protein mediates the degradation of soluble amyloid-β. It is very likely that in the AL amyloidosis patients, alpha-2-macroglobulin expression is significantly lower, contributing to the aetiology of the disease. It could also be that there is increased clearance from blood of alpha-2-macroglobulin due to increased amyloid formation on specific locations, or by competition with BiP at the Sepharose matrix for binding places of misfolded proteins.

Haptoglobin was identified in all three controls, and was not present in any of the patient samples. Haptoglobin in an acute phase protein and a chaperone, which is known to associate with amyloid deposits in vivo. Depletion of haptoglobin from serum renders proteins more susceptible to aggregation and precipitation. Plasma levels of haptoglobin are known to be elevated in RA patients and amyloidosis patients. It is very likely that in these patients most binding sites of misfolded protein for the chaperone BiP at the Sepharose matrix, are bound by the chaperone haptoglobin. This would result in the current result: no identification of haptoglobin in the various patient samples.

(Sero)transferrin was identified in the pooled plasma control sample of AL amyloidosis patient I and the serum control sample of RA patient III, it was however not identified in either of the samples of these patients. Transferrin is known to inhibit crossbeta fibril formation. It also localizes in the synovium of RA patients, were it is involved in the build up of iron deposits. The inhibition of crossbeta fibril formation points to crossbeta binding/misfolded protein binding properties. This could result in competitive binding and/or depletion from plasma.

Complement C4a, complement C4b binding protein and complement factor H isoform 1 were all three identified in the pooled plasma control sample, and not in the sample from AL amyloidosis patient II. As mentioned, C4b binding protein is known to bind crossbeta binding protein SAP and protein aggregates. The lack of C4b binding protein in the patient sample could indicate competitive binding, but also reduced expression. Complement C4a is a cleavage product from activation of the classical complement pathway. Factor H is a negative regulator of the alternative complement pathway by binding C3b and acting as a cofactor for the degradation of C3b. Factor H is also known to be present in amyloid plaques in patients with AD. Further studies showed that factor H binds the fibrillar crossbeta form of amyloid-β. Factor H could be cleared from the plasma in this patients, due to binding to aggregates, or it could not be accessible for binding to soluble misfolded proteins, which can bind BiP-Sepharose, due to precipitation onto insoluble misfolded protein deposits. It also could be competitive binding with BiP. Another option is reduced expression, which would in turn lead to a more active complement system, which in turn would contribute to the aetiology of the disease.

Clusterin isoform I was also uniquely identified in the eluate obtained upon contacting BiP-Sepharose with control pooled plasma as well as with the RA control serum. It was however not identified in the sample form AL amyloidosis patient II and the serum of RA patient III. Clusterin is a molecular chaperone, known to act in the Crossbeta Pathway. Clusterin is well known to bind amyloid proteins and is thought to be involved in the clearance of amyloid-β aggregates. It is very likely that clusterin is competing with BiP at Sepharose beads for binding misfolded proteins in the various patient samples.

### Experiment 2: Parallel Analysis of Six Different Synovial Fluid Samples of RA Patients

To further substantiate our above listed observations, we subsequently contacted BiP-Sepharose or negative control-Sepharose with six different synovial fluid samples of RA patients. Those samples are listed below:
1. RA patient I, IgM-RF titer 172
2. RA patient II, IgM-RF titer 515
3. RA patient III, IgM-RF titer 565
4. RA patient IV, IgM-RF titer 23
5. RA patient V, IgM-RF titer <11
6. RA patient VI, IgM-RF titer <11
   Samples incubated according to the above mentioned procedure are listed B1-6 for BiP-Sepharose, and C1-6 for negative control-Sepharose. One difference when compared to the protocol described above is that now with the twelve samples, elution buffer comprised 1 M NaCl. Again, a misfolded protein or a protein binding to misfolded protein is identified uniquely in the synovial fluid when no or a relatively very low number of fragments are detected in eluates of control-Sepharose. See Table 14 for an overview of identified misfolded proteins and/or misfolded protein binding proteins.

### Experiment 2: Results

The results of the analysis of proteins eluted from BiP-Sepharose, when contacted to six separate synovial fluids of RA patients, are summarized in Table 14. In Table 15, the summary and conclusions of the data analysis are given. Obviously, most of the identified proteins are known for their capacity to intertact with amyloid, or their propensity to fold in amyloid or amyloid-like conformation comprising crossbeta structure. Of special interest are those proteins that were identified in three or more of the six patient samples, and/or that were also identified in the previous analysis of AL amyloidosis patient samples and RA patient samples. Noteworthy, three chaperones were identified, i.e. HSP70, haptoglobin and clusterin. When BiP does not originate from the affinity matrix, also this chaperone is extracted from RA patient synovial fluid. Several proteins not only act in the Crossbeta Pathway, but have also been described as being part of the Complement Lipid Pathway (CLiP), a Pathway that is active in response to inflammation and infection, and during periods of increased risk for atherosclerosis.

### Concluding Remarks

In summary, by the use of the BiP-affinity matrix, as described in the current Example, we identified various proteins unique for AL amyloidosis patient plasma, and series of proteins were uniquely identified in samples obtained from serum of patients with RA. Moreover, misfolded proteins and/or crossbeta binding proteins were identified that are present in both fluid samples of RA patients and AL amyloidosis patients. Several proteins were identified in both AL amyloidosis patient samples. In addition, several proteins were identified in two or more RA patient samples. When applying BiP-Sepharose and enriched IgIV-Sepharose with the same sample, for all four patient samples of the first analysis at least one protein was identified in both eluates. This identification reflects a set of misfolded proteins that is present in a sub-population of the group of patients under investigation, for example RA patients or AL amyloidosis patients. Such a set of misfolded proteins present in many patients is a key basis for the development of general therapeutics and broad range diagnostics.

The identified misfolded proteins can bind to the affinity matrix directly or the identified proteins are crossbeta binding proteins, themselves. These proteins all together provide insight into the biology and pathophysiology of the disease, and form the basis for the development of a disease-specific diagnostic tool and/or are newly identified drugable targets for the development of therapeutics aimed at depleting patients from disease-modulating misfolded proteins in vivo (administering drugs) and/or ex vivo (e.g. extra-corporal device). Thus, the studies revealed insight into several identified crossbeta binding molecules apparently related to the diseases under investigation, i.e. AL amyloidosis and RA. The identified variable regions of Igs serve as a good starting point for development of synthetic affinity regions with affinity for misfolded proteins related to the diseases.

### EXAMPLE 13

### Influence of BiP on Crossbeta-Induced Platelet Aggregation

Platelet aggregation-studies with freshly isolated human platelets were performed as described above. For aggregation studies, platelets of four separate donors were used on subsequent days. For each donor, concentrations of misfolded crossbeta proteins were determined that result in sub-optimal platelet aggregation when compared to maximum aggregation induced by 8 μM synthetic thrombin receptor activating peptide (TRAP; SFLLRN, SEQ ID 10; positive control). The sub-optimal crossbeta protein concentration was then used to study the effects of indicated concentrations of BiP, sRAGE and negative control buffer. Since BiP-FLAG-His and sRAGE-FLAG-His were stored in PBS with 5% glycerol, one of the. buffer controls was this glycerol buffer diluted to the same extent as the BiP and sRAGE solutions. Crossbeta proteins were Aβ42t=0, HbAGE and BSA-AGE, used at indicated final concentrations. Misfolded proteins, buffer control or TRAP control were pre-mixed with buffer or BiP or sRAGE, and pre-incubated for 5 minutes at room temperature, before mixtures were added to the platelet suspension that was warmed at 37° C. and stirred at 900 rpm.

### Results

Human platelets of four separate healthy human voluntering donors are potently activated, resulting in agglutination and/or aggregation, when contacted to misfolded proteins comprising crossbeta structure (FIG. 21-23. See also patent application US2007003552). For the current studies, we activated platelets of four separate human donors with positive control for activation, TRAP, buffer as a negative control, or misfolded proteins Aβ42t=0 (donor a, b, c), HbAGE (donor c, d) and BSA-AGE (donor a, b, c). The crossbeta binding proteins BiP and sRAGE potently inhibited misfolded protein induced platelet aggregation, whereas TRAP-induced aggregated was unaffected by BiP or sRAGE (FIG. 21-23). BiP or sRAGE alone, without activator, did not induce platelet aggregation. It has been demonstrated that activation of platelets by misfolded proteins comprising crossbeta structure is at least in part mediated by multiligand amyloid receptors like for example CD36 and Scavenger receptor A.

The platelet aggregation experiments demonstrate that crossbeta-induced platelet activation is inhibited by chaperone BiP at the tested concentration. The positive control crossbeta binding protein sRAGE also inhibits crossbeta-induced platelet aggregation. These data show that the HSP BiP with affinity for misfolded proteins comprising crossbeta structure can inhibit or block misfolded protein mediated biological effects, i.e. induction of a pro-thrombotic state by misfolded proteins comprising crossbeta structure. Therefore, BiP, and sRAGE as well, is a target candidate as a starting point for refinement towards an effective anti-thrombotic agent directed against the pro-thrombotic potential of misfolded proteins comprising crossbeta structure. In addition, BiP can interact with the platelet receptor(s) involved in transducing the effects of misfolded protein on platelet activation. Candidate receptors are for example CD36 and Scavenger receptor A. Therefore, an effective BiP-based anti-thrombotic agent can also be obtained upon refinement of the best inhibitor of misfolded protein-induced platelet activation, targeting the BiP-binding receptor(s) and thereby inhibiting interaction of the misfolded protein with platelets.

### EXAMPLE 14

### Binding of Misfolded Proteins and HSP to Antigen Presenting Cells: Crossbeta-BiP-Dendritic Cells

### Materials & Methods

In vitro generation of peripheral blood human monocyte-derived dendritic cells, and analyses for binding of misfolded proteins and BiP

Human DCs are generated from non-proliferating precursors selected from peripheral blood mononuclear cells (PBMCs), essentially by published methods (Sallustro and Lanzavecchia [1994], J. Exp. Med. 179 1109-1118). Relative abundant presence of CD1a, CD32, CD36, CD40, CD54, CD86, HLA-DR and CD206 and relative low content of CD14 positive, CD16 positive, CD64 positive, CD80 positive, CD83 positive and CD163 positive cells serve as a quality measure for the immature DCs. After obtaining the immature DCs upon stimulation with GM-CSF and IL-4, direct binding of misfolded proteins and BiP to the DCs is assessed by incubating 200.000 cells (2*10⁶ cells/ml) for 30 minutes at 4° C. under metabolic arrested state, with 1) PBS, 2) 58 μg/ml BiP (830 nM), 4) 250 μg/ml BSA-AGE (3.6 μM), 5) BSA-AGE+BiP, 6) 250 μg/ml oxLDL, 7) oxLDL+BiP, 8) mixture of BiP-binding peptides 6BB8-11, 13 (see Table 5), 125 μg/ml+BiP, and 8) 62.5 μg/ml Aβ16-22, 62.5 μg/ml Aβ25-35+BiP. After the incubation and careful washing of the cells, binding of BiP, BSA-AGE and oxLDL is assessed with FACS, using primary antibodies monoclonal anti-FLAG antibody, monoclonal anti-glycated human fibronectin antibody 4B5 and rabbit serum with anti-human apoliprotein B-100 antibody (Dade Behring, Newark, Del., USA), and the appropriate FITC- or PE-labeled secondary antibody. Mean fluorescence intensity (MFI) ratio's are determined by measuring background fluorescence.

### Results: Binding of BiP and Misfolded Proteins to Dendritic Cells

Binding of misfolded proteins comprising crossbeta structure, i.e. BSA-AGE, oxLDL, Aβ16-22/Aβ25-35, or 1:1:1:1:1 (mass ratio) mixed BiP-binding peptides 6BB8 (YVDRFIGW), 6BB9 (LFWPFEWI), 6BB10 (HWDFAWPW), 6BB11 (FWGLWPWE) and 6BB13 (RRRAA), with BiP was assessed with FACS analysis and is summarized in Table 16. Notably, BiP itself binds to the DCs to a certain extent (MFI ratio 1.4). Either misfolded proteins are exposed at the surface of (a fraction of) the DCs, for example apoptotic/necrotic/dead/dying DCs, or (a fraction of) BiP molecules has/have bound misfolded proteins, originating from serum or cells used for expression of recombinant BiP, resulting in BiP—misfolded protein cargo complexes that are the binding partner for receptors like for example CD36, CD91, SRA, TLR4, LOX1, CD40. BSA-AGE and oxLDL bind to the DCs in the absence of BiP. When BSA-AGE and BiP are added to the cells together, the amount of bound BSA-AGE is only slightly increased, whereas the amount of BiP is about doubled, when compared to BiP added to DCs without BSA-AGE. This shows that BiP binds to BSA-AGE bound to the DCs and/or that BiP binds to BSA-AGE in solution and subsequently the complex binds to the indicated cell surface receptors. The opposite is seen for oxLDL+BiP; now the amount of bound oxLDL is lowered when compared to when oxLDL is exposed to the DCs without BiP, and the amount of bound BiP is also lowered to a similar extent. This reflects an inhibitory activity of BiP. Binding of BiP to oxLDL blocks binding of free oxLDL or oxLDL in complex with BiP to DCs. When the BiP-binding peptides are incubated with DCs in the presence of BiP, more BiP is detected at the surface of the DCs. The peptides can be bound to the cells, being an intermediate between the DCs and BiP, or complexes of BiP and the peptides are ligands for DC receptors. The same is true to a dramatic extent for the 1:1 (mass ratio) of Aβ peptides. Notably, upon exposure to BiP and the Aβ peptides, the DCs form clusters of 5-20 cells, whereas the PBS incubated DCs are single cells. These data altogether demonstrate that BiP binding to DCs is modulated in two directions, depending on the nature of the misfolded protein comprising crossbeta structure under consideration. With oxLDL, BiP binding to the antigen presenting cells is reduced, whereas the opposite is observed for Aβ, BSA-AGE and the BiP-binding peptides.

Now that we analysed the binding of misfolded proteins to DCs, and the binding of BiP, activation of the DCs can be determined after prolonged incubations at 37° C. The DCs will be analyzed for the following parameters: surface density (mean fluorescent intensity, MFI, or % positive cells) of CD83, CD86, CD80, CD40, CD91, HLA-DR, Scavenger receptor A, RAGE, CD36 and CD40 measured using FACS, as wells as cell death/cell viability, as determined by apoptosis marker 7-Amino-Actinomycin D (7AAD) binding. In addition, extent of IL-6 secretion and IL-8 secretion are determined in the cell culture supernatant using Pelipair ELISA (M9316, Sanquin Reagents, Amsterdam, The Netherlands) for IL-6 and a Cytosets CHC1304 kit (Biosource) for IL-8. Also IL-10 levels will be determined.

### EXAMPLE 15

Modulation of the Interaction of Misfolded Proteins with Cells by Chaperones.

Misfolded proteins comprising crossbeta structure are capable of binding to cells and evoke cellular responses, including but not limited to inflammatory responses and changes in cell growth or apoptosis. We addressed whether heat shock proteins modulate the interaction of such misfolded proteins with cells. We used human primary endothelial cells (HUVECs) isolated from umbilical veins and the HSP70 family member BiP.

### Materials & Methods

### Isolation, Culturing and Analysis of HUVECs

### HUVEC Isolation and Culturing

HUVECs are primary endothelial cells (ECs), isolated from umbilical cords using 0.1% collagenase (Sigma, C0130, 100 mg, dissolved in 100 ml M199 medium supplemented with 10% FCS (Gibco 10106-169) and Penicillin-Streptomycin (P/S, Gibco, 15140-122)), according to widespread used standard procedures known to a person skilled in the art. HUVECs have the typical features of ECs, e.g. cobblestone morphology and von Willebrand factor storage in Weibel-Palade bodies. HUVECs can regularly be cultured up to passage 5; beyond passage 5 HUVECs loose typical EC markers. The isolation is described here in brief. The umbilical cord is washed for less than 3 minutes in ethanol and subsequently with PBS. The vein is connected to canules and flushed with 10 ml PBS, followed by loading with the 0.1% collagenase solution. After a 15 minute-incubation at 37° C., the detached endothelial cell suspension is recovered by flushing the vein with 10 ml medium which is subsequently added to the collagenase solution. The EC suspension is centrifuged for 5 minutes at room temperature, at low g-force. Supernatant is discarded and the cell pellet is resuspended in 5 ml ‘rich medium’ (EGM-2; Endothelial basal medium (EBM-2, Cambrex, CC-3156) and Singlequots containing supplements for endothelial cells (Cambrex, CC-4176)). Cells (passage 0, P0) are seeded in a culture flask coated with 0.5% gelatin (Sigma, G1393). To facilitate the adhesion of the endothelial cells, human fibronectin is added to the cell culture at a final concentration of 2 μg/ml. EC's are cultured at 37° C., at 5% CO₂. The cell culture medium is refreshed every 2-3 days up to when the cells grow to confluency. Then, with the addition of trypsin-EDTA, the cells are detached from the flask, centrifuged at low g-force, resuspended in rich medium and seeded in larger 0.5% gelatin-precoated cell culture flasks.

### I. Adhesion of Cells to Misfolded Proteins

In 96-wells plates (Immulon high-binding plates, Thermo Labsystems 3355) proteins, i.e. BSA-AGE, control BSA, IgIV Gammagard, crossbeta dIgIV-86 at 5 μg/ml, 10 μg/ml misfolded oxLDL, or gelatin (Sigma G1393, 2% solution in H₂O or PBS, positive control for adhesion to ECs) were coated using 100 μl solutions, in six wells for each condition. Following incubation for 2 hours at 37° C. the solutions were discarded and the wells blocked for 1 hour at 37° C. with 100 μl/well of 1% polyvinyl-pyrrolidone (PVP, Sigma P5288) in PBS, filter (0.22 μm) sterilized. PVP is an inert polymer that does not support cell adhesion. Subsequently, the block solution was discarded. Next, the plates were incubated with 40 μl RPMI 1640 medium (Gibco, 52400) and 10 μl of the potential inhibitor, i.e. recombinant human BiP at approximately 200 μg/ml final concentration. HUVECs were obtained by trypsinization. After centrifugation cells were resuspended in RPMI 1640 medium with P/S and diluted to approximately 150.000 cells/ml. Each well was seeded with 100 μl of the cell suspension, approximately 15.000 cells/well. Cells were allowed to adhere for 1 hour at 37° C. Plates were carefully and gently washed with RPMI 1640 medium with P/S. The medium was removed by pipetting along the wall of the wells. Plates were washed until blank wells contained a steady number of cells as judged by eye (microscope), i.e. typically 1-3 times. Subsequently, 50 μl RPMI medium was added to each well, followed by the addition of 5 μl/well 10% (v/v) Triton-X100 in PBS to allow for cell lysis, and incubation for 10 minutes at 37° C. Next, 50 μl of lactodehydrogenase (LDH, Roche Applied Science, 11644793001) solution was added according to instructions of the manufacturer. The plate was incubated for 0.5-3 hours at room temperature in the dark. The absorbance at 490 nm was measured on a Versamax microplate reader at various time points.

### Adhesion of ECs to Immobilized Misfolded Proteins Comprising Crossbeta Structure Under Influence of Chaperone BiP: Results

### Adhesion of Cells to Misfolded Proteins

In FIG. 24 it is depicted that HUVECs adhere to misfolded human IgG (dIgIV-86). HUVECs also adhered to BSA-AGE to a similar extent as to gelatin (not shown). Preincubation of coated misfolded proteins oxLDL and dIgIV-86 with chaperone BiP results in an increased number of adhered HUVECs to oxLDL (from background levels to a cell number comparable to 50% adherence as seen with gelatin), and to dIgIV-86, as determined by measuring LDH activity after lysis of washed cells (FIG. 24). Incubation of BSA-AGE, gelatin, albumin or native IgIV with BiP does hardly increase the number of adhered cells. Data for BSA-AGE is not shown.

These results demonstrate that HUVECs can have affinity for misfolded proteins comprising crossbeta structure, as shown for BSA-AGE and dIgIV-86. The cells do not adhere to a higher extent to oxLDL, when compared to control albumin and control IgG. Pre-incubation of coated proteins with BiP induces increased HUVECs adherence. This shows that the ECs comprise receptors for BiP, when BiP is bound to a misfolded protein. -Known receptors for HSPs bound to cargo are CD36, TLR2, TLR4, CD40, SRA, CD91/LRP and LOX-1, of which at least CD36, TLR2, TLR4, CD40, LRP and LOX-1 are present at ECs.

### II. Binding of Misfolded Proteins to Cells Assessed by Fluorescence-Activated Cell-Sorting (FACS) Analysis

For these experiments HUVECs were isolated by trypsinization. After trypsinization cells were collected in RPMI 1640, containing P/S and 10% FCS and centrifuged. After centrifugation cells were resuspended in RPMI medium without FCS at a concentration of 1.250.000 cells/250 μl. Individual 4-ml tubes (polypropylene, Greiner), containing 250 μl cell suspensions were made. To each tube 75 μl of a sample, containing either,
a. buffer (PBS) only, or
b. 25 μl PBS+50 μl BiP (1.16 mg/ml), or
c. 50 μl buffer with 25 μl oxLDL (1 mg/ml), or
d. 74 μl buffer with 1 μl BSA-AGE (25 mg/ml), or
e. 24 μl PBS+1 μl BSA-AGE (25 mg/ml)+50 μl BiP (1.16 mg/ml), or
f. 25 μl oxLDL (1 mg/ml)+50 μl BiP (1.16 mg/ml),
g. 1.6 μl dIgIV-65 (20 mg/ml)+73.4 μl PBS
h. 1.6 μl dIgIV-65+50 μl BiP+23.4 μl PBS

was added. Subsequently, the cells were incubated with the sample for approximately 30 minutes at 4° C. Next, cells were pelleted by centrifugation and the supernatant was discarded. Cells were washed subsequently with FACS buffer (PBS/0.5% BSA/0.05% m/v NaN₃) at 4° C. and resuspended in FACS-buffer at approximately 1*10⁵ cells/100 μl for subsequent analysis. Cell death was determined by adding 3 μl 7-aminoactinomycin D (7AAD) solution (prepared according to standard procedures). Binding of sample BSA-AGE (see elsewhere in this application for preparation details) was determined with anti-AGE monoclonal antibody 4B5 (10 μg/ml) and, after washing, with goat anti-mouse PE secondary antibodies (Jackson Immunoresearch, West Grove, USA). Binding of BSA-AGE was also assessed using the intrinsic fluorescence of BSA-AGE in the PE channel. Binding of oxidized LDL (oxLDL, oxidized for 56% following incubation with FeSO₄; specific enhancement of Thioflavin T fluorescence) was determined with rabbit serum with anti-ApoB100 polyclonal antibodies at a concentration of 160 μg/ml and, after washing the cells, with FITC-labelled goat anti-rabbit antibodies (1:200, Jackson). Binding of dIgIV-65 was assessed by using anti-Human IgG antibody. BiP binding was determined using the earlier mentioned anti-FLAG antibody (M2, Sigma-Aldrich).

### Results: Binding of Misfolded Proteins to Cells

Using two methods, BSA-AGE was found to bind efficiently to a large fraction of the ECs with a mean fluorescence intensity (MFI) ratio of 13.8. dIgIV-86 did not bind to incubated ECs and displayed an MFI ratio of nearly 1. BiP had no modulatory effect on binding of dIgIV-86 to the ECs since in the presence of BiP, still the misfolded protein did not bind to ECs, neither BiP did bind. OxLDL in the presence or absence of BiP bound to a similar extent to ECs with MFI ratio's of 1.6 and 1.7, respectively. Like with the hDCs, BiP alone bound to the HUVECs to some extent; MFI ratio of 1.4. The MFI ratio increases for BiP when HUVECs are co-incubated with BSA-AGE and BiP; MFI ratio of 2.5. At the same time the MFI ratio for BSA-AGE is lowered to 10.2. The binding characteristics obtained with ECs incubated in suspension with BSA-AGE are in line with the observation that ECs bind efficiently to wells of cell culture plates that are coated with BSA-AGE.

Taken together, these results demonstrate that cells are capable of specifically binding to misfolded proteins BSA-AGE and oxLDL with crossbeta structure and that BiP modulates the interaction of BSA-AGE with cells.

### EXAMPLE 16

Depletion of Solutions from Misfolded Proteins Using BiP Immobilized to a Solid Support

We analysed whether recombinant human BiP with a C-terminal FLAG-tag and His-tag can be applied for depleting solutions from misfolded proteins comprising crossbeta structure. In brief, in an ELISA approach, BiP was immobilized, exposed to solutions with a spike of misfolded HbAGE and dOVA standard, and subsequently binding of the misfolded proteins to BiP was assessed.

### Materials and Method

BiP was coated at a concentration of 5 μg/ml at Greiner Microlon high-binding plates, for 1 h at room temperature with motion. As a negative control buffer was coated. ELISAs were performed essentially as described before. Blocked (Roche blocking reagent) wells coated with BiP or coat buffer were overlayed in duplicate with 0, 1, 10 or 100 μg/ml of either dOVA standard, or HbAGE. All binding buffers were PBS/0.1% (v/v) Tween20. Binding of dOVA was assayed using monoclonal anti-chicken egg albumin antibody (Sigma, A6075, 1:10.000 dilution) and RAMPO (Dako Cytomation, P0260, 1:3.000). HbAGE was detected using an advanced glycation end-products specific mouse hybridoma IgG 4B5, raised against glucose-6-phosphate glycated human fibronectin, and RAMPO. Background signals obtained with buffer coated wells that were subsequently overlayed with protein solutions (see below), were subtracted from signals obtained with wells with coated BiP. In addition, background signals obtained for primary and secondary antibody incubations with wells in which no dOVA or HbAGE was added (buffer control for binding), was subtracted from signals obtained with 1, 10 and 100 μg/ml misfolded protein.

### Results

FIG. 25 shows that dOVA standard can be extracted from solution by immobilized BiP. HbAGE was extracted specifically by BiP to an even higher extent, with already maximum binding to BiP when exposed at 1 μg/ml HbAGE. These results show that BiP, which has affinity for misfolded proteins comprising crossbeta structure, that is immobilized on a suitable solid support, can be applied for depletion of solutions from misfolded proteins comprising crossbeta structure, like for example dOVA and HbAGE.

Applications for this disclosed method for depleting protein solutions from misfolded proteins are in the field of for example, but not restricted to i) diagnostics for protein misfolding diseases, like for example renal failure, systemic amyloidosis, like for example AL-, AA- or ATTR amyloidosis, or RA, ii) quality control of protein solutions, like for example biopharmaceuticals and vaccines, iii) dialysis, using for example extracorporal devices, of patients suffering from protein misfolding diseases like for example renal failure, systemic amyloidosis, like for example AL-, AA- or ATTR amyloidosis, or RA, and iv) clearance of biopharmaceuticals from misfolded proteins bearing a risk for induction of (immunogenic) side effects. For all of the above mentioned applications, the specifications of the applied HSP with respect to preferential and specific binding to misfolded proteins, can be adjusted to ones needs due to the fact that numerous chaperones are available, with convergent and divergent specificities for classes of misfolded proteins (Table 4). With the methods and means described in Example. 7-10, those specific chaperones can be selected from a library of HSPs, that are required for certain aimed purposes like for example those listed above.

### ABBREVIATIONS

2D, two-dimensional; Aβ, human amyloid-β(1-40); AGE, advanced glycation end-product; APC, antigen presenting cell; aPTT, activated partial thromboplastin time; ATP, adenosine 5′-triphosphate; BiP/grp78, Immunoglobulin heavy chain-binding protein/Endoplasmic reticulum lumenal Ca2+-binding protein; BSA, bovine serum albumin; CD, Cluster of Differentiation; CNBr, cyanogen bromide; DC, dendritic cell; DTT, dithiothreitol; EAE, experimental autoimmune (or allergic) encephalomyelitis; ELISA, enzyme-linked immunosorbent assay; ERAD, endoplasmic reticulum (ER)-associated degradation; FACS, Fluorescence Activated Cell Sorting; FCS, fetal calf serum; FITC, fluorescein isothiocyanate; FP, fibrin peptide; g6p, glucose-6-phosphate; GM-CSF, GRANULOCYTE MACROPHAGE COLONY STIMULATING FACTOR; Hb, haemoglobin; HEK, Human Embryonic Kidney 293 Cell; HEPES, {2-(4-(2-Hydroxyethyl)-1-piperazinyl)ethanesulfonic Acid}; HGFA, hepatocyte growth factor activator; CHAPERONE, heat-shock protein; HLA-DR, D-related human leukocyte antigen; IFA, incomplete Freund's adjuvant; IgIV, immunoglobulins intravenous; IL, interleukin; IvIG, intravenous_immunoglobulins; IFN, interferon; LRP/CD91, low density lipoprotein related receptor; LOX, lectin-like receptor for oxidized low density lipoprotein; LPS, lipopolysaccharide; MBP, myelin basic protein; MOG, myelin oligodendrocyte glycoprotein peptide; MS, multiple sclerose; MTT, mitochondrial metabolic activity; NHS, N-hydroxysuccinimide; OVA, ovalbumin; PBMC, peripheral blood mononuclear cells; PBS, phosphate-buffered saline; PRP, platelet rich plasma; PT, prothrombin time; RA, rheumatoid arthritis; RAMPO, peroxidase-conjugated rabbit anti-mouse immunoglobulins; RPMI, Roswell Park Memorial Institute; SR-A, scavenger receptor; sRAGE, soluble extra-cellular fraction of receptor for advanced glycation end-products; TLR, Toll-like receptor; TMB, 3′3′5′5′-tetramethylbezidine; TNF-α, tumor necrosis factor-α; tPA, tissue-type plasminogen activator; ULS, universal linkage system; UPR, unfolded protein response.

### Tables

TABLE 1
Compounds interacting with crossbeta structure or crossbeta structure induced protein conformations

| Congo red | Chrysamine G | Thioflavin T |
|---|---|---|
| 2-(4′-(methylamino)phenyl)-6- | Dimethylsulfoxide (DMSO) | Glycosaminoglycans |
| methylbenzothiaziole | | |
| Thiofiavin S | Styryl dyes | BTA-1 |
| Poly(thiophene acetic acid) | conjugated polyeclectrolyte PTAA-Li | CpG ODN |
| Ilipopolysaccharide | endotoxin | Lipid A |
| Ca₃(PO₄)₂ | Dehydro-glaucine | Ammophedrine |
| isoboldine | Thaliporphine, thalicmidine | Haematein |
| Dextran-sulphate | Dextran-sulphate 5,000-500,000 Da | ellagic acid hydrate |
| ellagic acid | kaolin | Ammophedrine HBr |
| corynanthine | | Orcein |
| 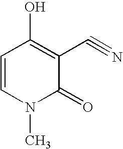 | 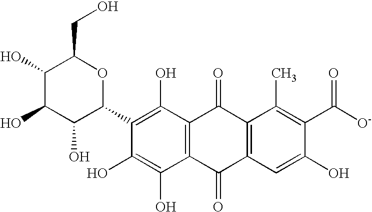 | 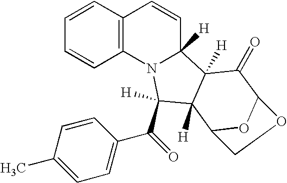 |
| 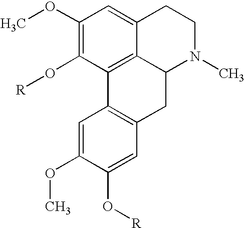 | glass-like surfaces (e.g. tantalum sputtered glass) | Long-chain fatty acids in complex with albumin |
| aporphine alkaloid compounds, with R | | |
| being a variable chemical group | | |
| cardiolipin | prion^{sc} | negatively charged lipids |
| Poly-vinylchloride (PVC) | Titanium nitride | Supercel |
| Titanium | Heparan sulfate | sulfatide vesicles |
| Barium carbonate | Liposomal vesicles | Glass |
| Sulfatides | Heparin-coated surfaces | Phosphatidylserine |
| Celite | geldanamycin | ANS |
| Bis-ANS | | |

TABLE 2
proteins that act in the Crossbeta Pathway by binding to and/or interacting with misfolded proteins

| | | |
|---|---|---|
| Tissue-type plasminogen activator | Finger domain of tPA | Apolipoprotein E |
| Finger domain of factor XII | Finger domain(s) of fibronectin | Finger domain of HGFA |
| Finger domain(s) | Proteins comprising finger domains, e.g. | Affinity regions |
| | tPA, HGFA, factor XII, fibronectin | |
| Factor XII | Plasmin(ogen) | Matrix metalloprotease-1 |
| Fibronectin | 75 kD-neurotrophin receptor (p75NTR) | Matrix metalloprotease-2 |
| Hepatocyte growth factor activator | α2-macroglobulin | Matrix metalloprotease-3 |
| Serum amyloid P component | High molecular weight kininogen | Monoclonal antibody 2C11(F8A6)‡ |
| C1q | Cathepsin K | Monoclonal antibody 4A6(A7)‡ |
| CD36 | Matrix metalloprotease 9 | Monoclonal antibody 2E2(B3)‡ |
| Receptor for advanced glycation | Haem oxygenase-1 | Monoclonal antibody 7H1(C6)‡ |
| endproducts | | |
| Scavenger receptor-A | low-density lipoprotein receptor-related | Monoclonal antibody 7H2(H2)‡ |
| | protein (LRP, CD91) | |
| Scavenger receptor-B | DnaK | Monoclonal antibody 7H9(B9)‡ |
| ER chaperone Erp57 | GroEL | Monoclonal antibody 8F2(G7)‡ |
| Calreticulin | VEGF165 | Monoclonal antibody 4F4‡ |
| Monoclonal conformational antibody | Monoclonal conformational antibody WO2 | Amyloid oligomer specific antibody |
| WO1 (ref. (O′Nuallain and Wetzel, | (ref. (O′Nuallain and Wetzel, 2002)) | (ref. (Kayed et al., 2003)) |
| 2002)) | | |
| formyl peptide receptor-like 1 | α(6)β(1)-integrin | CD47 |
| Rabbit anti-albumin-AGE antibody, | CD40 | apo A-I belonging to small high-density |
| Aβ-purifieda) | | lipoproteins |
| apoJ/clusterin | 10 times molar excess PPACK, 10 mM | CD40-ligand |
| | εACA, (100 pM-500 nM) tPA2) | |
| macrophage scavenger receptor | Affinity region with affinity for mouse d-γ- | BiP/grp78 |
| CD163 | globulins | |
| Erdj3 | haptoglobin | α2-macroglobulin-trypsin complex |
| α2-macroglobulin-α-chymotrypisin | α2-macroglobulin-bromelain | Rheumatoid factor |
| complex | | |
| Rheumatoid factor IgA isotype | Rheumatoid factor IgG isotype | Rheumatoid factor IgM isotype |
| B-cell receptor with alpha, or | Anti-cyclic citrullinated peptide | Anti-citrullinated protein (auto)antibody |
| gamma, or mu chains | (auto)antibody | |
| HSP60 | HSP90 | Non-classical MHC-I molecule FcRn |
| HSP104 | ClpA | ClpB |
| Affinity regions with affinity for | Anti-citrullinated protein/peptide antibody | Affinity regions collected from a |
| misfolded proteins | | composition of affinity regions |
| | | using a crossbeta affinity matrix |
| Affinity regions collected from a | Affinity regions collected from a | Affinity regions collected from a |
| composition of affinity regions using | composition of affinity regions using a | composition of affinity regions using |
| a crossbeta HbAGE affinity matrix | crossbeta dIgIV affinity matrix | a crossbeta BSSAGE affinity matrix |
| Affinity regions collected from a | Affinity regions collected from a | Affinity regions collected from a |
| composition of affinity regions using | composition of affinity regions using a | composition of affinity regions using |
| a crossbeta Aβ affinity matrix | crossbeta Aβ fibril affinity matrix | a crossbeta dHSA affinity matrix |
| broad spectrum (human) | Affinity regions with affinity for crossbeta | Affinity regions collected from patient |
| immunoglobulin G (IgG) antibodies | structure or crossbeta induced | serum/plasma/synovial fluid using |
| (IgIV, IVIg) | conformation, e.g. collected from a | affinity |
| | composition of affinity regions | region matrix with affinity for |
| | | crossbeta |
| | | structure and/or crossbeta induced |
| | | conformation |
| Affinity region with affinity for | Affinity region with affinity for misfolded | Affinity region with affinity for Aβ |
| oxLDL/ApoB-100 | ApoA-I | |
| Affinity region with affinity for Aβ fibril | Affinity region with affinity for non-fibrillar | Affinity region with affinity for fibrin |
| | Aβ aggregates | |
| Affinity region with affinity for | Affinity region with affinity for BSAAGE | Affinity region with affinity for |
| HbAGE | | glycated protein |
| Affinity region with affinity for | Affinity region with affinity for dOVA | Affinity region with affinity for dHSA |
| citrullinated protein | | |
| Affinity region with affinity for human | Macrophage scavenger receptor-1 (MSR- | Anti-cyclic citrullinated peptide |
| dIgIV | 1) | antibody |
| Glycoprotein Ib (GpIb, CD42) | aprotinin | glypican-1 |
| Macrophage scavenger receptor-1 | chaperones | Heat shock proteins |
| Pro-inflammatory | Anti-inflammatory | chaperokines |
| chaperones/chaperokines | chaperones/chaperokines | |
| HSP70 | | |

‡Monoclonal antibodies developed in collaboration with the ABC-Hybridoma Facility, Utrecht University, Utrecht, The Netherlands.
a)Antigen albumin-AGE and ligand Aβ were send in to Davids Biotechnologie (Regensburg, Germany); a rabbit was immunized with albumin-AGE, antibodies against a structural epitope were affinity purified using a column with immobilized Aβ.
2)PPACK is Phe-Pro-Arg-chloromethylketone (SEQ-ID 11), εACA is ε-amino caproic acid, tPA is tissue-type plasminogen activator

TABLE 3
Proteins that are part of the Crossbeta Pathway

| | | |
|---|---|---|
| Monoclonal antibody 4B5 | Heat shock protein 27 | Heat shock protein 40 |
| Monoclonal antibody 3H7^{‡} | Nod2 (=CARD15) | Heat shock protein 70 |
| FEEL-1 | Pentraxin-3 | HDT1 |
| LOX-1 | Serum amyloid A proteins | GroES |
| MD2 | Stabilin-1 | cystatin C |
| FEEL-2 | Stabilin-2 | CD36 and LIMPII analogous-I (CLA-1) |
| Low Density Lipoprotein | LPS binding protein | CD14 |
| C reactive protein | CD45 | Orosomucoid |
| Integrins | alpha-1 antitrypsin | apo A-IV-Transthyretin complex |
| Albumin | Alpha-1 acid glycoprotein | β2-glycoprotein I |
| Lysozyme | Lactoferrin | Megalin |
| Tamm-Horsfall protein | Apolipoprotein E3 | Apolipoprotein E4 |
| Toll-like receptors | (pre)kallikrein | CD11d/CD18 (subunit aD) |
| CD11b2 | CD11a/CD18 (LFA-1, subunit aL) | CD11c/CD18 (CR4, subunit aX) |
| Von Willebrand factor | Myosin | Agrin |
| Perlecan | Chaperone60 | b2 integrin subunit |
| proteins that act in the unfolded protein | proteins that act in the endoplasmic | Macrophage receptor with collagenous |
| response (UPR) pathway of the | reticulum stress response (ESR) | structure (MARCO) |
| endoplasmic reticulum (ER) of | pathway of prokaryotic and | |
| prokaryotic and eukaryotic cells | eukaryotic cells | |
| 20S | CHAPERONE16 family members | HSC73 |
| HSC70 | plasmin(ogen) | 26S proteasome |
| 19S cap of the proteasome (PA700) | hepatocyte growth factor/scatter | carboxy-terminus of CHAPERONE70- |
| | factor | interacting protein (CHIP) |
| Pattern Recognition Receptors | Derlin-1 | Calnexin |
| Thrombospondin | GRP94 | Endoplasmic reticulum p72 |
| (broad spectrum) (human) | proteins that act in the endoplasmic | The (very) low density lipoprotein |
| immunoglobulin M (IgM) antibodies | reticulum associated degradation | receptor family |
| | system (ERAD) | |
| Fc receptors (e.g. human CD16, CD32A, | BcI-2 asociated athanogene (Bag-1) | UDP-glucose:glycoprotein glucosyl |
| CD32B, CD64) | | transferase (UGGT) |
| multidrug transporter, variously called | translocation channel protein | Complement receptor CD11b/CD18 |
| MultiDrug-Resistance 1 protein (MDR1), | Sec61p | (Mac-1, CR3) |
| P-glycoprotein (pleiotropic-glycoprotein), | | |
| Pgp, or P-170 | | |
| casein, αs-casein, β-casein | NFκB | Vitronectin |
| cytochrome p450 | c3 | CD79 |
| GrpE | TLR2 | TLR4 |
| TLR9 | (pro)thrombin | Fcε-receptors |
| MAC-2 | ERdj4 | syndecan |
| HSP67Bc | HSP90 co-chaperone | Derlin-2 |
| HSP75 | Mannose binding lectin (MBL) | HSP10 |
| chaperones | Co-chaperones | chaperonins |
| Heat shock proteins | Small heat shock proteins | chaperokines |
| Pro-inflammatory | Anti-inflammatory | Fibrin(ogen) receptor |
| chaperones/chaperokines | chaperones/chaperokines | |

^{‡}Monoclonal antibodies developed in collaboration with the ABC-Hybridoma Facility, Utrecht University, Utrecht, The Netherlands.

TABLE 4
Literature survey of chaperones/(small) heat shock proteins/(co-)chaperonins/co-chaperones/
chaperokines/stress proteins, identified in various species^{‡}

| | | | | | | | |
|---|---|---|---|---|---|---|---|
| Name | Other name | Other name | Group | Identified in^{¥} | Type | Type | Cellular localisation |

Group: small heat shock proteins (sHSP)

| | | | | | | | |
|---|---|---|---|---|---|---|---|
| Group: HSPC (sHsP) | | | sHsP | Bacteria | Chaperone | | Unknown/uncategorised |
| Group: HSPD (sHsP) | | | sHsP | Bacteria | Chaperone | | Unknown/uncategorised |
| Group: HSPE (sHsP) | | | sHsP | Bacteria | Chaperone | | Unknown/uncategorised |
| HsP22 (Drosophila) | | | sHsP | Drosophila | Chaperone | Stress protein | Unknown/uncategorised |
| HsP23 (Drosophila) | | | sHsP | Drosophila | Chaperone | Stress protein | Unknown/uncategorised |
| HsP26 (Drosophila) | | | sHsP | Drosophila | Chaperone | Stress protein | Unknown/uncategorised |
| HsP27 (Drosophila) | | | sHsP | Drosophila | Chaperone | Stress protein | Unknown/uncategorised |
| HsP25 | | | sHsP | Eukaryotes | Chaperone | Stress protein | Intracellular |
| HsPB5 | alpha-B-crystallin | | sHsP | Eukaryotes | Chaperone | | Unknown/uncategorised |
| HsPB4 | alpha-B-crystallin | | sHsP | Humans | Chaperone | | Unknown/uncategorised |
| HsPB6 | HsP20 | p20 | sHsP | Mammals | Chaperone | | Unknown/uncategorised |
| HsPB7 | | | sHsP | Mammals | Chaperone | | Unknown/uncategorised |
| HsPB8 | HsP22 | | sHsP | Mammals | Chaperone | | Unknown/uncategorised |
| HsPB9 | (testis specific) | | sHsP | Mammals | Chaperone | | Unknown/uncategorised |
| HsP16.5 | | | sHsP | Methanocaldococcus jannaschii | Chaperone | | Unknown/uncategorised |
| Group: HSPB (sHsP) | | | sHsP | Multiple | Chaperone | | Unknown/uncategorised |
| Group: HSPH (sHsP) | | | sHsP | Multiple | Chaperone | | Unknown/uncategorised |
| HsP18.1 | | | sHsP | Multiple | Chaperone | | Unknown/uncategorised |
| HsP16.3 | HSPX | | sHsP | Bacteria | Unknown/uncategorised | | Unknown/uncategorised |
| IbpA | 16 kDa HsPA | | sHsP | Bacteria | Unknown/uncategorised | Stress protein | Unknown/uncategorised |
| IbpB | 16 kDa HsPB | | sHsP | Bacteria | Unknown/uncategorised | Stress protein | Unknown/uncategorised |
| HsP16.2 | | | sHsP | C. Elegans | Unknown/uncategorised | | Unknown/uncategorised |
| Alpha-crystallin (bovine) | | | sHsP | Cows | Unknown/uncategorised | | Unknown/uncategorised |
| HsP67Ba (Drosophila) | Heat shock gene 67Ba | | sHsP | Drosophila | Unknown/uncategorised | | Unknown/uncategorised |
| HsP32 | hemo-oxygenase 1 | | sHsP | Eukaryotes | Unknown/uncategorised | Stress protein | Intracellular |
| HsP67Bc (Drosophila) | Heat shock gene 67Bc | | sHsP | Drosophila | Unknown/uncategorised | | Unknown/uncategorised |
| L(2)efl | | | sHsP | Drosophila | Unknown/uncategorised | | Unknown/uncategorised |
| HsP26 | | | sHsP | Eukaryotes | Unknown/uncategorised | Stress protein | Unknown/uncategorised |
| HsP16.9 | | | sHsP | Wheat | Unknown/uncategorised | | Unknown/uncategorised |
| HsPB1 | alpha-A-crystallin | HsP27 | sHsP | Eukaryotes | Chaperone | Stress protein | Unknown/uncategorised |
| HsPB2 | HsP27 | | sHsP | Eukaryotes | Chaperone | | Unknown/uncategorised |
| HsPB3 | HsP17 | | sHsP | Mammals | Chaperone | | Unknown/uncategorised |
| Group: HSPA (sHsP/HsP10) | | | sHsP/HsP10 | Multiple | Chaperone | | Unknown/uncategorised |
| GroES | | | sHsP/HsP10 | E. Coli | Co-Chaperonin | Stress protein | Cytoplasm |
| Chaperonin 10 (CPN10) | HsP10 | | sHsP/HsP10 | Multiple | Co-chaperonin | Stress protein | Intracellular |

Group: HsP40

| | | | | | | |
|---|---|---|---|---|---|---|
| HsP47 (collagen specific) | | HsP40 | Eukaryotes | Chaperone | Stress protein | ER |
| HsP40 | DnaJ | HsP40 | Multiple | Co-chaperone | Stress protein | Intracellular |
| Erdj5 | | HsP40 | Mammals | Co-chaperone | Stress protein | ER |
| HsP40 (Plasmodium falciparum) | | HsP40 | Plasmodium falciparum | Unknown/uncategorised | | Unknown/uncategorised |
| Group: DNAJ (HsP40/DnaJ) | | HsP40/DnaJ | Multiple | Co-chaperone | | Unknown/uncategorised |
| DnaJB11 | Erj-3 | HsP40/DnaJ | Eukaryotes | Co-chaperone | | Unknown/uncategorised |
| DnaJB12 | | HsP40/DnaJ | Eukaryotes | Co-chaperone | | Unknown/uncategorised |
| DnaJB13 | | HsP40/DnaJ | Eukaryotes | Co-chaperone | | Unknown/uncategorised |
| Sec63p | DnaJ homologue | HsP40/DnaJ | Eukaryotes | Co-chaperone | | Unknown/uncategorised |
| Hdj1 (HsP25 and HsP70 cochaperone) | | HsP40/DnaJ | Humans | Co-chaperone | Stress protein | Cytoplasm |
| DnaJA5 | | HsP40/DnaJ | Humans | Co-chaperone | | Unknown/uncategorised |
| Erdj4 | MDG1 | HsP40/DnaJ | Mammals | Co-chaperone | Stress protein | ER |

Group: HSP40

| | | | | | | | |
|---|---|---|---|---|---|---|---|
| Erdj3 | Hedj | | HsP40/DnaJ | Mammals | Co-chaperone | Stress protein | ER |
| DnaJA1 | DjA1 | | HsP40/DnaJ | Mammals | Co-chaperone | Stress protein | Cytoplasm |
| DnaJA2 | DjA2 | | HsP40/DnaJ | Mammals | Co-chaperone | Stress protein | Cytoplasm |
| DnaJA3 | DjA3 | Tid1 | HsP40/DnaJ | Mammals | Co-chaperone | Stress protein | Unknown/uncategorised |
| DnaJA4 | DjA4 | | HsP40/DnaJ | Mammals | Co-chaperone | Stress protein | Unknown/uncategorised |
| DnaJB1 | DjB1 | | HsP40/DnaJ | Mammals | Co-chaperone | | Unknown/uncategorised |
| DnaJB2 | HSJ-1 | | HsP40/DnaJ | Mammals | Co-chaperone | | Unknown/uncategorised |
| DnaJB3 | HSJ-3 | | HsP40/DnaJ | Mammals | Co-chaperone | | Unknown/uncategorised |
| DnaJB4 | | | HsP40/DnaJ | Mammals | Co-chaperone | | Unknown/uncategorised |
| DnaJB5 | Hsc-40 | | HsP40/DnaJ | Mammals | Co-chaperone | | Unknown/uncategorised |
| DnaJB6 | HSJ-2 | | HsP40/DnaJ | Mammals | Co-chaperone | | Unknown/uncategorised |
| DnaJB7 | | | HsP40/DnaJ | Mammals | Co-chaperone | | Unknown/uncategorised |
| DnaJB8 | | | HsP40/DnaJ | Mammals | Co-chaperone | | Unknown/uncategorised |
| DnaJB9 | | | HsP40/DnaJ | Mammals | Co-chaperone | | Unknown/uncategorised |
| DnaJB14 | | | HsP40/DnaJ | Mammals | Co-chaperone | | Unknown/uncategorised |
| DnaJB10 | | | HsP40/DnaJ | Mouse | Co-chaperone | | Unknown/uncategorised |

Group: HSP60

| | | | | | | |
|---|---|---|---|---|---|---|
| Group: HSPB (HsP60) | | HsP60 | Bacteria | Chaperone | | Unknown/uncategorised |
| GroEL | | HsP60 | E. Coli | Chaperonin | Stress protein | Cytoplasm |
| TCP-1 (complex with multiple | CCT | HsP60 | Eukaryotes | Chaperonin | Stress protein | Cytoplasm |
| subunits) | | | | | | |
| HsP60 (helicobacter pylori) | | HsP60 | Helicobacter pylori | Chaperonin | Stress protein | Cytoplasm |
| HsP60 | Cpn60 | HsP60 | Multiple | Chaperonin | Stress protein | Intracellular |
| TRiC-P5 (part of TRiC protein | | HsP60 | Multiple | Chaperonin | Stress protein | Intracellular |
| complex) | | | | | | |
| Chaperonin 60 (CPN60) | HsP60 | HsP60 | Multiple | Chaperonin | Stress protein | Intracellular |
| HsP60 (Plasmodium | | HsP60 | Plasmodium falciparum | Unknown/uncategorised | | Unknown/uncategorised |
| falciparum) | | | | | | |
| Group: HSPB (HsP60) | | HsP60 | Bacteria | Chaperone | | Unknown/uncategorised |
| GroEL | | HsP60 | E. Coli | Chaperonin | Stress protein | Cytoplasm |
| TCP-1 (complex with multiple | CCT | HsP60 | Eukaryotes | Chaperonin | Stress protein | Cytoplasm |
| subunits) | | | | | | |
| HsP60 (helicobacter pylori) | | HsP60 | Helicobacter pylori | Chaperonin | Stress protein | Cytoplasm |
| HsP60 | Cpn60 | HsP60 | Multiple | Chaperonin | Stress protein | Intracellular |

Group: HSP70

| Name | Other name | Other name | Group | Identified in^{¥} | Type | Type | Type | Cellular localisation |
|---|---|---|---|---|---|---|---|---|
| HsP72 | HsPA1A | | HsP70 | Mammals | Chaperokine | Stress protein | | Unknown/uncategorised |
| HsP75 (C. Elegans) | | | HsP70 | C. Elegans | Chaperone | | | Intracellular |
| HsPA3 | HSP-3 | | HsP70 | Cows | Chaperone | | | Unknown/uncategorised |
| DnaK | | | HsP70 | E. Coli | Chaperone | Stress protein | Disaggregase | Cytoplasm |
| HsP75 | | | HsP70 | Eukaryotes | Chaperone | | | Cytoplasm |
| Heat shock cognate protein 70 | | | HsP70 | Eukaryotes | Chaperone | Stress protein | | Cytoplasm |
| (HSC70) | | | | | | | | |
| HsP70B′ | | | HsP70 | Eukaryotes | Chaperone | Stress protein | | Intracellular |
| SSA1 | | | HsP70 | Eukaryotes | Chaperone | Stress protein | | Cytoplasm |
| SSA2 | | | HsP70 | Eukaryotes | Chaperone | Stress protein | | Unknown/uncategorised |
| SSB1 | | | HsP70 | Eukaryotes | Chaperone | Stress protein | | Unknown/uncategorised |
| SSB2 | | | HsP70 | Eukaryotes | Chaperone | Stress protein | | Unknown/uncategorised |
| HsPA5 | HSP-5 | BiP | HsP70 | Eukaryotes | Chaperone | Stress protein | | Unknown/uncategorised |
| HsPA8 | HSP-8 | | HsP70 | Eukaryotes | Chaperone | | | Unknown/uncategorised |
| Mitochondrial HSP70 | mHsP70 | | HsP70 | Eukaryotes | Chaperone | | | Mitochondria |
| HsPA6 | HsP70B′ | | HsP70 | Humans | Chaperone | Stress protein | | Unknown/uncategorised |
| HsPA7 | HsP70B′ | | HsP70 | Humans | Chaperone | Stress protein | | Unknown/uncategorised |
| HSC73 | PRP73 | | HsP70 | Mammals | Chaperone | Stress protein | | Cytoplasm |
| GRP75 | Mortalin | | HsP70 | Mammals | Chaperone | Stress protein | | Intracellular |
| HsPA2 | HSP-2 | | HsP70 | Mammals | Chaperone | | | Unknown/uncategorised |
| HsPA4 | HSP-4 | | HsP70 | Mammals | Chaperone | | | Unknown/uncategorised |
| HsPA9 | Mortalin | | HsP70 | Mammals | Chaperone | Stress protein | | Unknown/uncategorised |
| HsPA14 | HSP-14 | | HsP70 | Mammals | Chaperone | | | Unknown/uncategorised |
| BiP | GRP78 | | HsP70 | Multiple | Chaperone | Stress protein | Chaperokine | ER |
| HsP70 | | | HsP70 | Multiple | Chaperone | Stress protein | | Multiple |
| HsPA1 | HSP-1 | | HsP70 | Multiple | Chaperone | | | Unknown/uncategorised |
| SSA3 | | | HsP70 | Yeast | Chaperone | Stress protein | | Unknown/uncategorised |
| SSA4 | | | HsP70 | Yeast | Chaperone | Stress protein | | Unknown/uncategorised |
| Kar2p | DnaK homologue | | HsP70 | Yeast | Chaperone | Stress protein | | Unknown/uncategorised |
| HsP70 (Plasmodium falciparum) | | | HsP70 | Plasmodium falciparum | Unknown/uncategorised | | | Unknown/uncategorised |

Group: HSP90

| Name | Other name | Other name | Other name | Group | Identified in^{¥} | Type | Type | Cellular localisation |
|---|---|---|---|---|---|---|---|---|
| HtPG | HsP90 homologue | | | HsP90 | Bacteria | Chaperone | Stress protein | Unknown/uncategorised |
| HsP90 (C. Elegans) | DAF-21 | | | HsP90 | C. Elegans | Chaperone | Stress protein | Intracellular |
| GRP94 | gp96 | TRA1 | endoplasmin | HsP90 | Eukaryotes | Chaperone | Stress protein | ER |
| HsP90 | | | | HsP90 | Multiple | Chaperone | Stress protein | Intracellular |
| HsP90 (Plasmodium falciparum) | | | | HsP90 | Plasmodium falciparum | Unknown/uncategorised | | Unknown/uncategorised |

Group: HSP100

| Name | Other name | Other name | Group | Identified in^{¥} | Type | Type | Type | Cellular localisation |
|---|---|---|---|---|---|---|---|---|
| ClpB | | | HsP100 | Bacteria | Chaperone | Stress protein | Disaggregase | Cytoplasm |
| SSE1 | HsP110 | | HsP100 | Eukaryotes | Chaperone | Stress protein | | Cytoplasm |
| Oxygen regulated protein 150 (ORP150) | GRP170 | | HsP100 | Mammals | Chaperone | Stress protein | | ER |
| HsP104 | ClpB | | HsP100 | Multiple | Chaperone | Stress protein | Disaggregase | Intracellular |
| GRP170 (ER) | | | HsP100 | Multiple | Chaperone | Stress protein | | ER |
| SSE2 | HsP110 | | HsP100 | Yeast | Chaperone | Stress protein | | Cytoplasm |
| APG1 | HSP-4L | | HsP100/HsP110 | Mammals | Chaperone | | | Unknown/uncategorised |

Group: ERAD (Endoplasmic Reticulum-assocoiated Degradation)

| Name | Other name | Other name | Group | Identified in^{¥} | Type | Type | Cellular localisation | Cellular localisation |
|---|---|---|---|---|---|---|---|---|
| Cdc48 | P97 | | ERAD | Multiple | Chaperone | ERAD | Intracellular | ER (eukaryotes) |
| Ubx2 | Sel1 | | ERAD | Eukaryotes | Cofactor | ERAD | ER | |

Group: PDI (Protein Disulfide Isomerase)

| Name | Group | Identified in^{¥} | Remarks | Type | Type | Cellular localisation |
|---|---|---|---|---|---|---|
| Protein disulfide isomerase (PDI) (ER) | PDI | Eukaryotes | | Chaperone | Stress protein | ER |
| Erp44 | PDI | Eukaryotes | | Chaperone | | ER |
| PDIp (pancreatic PDI) | PDI | Mammals | | Chaperone | Stress protein | Intracellular (microsomes) |
| PDI homologues | PDI | Multiple | | Chaperone | Stress protein | Intracellular |
| Erp46 | PDI | Eukaryotes | | Suspected Chaperone | | ER |
| Erp19 | PDI | Eukaryotes | | Suspected Chaperone | | ER |
| Testis PDI-LT | PDI | Eukaryotes | Identified in human, cow, fish and dog | Suspected Chaperone | | ER |
| Erp27 (human) | PDI | Humans | | Suspected Chaperone | | ER |
| Erp28 | PDI | Mammals | | Suspected Chaperone | | ER |
| Erp18 | PDI | Mammals | | Suspected Chaperone | | ER |
| Tmx | PDI | | | Suspected Chaperone | | Unknown/uncategorised |
| Tmx1 | PDI | | | Suspected Chaperone | | Unknown/uncategorised |
| Tmx2 | PDI | | | Suspected Chaperone | | Unknown/uncategorised |
| Tmx3 | PDI | | | Suspected Chaperone | | Unknown/uncategorised |
| Tmx4 | PDI | | | Suspected Chaperone | | Unknown/uncategorised |

Group: Unknown/Unidentified/un-categorized

| Name | Other name | Other name | Group | Identified in^{¥} | Type | Type | Cellular localisation |
|---|---|---|---|---|---|---|---|
| CIpA | | | Unknown/uncategorised | Bacteria | Chaperone | Stress protein | Cytoplasm |
| SecB | | | Unknown/uncategorised | Bacteria | Chaperone | | Unknown/uncategorised |
| SecB-SecA complex | | | Unknown/uncategorised | Bacteria | Chaperone | | Unknown/uncategorised |
| SecY | | | Unknown/uncategorised | Bacteria | Chaperone | | Unknown/uncategorised |
| SecY-SecE-SecG complex | | | Unknown/uncategorised | Bacteria | Chaperone | | Unknown/uncategorised |
| SecD-SecF-YajC complex | | | Unknown/uncategorised | Bacteria | Chaperone | | Unknown/uncategorised |
| Calreticulin | | | Unknown/uncategorised | Eukaryotes | Chaperone | Stress protein | ER |
| Clusterin | ApoJ | | Unknown/uncategorised | Eukaryotes | Chaperone | Stress protein | Multiple |
| CaBP1 | P5 | | Unknown/uncategorised | Eukaryotes | Chaperone | | ER |
| Erp72 (ER) | | | Unknown/uncategorised | Eukaryotes | Chaperone | Stress protein | ER |
| Calnexin | | | Unknown/uncategorised | Eukaryotes | Chaperone | Stress protein | ER |
| p72 (ER) | | | Unknown/uncategorised | Eukaryotes | Chaperone | Stress protein | ER |
| Sec61p | | | Unknown/uncategorised | Eukaryotes | Chaperone | | ER |
| Haptoglobin | | | Unknown/uncategorised | Mammals | Chaperone | Acute phase protein | Extracellular |
| Dertin-2 | | | Unknown/uncategorised | Mammals | Chaperone | ERAD | ER |
| Dertin-3 | | | Unknown/uncategorised | Mammals | Chaperone | ERAD | ER |
| GRP170 | | | Unknown/uncategorised | Mammals | Chaperone | Disaggregase | Unknown/uncategorised |
| Cyclophilin B (ER) | | | Unknown/uncategorised | Multiple | Chaperone | Stress protein | ER |
| Nap1 | | | Unknown/uncategorised | Multiple | Chaperone | | Histones |
| Histone chaperones | | | Unknown/uncategorised | Multiple | Chaperone | | Histones |
| Erp57 | ER-60 | GRP58 | Unknown/uncategorised | Eukaryotes | Chaperone N | Co-chaperone | ER |
| HsP90 co-chaperone | UNC-45 | | Unknown/uncategorised | C. Elegans | Co-chaperone | Stress protein | Intracellular |
| (C. Elegans) | | | | | | | |
| HsP70 interacting protein (C- | CHIP (C- | | Unknown/uncategorised | Eukaryotes | Co-chaperone | Stress protein | Intracellular |
| terminus) | terminus) | | | | | | |
| Mge1 | GrpE | | Unknown/uncategorised | Eukaryotes | Co-chaperone | | Mitochondria |
| | homologue | | | | | | |
| GrpE | | | Unknown/uncategorised | Multiple | Co-chaperone | Stress protein | Cytoplasm (bacteria) |
| Viral GP31 (replaces GroES) | | | Unknown/uncategorised | Bacteriophages | Co-chaperonin | | Intracellular |
| AHA-1 (HsP90 co-factor) | | | Unknown/uncategorised | C. Elegans | Co-factor | | Intracellular |
| C/EBP-homologous protein | GADD153 | | Unknown/uncategorised | Mammals | Transcription factor | Stress protein | ER |
| (CHOP) | | | | | | | |
| TIM (protein complex) | | | Unknown/uncategorised | Eukaryotes | Unknown/uncategorised | | Mitochondria |

| | | | | | | |
|---|---|---|---|---|---|---|
| Name | Other name | Group | Identified in^{¥} | Type | Type | Cellular localisation |

Group: Proteasome

| | | | | | | |
|---|---|---|---|---|---|---|
| 20S | | Proteasome | Multiple | Chaperone | Stress protein | Intracellular |
| 26S | | Proteasome | Multiple | Chaperone | Stress protein | Intracellular |
| 19S | PA700 | Proteasome | Multiple | Chaperone | Stress protein | Intracellular |

Group: Cytokine

| | | | | |
|---|---|---|---|---|
| Macrophage migration inhibitory factor (MIF) | Cytokine | Multiple | Cytokine | Multiple |

Group: Unknown/Unidentified/un-categorized (continued)

| Name | Other name | Other name | Group | Identified in^{¥} | Type | Type | Cellular localisation |
|---|---|---|---|---|---|---|---|
| TOM (protein complex) | | | Unknown/uncategorised | Eukaryotes | Unknown/uncategorised | | Mitochondria |
| HsP70 protein like 1 | | | Unknown/uncategorised | Eukaryotes | Unknown/uncategorised | Stress protein | Cytoplasm |
| SDF2-L1 | | | Unknown/uncategorised | Humans | Unknown/uncategorised | | ER |
| UDP-glucosyltransferase (ER) | | | Unknown/uncategorised | Mammals | Unknown/uncategorised | | ER |
| Hap50 | BAG-1L | | Unknown/uncategorised | Mammals | Unknown/uncategorised | | Unknown/uncategorised |
| Hap46 | BAG-1M | | Unknown/uncategorised | Mammals | Unknown/uncategorised | | Unknown/uncategorised |
| Mycobacterial heat shock | HsP65 | | Unknown/uncategorised | Mycobacteria | Unknown/uncategorised | Stress protein | Cytoplasm |
| protein | | | | | | | |
| Chaperone 72 | | | Unknown/uncategorised | | Unknown/uncategorised | | Unknown/uncategorised |

Reference to families, pathways, classes, groups, super-families of chaperones/(small) heat shock proteins/
chaperonins/co-chaperones/chaperokines, identified in various species

| chaperones | chaperonins | Heat shock proteins |
|---|---|---|
| Small heat shock proteins | Co-chaperones | Chaperokines |
| PDI-homologues | AAA+ protein superfamily | Stress proteins |
| Co-chaperonins | Molecular chaperones | (protein) disaggregases |
| proteins that act in the unfolded protein | proteins that act in the endoplasmic | proteins that act in the |
| response (UPR) pathway of the | reticulum stress response (ESR) | endoplasmic reticulum associated |
| endoplasmic reticulum (ER) of | pathway of prokaryotic and | degradation system (ERAD) |
| prokaryotic and eukaryotic cells | eukaryotic cells | |
| chaperones involved in the translocation | Anti-inflammatory | Chaperones in the protein |
| of (partially) unfolded/denatured | chaperones/chaperokines | translocation system in bacteria, |
| proteins across membranes | | the general secretion pathway |
| | | (Sec-pathway) |
| (small) heat shock proteins from | (small) heat shock proteins from | (small) heat shock proteins from |
| Alveolata | animals | fungi |
| (small) heat shock proteins from plant | (small) heat shock proteins from | (small) heat shock proteins from |
| | human | mammals |
| (small) heat shock proteins from archaea | (small) heat shock proteins from | (small) heat shock proteins from |
| | bacteria class A | bacteria class B |
| (small) heat shock proteins from | (small) heat shock proteins from | HSPs lacking ATPase activity, |
| Rhodophyta | Entamoebidae | such as haptoglobin and clusterin |
| Intracellular chaperones | Extracellular chaperones | |
| HSPs with ATPase activity, such as | Pro-inflammatory | |
| HSP70, HSP90, GroEL, BiP | chaperones/chaperokines | |

^{‡}Data are mainly obtained from search engine Google (www.google.nl). scientific literature database PubMed (http://www.ncbi.nlm.nih.gov/entrez/query.fcgi) and patent database Espacenet (http://ep.espacenet.com/quickSearch?locale=en_ep), using predominantly keywords like chaperone/chaperonin/chaperokine/co-chaperone/stress protein/co-chaperonin/disaggregase/heat shock protein/HSP/small heat shock protein.

TABLE 5
Peptides with a potential propensity to adopt amyloid-like crossbeta-structure
misfolded conformation

| Peptide | | | | tPA-Plg | | pl/MW |
|---|---|---|---|---|---|---|
| code^{‡} | Seq id | sequence | Peptide source | act./ThT/Congo red | TEM | (Da) |
| 6BB6 | 2 | GSNKGAIIGLM | Human Aβ (25-35) | −/+/+ | Thin aggregated | 8.75/ |
| | | | | | fibers | 1060 |
| 6BB7 | 3 | AGEYYAAL | Synthetic peptide that does | −/−/− | Very few small | 4.0/ |
| | | | not bind BiP (np53) | | aggregates | 857 |
| 6BB8 | 4 | YVDRFIGW | BiP binding sequence, | +/+/+ | Not determined | 5.84/ |
| | | | stimulator of ATPase activity | | | 1055 |
| | | | of BiP | | | |
| 6BB9 | 5 | LFWPFEWI | Synthetic BiP binding motif | +/−/− | Not determined | 4.0/ |
| | | | | | | 1137 |
| 6BB10 | 6 | HWDFAWPW | Synthetic BiP binding motif | +/−/− | Nothing visible^{†} | 5.08/ |
| | | | | | | 1144 |
| 6BB11 | 7 | FWGLWPWE | Synthetic BiP binding motif | +/−/− | Not determined | 4.60/ |
| | | | | | | 1120 |
| 6BB12 | 8 | KLVFFAE | Human Aβ16-22 | −/+/+ | Large dense clusters | 6.22/ |
| | | | | | of amorphous | 853 |
| | | | | | aggregates and few | |
| | | | | | thin fibers | |
| 6BB13 | 9 | RRRAA | Rheumatoid arthiritis related | +/−/− | Not determined | 12.30/ |
| | | | HLA-DR motif with affinity | | | 629 |
| | | | for HSP70 | | | |

Remark:
all peptides have an unmodified NH₂-terminus and unmodified COOH-terminus.
^{‡}2.5 mg/ml peptide stocks used for studies listed in this Table are 6BB 6-pH 12, 6BB7-pl, 6BB 8-pH 2, 6BB9-pl, 6BB10-pH 2, 6BB11-pH 12, 6BB12-pH 12, 6BB13-pl, 15-PH 12
^{†}In the Eppendorf cup aggregates are visible, which are apparently not coated onto the TEM grids.

TABLE 6
Determination of endotoxin levels in various
protein solutions, using a LAL assay

| | Endotoxin | Estimated LPS |
|---|---|---|
| Sample | level (EU) | content (ng/ml) |
| dOVA standard (1 mg/ml) | 115.6 | 250 |
| Octagam (lot 5024018434, 50 mg/ml) | 0.033, 0.147 | <0.25 |
| HbAGE (1.6 mg/ml) | 0.122 | <0.25 |
| CEALB HSA (Sanquin, lot 05C29H120A, | 0 | 0 |
| 200 mg/ml) | | |
| Fibronectin finger4-5-FLAG-His (290 | 1.7 | n.d. |
| μg/ml in PBS with 10% glycerol) | | |
| tPA (Actilyse, 50 μM; 3.65 mg/ml) | 2.7 | n.d. |

TABLE 7
Influence of crossbeta binding compounds/proteins on interaction
of BiP with misfolded proteins comprising crossbeta structure

| | Crossbeta binding compounds | | | | | |
|---|---|---|---|---|---|---|
| crossbeta | ThT^{‡} | Congo red | ThS | IgIV | K2P tPA | tPA |
| Aβ25-35 | ↑ | ↓ | − | ↓ | − | ↓ |
| HbAGE | ↑ | ↓ | ↑ | ↓ | −/↓ | ↓ |
| 6BB9 | ↓ | ↓ | ↓ | n.d. | n.d. | n.d. |
| 6BB10 | ↓ | ↓ | ↓ | n.d. | n.d. | n.d. |
| 6BB11 | ↓ | ↓ | ↓ | n.d. | n.d. | n.d. |
| oxLDL | ↑ | ↓ | ↑ | ↓ | − | ↓ |
| dOVA | n.d. | ↓ | ↓ | n.d. | − | ↓ |

^{‡}A ↑ sign refers to stimulated binding of BiP to the misfolded protein comprising crossbeta, a ↓ sign refers to inhibited abolished binding of BiP, a minus sign indicated that the compound did not influence BiP binding with the current experimental settings. N.d., not determined. With HbAGE, K2P tPA gave some inhibited BiP binding in a second experiment, indicated with a small _{↓} sign.

TABLE 8
Proteins uniquely identified in elua
tes of BiP-Sepharose that was contacted with AL amyloidosis patient plasma
(B2)

| Protein name^{‡} | IPI accession | # of peptides in |
|---|---|---|
| Sample B2 [AL amyloidosis patient plasma] | number^{†} | sample A#¹⁾ |
| Immunoglobulin lambda variable 3-25/V2-17 protein | IPI00550162 | 8 |
| (Fragment) | (also in B4) | |
| Hypothetical protein/immunoglobulin lambda locus/ | IPI00760678 | 1 |
| immunoglobulin lambda chain C regions (1/2/3)/ | | |
| immunoglobulin lambda variable V2-14/Ig lambda C3 protein | | |
| (C2 segment protein, C3 segment protein)/Hypothetical | | |
| protein DKFZp667J0B10 (Fragment) | | |
| Hypothetical protein | IPI00807428 | 1 |
| | (also in B3) | |
| IGLC2 protein (immunoglobulin lambda C2)/Immunoglobulin | IPI00555945 | 1 |
| lambda | | |
| Apolipoprotein E precursor/Apolipoprotein E/Apolipoprotein | IPI00021842 | 6 |
| E3 (Fragment) | | |
| Fc fragment of IgG binding protein (IgGFc-binding protein | IPI00242956 | 4 |
| precursor/FcgammaBP/Fcgamma-binding protein antigen) | | |
| Immunoglobulin heavy constant gamma 3 (G3m marker)/Ig | IPI00472345 | 2 |
| gamma-3 chain C region (Heavy chain disease protein, HDC) | (also in B3) | |
| 26 kDa protein/immunoglobulin kappa variable 1-5 | IPI00738024 | 1 |
| | (also in B3) | |
| Hypothetical protein/immunoglobulin lambda variable 4-3 | IPI00382938 | 1 |
| | (also in B3) | |
| 25 kDa protein/immunoglobulin lambda chain/rheumatoid | IPI00154742 | 1 |
| factor G9 light chain (lambda V3)/IGLC1 protein | (also in B3) | |
| Isoform Long of Antigen KI-67/Antigen KI-67 | IPI00004233 | 1 |

^{‡}Proteins are listed that are identified based on identified peptide masses. For peptide masses that are not unique for a single protein all proteins with the identified sequence are listed
^{†}A# series: analyzed eluate from enriched IgIV-matrix after contacting with indicated patient samples; The control serie C# displayed are the analyses of eluates from control matrix with affinity regions contacted with the same patient samples; background measurement.
¹⁾The IPI accession codes refer to protein entry codes for various protein/peptide databases. When the same protein(s) were also identified in one or more of the other analysed eluates after contacting affinity region-matrix with patient sample, these patient sample codes are given.

TABLE 9
Proteins uniquely identified in eluates of BiP-Sepharose that was
contacted with AL amyloidosis patient plasma (B3)

| Protein name^{‡} | IPI accession | # of peptides in |
|---|---|---|
| Sample B3 [AL amyloidosis patient plasma] | number^{†} | sample A#¹⁾ |
| Hypothetical protein/Ig kappa chain V-II region RPMI 6410 | IPI00550731 | 1 |
| precursor | | |
| Hypothetical protein | IPI00807428 (also in | 2 |
| | B2) | |
| Single-chain Fv (Fragment) | IPI00470652 | 2 |
| Hypothetical protein DKFZp686N02209/immunoglobulin heavy | IPI00384938 | 1 |
| constant gamma 1 (G1m marker) | | |
| Hypothetical protein | IPI00784983 (also in | 2 |
| | B6) | |
| V1-3 protein (Fragment) | IPI00552874 | 2 |
| Apolipoprotein E precursor/Apolipoprotein E/Apolipoprotein | IPI00021842 | 1 |
| E3 (Fragment) | | |
| Complement factor H-related protein 3 precursor/Complement | IPI00027507 | 1 |
| factor H-related 3 (FHR-3, H factor-like protein 3, DOWN16)/ | | |
| Complement factor H-related 3, isoform CRA_c | | |
| Dynein heavy chain domain 3/KIAA1503 protein (Fragment) | IPI00783464 | 2 |
| Immunoglobulin heavy constant mu/Full-length cDNA clone | IPI00748158 | 4 |
| CS0DD006YL02 of Neuroblastoma of Homo sapiens | | |
| CDNA FLJ90170 fis, clone MAMMA1000370, highly similar to Ig | IPI00449920 (also in | 2 |
| alpha-1 chain C region/Immunoglobulin heavy constant alpha 1 | B4) | |
| Immunoglobulin heavy constant gamma 3 (G3m marker)/Ig | IPI00472345 (also in | 2 |
| gamma-3 chain C region (Heavy chain disease protein/HDC) | B2) | |
| 26 kDa protein/Immunoglobulin kappa variable 1-5 | IPI00738024 (also in | 1 |
| | B2) | |
| Hypothetical protein LOC651928/Immunoglobulin kappa | IPI00440577 (also in | 1 |
| variable 1-5/IGKV2-24 protein | B4) | |
| 25 kDa protein/Immunoglobulin lambda locus | IPI00747752 | 8 |
| Hypothetical protein/Immunoglobulin lambda variable 4-3 | IPI00382938 (also in | 2 |
| | B2) | |
| 25 kDa protein/immunoglobulin lambda chain/rheumatoid | IPI00154742 (also in | 1 |
| factor G9 light chain (lambda V3)/IGLC1 protein | B2) | |
| IGLC1 protein (immunoglobulin lambda C1)/immunoglobulin | IPI00719373 (also in | 1 |
| lambda chain/immunoglobulin C1 segment protein (fragment) | B4 and B6) | |
| Isoform 1 of Plectin-1/Plectin 1 (intermediate filament binding | IPI00014898 (also in | 1 |
| protein 500 kDa, Hemidesmosomal protein 1/HD1) | B4) | |

^{‡}Proteins are listed that are identified based on identified peptide masses. For peptide masses that are not unique for a single protein all proteins with the identified sequence are listed
^{†}A# series: analyzed eluate from enriched IgIV-matrix after contacting with indicated patient samples; The control serie C# displayed are the analyses of eluates from control matrix with affinity regions contacted with the same patient samples; background measurement.
¹⁾The IPI accession codes refer to protein entry codes for various protein/peptide databases. When the same protein(s) were also identified in one or more of the other analysed eluates after contacting affinity region-matrix with patient sample, these patient sample codes are given.

TABLE 10
Proteins uniquely identified in eluates of BiP-Sepharose that was
contacted with RA patient serum (B4)

| | | # of peptides |
|---|---|---|
| Protein name^{‡} | IPI accession | in sample |
| Sample B4 [RA patient serum] | number^{†} | A#¹⁾ |
| IGLV3-25 protein (immunoglobulin lambda variable 3-25: synonym: | IPI00550162 | 10 |
| V2-17) | (also in B2) | |
| Ig heavy chain V-III region TUR/Ig heavy chain V-III region TIL | IPI00382478 | 1 |
| Ig lambda chain V-III region SH | IPI00382436 | 2 |
| Full-length cDNA clone CS0DL004YM19 of B cells (Ramos cell line) | IPI00328493 | 4 |
| of Homo sapiens (Fragment)/Unnamed protein product | | |
| Myosin-reactive immunoglobulin light chain variable region/Ig | IPI00024138 | 3 |
| kappa chain V-III region VH precursor (Fragment) | | |
| V1-3 protein (Fragment)/immunoglobulin lambda variable 2-11 | IPI00552874 | 1 |
| 187 kDa protein/Complement component 3 | IPI00164623 | 9 |
| Complement component 4 binding protein, alpha, C4b-binding | IPI00021727 | 7 |
| protein alpha chain precursor (C4bp) (Proline-rich protein/PRP) | | |
| CD5 antigen-like precursor (SP-alpha, CT-2, IgM-associated | IPI00025204 | 1 |
| peptide)/CD5 molecule-like | | |
| Dapper homolog 1 (hDPR1) (Heptacellular carcinoma novel gene 3)/ | IPI00171594 | 2 |
| Dapper, antagonist of beta-catenin, homolog 1 (Xenopus laevis) | | |
| GCN1 general control of amino-acid synthesis 1-like 1 (yeast)/ | IPI00001159 | 2 |
| GCN1-like protein 1 (HsGCN1) | | |
| Immunoglobulin heavy constant mu/Full-length cDNA clone | IPI00748158 | 10 |
| CS0DD006YL02 of Neuroblastoma of Homo sapiens (human)/ | | |
| Unnamed protein product | | |
| CDNA FLJ90170 fis, clone MAMMA1000370, highly similar to Ig | IPI00449920 | 17 |
| alpha-1 chain C region/Immunoglobulin heavy constant alpha 1 | (also in B3) | |
| Hypothetical protein DKFZp686I04196 (Fragment)/ | IPI00399007 | 14 |
| Immunoglobulin heavy constant gamma 2 (G2m marker)/Ig | | |
| gamma-2 chain C region | | |
| Immunoglobulin J chain, Immunoglobulin J polypeptide, linker | IPI00178926 | 5 |
| protein for immunoglobulin alpha and mu polypeptides | | |
| Hypothetical protein LOC651928/Immunoglobulin kappa variable | IPI00440577 | 1 |
| 1-5/IGKV2-24 protein | (also in B3) | |
| IGLC1 protein (immunoglobulin lambda C1)/immunoglobulin | IPI00719373 | 2 |
| lambda chain/immunoglobulin C1 segment protein (fragment) | (also in B3 and | |
| | B6) | |
| Ig kappa chain V-I region WEA/Similar to Ig kappa chain V-I | IPI00003111 | 2 |
| region HK102 precursor/Ig kappa chain V-I region AU | | |
| Phosphatase and actin regulator 4 isoform 1/Hypothetical protein | IPI00217851 | 2 |
| DKFZp686L07205 | | |
| Isoform 1 of Plectin-1/Plectin 1, intermediate filament binding | IPI00014898 | 2 |
| protein 500 kDa, Hemidesmosomal protein 1 | (also in B3) | |
| Alpha-1-antitrypsin precursor/Serpin peptidase inhibitor, clade A | IPI00553177 | 1 |
| (alpha-1 antiproteinase, antitrypsin), member 1/Full-length cDNA | | |
| clone CS0DE007YP21 of Placenta of Homo sapiens (human) (Full- | | |
| length cDNA clone CS0DI028YP15 of Placenta of Homo sapiens) | | |
| Vitronectin precursor (Serum-spreading factor/S-protein/V75 | IPI00298971 | 4 |
| [Contains: Vitronectin V65 subunit; Vitronectin V10 subunit; | | |
| Somatomedin B])/Vitronectin | | |

^{‡}Proteins are listed that are identified based on identified peptide masses. For peptide masses that are not unique for a single protein all proteins with the identified sequence are listed
^{†}A# series: analyzed eluate from enriched IgIV-matrix after contacting with indicated patient samples; The control serie C# displayed are the analyses of eluates from control matrix with affinity regions contacted with the same patient samples; background measurement.
¹⁾The IPI accession codes refer to protein entry codes for various protein/peptide databases. When the same protein(s) were also identified in one or more of the other analysed eluates after contacting affinity region-matrix with patient sample, these patient sample codes are given.

TABLE 11
Proteins uniquely identified in eluates of BiP-Sepharose that was
contacted with RA patient synovial fluid (B6)

| Protein name^{‡} | IPI accession | # of peptides in |
|---|---|---|
| Sample B6 [RA patient synovial fluid] | number^{†} | sample A#¹⁾ |
| Hypothetical protein | IPI00784983 | 1 |
| | (also in B3) | |
| Ig heavy chain V-III region CAM | IPI00382482 | 2 |
| Hypothetical protein/Heat shock 70 kDa protein 5 (glucose- | IPI00003362 | 31 |
| regulated protein, 78 kDa = GRP78 = BiP = HSPA5) | | |
| Immunoglobulin heavy constant mu | IPI00479708 | 1 |
| 26 kDa protein/Immunoglobulin kappa variable 1-5 | IPI00430820 | 6 |
| IGLC1 protein (immunoglobulin lambda C1)/immunoglobulin | IPI00719373 | 1 |
| lambda chain/immunoglobulin C1 segment protein (fragment) | (also in B3 and | |
| | B4) | |

^{‡}Proteins are listed that are identified based on identified peptide masses. For peptide masses that are not unique for a single protein all proteins with the identified sequence are listed
^{†}A# series: analyzed eluate from enriched IgIV-matrix after contacting with indicated patient samples; The control serie C# displayed are the analyses of eluates from control matrix with affinity regions contacted with the same patient samples; background measurement.
¹⁾The IPI accession codes refer to protein entry codes for various protein/peptide databases. When the same protein(s) were also identified in one or more of the other analysed eluates after contacting affinity region-matrix with patient sample, these patient sample codes are given.

TABLE 12
Proteins uniquely identified in eluates of matrix with
enriched IgIV with specificity for misfolded proteins, that was
contacted with AL amyloidosis patient plasmas

| | | # of |
|---|---|---|
| Protein name^{‡} | | peptides |
| Sample A2 and A3 [AL amyloidosis patient | IPI accession | in sample |
| plasma] | number^{†} | A#¹⁾ |
| dynein heavy chain domain 3 (gene name: | IPI00783464 | 1 |
| KIAA1503) | | |
| IGLC1 protein/immunoglobulin lambda chain | IPI00658130 | 1 |
| | (also in A4 | |
| | and A6) | |
| 25 kDa/protein/immunoglobulin lambda locus | IPI00747752 | 1 |
| (gene) | (also in A4 | |
| | and A6) | |
| Hypothetical protein/immunoglobulin lambda | IPI00382938 | 1 |
| variable 4-3 | | |
| 25 kDa protein/immunoglobulin lambda chain/ | IPI00154742 | 1 |
| rheumatoid factor G9 light chain (lambda V3)/ | (also in A4 | |
| IGLC1 protein | and A6) | |

^{‡}Proteins are listed that are identified based on identified peptide masses. For peptide masses that are not unique for a single protein all proteins with the identified sequence are listed
^{†}A# series: analyzed eluate from enriched IgIV-matrix after contacting with indicated patient samples; The control serie C# displayed are the analyses of eluates from control matrix with affinity regions contacted with the same patient samples; background measurement.
¹⁾The IPI accession codes refer to protein entry codes for various protein/peptide databases. When the same protein(s) were also identified in one or more of the other analysed eluates after contacting affinity region-matrix with patient sample, these patient sample codes are given.

TABLE 13
Proteins uniquely identified in eluates of matrix with enriched IgIV with specificity for
misfolded proteins, that was contacted with RA patient serum and RA patient synovial fluid

| | | # of peptides |
|---|---|---|
| | IPI accession | in sample |
| Protein name^{‡} | number^{†} | A#¹⁾ |
| Sample A4 [RA patient serum] | | |
| IGLV3-25 protein (immunoglobulin lambda variable 3-25: synonym: V2-17) | IPI00550162 | 1 |
| Hypothetical protein/immunoglobulin lambda locus/immunoglobulin | IPI00760678 (also | 1 |
| lambda chain C regions (1/2/3)/immunoglobulin lambda variable V2-14/Ig | in A6) | |
| lambda C3 protein (C2 segment protein/C3 segment protein)/Hypothetical | | |
| protein DKFZp667J0810 (Fragment) | | |
| Hypothetical protein | IPI00784519 | 1 |
| Hypothetical protein | IPI00784711 | 1 |
| Hypothetical protein | IPI00784983 | 1 |
| IGLC2 protein (immunoglobulin lambda C2) | IPI00555945 | 1 |
| IGLC2 protein (immunoglobulin lambda C2) | IPI00450309 | 1 |
| Isoform 1 of Centrosomal protein Cep290/Centrosomal protein Cep290: | IPI00784201 | 1 |
| synonyms (Nephrocystin-6) (Tumor antigen se2-2) | | |
| Isoform Gamma-B of Fibrinogen gamma chain precursor | IPI00021891 | 1 |
| IGLC1 protein (immunoglobulin lambda C1)/immunoglobulin lambda chain | IPI00658130 (also | 1 |
| | in A2, A3 and A6) | |
| 25 kDa protein/immunoglobulin lambda chain | IPI00747752 (also | 1 |
| | in A2, A3 and A6) | |
| 25 kDa protein/immunoglobulin lambda chain/rheumatoid factor G9 light | IPI00154742 (also | 1 |
| chain (lambda V3)/IGLC1 protein | in A2, A3 and A6) | |
| IGLC1 protein (immunoglobulin lambda C1)/immunoglobulin lambda chain/ | IPI00719373 | 1 |
| immunoglobulin C1 segment protein (fragment) | | |
| Isoform Long of Antigen KI-67/Antigen KI-67 | IPI00004233 | 1 |
| Sample A6 [RA patient synovial fluid] | | |
| IGKC protein (immunoglobulin kappa constant) | IPI00807413 | 10 |
| Hypothetical protein/immunoglobulin lambda constant 2/IGLV2-14 | IPI00760678 (also | 1 |
| (immunoglobulin variable 2-14/Ig lambda C3 protein (C2 segment protein/ | in A4) | |
| C3 segment protein)/IGLC1 (immunoglobulin lambda constant 1)/ | | |
| Hypothetical protein DKFZp667J0810 | | |
| Hypothetical protein | IPI00807428 | 7 |
| IGHA1 protein (immunoglobulin heavy constant alpha 1) | IPI00166866 | 10 |
| Single-chain Fv (Fragment)/Immunoglobulin heavy chain variable region | IPI00748998 | 2 |
| (fragment)/ | | |
| Beta-2-glycoprotein 1 precursor/Beta-2-glycoprotein (Apolipoprotein H) | IPI00298828 | 7 |
| Complement C1q subcomponent subunit C precursor/complement | IPI00022394 | 1 |
| component 1, q subcomponent, C chain | | |
| Complement C1r subcomponent precursor/complement component 1, r | IPI00296165 | 1 |
| subcomponent/Hypothetical protein DKFZp686O02154 | | |
| Calmodulin-like protein 5 (Calmodulin-like skin protein) | IPI00021536 | 2 |
| Complement factor H-related protein 1 precursor/Complement factor H- | IPI00011264 | 2 |
| related 1 | | |
| Isoform DPI of Desmoplakin (250/210 kDa paraneoplastic pemphigus | IPI00013933 | 1 |
| antigen)/desmoplakin | | |
| Isoform 1 of Gelsolin precursor/gelsolin | IPI00026314 | 3 |
| Hypothetical protein/heat shock 70 kDa protein 5 (glucose-regulated protein = | IPI00003362 | 2 |
| 78 kDa = GRP78 = BiP = HSPA5) | | |
| IGLC1 protein (immunoglobulin lambda C1)/immunoglobulin lambda chain | IPI00658130 (also | 1 |
| | in A2, A3 and A4) | |
| 25 kDa protein/immunoglobulin lambda locus (gene) | IPI00747752 (also | 1 |
| | in A2, A3 and A4) | |
| 25 kDa protein protein/immunoglobulin lambda chain/rheumatoid factor | IPI00154742 (also | 1 |
| G9 light chain (lambda V3)/immunoglobulin lambda C1 | in A2, A3 and A4) | |
| 26 kDa protein/immunoglobulin kappa variable 1-5 | IPI00738024 | 1 |

^{‡}Proteins are listed that are identified based on identified peptide masses. For peptide masses that are not unique for a single protein all proteins with the identified sequence are listed
^{†}A# series: analyzed eluate from enriched IgIV-matrix after contacting with indicated patient samples; The control serie C# displayed are the analyses of eluates from control matrix with affinity regions contacted with the same patient samples; background measurement.
¹⁾The IPI accession codes refer to protein entry codes for various protein/peptide databases. When the same protein(s) were also identified in one or more of the other analysed eluates after contacting affinity region-matrix with patient sample, these patient sample codes are given.

TABLE 14
Comparison of uniquely identified misfolded proteins and crosabeta binding proteins in
BiP-Sepharose eluates of six different synovial fluid samples of RA patients

| | | # of unique peptides | | | | | | |
|---|---|---|---|---|---|---|---|---|
| | | Accession | Sample | | | | | |
| # | Protein name | numbers | 1B | 2B | 3B | 4B | 5B | 6B |
| 1 | HSPA5 protein/BiP | IPI00003362 | 21 | 12 | 19 | 10 | 10 | 17 |
| 2 | fibronectin 1 isoform 4 preproprotein | IPI00022418 | 10 | 2 | | | 2 | |
| 3 | Apolipoprotein B-100 precursor | IPI00022229 | 9 | | 12 | | | |
| 4 | 26 kDa protein, immunoglobulin kappa variable 1-5 | IPI00419424 | | 2 | | | | 2 |
| 5 | Hypothetical protein, immunoglobulin lambda | IPI00154742 | | | 1 | | 1 | |
| 6 | IGHM protein, immunoglobulin heavy chain mu (heavy chain | IPI00385264 | 3 | | 1 | | | |
| | disease protein) | | | | | | | |
| 7 | IGKV1-5 protein, immunoglobulin kappa variable 1-5 | IPI00816787 | 3 | | 5 | | | |
| 8 | Vitronectin precursor | IPI00298971 | 1 | | | | 2 | 1 |
| 9 | Apolipoprotein A-I precursor | IPI00021841 | 3 | | 8 | | | |
| 10 | CDNA FLJ14473 fis, clone MAMMA1001080, | IPI00061977 | 2 | | 1 | | 1 | |
| | highly similar to Homo sapiens SNC73 protein (SNC73) | | | | | | | |
| | mRNA, immunoglobulin heavy constant alpha 1 | | | | | | | |
| 11 | Histidine-rich glycoprotein precursor | IPI00022371 | 2 | 1 | | | 1 | |
| 12 | Heat shock 70 kDa protein 1L | IPI00301277 | 1 | 1 | 1 | | 1 | 1 |
| 13 | IGHM protein, coagulation factor VII (serum prothrombin | IPI00382606 | 2 | 1 | | 1 | | |
| | conversion accelerator) | | | | | | | |
| 14 | IGL@ protein, immunoglobulin lambda | IPI00747752 | | | | 1 | 1 | |
| 15 | Hypothetical protein LOC649897, Hypothetical protein | IPI00736860 | | | | | 2 | 2 |
| | LOC649897 (some immunoglobulin characteristics) | | | | | | | |
| 16 | Isoform 1 of Fibrinogen alpha chain precursor | IPI00021885 | 3 | | | | | |
| 17 | IGHM protein, immunoglobulin heavy constant gamma 2 | IPI00399007 | 1 | 1 | | | | |
| 18 | IGHG4 protein, immunoglobulin heavy constant gamma 4 | IPI00550640 | 4 | 1 | | | | |
| 19 | FLJ00385 protein (Fragment), immunoglobulin heavy constant | IPI00168728 | 1 | 1 | 2 | | | |
| | mu | | | | | | | |
| 20 | Galectin-3-binding protein precursor | IPI00023673 | 1 | | 1 | | | |
| 21 | Isoform H17 of Myeloperoxidase precursor | IPI00007244 | 1 | | 1 | 1 | | |
| 22 | Haptoglobin precursor | IPI00431645 | 1 | | 3 | | | |
| 23 | Histone H3.2 | IPI00171611 | | | 1 | | | |
| 24 | Hypothetical protein, Hypothetical protein (immunoglobulin | IPI00807428 | 2 | | 1 | | 1 | |
| | characteristics) | | | | | | | |
| 25 | 187 kDa protein, complement component 3 | IPI00164623 | 2 | | | | 1 | |
| 26 | Clusterin precursor | IPI00291262 | 1 | 1 | 1 | | | |
| 27 | CDNA FLJ14473 fis, clone MAMMA1001080, | IPI00384952 | 1 | | | | 1 | |
| | highly similar to Homo sapiens SNC73 protein (SNC73) | | | | | | | |
| | mRNA, Hypothetical protein DKFZp686K04218 | | | | | | | |
| | (immunoglobulin characteristics) | | | | | | | |
| 28 | Isoform A of Proteoglycan-4 precursor | IPI00024825 | 2 | | 2 | | | |
| 29 | Histone H2B type 2-E | IPI00003935 | 2 | | | | | |
| 30 | Apolipoprotein | IPI00029168 | 4 | | | | | |
| 31 | Apolipoprotein E precursor | IPI00021842 | 1 | | | | | |
| 32 | Apolipoprotein A-II precursor | IPI00021854 | 1 | | | | | |
| 33 | Ig kappa chain V-IV region Len, immunoglobulin kappa | IPI00026197 | | | 1 | | | |
| | variable 4-1 | | | | | | | |
| 34 | Neutrophil defensin 1 precursor | IPI00005721 | | 1 | 1 | | | |
| 35 | Single-chain Fv (Fragment), Ig heavy chain V-III region TRO | IPI00382474 | 2 | | | | | |
| 36 | Apolipoprotein A-IV precursor | IPI00304273 | 3 | | | | | |
| 37 | 7 kDa protein, 7 kDa protein (immunoglobulin characteristics) | IPI00384404 | 1 | | | | | |
| 38 | Serotransferrin precursor | IPI00022463 | | | | | 1 | |

TABLE 15
Summary of data analysis after identification of proteins in the eluate of
BiP-Sepharose after contacting with synovial fluid of rheumatoid arthritis
patients

| | Identified | |
|---|---|---|
| identifier | proteins^{‡} | remarks |
| Known as amyloid binding | 1, 2, 6, 8, 9, 12, 17, | Prominent members are fibronectin, |
| protein and/or interacting | 19, 21, 22, 23, 25, | chaperones clusterin, haptoglobin, |
| with amyloid, i.e. part of the | 26, 28, 29, 31, 38 | HSP70 (see Table 2, 3) |
| Crossbeta Pathway | | |
| Known as amyloid forming | 3, 5, 9, 10, 14, 15, | Prominent members are the proteins |
| protein | 16, 18, 23, 24, 27, | already related to RA, i.e. for |
| | 29, 35 | example apoB, apoA-I, fibrin |
| Identified in three or more | 2, 8, 10, 11, 12, 13, | Based on the present results, at |
| patient samples (see Table | 19, 21, 24, 26 | forehand these proteins are of |
| 14) | | interest as general biomarkers for RA |
| | | diagnosis |
| Identified in separate | 1, 4, 5, 6, 7, 8, 10, | Haptoglobin (22) and serotransferrin |
| experiment (see Table 8-11) | 14, 17, 19, 24, 25, | (38) were identified in control plasma |
| | 31 | and serum in the separate analysis |
| Part of the Complement Lipid | 3, 8, 25, 26 | See patent applications |
| Pathway (CLiP) | | US2003165458 and US2003143223: |
| | | C3, vitronectin, clusterin, apoB |

^{‡}The numbers refer to the identified proteins listed in Table 14.

TABLE 16
Binding of misfolded proteins comprising
crossbeta structure and chaperone BiP to human dendritic
cells

Binding to DCs
(MFI ratio)^{‡}

| Putative DC ligand^{†} | Misfolded protein | BiP |
|---|---|---|
| BiP | — | 1.4 |
| BSA-AGE | 4.0 | — |
| BSA-AGE + BiP | 4.5 | 2.7 |
| oxLDL | 1.7 | — |
| oxLDL + BiP | 1.1 | 1.1 |
| HSP70/BiP-binding peptides + | — | 1.8 |
| BiP | | |
| Aβ16-22/Aβ25-35 + BiP | — | 261 |

^{†}HSP70/BiP-binding peptides are listed in Table 5 and are 6BB8 (YVDRFIGW), 6BB9 (LFWPFEWI), 6BB10 (HWDFAWPW), 6BB11 (FWGLWPWE) and 6BB13 (RRRAA)
^{‡}Mean fluorescence intensity ratio's are determined by dividing the signal obtained with a sample comprising a misfolded protein and/or Bip, by the corresponding background signal of the sample.

### REFERENCES

1. Asatryan, L., R. T. Hamilton, J. M. Isas, J. Hwang, R. Kayed, and A. Sevanian. 2005. LDL phospholipid hydrolysis produces modified electronegative particles with an unfolded apoB-100 protein. J. Lipid Res. 46:115-122.

2. Bouma, B., L. M. Kroon-Batenburg, Y. P. Wu, B. Brunjes, G. Posthuma, O. Kranenburg, P. G. de Groot, E. E. Voest, and M. F. Gebbink. 2003. Glycation induces formation of amyloid cross-beta structure in albumin. J. Biol. Chem. 278:41810-41819.

3. Citterio, S., M. Rescigno, M. Foti, F. Granucci, D. Aggujaro, C. Gasperi, M. K. Matyszak, G. Girolomoni, and P. Ricciardi-Castagnoli. 1999. Dendritic cells as natural adjuvants. Methods 19:142-147.

4. Kayed, R., E. Head, J. L. Thompson, T. M. McIntire, S. C. Milton, C. W. Cotman, and C. G. Glabe. 2003. Common structure of soluble amyloid oligomers implies common mechanism of pathogenesis. Science 300:486-489.

5. Kranenburg, O., B. Bouma, L. M. Kroon-Batenburg, A. Reijerkerk, Y. P. Wu, E. E. Voest, and M. F. Gebbink. 2002. Tissue-type plasminogen activator is a multiligand cross-beta structure receptor. Curr. Biol. 12:1833-1839.

6. Maas, C., S. Hermeling, B. Bouma, W. Jiskoot, and M. F. Gebbink. 2006. A role for protein misfolding in immunogenicity of biopharmaceuticals. J. Biol. Chem.

7. O'Nuallain, B. and R. Wetzel. 2002. Conformational Abs recognizing a generic amyloid fibril epitope. Proc. Natl. Acad. Sci. U.S.A 99:1485-1490.

8. Rappsilber, J., Y. Ishihama, and M. Mann. 2003. Stop and go extraction tips for matrix-assisted laser desorption/ionization, nanoelectrospray, and LC/MS sample pretreatment in proteomics. Anal. Chem. 75:663-670.

9. Sallusto, F. and A. Lanzavecchia. 1994. Efficient presentation of soluble antigen by cultured human dendritic cells is maintained by granulocyte/macrophage colony-stimulating factor plus interleukin 4 and downregulated by tumor necrosis factor alpha. J. Exp. Med. 179:1109-1118.

### DESCRIPTION OF FIGURES

FIG. 1. Over-expression of recombinant human BiP with a C-terminal His-tag, in HEK 293E cells, as established on a Western blot using anti-His-tag antibody.

Expression of recombinant human BiP was assessed using SDS-PAGE and Western blot analysis with an anti-His-tag antibody. The calculated mass of BiP based on amino acid sequence is approximately 71 kDa. BiP from two different transfections runs at a slighter higher molecular weight, due to the C-terminal extension with various tags.

FIG. 2. Binding of recombinant human BiP to crossbeta structure comprising proteins.

A. In an ELISA set-up BiP binds to glycated haemoglobin (Hb-AGE) and to a lesser extent to haemoglobin, but not to amyloid-β(1-40) aggregates (Aβ). B. BiP binds to glyeated albumin (BSA-AGE) and to a lesser extent to reduced and alkylated albumin (alkyl-BSA), and not to native BSA. C. BiP binding is more pronounced with organic solvent/heat denatured human γ-globulins (amyloid Ig) and heat-denatured lysozyme (d-lysozyme) than with native γ-globulins (native Ig).

FIG. 3. Binding of BiP to protein-AGE adducts with crossbeta structure is inhibited by tPA.

Binding of over-expressed recombinant human BiP in cell culture medium to immobilized Hb-AGE and BSA-AGE is inhibited by tPA and not by K2P-tPA that lacks the crossbeta structure binding finger domain, as determined in an ELISA set-up. Binding of BiP in threefold diluted cell culture medium is set to 100%.

FIG. 4. TEM analysis of misfolded glycated albumin (BSA-AGE) and hemoglobin (HbAGE)

The images show that BSA-AGE (A.) and HbAGE (B.) form non-fibrillar amorphous aggregates.

FIG. 5. Misfolding of Octagam IgIV induces crossbeta structure

A. TEM analysis of misfolded dIgIV-65 Octagam at 20 mg/ml in 10 mM NaPi buffer pH 8.1. B. Thioflavin T analysis of misfolded Octagam dIgIV-65, compared to dOVA standard and native Octagam.

FIG. 6. Misfolding of Gammagard IgIV induces crossbeta structure

Thioflavin T (A), Congo red (B), ANS (C), Bis-ANS (D) and Thioflavin S (E) fluorescence of misfolded dIgIV-86 Gammagard preparation. F. Tryptophan fluorescence of the misfolded dIgIV-86 Gammagard preparation when the fluorescence intensity at 375 nm is measured upon exciting at 283 nm.

FIG. 7. Misfolding of Gammagard IgIV induces aggregation, accompanied with ability to activate tPA/plasminogen.

TEM analysis of A. native IgIV Gammagard, and misfolded IgIV Gammagard, i.e. B. dIgIV-86, C. tPA mediated plasmin generation upon exposure to the denatured IgIV Gammagard preparations at a final concentration of 100 μg/ml. Co-factor stimulation of dOVA at 40 μg/ml was set arbitrarily to 100%. D. Misfolded base-denatured human γ-globulins IgG-base appear as aggregates on a TEM image

FIG. 8. ThT, Congo red and TEM analysis of Aβ42t=0.

FIG. 9. Strucural analysis of HSP binding peptides, Aβ16-22 and Aβ25-35.

In the figures, the 6BB# peptide series (see Table 5) is referred to by their last digit. A. Thioflavin T fluorescence of indicated peptide preparations. dOVA standard is shown as reference. B. Congo red fluorescence of indicated peptide preparations. dOVA standard is shown as reference. C. tPA/plasminogen activation assay with 100 μg/ml of the indicated 6BB# peptide samples, 25 μg/ml dOVA standard, 12.5 μg/ml HbAGE and 10 μg/ml oxLDL. D. TEM image of Aβ16-22 (peptide 6BB12), showing large and small peptide assemblies with a few tiny fibers.

FIG. 10. Activation of factor XII and prekallikrein by oxidized LDL with misfolded ApoB100.

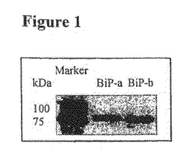

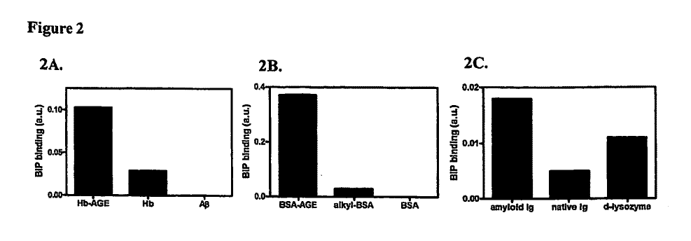

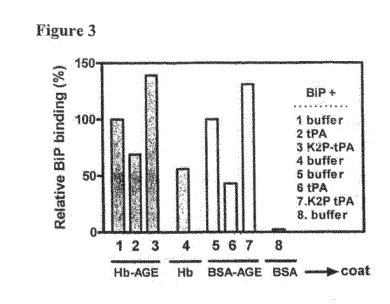

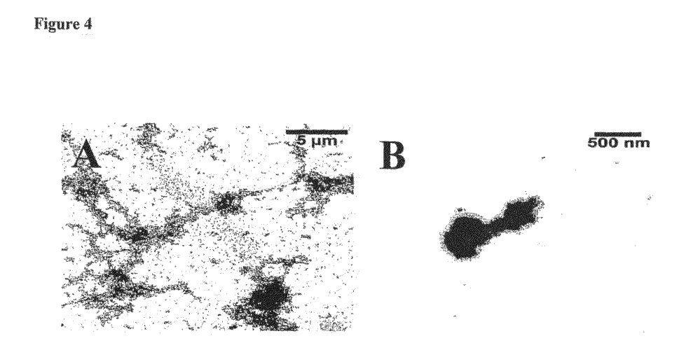

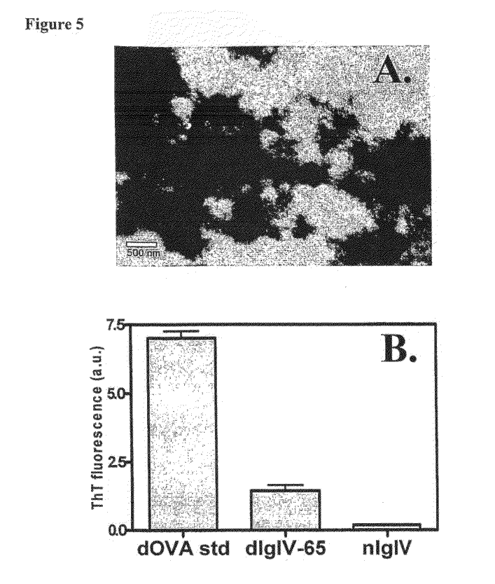

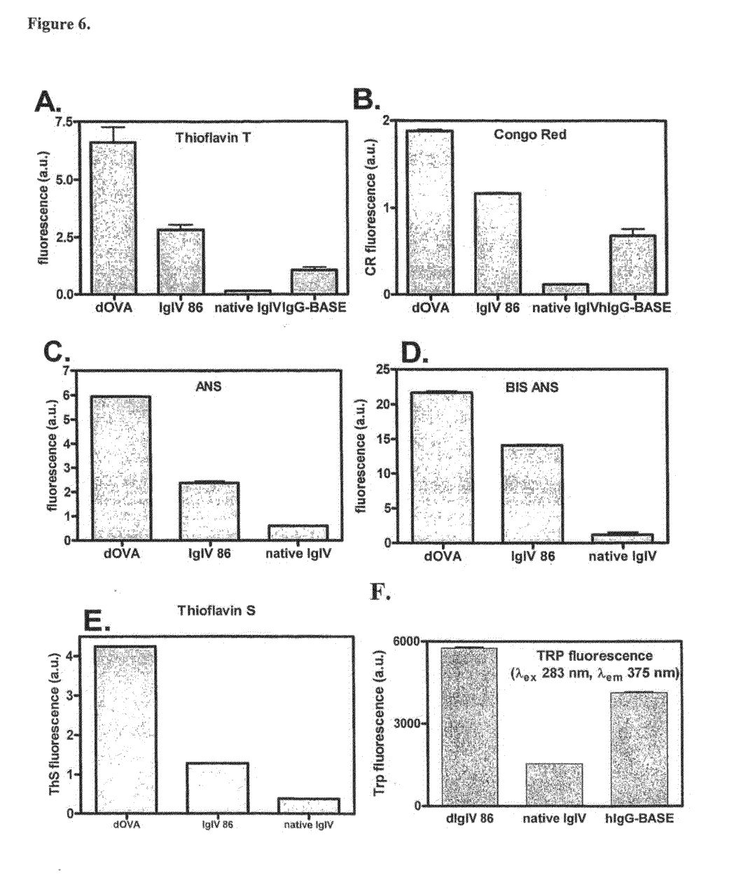

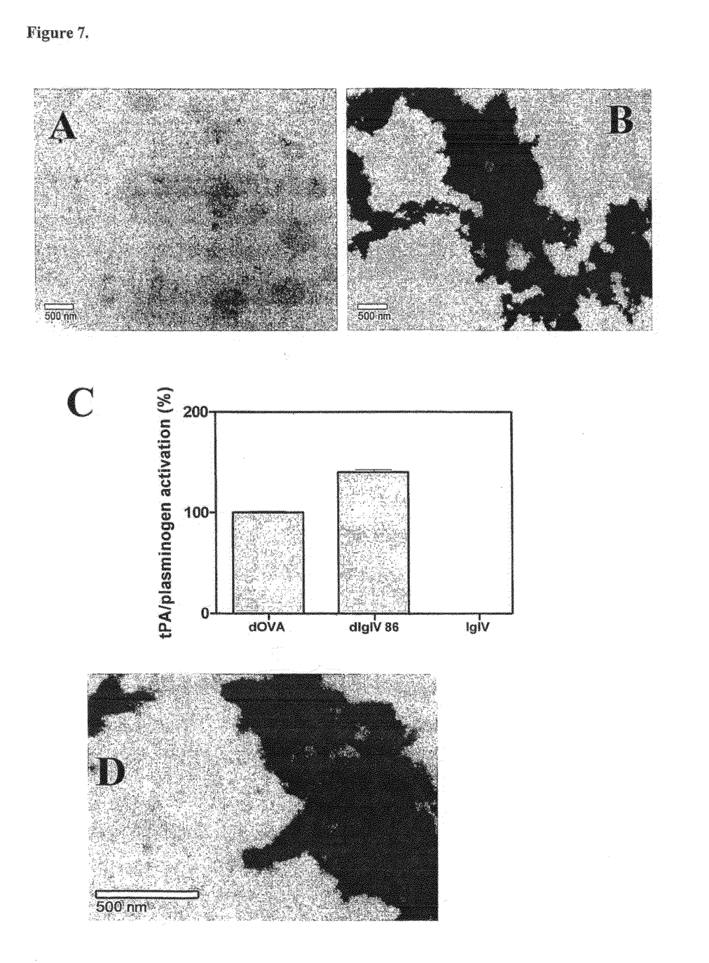

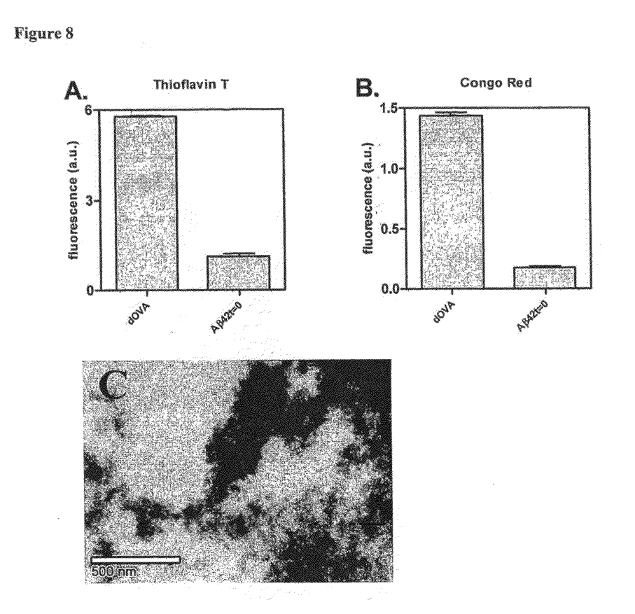

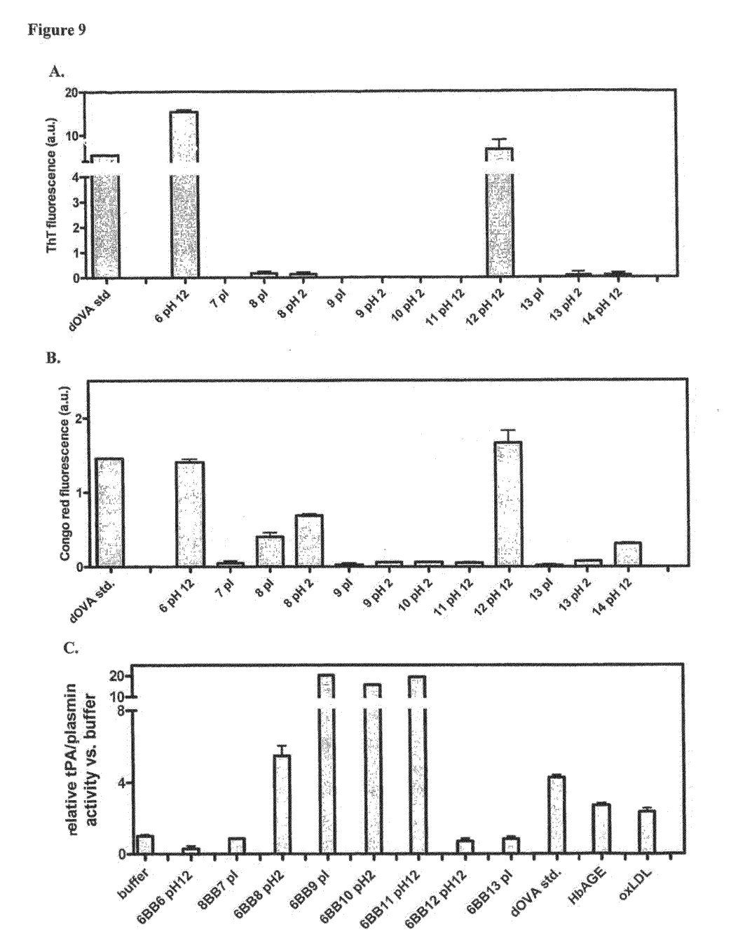

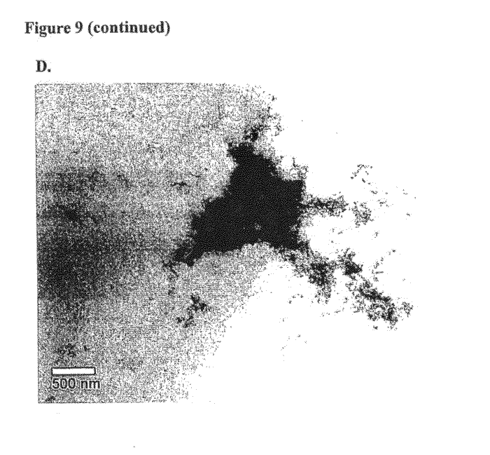

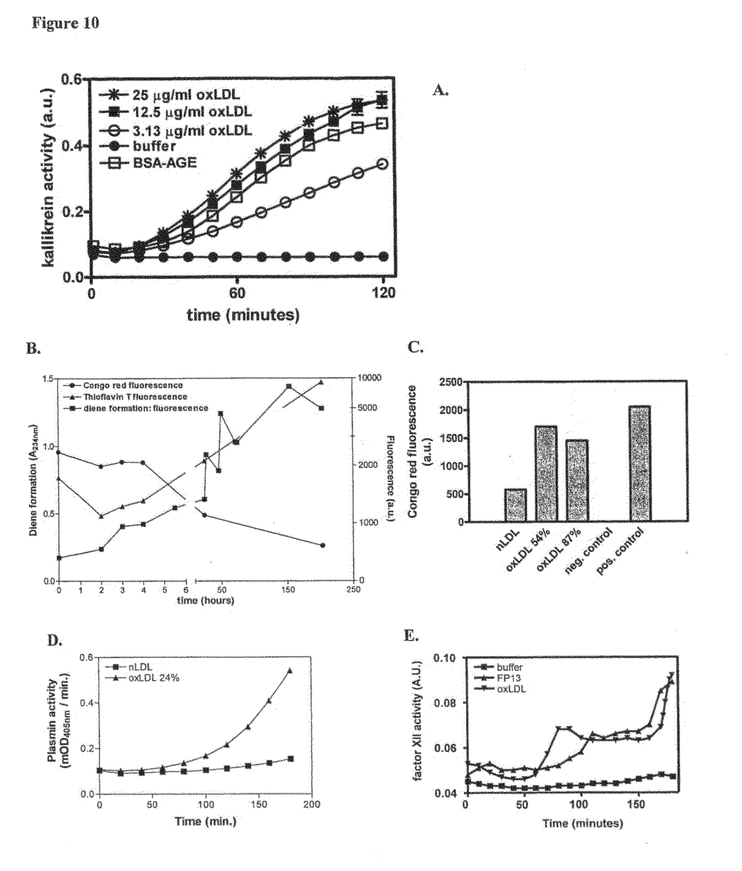

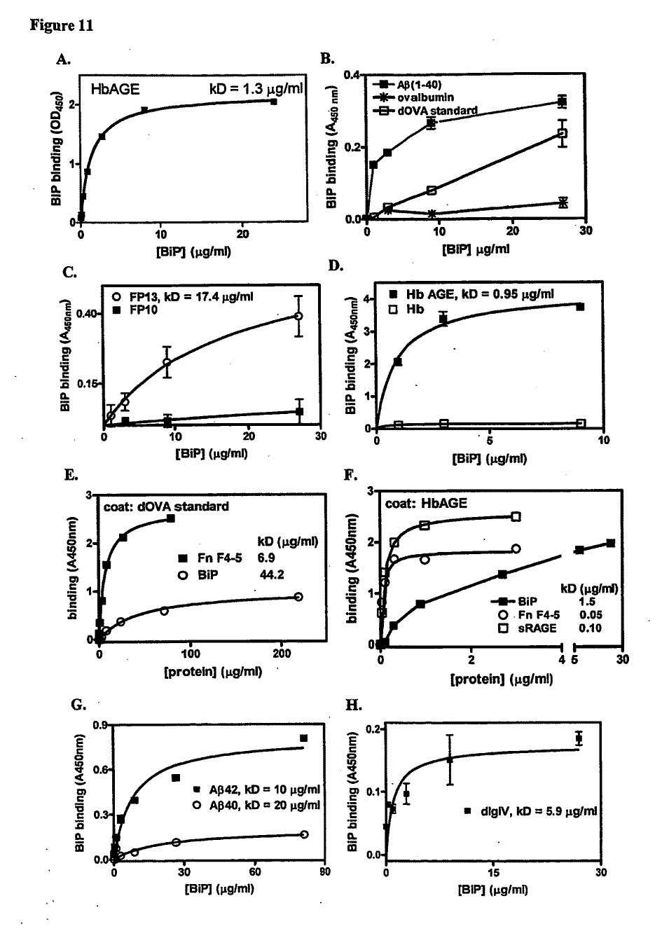

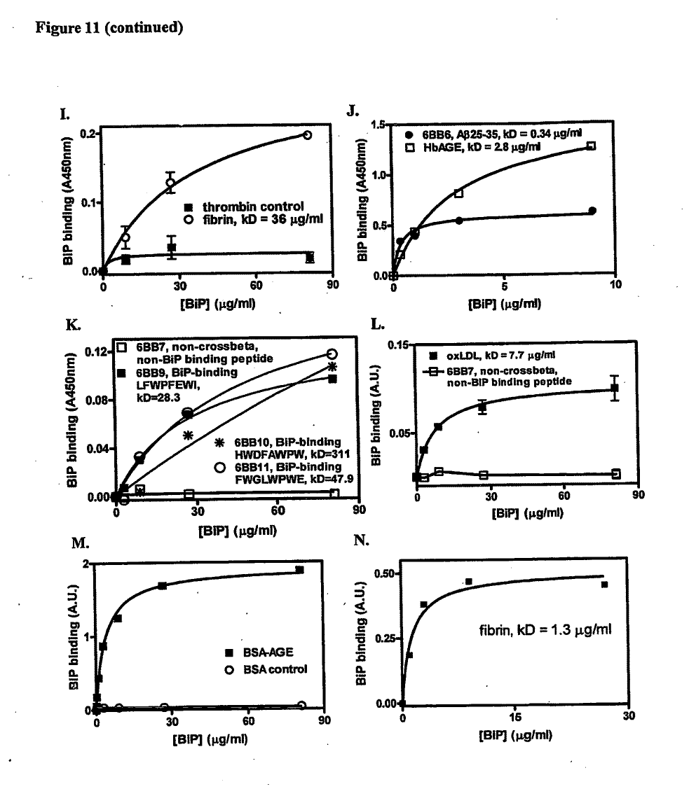

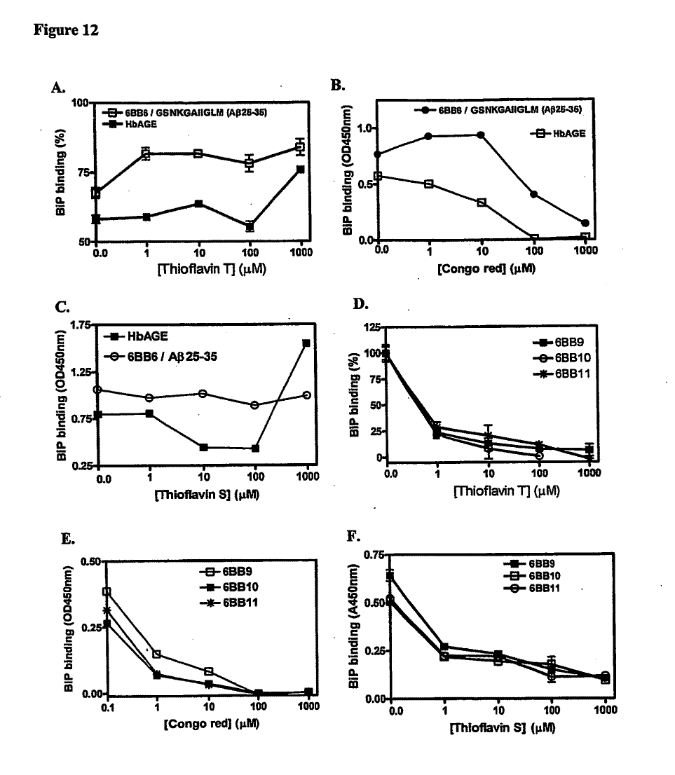

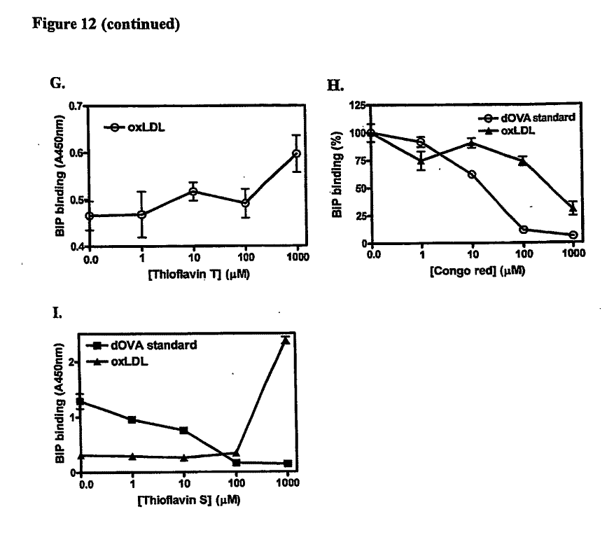

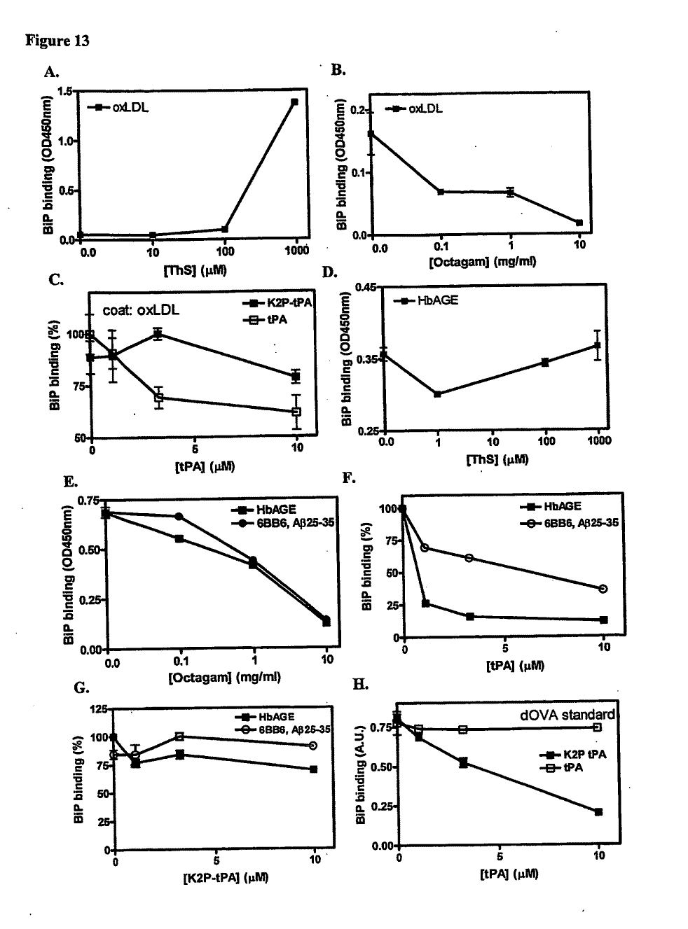

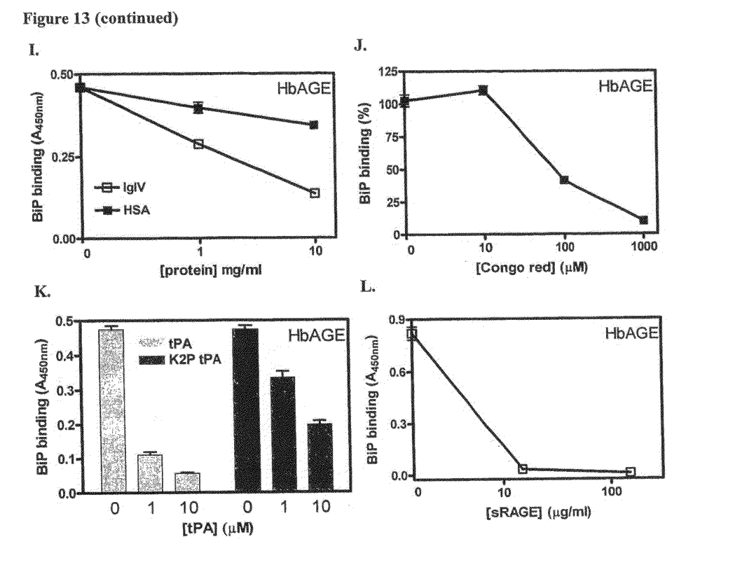

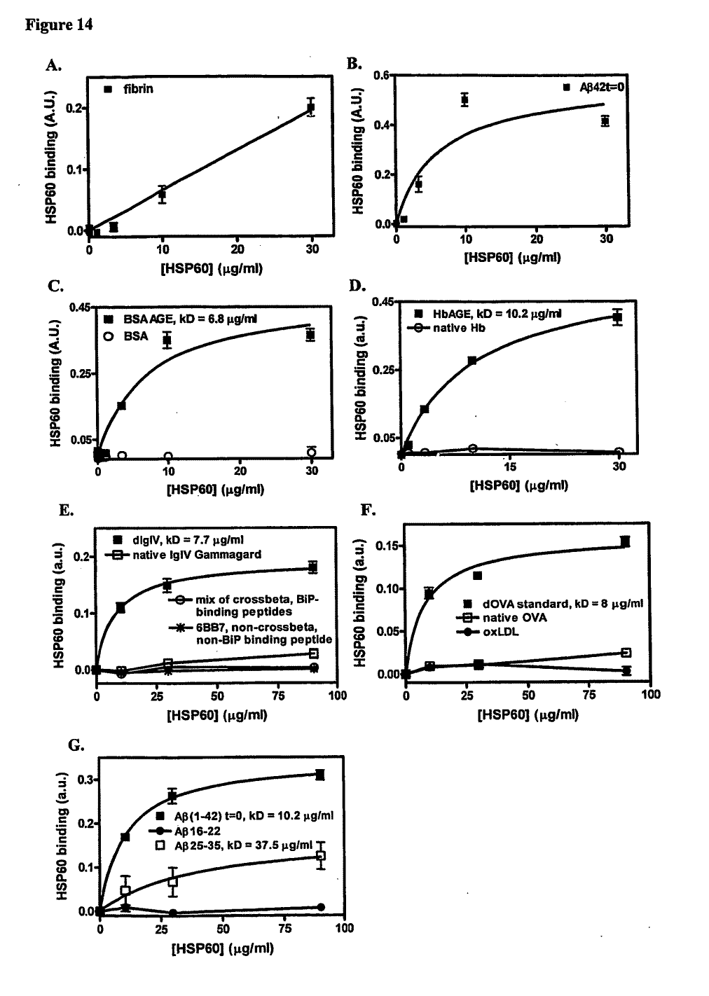

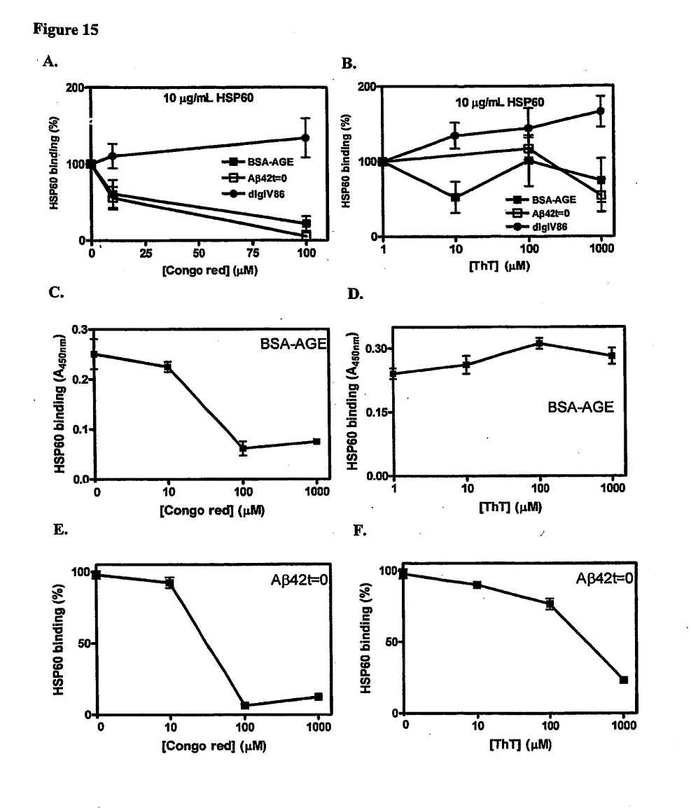

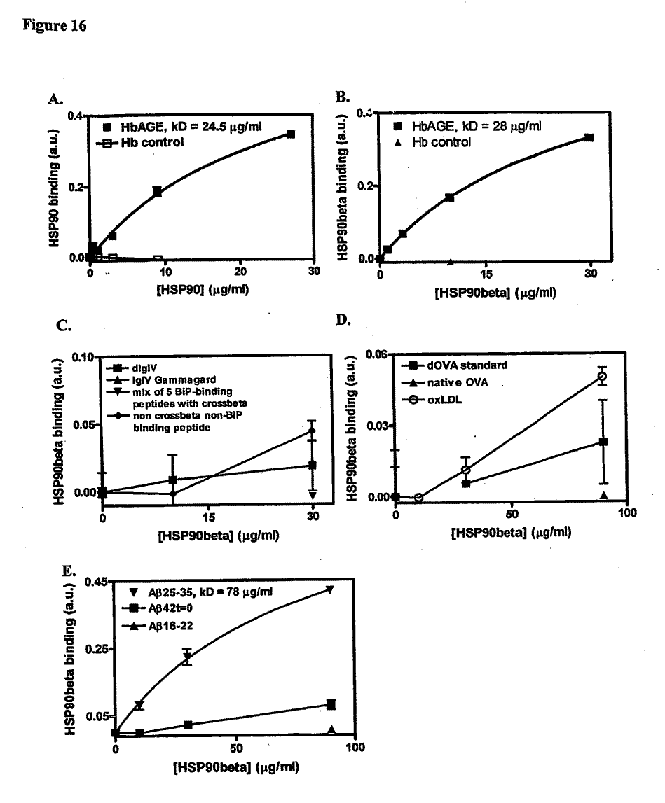

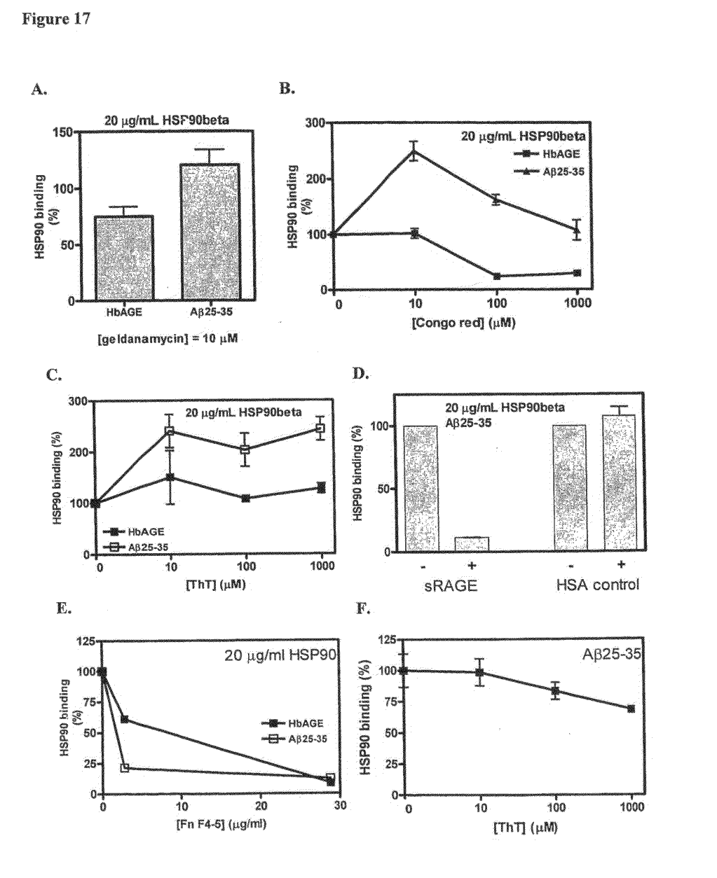

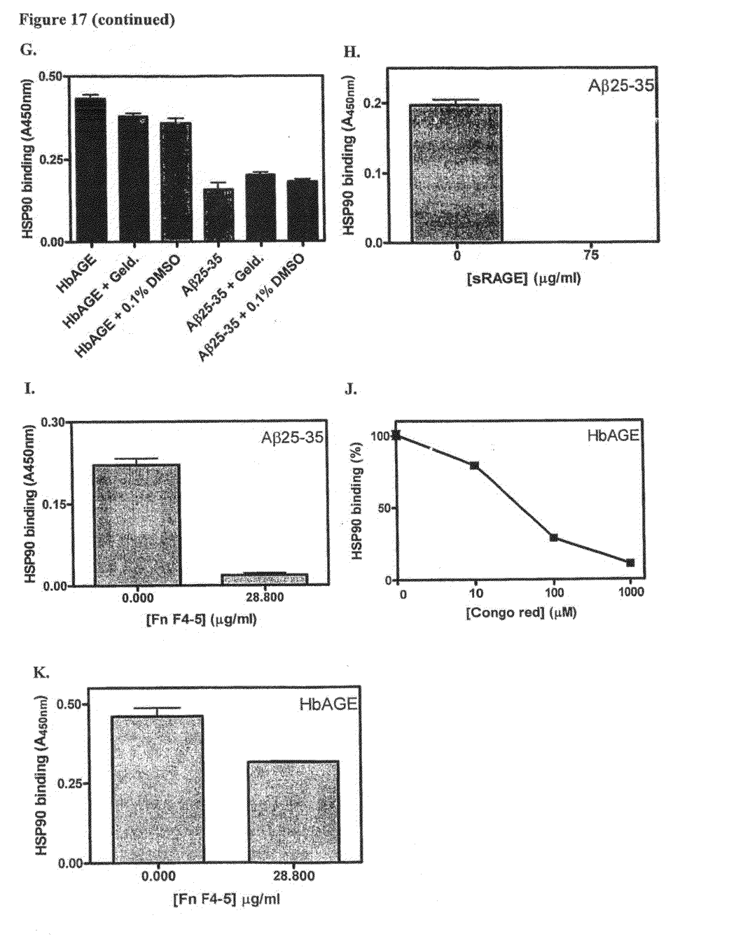

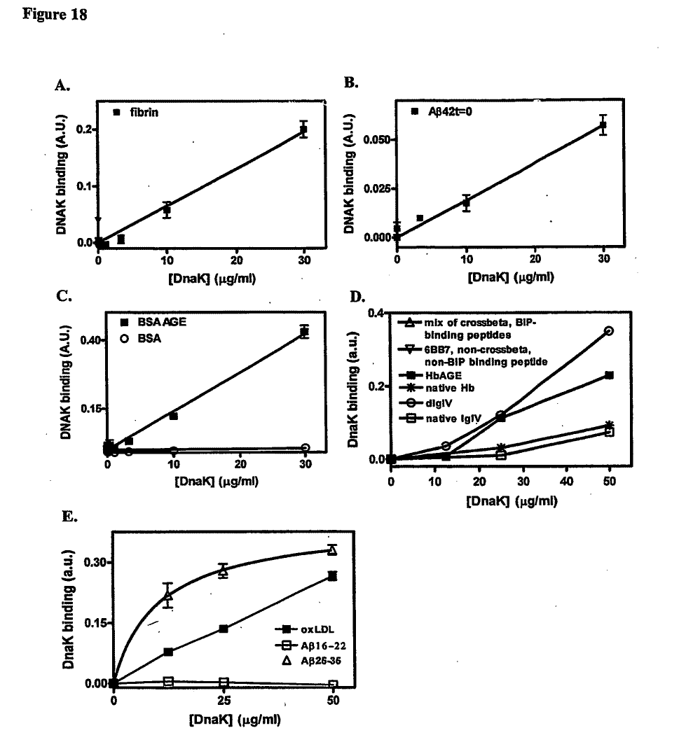

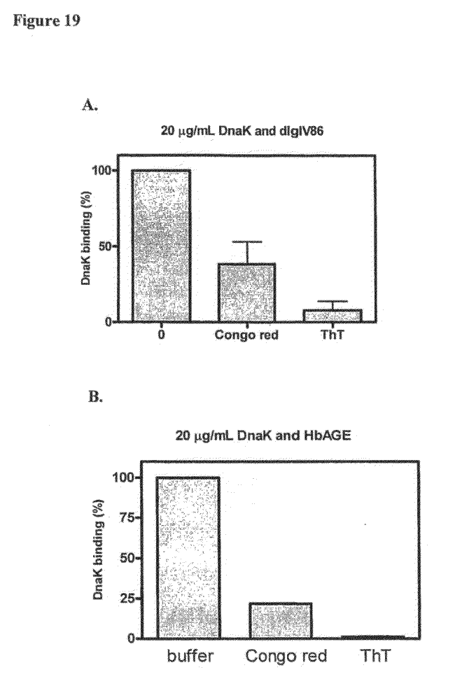

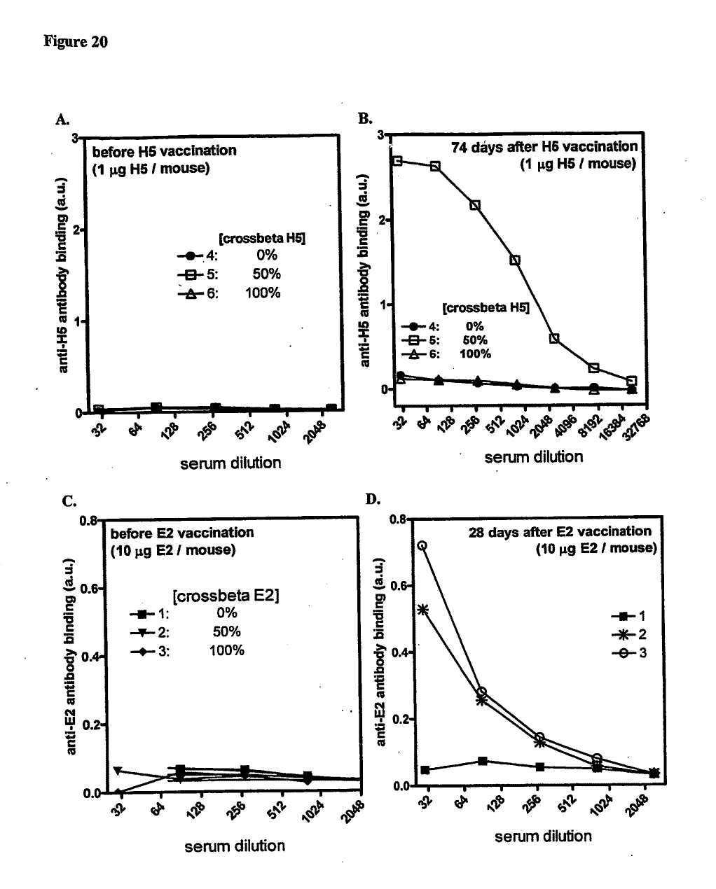

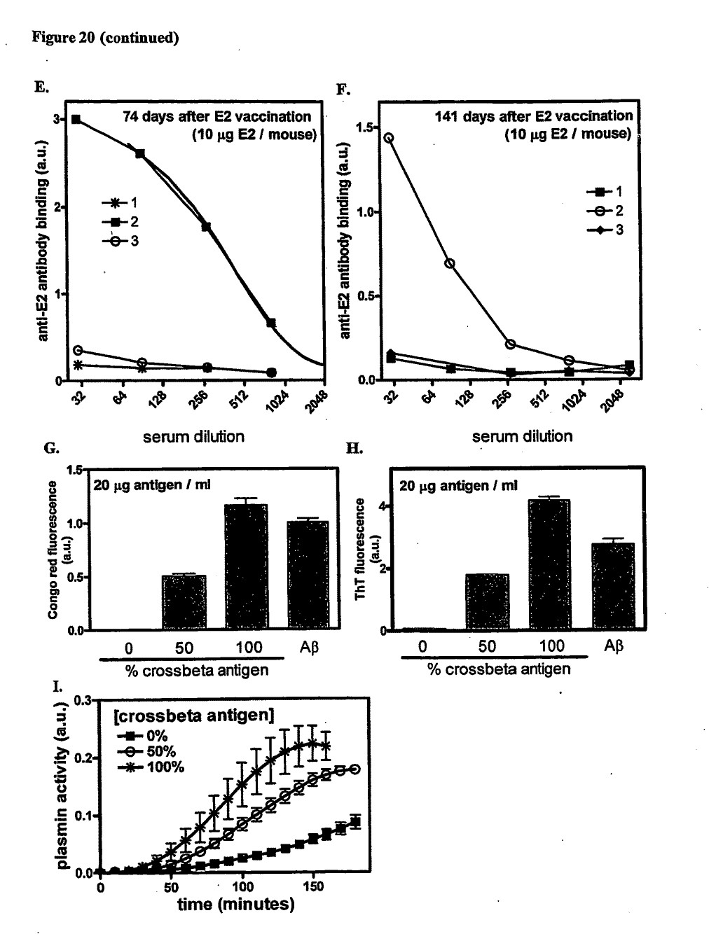

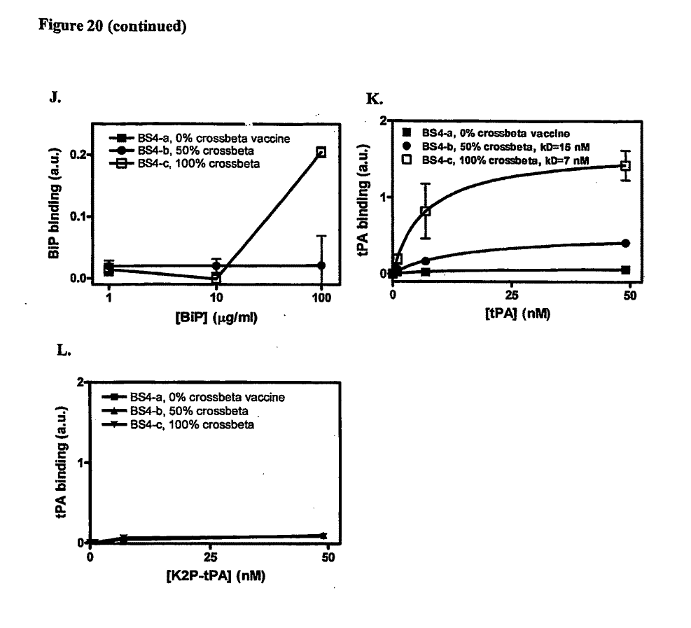

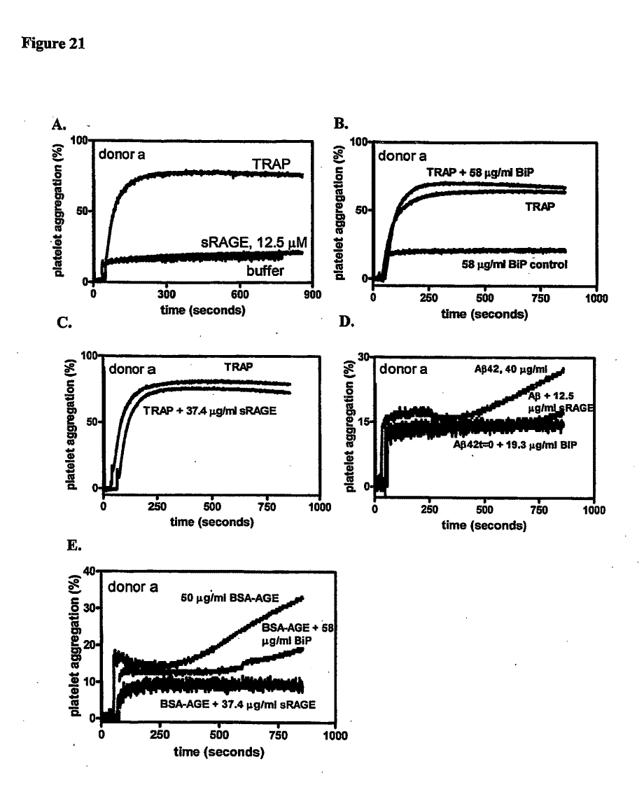

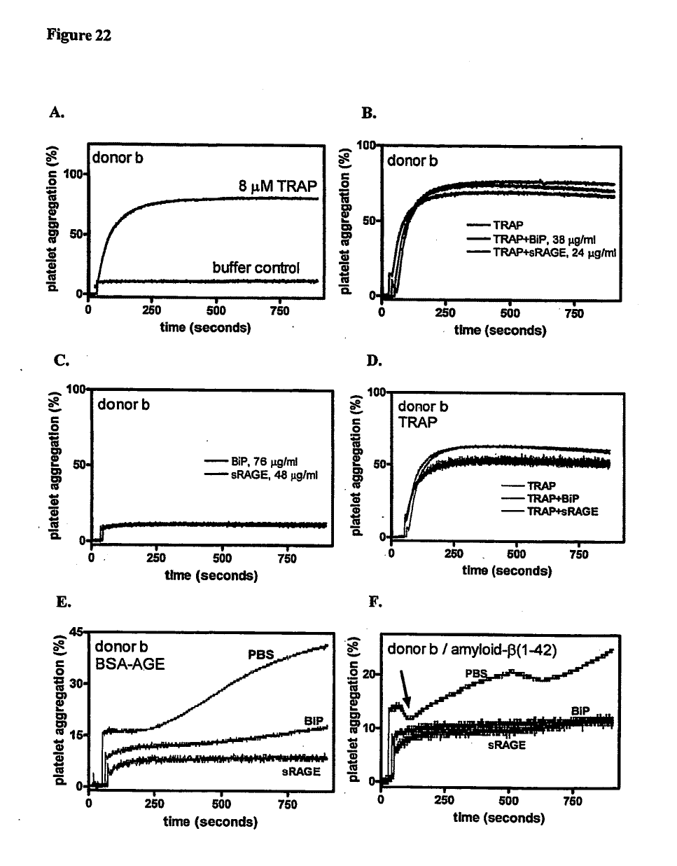

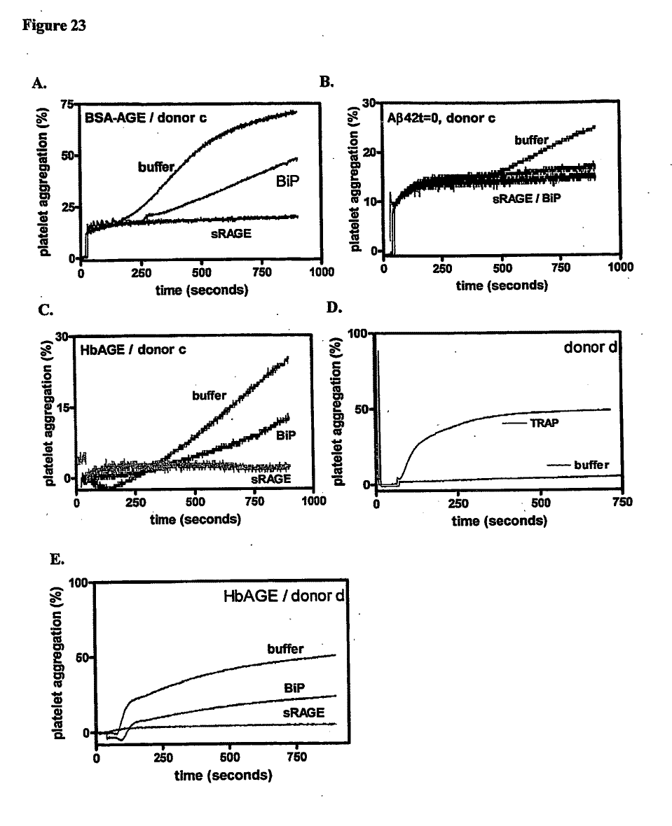

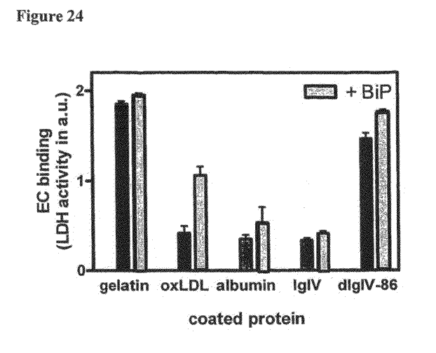

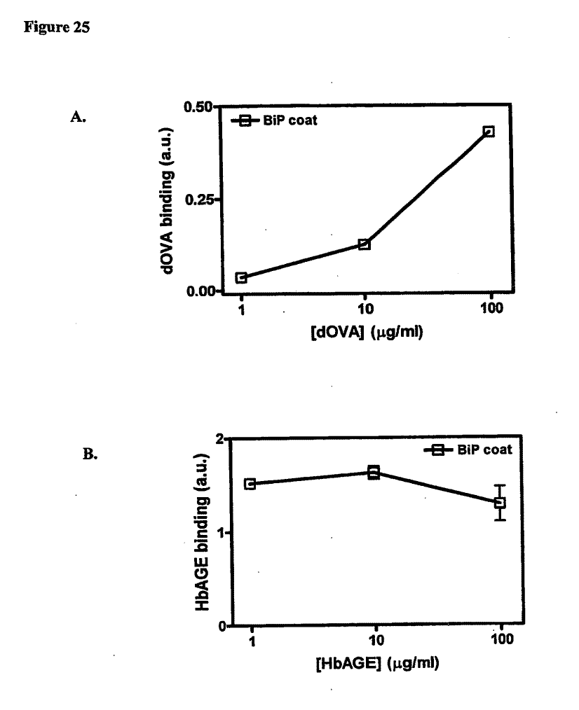

A. Activation of factor XII, a serine protease resembling tPA and activatable by misfolded proteins with crossbeta properties, is determined indirectly by measuring the activity of kallikrein, which is formed from pre-kallikrein by activated factor XII. The propensity of oxidized LDL with misfolded ApoB (ThT fluorescence; see also (Asatryan et al., 2005)) is determined at various concentrations. Positive control: 100 μg/ml glycated albumin(Bouma et al., 2003). B. In time, an increase of the oxidation of LDL, as measured by specific diene fluorescence at 243 nm, is accompanied by an increase in Thioflavin T fluorescence and a decrease in Congo red fluorescence, indicative for structural changes in the apoB protein part of the LDL. C. Congo red fluorescence of 25 μg/ml oxidized LDL is similar to the Congo red fluorescence of the positive control, 25 μg/ml Aβ. D. In the chromogenic tPA/plasminogen activation assay, 24% oxidized LDL shows cofactor activity for the tPA-mediated conversion of plasminogen to plasmin, whereas native LDL has hardly any effect on tPA activity. E. Factor XII in plasma is activated by oxidized LDL and by amyloid peptide FP13, as determined with the direct chromogenic factor XII activation assay using chromogenic substrate S-2222.

FIG. 11. Binding of BiP to misfolded proteins comprising crossbeta, using an ELISA approach with immobolized misfolded proteins and native controls.

Binding of concentration series of BiP to coated (misfolded) proteins is shown for A. HbAGE, B. native ovalbumin, dOVA standard, Aβ1-40, C. fibrin fragment FP10 lacking crossbeta, FP13 with crossbeta, D. native haemoglobin, crossbeta glycated haemoglobin, E. dOVA standard, F. HbAGE, G. Aβ1-40, Aβ1-42, H. misfolded denatured IgIV, I. thrombin negative control, fibrin, J. Aβ25-35, HbAGE, K. 6BB9, Synthetic BiP binding motif LFWPFEWI; 6BB10, Synthetic BiP binding motif HWDFAWPW; 6BB11, Synthetic BiP binding motif FWGLWPWE; 6BB7, negative control peptide without crossbeta and according to literature without affinity for BiP, L. oxidized LDL and non-crossbeta, non-BiP binding peptide 6BB7, M. native bovine serum albumin and glycated albumin with crossbeta, N. fibrin. For comparison in E. and F. binding of misfolded protein binding protein fibronectin finger4-5 is shown. In F. binding of the soluble extracellular fragment of RAGE, that has affinity for misfolded proteins, is depicted. BiP concentrations that result in half-maximum binding signals are depicted as kD's when appropriate.

FIG. 12. Competition ELISA with BiP and crossbeta binding compounds Congo red, Thioflavin T and Thioflavin S.

Binding of a sub-optimal concentration of BiP to indicated misfolded proteins under influence of a concentration series of Thioflavin T (A., D., G.) or Congo red (B., E., H.) or Thioflavin S (C., F., I.). Misfolded proteins: 6BB6, synthetic human Aβ25-35; 6BB9, Synthetic BiP binding motif LFWPFEWI; 6BB10, Synthetic BiP binding motif HWDFAWPW; 6BB11, Synthetic BiP binding motif FWGLWPWE; HbAGE, glycated human hemoglobin; dOVA standard, misfolded hen ovalbumin; oxLDL, oxidized human low-density lipoprotein. Note: For clarity, BiP binding without added crossbeta binding compounds is positioned arbitrarily at the x-axis at position 0.1 when a log 10 axis is given.

FIG. 13. Competition ELISA with BiP and crossbeta binding compounds ThS, tPA, IgIV.

Binding of a sub-optimal concentration of BiP to indicated misfolded proteins under influence of a concentration series of Thioflavin S (A., D.) or IgIV Octagam (B., E.) or tPA (C., F., H.) or K2P tPA that lacks a misfolded protein binding finger motif (C., G., H.). Misfolded proteins: 6BB6, synthetic human Aβ25-35; HbAGE, glycated human hemoglobin; dOVA standard, misfolded hen ovalbumin; oxLDL, 59% oxidized human low-density lipoprotein. I.-L. The indicated competition ELISAs with coated HbAGE and 1 μg/ml BiP in the presence of binding buffer (positive control for binding) or potential inhibitors, are duplicated. IgIV Octagam, HSA (I.), Congo red (J.), tPA and K2P tPA (K.) and sRAGE (L.)were tested for their potential inhibitory activity towards BiP binding to HbAGE. Note: For clarity, BiP binding without added crossbeta binding compounds is positioned arbitrarily at the x-axis at position 0.1 or 0.0 when a log 10 axis is given.

FIG. 14. Binding of human recombinant HSP60 to various misfolded proteins.

Binding of human recombinant HSP60 with an N-terminal His-tag to immobilized fibrin (A.), Aβ42t=0 (B.) and glycated albumin (C.), as determined with an ELISA with coated misfolded proteins. Negative controls were buffer-coated wells and native BSA (see C.). For the experiments depicted in figure A-C, rec. human His-HSP60 lot 1 was used. For subsequent experiments lot 2 was used. Binding of HSP60 was assessed with HbAGE and native Hb (D.), misfolded dIgIV, native IgIV Gammagard, a mix of four BiP-binding peptides with crossbeta (6BB8, 6BB10, 6BB11, 6BB13, see text for sequences) and a peptide without crossbeta, that does not bind BiP, i.e. 6BB7 (E.), dOVA standard, freshly dissolved lyophilized ovalbumin and oxLDL (F.), and Aβ42t=0, Aβ16-22 and Aβ25-35 (G.). Binding of human HSP60 was established for fibrin, BSA-AGE, HbAGE, dIgIV, dOVA standard, Aβ1-42 and Aβ25-35.

FIG. 15. Binding of HSP60 to misfolded proteins under influence of crossbeta binding compounds ThT and Congo red.

A. To study the influence of Congo red on the interaction between HSP60 and indicated misfolded proteins, 10 μg/ml HSP60 was pre-mixed with buffer (100% binding control) or a concentration series of Congo red. B. Binding of HSP60 to misfolded proteins under influence of ThT. In C.-F., the experiments depicted in A., B. are duplicated for Aβ42t=0 and BSA-AGE, now with 30 μg/ml HSP60. Binding of HSP60 to BSA-AGE under influence of Congo red (C.) or ThT (D.). Influence of Congo red (E.) or ThT (F.) on binding of HSP60 to Aβ42t=0.

FIG. 16. Binding of recombinant human HSP90 to misfolded proteins with crossbeta structure, as assessed with ELISAs.

Binding of recombinant human HSP90beta was analysed for immobilized HbAGE and freshly dissolved lyophilized Hb (A.), using a rabbit anti-HSP90 antibody. Subsequently, binding of HSP90 was assessed with a mix of two anti-HSP90 antibodies, with immobilized HbAGE and Hb (B.), with misfolded dIgIV, native IgIV Gammagard, 6BB7, a non-crossbeta non-BiP binding peptide, and a mix of five crossbeta BiP-binding peptides 6BB8-11, 13 (see text) (C.), with dOVA standard, OVA and oxLDL (D.), and with synthetic human fragments crossbeta Aβ1-42, freshly dissolved at t=0, Aβ16-22 and Aβ25-35 (E.).

FIG. 17. Binding of HSP90 to crossbeta proteins under influence of crossbeta binding proteins/compounds and geldanamycin.

To study the influence of crossbeta binding compounds on the interaction between misfolded proteins and HSP90, 20 μg/ml HSP90 was contacted to the indicated immobilized misfolded proteins, in the presence of indicated compounds. A. Geldanamycin is a well-known HSP90 inhibitor. It has been described as a blocker of the binding of HSP90 to unfolded protein ligands. Now, the influence of 10 μM geldanamycin, an effective concentration recommended by the manufacturer, on binding of HSP90 to coated crossbeta HbAGE and Aβ25-35 was analysed. Binding is given as a percentage of the binding in the absence of geldanamycin. B. Influence of Congo red on the binding of HSP90 to HbAGE and Aβ25-35. C. Influence of ThT on the binding of HSP90 to HbAGE and Aβ25-35. D. Influence of 79 μg/ml (2 μM with MW(sRAGE)=40 kDa) sRAGE or 10 μM HSA negative control on binding of HSP90. E. Influence of Fn F4-5 on HSP90 binding. Duplications of a subset of the experiments depicted on A.-E. are given in F.-K. F. Binding of HSP90 to Aβ25-35 under influence of ThT. G. Binding of HSP90 to Aβ25-35 or HbAGE under influence of 10 μM geldanamycin or the corresponding final concentration of 0.1% DMSO. H. Influence of sRAGE on binding of HSP90 to Aβ25-35. I. Influence of Fn F4-5 on binding of HSP90 to Aβ25-35. J., K. Influence of Congo red or Fn F4-5 on binding of HSP90 to HbAGE.

FIG. 18. Binding of E. coli heat shock protein 70 DNAK to various misfolded proteins.

Binding of E. coli DNAK to immobilized fibrin (A.), Aβ42t=0 (B.) and glycated albumin (C.), as determined with an ELISA with coated misfolded proteins. Negative controls were buffer-coated wells and native BSA (see C.). For the experiments depicted in figure A-C, E. coli DnaK lot A was used. For subsequent experiments lot B was used. Binding of DnaK was assessed with misfolded dIgIV, native IgIV Gammagard, HbAGE, freshly dissolved lyophilized Hb, a mix of four BiP-binding peptides with crossbeta (6BB8, 6BB10, 6BB11, 6BB13, see text for sequences) and a peptide without crossbeta, that does not bind BiP, i.e. 6BB7 (D.), and with oxLDL, Aβ25-35 and Aβ16-22 (E.). Binding of Escherichia coli DnaK was established for fibrin, BSA-AGE, HbAGE, dIgIV, Aβ1-42, and Aβ25-35.

FIG. 19. Binding of E. coli DnaK HSP70 to misfolded proteins under influence of ThT and Congo red.

A. Binding of 20 pg/ml E. coli DnaK to immobilized misfolded IgIV-86 was assessed with an ELISA. The influence of crossbeta binding dyes Congo red and ThT was determined with 1 mM of the dyes. B. Similar to A., now with coated HbAGE.

FIG. 20. Avian influenza H5N1 virus sub-unit H5 vaccine with crossbeta and classical swine fever virus E2 envelop protein vaccine with crossbeta elicit antibody titers in mice, and bind BiP and tPA.

Previously, we have shown that mice immunized with crossbeta H5 and pigs immunized with crossbeta E2 are protected against infection with H5N1 avian influenza virus and infection with classical swine fever virus, respectively. When mice are now immunized with 1 μg/ml H5, only with 50% crossbeta-adjuvated H5, antibody titers against native H5 are detected at 74 days post-immunization (A., B.). Mice immunized with 10 μg E2/mice develop titers against native E2 at 28 days post-immunization, when 100% and 50% crossbeta-adjuvated E2 are injected, and only with 50% crossbeta-adjuvated E2 when titers are determined at 74 and 141 days post-immunization, and not with 0% crossbeta-adjuvated E2 (C.-F.). Antigen solutions comprising crossbeta-adjuvated H5 and crossbeta-adjuvated E2 display markers for the presence of misfolded antigen with crossbeta, i.e. enhanced Congo red fluorescence (G.), enhanced Thioflavin T fluorescence (H.) and enhanced tPA/plasminogen activation (I.). In the fluorescence assays, Aβ was used as a positive crossbeta control. J. Binding of BiP to coated E2/H5 vaccines BS4a (0% crossbeta-adjuvated E2/H5), BS4b (50% crossbeta-adjuvated E2/H5, 50% native antigen), BS4c (100% crossbeta-adjuvated antigen). For control purposes positive control tPA and negative control K2P tPA were contacted with the same three vaccine solutions (K., L.).

FIG. 21. Misfolded protein binding proteins BiP and sRAGE inhibit misfolded protein induced platelet activation. Donor a.

A. Freshly isolated human platelets of healthy donor ‘a’ are stimulated with 8 μM TRAP and aggregate readily, as determined in an aggregometer. sRAGE at the indicated concentration hardly stimulates platelets to an extent also seen with negative control buffer. B. BiP at the indicated concentration does not influence platelet aggregation induced by TRAP, and BiP does not activate the platelets alone. C. TRAP-induced platelet aggregation is not influenced by sRAGE. D. Platelet aggregation induced by amyloid-β1-42 is strongly inhibited by adding BiP or sRAGE. E. Platelet aggregation induced by crossbeta BSA-AGE is strongly inhibited by adding BiP or sRAGE.

FIG. 22. Misfolded protein binding proteins BiP and sRAGE inhibit misfolded protein induced platelet activation. Donor b.

A. Platelets of donor ‘b’ are stimulated with TRAP and aggregate readily. B. BiP and sRAGE at the indicated concentration do not inhibit TRAP-induced aggregation. C. BiP and sRAGE at the indicated concentration do not induce platelet aggregation. D. BiP and sRAGE at 76 and 48 μg/ml, respectively do not inhibit TRAP-induced aggregation. E. Platelet aggregation induced by crossbeta BSA-AGE is strongly inhibited by adding BiP or sRAGE. F. Platelet aggregation induced by Aβ1-42 is inhibited by adding BiP or sRAGE. With buffer control, a shape change of the platelets is detected, whereas this shape change is absent when platelets are stimulated with Aβ1-42 in the presence of BiP or sRAGE (shape change indicated with an arrow). The BSA-AGE concentration was 33.3 μg/ml, the Aβ42t=0 concentration was 26.7 μg/ml.

FIG. 23. Misfolded protein binding proteins BiP and sRAGE inhibit misfolded protein induced platelet activation. Donor c, d.

A. Crossbeta binding proteins BiP and sRAGE inhibit BSA-AGE induced platelet aggregation. For the aggregation studies, platelets obtained from donor ‘c’ were used. B. BiP and sRAGE abolish Aβ42 induced platelet activation. C. Similarly, also HbAGE induced platelet aggregation is effectively blocked with sRAGE, and inhibited by BiP. Control aggregation studies with buffer, TRAP, TRAP with BiP or sRAGE or buffer all revealed similar results as those depicted for donor a and b, and are not shown here. D. Control platelet aggregation with TRAP and buffer control, using platelets of donor d. E. BiP (58 μg/ml) inhibits and sRAGE (37.4 μg/ml) abolishes HbAGE (80 μg/ml) induced platelet aggregation.

FIG. 24. Adherence of HUVECs to misfolded proteins is increased in the presence of BiP.

Coated misfolded proteins comprising crossbeta structure, i.e. oxLDL, denarured dIgIV-86 and BSA-AGE, as well as gelatin positive control for adherence, and negative controls native albumin and native IgG were overlayed with buffer or BiP, before HUVECs were allowed to adhere. After 1 h, the number of adhered HUVECs was quantified by measuring LDH activity.

FIG. 25. Depletion of solutions from misfolded proteins using immobilized BiP.

A-B. Extraction of misfolded dOVA (A.) or HbAGE (B.) from a protein solution by using HSP70 family member human BiP that is immobilized on a solid support, i.e. the wells of an ELISA plate. Negative control: buffer only, immobilized on the solid support.

1. A method for binding a cross-beta structure comprising protein, comprising contacting said protein with a chaperone or a functional equivalent and/or a functional fragment thereof.

2. A method according to claim 1, wherein said chaperone or a functional equivalent and/or a functional fragment thereof is an extracellular chaperone or a functional equivalent and/or a functional fragment thereof.

3. The method according to claim 2, wherein said chaperone is an extracellular chaperone is BiP, haptoglobin, hsp72, clusterin, or a functional equivalent and/or a functional fragment of any thereof.

4. A method for increasing protein degradation and/or protein clearance and/or protein neutralization in an individual, comprising administering a chaperone or a functional equivalent and/or a functional fragment thereof to said individual.

5. A method for at least in part inhibiting crossbeta structure mediated effects in an individual, comprising administering an effective amount of a chaperone or a functional equivalent and/or a functional fragment thereof to an individual.

6. A method for at least partial prevention and/or treatment of a crossbeta structure related and/or associated disease, a blood coagulation disorder and/or a microbial/pathogen/bacterial/parasite/viral infection in an individual, comprising administering a chaperone or a functional equivalent and/or a functional fragment thereof to said individual.

7. A method for at least partial prevention and or treatment of an HIV-related opportunistic infection in an individual, comprising administering a chaperone or a functional equivalent and/or a functional fragment thereof to said individual.

8. A method of diminishing accumulation of a misfolded protein having a cross-beta structure, the method comprising:
interacting the misfolded protein with a chaperone or a functional equivalent and/or a functional fragment thereof so as to diminish accumulation of the misfolded protein.

9. The method according to claim 8, wherein said misfolded protein is involved in a conformational disease selected from the group consisting of an amyloidosis type disease, atherosclerosis, diabetes, bleeding, thrombosis, cancer, sepsis, encephalopathy, encephalitis, inflammatory diseases, Multiple Sclerosis, auto-immune diseases, disease associated with loss of memory, Parkinson's disease, other neuronal diseases, and epilepsy.

10. A separation device, said separation device comprising:
a system for transporting fluids, said system comprising:
means for connecting to a flowing fluid,
means for entry of fluid into said system and return of fluid from said system and
a solid phase comprising a chaperone or a functional equivalent and/or functional fragment thereof.

11. The separation device according to claim 10, which is a dialysis apparatus.

12. A method for interfering in coagulation of blood and/or in aggregation of platelets and/or in fibrinolysis comprising providing to blood a chaperone or a functional equivalent and/or a functional fragment thereof.

13. A method for removing a crossbeta structure and/or protein comprising a crossbeta structure from a pharmaceutical composition or any of its constituents comprising a protein, said method comprising:
contacting said pharmaceutical composition or any of its constituents comprising a protein with a chaperone (or a functional equivalent and/or a functional fragment thereof);
allowing binding of said crossbeta structure and/or protein comprising a crossbeta structure to said chaperone (or a functional equivalent and/or a functional fragment thereof); and
separating bound crossbeta structure and/or bound protein comprising a crossbeta structure from said pharmaceutical composition or any of its constituents comprising a protein.

14. A method for decreasing and/or preventing undesired side effects of a pharmaceutical composition and/or increasing the specific activity per gram protein, said method comprising detecting and removing any unfolded protein or peptide and/or aggregated protein or peptide and/or multimerized protein or peptide comprising a crossbeta structure from said pharmaceutical composition or any of its constituents comprising a protein, said method comprising the steps of
contacting said pharmaceutical composition or any of its constituents comprising a protein with a chaperone (or a functional equivalent and/or a functional fragment thereof);
allowing binding of said crossbeta structure and/or protein comprising a crossbeta structure to said chaperone (or a functional equivalent and/or a functional fragment thereof); and
separating bound crossbeta structure and/or bound protein comprising a crossbeta structure from said pharmaceutical composition or any of its constituents comprising a protein.

15. A method for controlling a manufacturing process, and/or storage process of a pharmaceutical composition or any of its constituents comprising a protein, said method comprising:
contacting said pharmaceutical composition or any of its constituents comprising a protein with at least one chaperone or a functional equivalent and/or a functional fragment thereof resulting in a bound protein and/or peptide comprising a crossbeta structure, and
detecting whether bound protein and/or peptide comprising a crossbeta structure is present in said pharmaceutical composition or any of its constituents comprising a protein at various stages of said manufacturing and/or storage process.

16. A method for determining the amount of crossbeta structures in a vaccine composition, the method comprising:
contacting said vaccine composition with at least one chaperone or a functional equivalent and/or a functional fragment thereof; and
relating the amount of bound crossbeta structures to the amount of crossbeta structures present in the vaccine composition.

17. A method for determining whether a protein and/or peptide comprising a crossbeta structure is present in an aqueous solution comprising a protein, said method comprising:
contacting said aqueous solution comprising a protein with at least one chaperone or a functional equivalent and/or a functional fragment thereof, and
detecting whether bound protein and/or peptide comprising a crossbeta structure is present therein.

18. The method according to claim 17, wherein said aqueous solution comprises a detergent, a food product, a food supplement, a cell culture medium, a commercially available protein solution used for research purposes, blood, a blood product, cerebrospinal fluid, synovial fluid, lymph fluid, a cosmetic product, a cell, a pharmaceutical composition or any of its constituents comprising a protein, or a combination of any of these.

19. (canceled)

20. A method for selecting a compound capable of binding to a crossbeta structure in a protein, comprising:
contacting said compound with a first protein comprising a crossbeta structure and allowing said compound and said protein to interact;
determining with a chaperone or a functional equivalent and/or a functional fragment thereof whether said compound at least in part binds to said crossbeta structure; and
selecting the compound that at least in part binds to said crossbeta structure.

21. The method according to claim 20, wherein said determining step comprises a competition assay between said compound, a first protein comprising a crossbeta structures, and a chaperone or a functional equivalent and/or a functional fragment thereof.

22. A method for determining a difference in crossbeta structure content of a protein in a reference sample compared to protein in a test sample, wherein the test sample has been subjected to a treatment that is expected to have an effect on the crossbeta structure content of said protein, the method comprising:
determining in a reference sample the crossbeta structure content of the protein;
subjecting said protein to a treatment that is expected to have an effect on the crossbeta structure content of said protein, thus obtaining a test sample;
determining in the obtained test samples the crossbeta structure content of said protein; and
establishing whether the crossbeta structure content of the reference sample is different from the crossbeta structure content in the test sample,
wherein at least one determining step is performed by using a chaperone or a functional equivalent and/or fragment thereof.

23. A method for selecting a treatment that essentially preserves the structure of a protein, the method comprising:
determining in a reference sample the crossbeta structure content of said protein,
subjecting said protein to a treatment that is expected to have an effect on the crossbeta structure content to obtain a test sample,
determining in said test sample the crossbeta structure content of said protein, and
selecting the treatment that essentially preserves the structure of said protein,
wherein at least one determining step is performed by using a chaperone or a functional equivalent and/or fragment thereof.

24. A method for producing an immunogenic composition, wherein said immunogenic composition comprises at least one protein, said method comprising providing said protein with at least one crossbeta structure.

25. A method for producing an immunogenic composition, wherein said immunogenic composition comprises at least one chaperone or a functional equivalent and/or a functional fragment thereof and at least one protein, said method comprising providing said protein with at least one crossbeta structure.

26. A method for producing an immunogenic composition, wherein said immunogenic composition comprises at least one chaperone or a functional equivalent and/or a functional fragment thereof and at least one protein and at least one linker molecule, said method comprising the step of providing said linker molecule with at least one crossbeta structure.

27. An immunogenic composition obtainable by the method of claim 24.

28. The method according to claim 1, further comprising:
identifying protein bound to the chaperone.

29. A method for identifying a misfolded protein, a crossbeta structure, or a protein comprising a crossbeta structure in a sample comprising a protein, said method comprising:
contacting said sample with a chaperone, resulting in bound misfolded proteins, crossbeta structures and/or bound protein or proteins comprising a crossbeta structure, and
identifying a bound misfolded protein, bound crossbeta structure and/or bound protein or proteins comprising a crossbeta structure.

30. The method according to claim 29, wherein said sample comprises an aqueous solution.

31. The method according to claim 30, wherein body fluids originating from healthy individuals and body fluids from individuals suffering from, or suspected to suffer from, a disease related to and/or associated with a crossbeta structure's presence, are compared.

32. The method according to claim 29, wherein a sample from an individual suffering from or at risk of suffering from AL amyloidosis and/or arthritis is used.

33. A medicament comprising:
a compound capable of specifically binding to a compound depicted in any one of Tables 8-11 or 14-15, and/or capable of diminishing the amount and/or activity of a compound depicted in any one of Tables 8-11 or 14-15.

34. (canceled)

35. (canceled)

36. A method for treating a subject suffering from, or at risk of suffering from, a misfolded protein related and/or associated disease, a blood coagulation disorder, sepsis and/or a microbial/pathogen/bacterial/parasite/viral infection, comprising administering to said individual a compound capable of specifically binding to a compound depicted in any one of Tables 8-11 or 14-15, and/or capable of diminishing the amount and/or activity of a compound depicted in any one of Tables 8-11 or 14-15.

37. A method according to claim 36, wherein said disease comprises AL amyloidosis and/or arthritis.

38. A dialysis apparatus comprising:
a system for transporting circulating fluids ex vivo, the system comprising:
means for connecting a flowing fluid to an individual's circulation,
means for entry of fluid into the system and return of fluid from the system to the individual's circulation, and
a solid phase comprising a chaperone.

39. A method for binding a cross-beta structure comprising peptide, the method comprising:
contacting the cross-beta structure comprising peptide with a chaperone so as to bind the cross-beta structure comprising peptide with the chaperone.

40. A method for increasing protein degradation, protein clearance, and/or protein neutralization in an individual, the method comprising:
administering a chaperone to the individual in a manner that increases protein degradation, clearance, and/or neutralization in the individual.

41. In a method of treating a microbial infection in a subject of the type comprising administering a compound to the subject, the improvement comprising:
using a chaperone as the compound to treat the microbial infection.
